SEQUENCE LISTING (321 sequences)

SEQ ID NO: 1 - DNA, 105 bp, Artificial Sequence
  subsequence of PAX9
cagcgggtcgctgactacctcgccctagcactcggtccgcgccccgggaaggcttcagcatcaccctcttctgctgggatcccgctgaccctgtctgcagcttcg

SEQ ID NO: 2 - DNA, 98 bp, Artificial Sequence
  subsequence of HOXD10
cctggagaggcggacaggagggcggcggagagcgctgggccggttgtctccagcgcgcactatcgcgggcgcgtagtagatgtcgctgttgtccgtgc

SEQ ID NO: 3 - DNA, 104 bp, Artificial Sequence
  subsequence of TBX20
gcgacgcgtagtgggagagtgcgaaccttatggtagcgcccggactaagcggacacagacccctctggttttacaaaggcggctgtgacgaggacttcccacgg

SEQ ID NO: 4 - DNA, 105 bp, Artificial Sequence
  subsequence of CACNA1B
ggccagcgggtcctctacaagcaatcgatcgcgcagcgcgcgcggaccatggcgctgtacaaccccatcccggtcaagcagaactgcttcaccgtcaaccgctcg

SEQ ID NO: 5 - DNA, 83 bp, Artificial Sequence
  subsequence of ZIC1
tctcttggctctcggcttggggtcgggcggcgagtggcagacaggcggtggcgggagccagaggaagcgggaaggcaaaggtt

SEQ ID NO: 6 - DNA, 91 bp, Artificial Sequence
  subsequence of SIM1
tggccaatagggctgagtgacacgagtcggccccgagccgccctccgcgcgcccaggctccgggctctgaatcttactacccgcgggaccg

SEQ ID NO: 7 - DNA, 89 bp, Artificial Sequence
  subsequence of DLX1
gaggaaacgcgggctcctatatccccagctacacatcgtggtacccttcagcgcaccaagaagctatgcagcaaccccaacttatgtga

SEQ ID NO: 8 - DNA, 112 bp, Artificial Sequence
  subsequence of LMX1A
gcttccctatcgcggaccaggtcccagagagcggggctccagagctcagcgccaagcggaaagagagtgcgcccaggacgcacggcctgaacactcacccagcagcgaggag

SEQ ID NO: 9 - DNA, 102 bp, Artificial Sequence
  subsequence of DLX6AS1
gccacgtctacctgctggtattgaggacattacaaacagtctcggcgcgcagcccgcgtgtaccgagagcgcactagggttgggagtgggaaggtggggtgg

SEQ ID NO: 10 - DNA, 88 bp, Artificial Sequence
  subsequence of MIR196A1
ccgcctacacggaaaatccacagagccccgcgcagaccctatgatcggctcggcccgccgggcttgggatgaggctccctgcttctcg

SEQ ID NO: 11 - DNA, 107 bp, Artificial Sequence
  subsequence of FEZF2
aagcgtcacggagagcaaggccgctcgccggcatctcgggggcctcggcgccgcgcaggtgcgcctccagctttatggccagctctgggtctgaggctgggggagac

SEQ ID NO: 12 - DNA, 97 bp, Artificial Sequence
  subsequence of STAG3
agggcaggacgcctgagagcttgaggccacacgaggctgtggagcggcgtgactcaaacgtggcgcgcatcagctcgcacacttccaaacctcgcga

SEQ ID NO: 13 - DNA, 89 bp, Artificial Sequence
  subsequence of PTPRN2
gaagcccgacccacaacaaagctagtgacagttcatttccttcctgcttacgggggccggcgagaggaaaggatggcggatgtgagagg

SEQ ID NO: 14 - DNA, 92 bp, Artificial Sequence
  subsequence of BARHL1
tggaggatggcaggtgcagcctgctccagccggcagggcgtgcgagccgggagcgggtcctctccggttgccatttcaggcctgctcgcttc

SEQ ID NO: 15 - DNA, 96 bp, Artificial Sequence
  subsequence of CCDC140
gagtcttgggcctcccctttatttctttctgaagtctctccggagcccaagccaccccacacccaaaccccgcagctggatgggagtccaggccac

SEQ ID NO: 16 - DNA, 90 bp, Artificial Sequence
  subsequence
gaagcgagctgggctgtgagcaagggacacccacagcctggagaaacagccccgctctcttgcgcgctgtctgctccagccgctactggg

SEQ ID NO: 17 - DNA, 106 bp, Artificial Sequence
  subsequence of GATA2
cgtccagccagggcaaacccgggctgtgcaacaagtgtgggcggcacatctggcctccggtcaggcgggctgcgggcaaagagagagaggatcagggtgggcagaa

SEQ ID NO: 18 - DNA, 100 bp, Artificial Sequence
  subsequence of ADCYAP1
gggctctccggttactttctctgtatgtcgcgggtgagaggaacagcgaggacaatttagcgcaaacacacgaagggtcggatctcaagggggcagcgct

SEQ ID NO: 19 - DNA, 107 bp, Artificial Sequence
  subsequence of TBX5
aatgtgcagggaggcagtcggccggcagagctgcggcgggagccaaggtcaggcccgcggggagagcgggcagcttccagcgccggccacaagctcccaggccagct

SEQ ID NO: 20 - DNA, 93 bp, Artificial Sequence
  subsequence
acagcagatggcacgagcacgtgcggcgctcagcttaggctctcggaggcagctgagttggaaatcccgacggaaagcacccacaagctccca

SEQ ID NO: 21 - DNA, 81 bp, Artificial Sequence
  subsequence of NECAB1
aacaatccttgccccatagcaaacgtttcgaagcctactctaggcagagttcatagcaccgcctttgtcagcagcactgcc

SEQ ID NO: 22 - DNA, 93 bp, Artificial Sequence
  subsequence of ADRB1
cgtcaccaaccgggcctacgccatcgcctcgtccgtagtctccttctacgtgcccctgtgcatcatggccttcgtgtacctgcgggtgttccg

SEQ ID NO: 23 - DNA, 90 bp, Artificial Sequence
  subsequence of OTX2
ccgcctacagaactccacgttcgcagaggggcatcggtcggtctattggaggtactggggtgatggccgacagacacggggacacgtaga

SEQ ID NO: 24 - DNA, 93 bp, Artificial Sequence
  subsequence of NKX2-6
agaagggggtggaggtgacggggctcagcagcatcccgaaggcggatggggcggggccgaggaggtccgggtgaggagcggcaccctgaactt

SEQ ID NO: 25 - DNA, 103 bp, Artificial Sequence
  subsequence of SIM1
accccaaatcccatgtatcctgccgggcctgcacatcctcgctaaatgtgtactgtgcaagtcttccccgggctgaggcgtcgagtggccttcgtgttcagcc

SEQ ID NO: 26 - DNA, 103 bp, Artificial Sequence
  subsequence of DRD4
gaccagcagcacgggacacgcgtgacctgtgctcctttcgagccgcagacgtcacagtgacgacgtttaagctcctaatctccccaaatcggcgggaaggatt

SEQ ID NO: 27 - DNA, 90 bp, Artificial Sequence
  subsequence of TBX15
tggctgttcagcaggagcaccttccagcatggttttagaagtccgggctggaaagcttctgaaaccgctaaaccaagttgcgagcgttgt

SEQ ID NO: 28 - DNA, 80 bp, Artificial Sequence
  subsequence of TRH
actcctgacctgccgactgcggatcccgagtccccggatcccggacccatcctgtggagcccactcctggcaggtaaccg

SEQ ID NO: 29 - DNA, 94 bp, Artificial Sequence
  subsequence of FBP1
accgccgaagagatggctttgactgctgtgcagagcgagttgagcagctgggtcaactcgcccgtgccgcgggccttcctgccctcctccatga

SEQ ID NO: 30 - DNA, 91 bp, Artificial Sequence
  subsequence
gtgtgatgcccggtgtcctaccctaggccacgcggagctctctcccggcgtgtcgggccctgcgcccctgcccagtggctcacctcggctc

SEQ ID NO: 31 - protein, 120 aa, Artificial Sequence
  subsequence of BCAN
GCGTTCCGGATCAGTCCTTGCCTCCGTGCCCCCACGCACAGGCAGCTCCAGGAGCAGCGGCCAGCAACCCTTCTGGGGACACCTTACTGTAGTCCGGCAGGACAGCCGATCAGAGCCGCT

SEQ ID NO: 32 - DNA, 113 bp, Artificial Sequence
  subsequence of TP73
cgccacaccatcaccatccccaaccgcggcggcccaggcggcggccctgacgagtgggcggacttcggcttcgacctgcccgactgcaaggcccgcaagcagcccatcaagga

SEQ ID NO: 33 - DNA, 87 bp, Artificial Sequence
  subsequence of MIIP
cagcctccctagtagctgggattacaggcgccggccacctcgcccggctaatttttgtatttttaggtttactggcgtgagcaaccg

SEQ ID NO: 34 - DNA, 90 bp, Artificial Sequence
  subsequence of HOXC4
agaaaagcgcttggctctccccaaatgggcccagcccgccgcctgctcaggctgccgcgcgctctcccgccgctcaggcacggcccagtg

SEQ ID NO: 35 - DNA, 84 bp, Artificial Sequence
  subsequence of HOXA7
cctccgccaatctcagcagtcctcacaggtggtctcgctccgcagggcccgcagccgcctagaatggaagggcaagaggctcaa

SEQ ID NO: 36 - DNA, 109 bp, Artificial Sequence
  subsequence of BST1
gaacgggaaacggcaaacagcgagatatccgagcgagagtcccgccctgcatcagtttgcggaaccgccttggtagaaggagagaaggggagtggaggaagcacgggac

SEQ ID NO: 37 - DNA, 91 bp, Artificial Sequence
  subsequence of C1orf114
gtgcctctgcgtcctccatccgggcctctctaactacgagagcgacaaaaatctgcgcaggcgcatgcaatatcctcagaccaccgacccc

SEQ ID NO: 38 - DNA, 90 bp, Artificial Sequence
  subsequence of FOXG1
acaagcagggctggcagaactccatccgccacaatctgtccctcaacaagtgcttcgtgaaggtgccgcgccactacgacgacccgggca

SEQ ID NO: 39 - DNA, 113 bp, Artificial Sequence
  subsequence of ATP4A
gcgcacctggaacttgttggtggagttgaagggtatctcgcagacttttgggaagcggtcccggtagcccatggcgttgcccagcgtcagctccgagaacttgagcagcgccg

SEQ ID NO: 40 - DNA, 86 bp, Artificial Sequence
  subsequence of NOTCH1
ggaactgggctgcggtcacgggtgggaccagcgaggatggcagcgacgtgggcagggcggggctctcctggggcagaatagtgtgc

SEQ ID NO: 41 - DNA, 109 bp, Artificial Sequence
  subsequence
acggctcggccatctacacctacctgcagcccgcgcagcgctacaaccaggcacggggcaagttcgtatcgctcttctacaccgtggtcacacctgctctcaacccgct

SEQ ID NO: 42 - DNA, 96 bp, Artificial Sequence
  subsequence
agcgggcacagacacaagcggttggacatgaagaggaacacaccggcgggagaacacagaacacccaccctctctttagtcaccctggaggcctcg

SEQ ID NO: 43 - DNA, 84 bp, Artificial Sequence; identical to SEQ ID NO: 35
  subsequence of HOXA7

SEQ ID NO: 44 - DNA, 107 bp, Artificial Sequence
  subsequence of ZNF529
gtgctgaggaggccaaggagggcactgggagctccgttccgggagagctgtctaaggacgcggaaccgggtggggaggccagggaggtaaccaagagtccgagccgg

SEQ ID NO: 45 - DNA, 92 bp, Artificial Sequence
  subsequence of PRLHR
acacgagccccctgcttggctgcagcgcgctcagcgggcagcgggtccggccaagccaaaggcaggagtcagcaccacggacagcttccgct

SEQ ID NO: 46 - DNA, 93 bp, Artificial Sequence
  subsequence of ARL5C
cgggacggtggaaaagacagctgagcccccacctcccttcacattccagaaaagtgtctgaaaggcccggggcgcttcggggcttgccaagag

SEQ ID NO: 47 - DNA, 1006 bp, Artificial Sequence
  subsequence of PAX9
cggagtccggtcgatccgatgctggggtctaaagcttccccagccacacgcctgggccgagtcgcccccagagtccccggcccctgctccccttgggcgggtgtacacaaggatccggccagcgccgagtgtactggttcctcccacccagtgtagccccctccttcacactctcggcccggcttccagtctcccactgagtaaatatttatccagagagcgagtcgcccctcggaggaggcctcgacgcggctagggaaaggctttttggcttctccctggcctgagccagctgcaggccttactggtgttttggttttggtcttaaatttctccttttagtcgaaattcttccgatctctttgagatcttctgagtccagagtgagcagtgtaaccaggcggcagactccagctgacccttgttcttgttctaggtcctgcatccttacattcatcgggactgaccagaccttagtgttctctcttttccttccttcatcctctttctctgcctcagatccgtcgtctctcggtcggtctcagcgggtcgctgactacctcgccctagcactcggtccgcgccccgggaaggcttcagcatcaccctcttctgctgggatcccgctgaccctgtctgcagcttcggcttctcttgctcccagagcggggtcttcggcttgccgcggatacttagtggcccaaaacacctaaaattgactcttcctgcgctcttgagaatgccaagagcacccagtacctgcaaccgccggcatgagtggggcctgcggggagccgccagagctgcggacaggccccctgcccactgagttgctacccctggcacgcccaggggatccgcagcagctgaggccacatggtcacagccaagttcagcaggtggaaagggagagatggcattgaacctatgtgagaactgggaagatgtgtggcagctgaaggagttggtaggaacaagagggagttgtatgttacctccatataac

SEQ ID NO: 48 - DNA, 1001 bp, Artificial Sequence
  subsequence of HOXD10
tggttcctctgagagccgaagcctcgtgtagccctccggggcgcataggaacccggagccaggcaggaggaccgagcgggaggacaacgcagggaccaacccatccatggggatccgggcccggccagctgcttggcctcccggccctcgcggccctttttgcctccgccctcctagagacaccttggccgcttcttagccccaagggatctttcctttggacccctggggtgggatgtctcagggcccgcggaatccgactcaccttcccgctgggctgcccgggactaaattaaccagcctgcgcccccacccgcttgtcctggacccggccctcttaagcgcgttctgcctggttgtgtgtggggggaatgcttctgtgcgctggcgccagggcactctggcttccctccccgtccgtgcgtgtccacttggaggcccctagagctgagactttccttccgccggttgggcccaggggccgaagcgggggacgcgagtggggcgggctggccgagcgagccctggagaggcggacaggagggcggcggagagcgctgggccggttgtctccagcgcgcactatcgcgggcgcgtagtagatgtcgctgttgtccgtgcttacccggccggccggccaggctctggagcacgtgacccgagaggaggctgcggctcaaggccattttcaaatctcattggcttggttgtcatgtggtcggcagaggcatccacaattacacggggaatgttttcctagagatgtcagcctacaaaggacacaatctctcttcttcaaattcttccccaaaatgtcctttcccaacagctctcctgctgctaatacttttttagtagattccttgatcagtgcctgcaggagtgacagtttttattccagcagcgccagcatgtacatgccaccacctagcgcagacatggggacctatggaatgcaaacctgtggactgctcccgtctctggccaaaagagaagtgaac

SEQ ID NO: 49 - DNA, 468 bp, Artificial Sequence
  subsequence of TBX20
gtggcttctgctcctcaccagtgcgtctctagcctggggctcctattttccctagagggaccctggaaaatgggacggctgccgaagagaaggcgaaggtcagggcgcggagctctacggtaaccacgcgacgcgtagtgggagagtgcgaaccttatggtagcgcccggactaagcggacacagacccctctggttttacaaaggcggctgtgacgaggacttcccacggccttgttggagggcgtcctgttagccctgctcagcctgaataactcaaattctccgaattcgcagggtaatccgcttcgcttggagccgtgtaaactcaggctgacatacacactcaagctgtatatgtaaatatttgttctcttttccagccagtcggtctggctattagaagcctcttcgtgggtcttaaaattaaaataataataataatttttaaaactccaacttttcactg

SEQ ID NO: 50 - DNA, 1003 bp, Artificial Sequence
  subsequence of CACNA1B
ccgagggcacccaggggcggtgcggccggcagagtgggctccggggacggcccgggctggcgctgagcggccgtcgaggacccacaggctcgtccgctcctgggcccggcccagccccggggtgggaggcgcggggcgagggaggcgcagccggaggggccggggcggggcgggggcggccgcggggcggggccggcgcggcagggtgggcggggccggcgcggattggccgggccccgccacgtgaccggccccttatagggcgtggcgcggggccgcggagtcgggtgaggcggcggcggctgcggcggtggggccgggcgaggtccgctgcggtcccggcggctccgtggctgctccgctctgagcgcctggcgcgccccgcgccctccctgccggggccgctgggccggggatgcacgcggggcccgggagccatggtccgcttcggggacgagctgggcggccgctatgggggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggccatatcctgccgggcggggccgggcggggccgggcggggaccggggtgggggccggggccggggccatcttcccggtggccgggagggcgcgacctggggaggcgtcgggagacgggcgaggggggtacggagcgccccagggcccctcggagcggcgggtgcgggagcatttccgtgagtgcggcgggcgcggggcggagcccctctgctccggactcgtccctccgcacgccgggtcctggggatcctggtcggtgcacagtgcgcggctagcctggaggg

SEQ ID NO: 51 - DNA, 1058 bp, Artificial Sequence
  subsequence of ZIC1
aagcaatagggcaggagaataagccgcaaacgtgaaaactagaaaccaggagccaggtggaagcaatccgaaatcgtgtgtagctctgggtaatacgagtttccagatctgatgctaaaaaccgactcctcctactattaatttttttaaacataaaaataaaatgtagatccaattgtattagggattacaatagagttaagatatttataattccactgcagatgtaagaatttattgacgttccgggtttttaagcttgcaaagtgctaatcctgggctgctggctttttgttttattttttttctatttgtttagtttttgaaaaacggccgcggtatttttttctaattagacccctctcattctactttggcaccaggggagaagcccttcaagtgcgagtttgagggctgtgaccggcgcttcgctaacagcagcgaccgcaagaagcacatgcacgtgcacacgagcgacaagccctatctttgcaagatgtgcgacaagtcctacacgcatcccagttcgctgcgcaaacacatgaaggtaatcgccgcactctcgtcgccccctttgaggcaggagctctcttggctctcggcttggggtcgggcggcgagtggcagacaggcggtggcgggagccagaggaagcgggaaggcaaaggttccactcagtggaactgggcagagaaggtagggcgggacctggaaaaggggatgggagtgtccaaggccgttttaaatttttggtttaataataaaagttaaaagaggcgagtatgagcaaaggccgccactgaatttggcccagagctagggcttttgcagggggttgacgctgcggaaactgctgcccttgcagctgccagtgagacgcaagaattggactcctgcgcccaggcctgagctctggctcacctaaacatggacaagcaaagttcagggaaagaaagtgcagtctctggcctcatatccagtctccctcttcccagacctatttttcttttgtgagtcttggggaccctgactttcccaggagcacggtttctgcaggc

SEQ ID NO: 52 - DNA, 851 bp, Artificial Sequence
  subsequence of SIM1
cttctgcttcacctaccttcagtccccacaagcggcagaggtttcgggggcgcccatctcttaactgttctcatgcctaccgtccaacccagcgacaccgccggcctctccgcttcccaccggcagagaggagcagagcattcccggctcccttttctggatcatgaattggaggaggggtgcgtggcagaaagaggaggatgcaagctttgggacgagtgaggtgattttagaatcctggtcctccgtagttgctacgaagccagttcactattattcctaattggaagccgaaaactgccccccgtaacttctttatagcttaagggtttagcttcaacttcactccggggccgcctctctcccgccctccctcaatgccactcacaagccccctcgcctcccattggcctggcgcggccaggggcgaggcgctcattggccaatagggctgagtgacacgagtcggccccgagccgccctccgcgcgcccaggctccgggctctgaatcttactacccgcgggaccgctggactcctaatgagctcagggtgccgggtcggcgctgtccggttgccgccgctgctcgcggcctcctgggccttcccgacgtcgtgcgcgacccgcgctcctcgagctccctggggtcgggtggttgagcgctcagtccagcgcggcggcccagcagaaccgggaacgtccggaggcagcggaggtcgggcctgcgggttaggtctaccggcggaggaggcagggttcgcccaaaggcccatcccgctggcagtcgctggccgacggcgtgcgcgaggccggtttggggagttattttctctttctcgtttctctgaaa

SEQ ID NO: 53 - DNA, 1068 bp, Artificial Sequence
  subsequence of DLX1
tccgcaggcggtagccacggtcggactgagcccctggcaggctaaggcctggatcccggttctcccctcatcacaactcctccctggctctctgagagctgcccccgtgggctgacggcggcgggaggttcggccttcgagcctttggctcttatgggggaagggaggaaggagggatgtctctgcttctctggcagggagctgccagctggcgcagggttgggaggtcctatctctgctgttctgcggtcccttttttcctccctgtgacctaggtaggctcagtggtcccagcctgagtcacgttgttgtccgctcttgcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgg

SEQ ID NO: 54 - DNA, 1008 bp, Artificial Sequence
  subsequence of LMX1A
ctaggccgcgagaagagggcggcatttatgagacgtcgatcagctccgagattaaaaatccagccctgggtatttttaatcacttgccactcagacgcctacgaacaagctcgcccgcccctcgcggctttggggaattcggtgcccagtgcgtgaggcctggggcggcttgttggatttatttgggtagggacgacccacaagcactgacggacagatagtgatggggctcaatttagtgtatgaagaggggcgctagcttccctatcgcggaccaggtcccagagagcggggctccagagctcagcgccaagcggaaagagagtgcgcccaggacgcacggcctgaacactcacccagcagcgaggagaaggaggccgaggtgtcgatcgcgctttggaagttctcctccatctttaggccgtccagcatgttcgggccgggccgggaggacctgtagaggagaagaaacgatgcgtctgacgtccgtgcccgctgggactcggcgccagcagccaccgcactcctgggaaagaactgagggagtgtccagggcgaccagaatcagccaggaggatagggtccagccaagagaatgtagggtgggaggagagatcagtcacagcgaactgctctggctgatctgatttcacttgaagtcaacacgttatgtacttaggcctccgccccccaactgcgtttctccttctcctgcccccctcacccccacctacatcccttgccccaggttttccatcccgaatccgactccgccccaacctatacgaaggtgggccctcgggacgtctctgcaggaacgcagctactggggtatattgggtatataaagagtgggtaccctccctcgagcgaccgggtccaggcacgcgggacgatggggtttgcaatcccgcgtcccagccgccccgttgcggccctcacctgccccaggtcgagaaggggcactgtaagggacccggagggcgtcccgcccgcttctggactcct

SEQ ID NO: 55 - DNA, 1007 bp, Artificial Sequence
  subsequence of DLX6AS1
tcgtggcgcgccaccttggcatgttgtgtgcgggtcacactatctccagttttccattctcgatactggcaggaaagtctgatagtcaggcctgaagtttagattccatcccgcaagcgcagagatatcacaccaagtgggacctctcgctcgttccacttcccagcccctgactgtctctgagctcagactgggtcgcccagcaggtttcagcaggacctttaccatgcaccacaactaccacctcagtgcagctgcttcactggaacagagtgcaaggattttagccactaactattaggggaaggttgagagccctgaagctacaacccggacaatcccagccttcccggcttcctcagcaatcaaacaaatgagaaaaaaaaatccccttgcaagtagaggcagctgcactctggatacagtgctcaattgcagggaccttccgaggccctgccacgacatactctaattggggtgcagccagagttgcaggaagctggcgaggaaagcggtgcacctgtgagcataaagcgaaaaacatagacaagcacagggtgagggtgtagggggtgaaacgccttcacggtgttaacaacaagctgcagggtataaagcaataaaactgccacgtctacctgctggtattgaggacattacaaacagtctcggcgcgcagcccgcgtgtaccgagagcgcactagggttgggagtgggaaggtggggtgggaaatcttctcttttcctgacattcccttctgtcccccaccccccacatctcccccttacttagcccacctttctttttccatgaagcttttctgttgttgttttaattcattgccgatttgtaaacgtgtagatcgtgttactggttcgcgtgttaaagtgcacctcttgttaaaaggtggggaacagggcacgccacttaaaaatgaattcagagttactgaaccggctccccagactcaagaacttaagcgcaacgggctgggaaaaggaggggg

SEQ ID NO: 56 - DNA, 973 bp, Artificial Sequence
  subsequence of MIR196A1
tcacacccgcacccatcgcaaaaacgcagctgtgcaggccgagggcggcggcggacttacctgggcggcggcggtgccggcagtgcgcggccgtcggaaggtgcggagccagcggccgcggcccagtccaggctccctcccgccctcgcccagaagcgagggtccgagtggccacgtgtccggcagggaagtccgaggtagcgagggagtccacctggccgcccgctccccctcccgggtataaatcttggggaccggaaatacaataaaacttcaccgtgttgatgaacttcaaacggtttacatcccctcctgtcctgaaaagaaagacgtccgggggcggattaaaaggaaggggaaacaaatttacgagcggcagaagcacccgcctcggcccctttctttgcccgcacactttgtccgcgtggagagaagcagccccagcccccgggacttggcctccgccgaaccggccgccaggcgcgctcttctctcggaggagtcggcgctgacccagcccgcgcccggcagagactggaagccgccgcctacacggaaaatccacagagccccgcgcagaccctatgatcggctcggcccgccgggcttgggatgaggctccctgcttctcgctagagcagaactgggctccctggcgcttccttgcgaaagcccaggctctcccaaccacgcccggtggcctccattttttgcatctttccgctgaaagcgggttttttcttttttctttttttttgacagagtcctgctgtgtcacccaggctggagtgcagtggcgcgatctcggctcactgcaacctccgcctcccggttcaagcgattctcctgcctcagcctcccgagtagctggaactacaggcgcccgccaccacgcccggctaatttttgtatttttagtagagacggggtttcacaatattggccaagctgatcttgaactcctgacctcgtg

SEQ ID NO: 57 - DNA, 1050 bp, Artificial Sequence
  subsequence of FEZF2
ctttaaaaggaggcaactggcgcccgcgctcctctcgggaaagtgcgagcggtttgcgaatagggaaagagggaagagacgggagggggagggggaggaatcgtgatggttttgccggtggaaggaaagggggggggggacccaaacccaaacctaggcagagttccgtgcgtgattcacaactttggaggcctgcaggtttgggggacgcggggagggcggagggtcccggacgcggcgcccccaaagggcccctgggtatcagtctagtagccgaagtatcccttagccctccgcagccgagctgggcatcgtgctaataaactgccatttacccacggaaccggcgccagccccgaacacgcgcaaatgataattacctcattaggatgtggcgcatggacgcgggcgccgcgtttggaggggggaacttgttaatggggaaatattaatttatgcaaaagcaatgagctcgccttggctggcgtcggaggtcgggagaaagctcccactgactaaggacgaccccgaagcgtcacggagagcaaggccgctcgccggcatctcgggggcctcggcgccgcgcaggtgcgcctccagctttatggccagctctgggtctgaggctgggggagactaaaccgagagctggaagactttcggagatcctaatagatcagaattgttgatttatgatcgggtttctgaggcaaagccgagttctgcttctggtttagggctgcgtgcggggaggattcggagggcagctctcccagctgatgggtacttactgtttgtataattgagaaggtcccatctccacctgtttaaggagcagattgaaacagcagaacctccccctggatcctattaaaacgtttttcctcctaaggccattcagtcgaaccaattttccgaagtgattcatagagctgaattctgccaaggcagctctttcacttttagccactaaagcaccgcgtggaaccgggttttgtgtttttctaaaataaagagaaggattatcagagaggagagggagggggcctg

SEQ ID NO: 58 - DNA, 974 bp, Artificial Sequence
  subsequence of STAG3
cggacagggggcggggcccgaggcggggccccgccaataatggctcggagccggcttcaggggcgggggcaaggaagaaattaagccaatgtagcgcggaagctcggcaggggggcgggacccggctggcactcaggaggttggtgaaataggggcgtggtctccctcgggggcgtggcgatccggctcaaaaggcggggcctgggtggaacctagcctatggcaattgagaagccagattaaaggggttactagcagtctggtccaaaacccgcccaatggagtaggagattgagggtagcgggggtgggacctgggtgggggcggggctaaagcgggccgcccgcgtgcccaagcccacgtgagagggcaggacgcctgagagcttgaggccacacgaggctgtggagcggcgtgactcaaacgtggcgcgcatcagctcgcacacttccaaacctcgcgatagctactggccctgggcgagccgttgggattgcgcttgcgcacagcgtgtcttgtacgttcccgcgcttttctggaatctttcgcccccggaagggcagcggcgggcgcctgtgtggaaggtggggtggccagagacacccgagggacctgaggtggggaccttccctgaaccctgctcggccagaagtcactcttccaggctctgacaggtttctccttgacccaagaataggccctctcgcgaccctcccggccctgacactaggagccttcccgggccgtgccccaatggcctccgccacagagctggagaatcctctccccaggcctccatagactgagagtccattctcttaagcgcttggaaggctctttatcctggagccgcttaccccaggggcagtcgtagatatttttaccttctggaaaaataggaaacgcgcacctgcctttcctccttttgcgtttccagccctattctcatggcgcctccacccccaaatccttaa

SEQ ID NO: 59 - DNA, 981 bp, Artificial Sequence
  subsequence of PTPRN2
caacaacactgaaggacccgagggtggcttctccgtggctctgaggttaacgctgcagcgtatgcagcaaacacctgtcaaggacccactgtgtgctcggcacaccaaggcaggtaacaaaggacccagtgcatgctcggcacgcagaggcaggtaacaaaggacccactgcaggctcggcacgcagaggcaggtaacaaacgaccagcctcagaagcagatgtgcttgctgcaaaccaccttcctacggcccagcagaggcacaacaagcagcgaattttttccagggcctcccgtgaacacggcaccctacgttgccatagcgacgccatcccagctctgagccgcaacaaagcggcagtgtctgtgtctcacctttggccatcttgttgagaaccatgtcgatcaggacgtaggtgccgctccggcctgcaccgtcactgccaacagaaggagagaaatgaaagtgctgccgtccacacggaagcccgacccacaacaaagctagtgacagttcatttccttcctgcttacgggggccggcgagaggaaaggatggcggatgtgagagggggaggaaggacgcgggctgggagaatccaagtgaggggagaagggaggcccctgggggcttcgagcctctgagcagcttgtagcttgtgggatgagctgctgtgctcagatgtgactccctcttgcttgttcccccctctttggccaaagatgtggtaaattttgccagaacaaaatgaactaagcgtttgactgtgtcagagttgatggcatttgtattgaaaatatctgcaaggtgccttctctagtgttgccgggcaacctcccgagcggagaggggagggcgctgggcggctgggccccgctggctcatgcacgcagggacacttcaggcacatccccctaggctgcctgccaccccttctaaacgcaggtcctccgtctcagaaggagaaattgacataacag

SEQ ID NO: 60 - DNA, 1004 bp, Artificial Sequence
  subsequence of BARHL1
ggggcaccggacttgaactcgtttcctaatttccctcccgaacacactcaccagacgtagatgcaaatgtacaaacacagaggcatccacacactcaacccaaatagccacacaccgacacacacatcaatacaacttgcagtcacacacgcacgcacgctcagccccacagtcactcgcacgctcttagcagaggcaggaatggtgcttttcataaacaagcctcagaacacatcgccccattaataataaatactgattactgctccgggcatcaataattaaggcctgttacagtaattacacaattatgatgatgatgaataattcagggagggaaacagggctggtgtggctgagcccaccttagtgggaccctggctggtccgcctagcctcacctgcaggtgagcctagggtccaggcctgacttctccaaaccccaggccgttggctggccaaggcctctggactttggggccagacagtgcgcagggctcgcagtgcgagccctggaggatggcaggtgcagcctgctccagccggcagggcgtgcgagccgggagcgggtcctctccggttgccatttcaggcctgctcgcttctgctagcttgcagcccggccagcctgcagaggaatcaatcgcattcccttccctgaagagcacccccccatcccctcaggcctctccagggtgatcctaccttcataaagaggcacgcgcagggaggagcaccccctacccagctccaagggttcgttctgcgatctcctgggcagggtaaggcgcagttagagccgggctttggggtcagacagcagggcttaaaacccaggctcctttttgaaaaccggcccagacctcagttttcctctctgtaaagtggggtaagagccacggaaggccggtggaagataaaaggagaggatgcacgtaaggcgtgcgatggtgcctggcactcggttatttattaacaaatgagtacgatccctcttggcctccc

SEQ ID NO: 61 - DNA, 1064 bp, Artificial Sequence
  subsequence of CCDC140
tgcccatgggttcataaactagctgtcacttgctataaacatgtacaaatggaggccacaaggatgtgggggtgctggggacctaaagctccggaatcaggaggcgactcattttctctcctttccggcctctttcttttctgccgagcgctctgagattcatatgtacccagatgatcagcccggcccttctcaccggccagtggccagaggtccctggacacccggagaggggtgggagggcacccttaggctctggacagctgggccacatccaaagacttaatgaatctaaatttcttgtggcctgtgtccccgaagtccgacctgggatggccaggacagcaaagttggctgaggctgcctggcctagggcgcccgcacgccgccaaacctaacccggctcagagccgtgactccgcagaacagggcgcaacaggtctcaggaatgccgggtgattccagccactctttctctgtctccgagtcttgggcctcccctttatttctttctgaagtctctccggagcccaagccaccccacacccaaaccccgcagctggatgggagtccaggccacttccctatccgcagccggtgggccaaggagtcctggtgccaaatttggccgcggtacaggcgctaattggtaaagggatgacccagcggtggggcttggaaggcgagggcccgcgccctgcatccttgaaccctggtttgggaggcagtgacttttacctgggctaacaaattacccagaaagcatcagtgggactctccacaacggtgttgtgaacgagtgtagggtccactcacgctctgtgcacgttcgaaatgaaacgggagccttgaagacgatatttggataatttgacaaaaaaaaaaaaaaggtgggcgggggagatagcttctattgtcccctctgcgcctctgcccccagtttcagagacactacaggtgaaaataacagcgggctggctgtccaggcctgagtggagcgtgcccttgttagcttgaaagttctccctcgcagcccgtttggatgcgtgcgtctacagcccagt

SEQ ID NO: 62 - DNA, 1780 bp, Artificial Sequence
  subsequence
cgtttcgctggggctccctccgctctgcaaagggagcggggagctgggggagccttgcacaccagtcttcctggagctgcagacgaggagccccgcgccttcgggagcctctcggcggcttcccctctaacgcgcgactgtgcagcctctccccgccgcccccacccgcggctcgccctcgctccctccctccagccccgccagccggagcgcgcacaccgggactccggcacgtgggcgcgcgggaggtggcggccgctgctgccgctgctgccgctgccggagcccgaacccaagccccagctggagccggagtcggagccggcgagatcgccctccatgctgcgggagctgcggccgccttccgctcagcctctggtcccaggcgagcctggcgagccggcgaagcaccggcggggaggaggactagaacaggaggaggggcacggcggattgaagcgagctgggctgtgagcaagggacacccacagcctggagaaacagccccgctctcttgcgcgctgtctgctccagccgctactgggggctctaagcagcgcgatgctgcttcgcttcttctaggcggcggccggcggaggctttccgcagccgcttggccggcgccggcccctattccgttggcaagtcccttgtctatcccggagggcgcacccggacgctcgagccggagcgagcgcgaagtccgaagtccgcccccagagccgccaacttccctgtgagcccctctccccgccgcagcctgcgccagacctgggagcgatgcgcccccacccgggcggcgcgccctctgccaactcgccacccatgggatcgactgccctcagctgcccggcttggagtgcgtagccttcgccgcgtggcccaggagaggagcgagacaccaaggcgtttcgcggactccgcgcgcaccagtcggcttggtggccctgcgggatagcgtttatacccaggggtgagtaggccgctaatggaggccccctttacccccagctagcggcttgcatggacggcaaagagacgggagaaagttgggtctccttgcgcttaatctggcattggcagaggaatgcaacactgtttcctccaactcgattgccgcgggggtttccccagttccctccatctctaagccaaggagggagtccccttccgcttcctaaggagaaaaggggctccctagagcagatcgctgacttcctcaccaacgtgccatttggggcttttcagccgcccgcgcgttaagctagctcccagcataagcgcttagagatccgcagccgcctgggcgcgctaagagatcgtgggcagacctagcccttagggcagcctaagcgcgcttcactaaagaaaaaagactgttggtatattaaagaagtgtgtgtgtgagagagaaagagacagagaaagagacagagagacatgggtgggggcaccaggaaaggtagcccgagtaggaggtgctagtgaaatttatcttttgctgacggatccagcctccggaatgcccgattgtcttgatccgcggtgcggtgcgggttttaggggcgcacaatacagggtgtccagacagagtccaacaggtggcctgcgcgggcatcctgtggctaggcgcgggggaggggaacggctccctccccgagccccgggctgcctctgctgcttctaggggtgcatctgaacatggataaggaaatgcgctccctgctctcaggccctatttggggtgagtgtgtgaggcatccaaatgcctcctgtcgga

SEQ ID NO: 63 - DNA, 1038 bp, Artificial Sequence
  subsequence of GATA2
gccggcacataggaggggtaggtggggatggggtggtgtgtagcaggctgggtgcccatagtagctaggcctgggcgcaggggactgccactttccatcttcatgctctccgtcagtgacacctggtacttgacgccgtccttgtcctctcctcgggctgcactaccccccgcggaagatgaggctggagacgcagcccccgtggtgctagggtcaggagacacttctttgggtggcgtgggtgggaagccgaaaaggtgggagccagagtgggctgctgtaggggtgagggaggccactgagctcccgctgcctcccccgctcccacccccagcccctgggtacacagagagtgggcctccagggcctccagcagctgaggggtgcagtggcgtcttggagaaggggctcacggtccaggggttgtggtggtgggccgcagcggcagagagggctgctttgcccccgtccagccagggcaaacccgggctgtgcaacaagtgtgggcggcacatctggcctccggtcaggcgggctgcgggcaaagagagagaggatcagggtgggcagaaagatcagggtaggcagagctagggacgcccctgacagacattgagatcacgactcccagaaccagcagtcatcccctccccaaagaaagccagaaacataataccccaccggtaataatcaggaatgtcagtccaagctgaaggacaagtggcatagaaggaaccccaccggacagaccctacagggaaccctcacaggccagctggaagtgggcagaaaccctgtgggtcccagaccctccccaatcggccgctgctcccacctctcccgccccaatttttcagcagctcgattcctgcggatcctacatccgggaagcaagcagacgggccctcctcccctccctcgcctggcgcgcggcgcctgggttctcatcaccacgggcccagtgctcaccgtgcgcggggctgtaggagacgcgcgcccgcgcgtgagcggggttggcatagtaggggttgccctgcg

SEQ ID NO: 64 - DNA, 1100 bp, Artificial Sequence
  subsequence of ADCYAP1
gaggcaaaaagggaacgcgagccagggagtttgatccgttttgaatgaaaagaaagagaaaccaaaccaaacctctcagtcatccaaaaccttcaggcttccagggaggttttgctataattttctctaagcatgactgtttctgggggaggggaaaggggtggttgtatttactgaaaattcaaatcgaaataataaatggccaaatgtggacacttatggacccaaacagttttgctcacgccagagaaactgagagcacagggcttgtgtgaagcctatctcggcagaaggcaacattctaataaagcccgtgggaaaacagattacattttcgccatgaataagtcatgcagtgaaaaatattgcctacagcctgtcgacttatattattatcacgtttttcaactcggcgtgaggagggagaggagtgttcatatttgactaggaattgcaggatcgatgcaaactccagggcagcagccagactggcatatgtagggctctccggttactttctctgtatgtcgcgggtgagaggaacagcgaggacaatttagcgcaaacacacgaagggtcggatctcaagggggcagcgctgggagaaaggttaggcttgaagcgcgcgtcgcctgcccggatcttatcccgggccccctccgcagggtttggtgccaggagatcctgcgtggggaggggggcatcgaggggctgccgtctcggccctccccacggctgcttccaggcagaggcgggcgacgcggtgggcagtgcgagccccgggccctccccgaaggctcccgcgtggggtggggcccgcctgctccccgcggcgattgaacctgtgtctcccgccccgccaccctcttcccgacccctttgcttgcagtgggagcctcggcggcggcgcgggggacgacgcggagccgctctccaagcgccactcggacgggatcttcacggacagctacagccgctaccggaaacaaatggctgtcaagaaatacttggcggccgtcctagggaagaggtataaacaaagggttaaaaacaaaggacgccgaatagcttatttgtagcgatgggttaccagctaccct

SEQ ID NO: 65 - DNA, 963 bp, Artificial Sequence
  subsequence of TBX5
gtagagaataattattgtaccagggcccgccgaggcacgaggcactctattttgttttgtaatcacgacgactattatttttagtctgatcaatgggcacaatttctaagcagcgcagtggtggatgctcgcaaacttttgcgcaccgctggaaacccactaggttgagttgcaaaacgtaccgcgtagacgcccctggtggcgccgagagaagagctaggcctgcccagcacagagccggagagcgtcgggccttccggaagggtaagttctccgccaaggggtcccgagggagctggacgtctgaatctggacttgcccccagcttcggggttcgattctgggttttgcgcgtccccaacccccagggctttccgaagcatggcctggctccaggcccggtcctgtaaggactggaacggcagcaaaatgtgcagggaggcagtcggccggcagagctgcggcgggagccaaggtcaggcccgcggggagagcgggcagcttccagcgccggccacaagctcccaggccagctgggccgcagacccctttgcttccagagagcacaacccgcgtcctttctctcagccaggctgcagtggctgccccgagcttcgctttcgtttcccaagctgttaataacgatatgtccccaaatccgaggctcgtgtttgctcccagatgccaagaacgcaacccgaaatccttctcccaaaccctaggtcgacgagatgagttcctacttgacctctgagccgaggtgggccggaaaccgaggcctaggccccgccggggctgcaaggaaaaggggaaactccgagcgtagcgtcttttccttgtggttcctttctccggcatcccggactgcgggccctgcagccacctggaccggcattcaaaggattctgcaagtccagcttcacagactggctttcccagacgctccgaagcccgcaccacgaa

SEQ ID NO: 66 - DNA, 837 bp, Artificial Sequence
  subsequence
ccagcccttacccacacctttgtcacgacgggcaattgggtatgtgtaggggaaaaacagcaacgttaaaacgcaactgtgtaaatgaggatagagagtgcgaaaggagggagaggcgaggagctgctctatttctagggaggttttggggagactgatcagctccaaggacagaccgctgggaagggaaaaacggcccacatcgaactggatgccggatggaaacctctctgcgctattagactgcgtccagtacagcagatggcacgagcacgtgcggcgctcagcttaggctctcggaggcagctgagttggaaatcccgacggaaagcacccacaagctcccactctgcgctggcccacccgcgtgcacgcccaccccccacgcgcgtccctggctcagaagcgcacagatgtttactgcttagagccggtaccgctggggagatcgagcgacttgcgcggcgcacagtgcggcgctggcagggctctgggctcccggtcgggggttcgagcggccaagggatgggggtgggggcggggagagtggggggagggcgaaagaccgccgagaggaggggggagtgggtggactaatgatgaaaaagtctcctccatcccagttccttaattaaatgcatggaaagaaccgaggcgagcacatctggtttcaatctacagccctttgatggcatcaaatgttcttttcccagatcagggctggaagttctgggctaactatggccgtttggagcccagaaaccatttacacacactcgtacccttctttctctccagtcgagcctcttgactataggacgaaaaaaaaaaaaagtc

SEQ ID NO: 67 - DNA, 1012 bp, Artificial Sequence
  subsequence of NECAB1
cttgtgactttgggaaacttcatatttgaacattacctaacaatccttgccccatagcaaacgtttcgaagcctactctaggcagagttcatagcaccgcctttgtcagcagcactgccataggtaagcctcagaaccctctggggtgagaccggtgtaaccattttactgatgaggaaaagtgaggcttacaggttaagtccttcgagcgcaagataacaccgaatggcttaatccctacaccaaaccagctgctcctcgattgctggtattcgtgaaccctgcccccgcctttggggaaggtcgggcagacaggccactgggaccaaaagcagagtaggtagaacctctctgagttcctccctatttatggcatccactcaggtgaggctgggggcgtccttaccttgcgctttggctgctcaggacaaacgccctgccattcgtcccttctctctgtccggacgccctcctccatccctgcacttggggccgtcgtggggctggtaggggtccccaggctatctccagccgtggccgatggccgtgccgggggcagagcccgctcccctccgcgcgttgctacgtgcggggatgacatggatccagacgccgtcaactacgggaaggaggaggcgagagttggagcgcgtgggagcgcagaggccggaggagggagggggggacaccgagcgcggagagcgcggagagcgcggagggaggcgcgcgcgggagcgaacaccctcccggatccagagcccggcggcggcgaagcagcagctgcggccgcgcccttgccagagccggtgcgtccgcctagccccgctccgcctgaggccgtcagggctcccgaggatggaagattcccaggagacatcgccgtcctccaacaactcctcggaggagctcagctctgctctgcacctgtccaagggcatgtcgatcttcctcgacgtaagtacagatggtgggagctgggcggttgcttcccagcacctttcttttccccgaa

SEQ ID NO: 68 - DNA, 1017 bp, Artificial Sequence
  subsequence of ADRB1
cccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccag

SEQ ID NO: 69 - DNA, 1030 bp, Artificial Sequence
  subsequence of OTX2
ggcggcccccggcccaggccgacccgcaagcgaaccgagcttccggcgcgcgggcccaaggaggcgcctggctttttattattgttgtttcaatccatccatctagttacatctgcatctttttgtctcggactctaaaaaggtccctgggatccatccaaacgaccccaaccaaatctgggggccaaaacgcaaagatcgcgggagaagcccagaacggcgttgacataaaaacaaaaccacaaacaaaaccttccaaaacaccccagattacattcgcagcgtttcgacgacgttttgcagaagcgacgacccccaggagcacgctctctgcctctctcccactaccgctctcatctctagatcacatttttctttttgcaacgatcgttattacacctcaaaatttgtaagagaaaaaatacatccgcctacagaactccacgttcgcagaggggcatcggtcggtctattggaggtactggggtgatggccgacagacacggggacacgtagatatggatatagacaagacagacggacacttaccttacagcctcatgggaggttagaaaaagtcaaatatcctttaaattccaaatagccagctatcctaaaagaaatcaagagtattgagttagaaaccttgcagtcttcacccttccatgaaaagtcaaacacaaccaaaaaaaattttttttaaagaacgaaaatgaggaaggcggctagagttctaaacacgcgtgggcccctctgcaatacatacaaagtagatgtggcgagtgagggctgctctagcaatttttatttttaatcacagtggaggctgtgttggagctgaggccggtcccgctctcagggagatttgctgagaaagcgcctctctggcaactttttgacgcaaactctccaaccaatggcagggagagaatgattgacacatctaagccagagggaaaataataaaaacacacaacagggggaggaaaacaggccgctgctgcacggggggacgagcaggcaacaaatc

SEQ ID NO: 70 - DNA, 1076 bp, Artificial Sequence
  subsequence of NKX2-6
aagtgaatccttgagattcagggccttactttcgttaggggtgtgtgaagcacactgggtgtcaggagaggatgggaatgggattcgagaggcctttttttggactcctcgagagaaaatggagagagtctcgaacccaggagataggaggcgtattttccccatgcacccatggatcacgcccccgcccccacattccccccgtaggaggcaagacctgagcgcttactcacgtggctcccccatccgttccgcgtccatctccaagactgcctcacagggacccccaggaggctccgaaccatccagctttctgtcaccgccgccgccaccagcgttgtgaacctctgaccctcgcggctctgcgtccattctcaggtactgaaagttttccgggctcttccgcacccgcggatgtggcgaagccgcggggcagctccgctcgcgctccagtcgcaggatgtccttgaccgagaagggggtggaggtgacggggctcagcagcatcccgaaggcggatggggcggggccgaggaggtccgggtgaggagcggcaccctgaacttcccgtcttgtcgctgcaggccccgcagacagacccaagctctgggacagacgcccagcgtcccagacagcgccttcctctgggccatgctggtaggcccgggtccagggccgggtgacgagaccgtagccccccattggttctcgcagaaaccacgtgctagctctgcaccttcctcccccagcgctttcttcccgcgcccgtcgaatcctctccagtccccagtcgccttcctgggaggtttggttcgtattgacggttctcaaagcaaagaaaacgtggagagacagtgctgcgcgcggcttggatctccgtcaatacggtactagcgggagcttccgaggtcaacctagggtccacggggaccctcctagtggacatgcggggattggaggtggagtccttgctgtgcaacccgatccggtccatccagcctggtcttagctcaactctgcattcttatggcggaggaaagctaagatagagacagctgggacccgcggaatttgag

SEQ ID NO: 71 - DNA, 927 bp, Artificial Sequence
  subsequence of SIM1
ttgatagagcttttccttcggaatgaatttaatacccttgacaaaatatatattacagacccgacatcatcaccgccctcactctgacaggaagaggaggaagataagcgtgtgttctgttcttttaaaatttttctttattttaataaataaacgctacatctcaatagaaatgtatcctccttgcagtccttcccgggaaccttcacctccccaactaattgaggtgccttgtaggcaatgggggagggtggcggaggccctgttccatgacctgactacgacttggtgactgaggaggggaaagccctcctaggccgaggccactgcggttcctgaacagcttgtctcaaccaactgtgtatcccttgagagagtgtaggagagtctggcccagggggtaatcctgaaaaccccaaatcccatgtatcctgccgggcctgcacatcctcgctaaatgtgtactgtgcaagtcttccccgggctgaggcgtcgagtggccttcgtgttcagcctccgggcgcctccgcagccgagctctttagagaaaaaaagagagggaaattggtgcaaccagcccgaatcaagctcctagagcagtcactggtaggaggtcccttcacgcaccaaggcgcacccgaccccacctctggcactgctgcccgccctgcctgcggccctctccggctaaggagcctgtgtggttgacctgagggcatcggcgagcgcgccgggccaggtcttgttccacgcagcagcctggaccccacagtgtgtgcctgcactcggcagtgccctgatctctcctgcctgctgatcccgggccagagacaccgcggccgcacccagggaatccactaatgttactgtaaatgtggggtggaggccgaaggtcacaccgctgtctgtctctctctttctctctct

SEQ ID NO: 72 - DNA, 927 bp, Artificial Sequence
  subsequence of DRD4
acaggcagatgcaggctcagccccctggctgccgtgggacacacacacacacacactgccacagccactgcccaccacacacacctagtgcagatgctggcacacccccagaaggaggctcacagctcgcaggggagacctgggctggacaaaacccaggggaggggagggtgtgtggggaccaggcccctgctgagaaccctggggggaagcctgagggggaattgggggatggagcccacactccacaccaggtctggccctcgagtgggtcggccttggtgccagcccctctgcggccagagaaaagcagcttagggctgagctggagacgcggtgtccccgactgtgggggagggggactcgaggtttccccttgatggacacagtgaatccaggcggctggggcagagaccagcagcacgggacacgcgtgacctgtgctcctttcgagccgcagacgtcacagtgacgacgtttaagctcctaatctccccaaatcggcgggaaggattagaggggctgcctgctcctttgcccttggagagagtcactccacgtggagtcctacgctgggcactgggcacggtcttcatatttttaattataatttatcttcaagacaattatgatgtggatactatcatcacccttgtcttacagatgaagaaacagaaatgaattaacgtgcccgagtctcccacagagaacttagccaacagagcagccagcttcgcttctaggcccagcggggaccctgcctaccccaggctggggaggggctccctttgagcccgatgatatcaggcccgcccccagggaattccccaggccgttctcatgtttgtaacaaaagggagaccaaggccagaagccgtgattcacgcctgtaatcccaggactttgggaggcaaaggcaggaggata

SEQ ID NO: 73 - DNA, 810 bp, Artificial Sequence
  subsequence of TBX15
gaaaaggcttggacggtcgaaataaatctccttttaattttcttttcatcgactaataaaaataattccccagcactaaactcaaataccgtaacgggccacaaaaacacggagaattcataaaactctatctctgcaggtcacccgctaatcgcattattattagcctcgggagcatggaaattgaactgtcactgcctaaagagaaaatgtaagcgacagctgtccctcctctgagttggacagctttgtggctgagatccccaagctcctgagccccagaccgtctctatcccagtgcaggccgctgccccctccccgggcttgtcagcgtgtgaagcggggtctactccccagctctggctgttcagcaggagcaccttccagcatggttttagaagtccgggctggaaagcttctgaaaccgctaaaccaagttgcgagcgttgtatgtgcgcgcgcgcacactcaagattcttccgacaacagctgttcccccgggctgtttttgcacagttgaaatcttggcacttaacacaaaacgttaaagttggcttctcccctcctagggttctgttaaaatacagacatgacatcccccctgctttttacatgttccgtgcacataatatacatgggttactgtaggatctgagacacaaatcaatcgtacccacaccctaaatacacagagatgtgggccctgaagagtgacccaaggagtaaggggtctaacatccattcttgtttccgtaaacgccaaaggagtagaaatataattatgttccaccatcgaaaagttggtgggag

SEQ ID NO: 74 - DNA, 1080 bp, Artificial Sequence
  subsequence of TRH
tttctctctctctctctcttgctctactctcgcccgtctgcgttctacattggttctgcggccgtgtccctttcgtgtgtgtctgccctgtgtttctccgtcgtctccgtgtggatctcggtctttgtctcgccctctagctctctcgtgcgcctgtgtttaatcgtgcgcttctctcaatctctctgtccctccctttgccccctacacgcgtgttctcgtcaacagatctccagaggtggtgcagaaacgaccccgcgccggcgccccatcctgcggccagtgcctccgcgccccggctccggtccccaccgtccccgccccagatttccggaggagcaggcgggcggggtcccgcggggccggctgccgtcagcgccccttcccggcggccgcgacccctccccgctgacctcactcgagccgccgcctggcgcagatataagcggcggcccatctgaagagggctcggcaggcgcccggggtcctcagcgctgcagactcctgacctgccgactgcggatcccgagtccccggatcccggacccatcctgtggagcccactcctggcaggtaaccgccccaacccctctccttccgcagacggtgtccgggagcactggaaagggagccctctcggcccgccctcaccttcggttctctgagagcttcctggagaacagggccggtgcacaggcgggcgctgtgctcgcacccccgggtcctccccgagcgcaggccggggacccgacgggaggcacggcgcgggcacatcggaggctctcgctgctgggcactcaggtcgccagagtccgcagcgcggggtgggagctccgcggcaccccctgcctgggtgccgccggacagagctgggtattcagggcgtcggcctgagaacctgggagttcgaatcccaatctcaacgctagaaatagaattttagcgttgaagttaggattttagcgttagaatcagattcctggcttagtcatgaaagcttagcattagaaatagaatcttagctttgcaaatagactttgagctttagaacctgaatcttagcgatagaaatgggatctt

SEQ ID NO: 75 - DNA, 1050 bp, Artificial Sequence
  subsequence of FBP1
gcaagaggattgcttgagcccaggagtttgaggctgcagtgagctagtgagaaacctgacttgggtccctaattgcagaaagagtgaaggaggacaaagaagaaatggggaaggaaggcacctgagctagcactttcctgtctgaggctagggcgggtatgacgggcaggccaggtgcccacacttggccggctgagaggggtacgaggcaggctgctcagggcagagaagaggaatgcaagatggaatcaggtgtacacctgtgagacggacccccaggtgggcccctccaaggtcaagggaggagatgaggacttgagtgaggctgagccagcttctggagagaggggctaccagacagagagcgagagaggcgctcagacatcagacggacagacagaggctgacggcaggaggctcagagggcccaacgtcagcccgccgggcaggctccccaggcagacagacaggacggggcccaccgcccaaggcccgactcacaggtgcgcgatgcccgccttgcgcaccgccgaagagatggctttgactgctgtgcagagcgagttgagcagctgggtcaactcgcccgtgccgcgggccttcctgccctcctccatgacgaagcgggtcagggtgttgacgtccgtgtcgaagggcgcctggtcagccatgcttgaaccgggtagagcgcggggctgcaggtgcaagcggcaggtgcggggctgcaggtgcgggcggcaagagagggcagtaggcactggccgcaggtgcggagctgcaggtgcgggcggcaggtgcgggccgcgggacctggcgggaggactgacagaccgactgccgccccgagtgtccgcagcgctcgtggtgcaactgggccaggccaggcgccggcgggtgaatcgcggaaacctttagacgcgggtcggccccccgcccccgggaacactcttgcgccccgcctaccccgcggaccagaccgcggctccgcctgcttggatcttcagacaaggtcggggatcctccctgcggtctcccaacccccgcccccac

SEQ ID NO: 76 - DNA, 968 bp, Artificial Sequence
  subsequence
caccccgtggtgccttccctccccgccctacagccaagcccagaggaacagggcatcgcctcgcgcggtcccggagccacatccggacttgggaaccccgtgtcgggcttagaggacttcactgggcgggttggggtttattttcaataaattcagctgttcacatacacccccaccaccatttccacgcgcgcgcgcgcacacacacacacacacacacagagggacttgtcaactattgagacagcgacccagaaaggtgtaaattcacctggtgcccacccaattgcaaagctcccagacccacacaaaacactagcgagccctggaggtctgagacgccccgcgcccgactcacggcgcgaacaccagcgctccggccgggtcgcaggggccggtgcccagcgggccggggtctggcgagccaggcagcgggccggctgagctcctgcgccccgcggtcgcgtggcctactcgcctactcccttattattggctgggacctgttcctttccaaagtgtgatgcccggtgtcctaccctaggccacgcggagctctctcccggcgtgtcgggccctgcgcccctgcccagtggctcacctcggctcgctgcgcccgccttcgccccaagtgcccaggacggagcgaggggctgtgggtcggcgcagggacgctggctgctcacctccaggagccgacagaaggtgccttcggcgtcacgcgtccaaccctggggctgatttaaataactaataagctcagagaaattttacgaggggaacttttccttttagtatcaataaagcctagtaattgggtttgttggttacctaagctcgagttcgactttctcagtctctctctctctctctctttctcattcagaggcaacgttttcaaaaaccggcaattgcgtgtgcgtttggggtgtgcaagtgtgagtgtagatgtgtgaaagttatttgtt

SEQ ID NO: 77 - DNA, 1080 bp, Artificial Sequence
  subsequence of BCAN
cagcctctcctcacgctcgcgcagtctccgccgcagtctcagctgcagctgcaggactgagccgtgcacccggaggagacccccggaggaggcgacaaacttcgcagtgccgcgacccaaccccagccctgggtaggtgagtgcctccgcagccccgccgcccgccgtggggtcggggacagggagaagggagtgcctgcctggtctgcgccccccgcctgtcagcccttgcctcgaggctctggggcacccaactcgtcgactcctgacaccgcagcggggtaggctgctggacagccccgagcgcctgcagctgctgctgccatctctgatctacatgcttccagctctgccagtggcagcccccctgctgctcgcagtctgatcagcaacccctcgggtcctcgcatcttccctgctgcgcgctcctgtcccagcgcggccgccgagatttccgaccttgtcccaggcagggcggtagcgttccggatcagtccttgcctccgtgcccccacgcacaggcagctccaggagcagcggccagcaacccttctggggacaccttactgtagtccggcaggacagccgatcagagccgctctagggggtggtcggagtgcccatttcggctggaatcccaactcggctccttgctctgtgaccttggggtagttagttgacttctctgaacctcgggttcctcatcggtgtaatgagcaaaacataaacaatctttttctcacagttgaggttatccacaggaagcatttaggagaactggctcaggtagacatcagtaactattagctggaattcggttaccactattgcattccttcctagcactgccaggggcctgggaattccctctcccaggtatttccatatcctgtcaggtattcccccacctattggaatagctctgcctacatggttctaataggaccaagagattggcgtcaagtccaaggggcaccattattatctgcattacttagattgcagatgatgacagtaagatcagggaacagtgttacccttagacccacccacagtattaccagtgcctatgttgctatg

SEQ ID NO: 78 - DNA, 1017 bp, Artificial Sequence
  subsequence of TP73
ctcccaggtcagggcccaggccccgcacaggccaggagtgactctggtgggctctccccctcccccgtctcctgcctactctggttgggggtgtaggggccagggtgtggtgtggccagacctccaggcccagggcgaccccccctgctctccctgctccactgccccctgcccctaatgcgccggcctctcgcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccggggaaaggcaaggtccggcccatccccaggcacctcacaggccccaggaaaggcccagccaccgaagccgcctgtggacagcctgagtcacctgcagaaccttctggagctgccctagtgctgggcttgtggggcgggggctggcccactctcagccctgccactgccccggcgtgctccatggcaggcgtgggtggggaccgcagcgtcggctccgacttccaggcttcatcctagagactgtcatctcccaaccaggcgaggtccttccaaaggaaaggatcctctttgctgatggactgccaaaaagtattttgcgacatcttttggttctggatagtagtgagcagccaagtgactgtgtctgaaacaccag

SEQ ID NO: 79 - DNA, 1058 bp, Artificial Sequence
  subsequence of MIIP
gcctcccgagtagctggactacaggtacgtgcctggctcattttttgtagagacggggttttgccatgttgcccaggctgggtatcttttttttttttttcttttttgagatggagtcttgcgtcttgctctgtcatccaggctggagtgcagtggcgcgatctcggttcactgcaatctctgcctccctagtagctgggttcaagcgattctcctgcctcagcctccctagtagctgggattacaggcgccggccacctcgcccggctaatttttgtatttttaggtttactggcgtgagcaaccgcggccggccccaatactacactttcttgtttactgtgtagtccttatcttctggcgcaaagcgggcactcgctaaaaacctgaatgaaagtataaagcaggccgggcgcagtggctcatgcctgtaatcccagcattttgggaggccggggcgggcggatcacgaggtcaggagttcgagaccagcctggccaacatagtgaaaccctgtctctactaaaaatacaaaaaaaaaaaaaaaaaaaaaaaaaaaaaaaaaaaaagccgggcatggtggcgctcgcctgtagtcccagctactcgggaggctgaggcaggagaatcaatcgcttgaactcgggaggcagagattgtggtgagccgagatcgcgccactgcactccagcctgggcaacagagtgagactccgtctccaaaaaaaaaaaaaaaaagaaaagtatgaaacgatgagtacaatgagtagccagccgcctgccccacctaccccgccgttccggactgccgcctgcgccgagcctaggccgcccagcctgactgaaactgccgctgctgaactcagcgtatccagtcccgcagctgaccaatcggagctcgcccttccggggcccgccccattccaggaatcgacctccgcaccggcctcgaaaattgaccaatcggttgtgtagctccgctcttagtcccgcccccacgctgaaactgacacttcggatcgccgagtaacgctcaccagacgtcccggccctgcccct

SEQ ID NO: 80 - DNA, 1166 bp, Artificial Sequence
  subsequence of HOXC4
cccggcctgggtccgaggctccgaggcggccccataagtgtgaatttctgtttataaacggcgacgcacacgcaaacgcctacacacccaggcagacacacccggaggcgtgggggaacatattccagacgaggtccgagagaacctgggggccagacttaccttaatctaagccaagacaccccctctacccccaccctctcccagaggcgcgactgggattctgcgccaagtcccagtgggggaggggatcgctccacgatttcctgtttaaaccctgacccgtcgcctgtgggggggcggggatgggggaagaaacctgtttttatcaaatttttaaaaatacagtttgcattcagatcaaaccgtttgggccagagctgacggtgaattttatctcccctccccccacaacccccttcccttccttttctgcccccgctaaattcccctttcgcagccatcataaaggcctctgaggtattctcccgcgggaagaaatggcattttctctccccctcccccacccccccacccccagtaggacttgtgtgtcggcagaggcgtctgacgaggggctaatttcgctttccttgtttacgatcgagaaaagcgcttggctctccccaaatgggcccagcccgccgcctgctcaggctgccgcgcgctctcccgccgctcaggcacggcccagtggctgatgccccgcggatccagcctgcggcaggccggaacccggagtctgtgggacctgaaaagggcaaagggagtgcggtgggacacaagaggggcccgagaggcccgaaaaagggagcctttctcctggagtaatgtgggaatcgccgaccggtgaggcctctatttctctctttcgcctgcattttgtccgcccccgcccctgtggatggccgctgatgggggggaacacaagtgtcctttgtccgtccccgaaagaaaaccttttccagagaaggccattcaggccagtggagattcacctagaatattagaagggccccctcccacccgaggggcgggcctgggcgaggctgaggtatttctcttaatcctagaacgacctcactgagtgtacccacagcgagatttggaggtgctagagttcgaggttggccgcttaacagcaccatcctgaggtgtctttatat

SEQ ID NO: 81 - DNA, 1334 bp, Artificial Sequence
  subsequence of HOXA7
ccggccccgcgccccgcgctccgcgctccccagcgctgcgctccccgctcccggtcccgctccgccagcctggcccgcctagcgactgcgcctacctgaagaccgcatccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccgactcctgctagtgagccctggaccaagcttgggatcctccccatccctctcctgtccgcctgcccagaccctggaagggtctctgtcccccgcaacagcctgccccgcggtggccttgtgggcaggactcagctatgagcagatcgactctgcccaagtcttctctcacccaggtccagtgggcgacaggccggacttagactcggatccagacggggaaggcgcagcatctcttgcagctgcagagagattgccaccgcaaactggagccatgtggttcgaataaagtcaacgtctcccagcttcctttccttaatcggaggcacactgtttatccgccctaaaggaagcagtgaaatatttatctattaatgagactcatttgccaacagatttattaacgtggggttcccctccctcctcccggacgctgtagtgctgcaggctctgtgccttc

SEQ ID NO: 82 - DNA, 1181 bp, Artificial Sequence
  subsequence of BST1
aaagagggcatctgtcaaagttgtcgtcctttggagccagaaaagtatttcttctgatgatgactgacctctcctcacctctccttcaaccttgcggcttctttggggctccctccccgtggacagcggtcaagattagcctgggccacagtgcactggtcctgtcctgggtgatgttccaagctcactcttttttaattttatttatttattaattttttttttagacagagtctcactctgtcgcccagggctggagtgcagtggtgcaatcactgctcgctgcagcctcgatcccctggggtcaagcctcctcacatctcacctcgcaggtagctgagactacagatgcgcaccaccaggaccagctaagttttgtatttttttgtagagaggaggtttctcacctgtatgttactcaggtttgtctcgaactcctatactcaagcgatccgcccgtctaggcctctcaaagtgctgggattacaggcatgagccgccgcgccccgccccacgctcagtcttgaaattgtctggaacgggaaacggcaaacagcgagatatccgagcgagagtcccgccctgcatcagtttgcggaaccgccttggtagaaggagagaaggggagtggaggaagcacgggactggagggaccaaagttccccgatggcggcccaggggtgcgcggcatcgcggctgctccagctgctgctgcagcttctgcttctactgttgctgctggcggcgggcggggcgcgcgcgcggtggcgcggggagggcaccagcgcacacttgcgggacatcttcctgggccgctgcgccgagtaccgcgcactgctgagtcccgagcagcggtgaggcagtcggccgggtggaaggggagccggaaagaggcaacggtggggagggcctggggaggggaaaactggcgctaaagttcggggtgagggggcaatgagagaggccttgaggggagaggtgagtgtggagacagcgatggtcctgaacgatgggggcgagtgaggagggggctgtgaagtgtgtctgcaggtgaggggtgcgctgtgcggggtctagagatgagtgtgtgtgtgtgcgcgcacacacacactgtagaggtgaggggtgtgtgcgtgtgttctagaggtgaggggggtgtgtgtgtgcgcgcgctctagagg

SEQ ID NO: 83 - DNA, 1039 bp, Artificial Sequence
  subsequence of C1orf114
aggtaccaccaacgaaatgaataccattgcctcctcatggaagctacaatttcgggagtgactttttctcccagggacaaaattattactttccacgttcaagaactttgagaggggaaagaatatgacaaaaatttagtctaagctactctcagacgagtatccttcatacatagtgaagaaaggtgttaccagacttccccaccagtgccagactcggtggacctcggactccagaggataaggcgccaccagaaagcttcttccctcactacctaccctccctcccagctataccagacgtttatacctttaccctctatcccaccattaacatctccgcatcgtccttcccttctctccctctatatccctcttgcctgtgtaaaatgcggcggctgggatcacagagatggccgggggagttctcctctcctcctctttctaagctcttcacattgaggcaaaggagaccccggcacctgcctccgcggcagccaggaccacactgccatggcaacagcgtgcctctgcgtcctccatccgggcctctctaactacgagagcgacaaaaatctgcgcaggcgcatgcaatatcctcagaccaccgacccctcccggcacccatcccggtgcttgcgaagggccagtgaagctgaggtttctaaggggacgactaaggaatccacgcatcactaacgttatagagactcgcgaagtgccgctgtgttggttgtgaaatttccctcttgtagctgtctctcgtcagatttagaatttcttaggacaacttctaaaactatatttctatatgttatgtgcttcacagttaacgtagaaaagtagtttttagttttcattagtagataagcaaattgctaatgagttaccatatttttgaccctcagcttctttttttcctaatcaaaaccaaaactttacccaagagagttgccaaagaaaataggatttttgctctagctttgaggcatgtcaaaatggtcaactagaattttctcaaattctgcttatctttatg

SEQ ID NO: 84 - DNA, 1027 bp, Artificial Sequence
  subsequence of FOXG1
ctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgc

SEQ ID NO: 85 - DNA, 1017 bp, Artificial Sequence
  subsequence of ATP4A
acgagggaacggggcggggctgaggagaggggcggggccgagagctcggtgcgggctctgagagctgcggggaagggtgaaggtggaagatggaggccttgtgtggaggggtccttggtagaaggtaagcgttaaggcggggctaggtgcagatttggagtcctggacgcagggaatgaacagaattagggtgcggagttgggctgggggcggatttggagagcgaggtgctccccatggacagtcccgccgaggagaagctgtgggcggggctgggtggtgggcggggccttgcctctggtggacggggccataggcggagcgggagatggggtggggtttggctgcggagagaaggggcaaggagcgaagcccctcgtggcccgctgatgtgggtgtggcctggggcggggcccgaggtgggcgggcccaggccgtgggcggggccggctgcgcacctggaacttgttggtggagttgaagggtatctcgcagacttttgggaagcggtcccggtagcccatggcgttgcccagcgtcagctccgagaacttgagcagcgccgtctccgatgcgtctccaatcacgatgcgctgggagcggggaccggtgtcaggggcgaagccggctacaccagcctcccgggattccctggaggccccctggctctcaccttgggcacaggcactgcatcctggccggacttgaaggcggcgcggttgcacagggtgagcacccggcacagcgcccgccacgtctccgaggactggtcaaacgtctgccctgcagaccaggcgtccaggctgggtcccgcacggcggctctcccggaccagaaccgagccccctcctcctaggctcatatcgcgggccccctccccagacctgggtggaattgggttccacccaaccctgagggacccagcccccggatgacccttccctctagacccggtagcgagtctcctttgagacctggagccgagccgccccgccttcgtacaaagccctccctacc

SEQ ID NO: 86 - DNA, 1161 bp, Artificial Sequence
  subsequence of NOTCH1
tttataaacacagaagaatcttttcatcctacgtaggaaaaccctggctctcagaacttgcttgttttctcagaatagataaaagtttctacctggggccagataaaacagtacatataaataaaaaggcagtgtttctgtgtaaaataaaagtacataaataaatactaaaaaaaattaaaatcctcgttcttattttgtataaaaacatgtgttttaaaaaggctcctctggtcggccctggcatccacagagcgcacacagacgcccgaaggcttgggaaaggaagccggggtctcgtggggcgcgccgtttacttgaaggcctccggaatgcgggcgatctgggactgcatgctggtgggagggctggagacgccctcggaccagtcggagacgttggaatgcggggacgagctggaccactggtcaggggactcaggggacggggtgaggaaggggtgctcaggcacctgtagctggtggctgggggtgttgtccacaggcgaggagtagctgtgctgcgaggggggcgtcaggaactgggctgcggtcacgggtgggaccagcgaggatggcagcgacgtgggcagggcggggctctcctggggcagaatagtgtgcaccgccaggctgctggggcccagtggctgcacgtctgcctggctcggctctccactcaggaagctccggcccaggtggccgctggctgctgagctcacgccaaggtgcggctgtggtggtggtggtggcggctgcaggctttgctgctgctggatgtttgctggctgcaggttctgctgctgcatctgtaagttttgtggctgcacctgctgggtctgcaccaggtgaggctgggtggccagccgggtgctgggcaggccctggtagctcatcatctgggacagggcgctggcagcaaggctactgtgcagcgggcctaccatgccatgctgcagggagggggcctgtgtgctcagggggcctggtgccacactcccccgcagagggttgtattggttcggcaccatgccgctctgcagccgggacagccactcgcattgaccattcaaactggtggacccgcccacagtgaaattcagggcccctccgctgctggagcccaggacggtgctggtgccagaggccacaggcaggtggg

SEQ ID NO: 87 - DNA, 981 bp, Artificial Sequence
  subsequence
ggttccgccgaatgcgtcctcctggcggtgatggctctggaccgcgcggccgcagtgtgccgcccgctgcgctatgcggggctcgtctccccgcgcctatgtcgcacgctggccagcgcctcctggctaagcggcctcaccaactcggttgcgcaaaccgcgctcctggctgagcggccgctgtgcgcgccccgcctgctggaccacttcatctgtgagctgccggcgttgctcaagctggcctgcggaggcgacggagacactaccgagaaccagatgttcgccgcccgcgtggtcatcctgctgctgccgtttgccgtcatcctggcctcctacggtgccgtggcccgagctgtctgttgcatgcggttcagcggaggccggaggagggcggtgggcacgtgtgggtcccacctgacagccgtctgcctgttctacggctcggccatctacacctacctgcagcccgcgcagcgctacaaccaggcacggggcaagttcgtatcgctcttctacaccgtggtcacacctgctctcaacccgctcatctacaccctcaggaataagaaagtgaagggggcagcgaggaggctgctgcggagtctggggagaggccaggctgggcagtgagtagttggggaggggagaaagtattaagccagaacccaaggatggaaataccccttagtgagtcagtttagacttcaggctgttcatttttgtatgataatctgcaagatttgtcctaaggagtccaatgggggatatgttttcctcccgtgaggaaatgtttagttcttgagggaaaatccctaaatcctctatatactcaggtttagggaaggaaaacctacccctcacaactccacgcgcagggaaaatgatggacgtgatgctcgcctttagcttcctccctatctgatggaagaccatggaagacctcttggtctctgcaatcagaagtctcaagttgacaagaaa

SEQ ID NO: 88 - DNA, 864 bp, Artificial Sequence
  subsequence
aatgcccttctttgaggcagctggaggcctcctcctcctgagagcttaattgtggacacagccttgagtagaggcgaaaaggaaagaaggctgggagacaatcggggaaaaacacacacctttggtctggtttgaaagctggcgcccgaaaaggaacaagaaaaaaaacacgcttcggaagtgtctgagattgctgcggccataaagcagagcactaaccctggtactgatacgggaggggagcaagactccacccccgggcctgtgcccatgaacctaggtgaggaggggcacttcttcctccgcgcccaaatgttgcatttcccaagaccaccctggcccgccacgcccccatcctgtgcctataaaaacccccgagaccctagcgggcacagacacaagcggttggacatgaagaggaacacaccggcgggagaacacagaacacccaccctctctttagtcaccctggaggcctcggagaaggcggtttcagtgatccgtgaagagaccctgagtcttttacatcccggagaatgacaagagagtacctgggcgacgtaactttctgggttttccggagatgcggttttctacacgccaggggccaagcctgagaaataggtggacggcaaggacaggagctctctccgcgccacaaacgcccctgctcacacctgggttccttgattttcctgcctatataaccgagccacccatggccgggctccgagctctcattctgcggactgggacaaaggggttaactgtgatgtgctttgtataaccccaggattctgcacttgcccaagggcgaggtgcaaatccaaaatcacgcacgactgaacgccgagatcagctgag

SEQ ID NO: 89 - DNA, 1004 bp, Artificial Sequence
  subsequence of HOXA7
catccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccgactcctgctagtgagccctggaccaagcttgggatcctccccatccctctcctgtccgcctgcccagaccctggaagggtctctgtcccccgcaacagcctgccccgcggtggccttgtgggcaggactcagctatgagcagatcgactctgcccaagtcttctctcacccaggtccagtgggcgacaggccggacttagactcggatccagacggggaaggcgcagcatct

SEQ ID NO: 90 - DNA, 963 bp, Artificial Sequence
  subsequence of ZNF529
cactcacaccaccacacacggacgcagccacagaacggcacacacaaacacaatcacacagtctccctccccagcaggtgacttgcacacaggcctggggcttctgcctccacaaaccctctccgcgtcccatgaacccaggtcccctgccgtgagctcttctcgcatccagacccgggtcacctcaactcaccacatggaaaacgaacaccaggtcaacggaaaagggagccggggtcctggcagaagcgtagtgccagccgcaatagggcggccgtgggtgcaaacggaggggagcgccggcagctagcaccgcgcggcgactaacgggccgccccggagactcctgggagctcaggcccacgcgcgagtgcgcaggcgcagacgactcaccctgggccgggggtgaggcttggtgggtgccaggggtgctgaggaggccaaggagggcactgggagctccgttccgggagagctgtctaaggacgcggaaccgggtggggaggccagggaggtaaccaagagtccgagccgggcctggaaattctgaggaggaaaccgggaccgcggtatccagacgctccgatctgtacgagccgggcctcgggcgtggtccatgggcgtgaccttgagggtgacccggagcgagctccgcgaagggggattttgcccgttctgatcccgcggaagcgccatcgctggcggcgggtccccgcgcggagcgggcgctgtgtgttcgcgcgcgcgggcgcgcgctgaataggggtggcctcctgcgcccaacctgctgcattttaggtgcttgcctctctttgggatgttgtggccgattgtgtgtgaaaagtgaacggagtagtgttgctgcgcgaggtttcctctgcgtgtgtctatttttagattaggtgtgagtcgcatttgagactgcctttgagtgtggagaattgcaagtgatttgtaggtgtg

SEQ ID NO: 91 - DNA, 956 bp, Artificial Sequence
  subsequence of PRLHR
ctccgcgggagctgaacggatccaaatgtttcccagtgggctagcagcttcttagagggactgggagcgcctcccctcagctccgccccccactccagctcccatgtatggaaaataggggtggagcgcgctctgccatgctcggtgcttagagaactagtgaagaggacagtgtggggaggatggaatccgaaacgggaggaggggccggcaaacctggccagagcagcgaagaggacgaatctctactgcaggagcttgcacggcccctggagggtcagggtccggaggagcgcacagtggaggagggcggaggagaggcgcgaagcgtcaggctatcgggaggagccggtgcagtcttaggagggaaggagcggggaaggaagtagcggtgggagattgcaatgcagctgggggcttgagagaggcacgactgacccgttcttaaaggctggatcctgcctagcggccaggttgcggagggcagagacgctagagggctcgggctgggatttaacagcagagcctggagctggtcgcggacctgaggaccctcaccctcccgtggccccgccacacgagccccctgcttggctgcagcgcgctcagcgggcagcgggtccggccaagccaaaggcaggagtcagcaccacggacagcttccgctggatctgccgcccctcgggcggatgatgtggggaggagcaggggaaaatgactagggaattcctgcccctctctggatccgggtgtttggggaaggaacctggtccggttcaaagacctgcctcgctgcctgctttctgggctggggctgagaagagtagggtggagtggaggcagctgagccgggggcctgggggtcggagactcatgacttttgctcctcttggtggtcaccgcactcagtgccaaggacggcgtcgtcttttacctcccggggtcagaaaatgcag

SEQ ID NO: 92 - DNA, 1037 bp, Artificial Sequence
  subsequence of ARL5C
gagggagcgaagagcagggaggcagtttgggacacttggcaggcctggagctgggtttggcagggcccccgggaagatggaggaaagcagtgggagctagggttgcctgtacggtgcaggatttgaatgccagaccgaggagccgcagtactaggaaggtccaagcagcgtgcccaatcgctcctccggactacagaggagcccgggaacccagggaagagctccagtgattggtccccctgcctgtcagtctggtgggaggcccagctctctaccctccccgcccgagagtcacttacaaccggtagagaatggtggtctttccttcattgtccagtcccacgatgatgaccgtgtgctctggaagcgtcggaggaagggcagcgtcagggccaaacccgaagaccaagggactgtgtgcccccaccagacctcccaggaaggagatagcgctccccgcaggctggggacctctgcccaccgtaccgcgtctgcttcccacccagctccgacacctgccccactccgggacggtggaaaagacagctgagcccccacctcccttcacattccagaaaagtgtctgaaaggcccggggcgcttcggggcttgccaagagacggtgtttagagaaagagcataacgcgaagtcacaatcgcaggaaactcgcagcagccccccatccccgccgctggctccgtttagcggggagaaaggagggtcgcccagctttgcgtcctggggcgcaccgaagcgccgggacccaagaggagcaggcagggacgggggcaggggaggccgcggggcagtcgaggagcgcccatatacgaccctcggagcccgaggtccccccgtgcgtccgaccacagatcagaggagtcccagaagagggcgagatcaaagcgctcgcttggatgccctgtgcgccccttaccctggttcccgaagatgctcattaacttggcgatcagctgtcccatggcacttcccgggccggacaggtgcaggagggctcagcggcctcaggagcggagtc

SEQ ID NO: 93 - DNA, 7709 bp, Artificial Sequence
  subsequence of PAX9
ctccgcttcctgttttcgagggattagctaggactgtgcaacaccagtctccgctctcggagtcggcacgggggctggtgggtggcgggatgaactcggggaaggcgacaggctggaaacgcagctcctcgactttgcagcgcaccaaccccctatcgccccgtccgctgctcaggtgcgaggattcgccagacgccacaggactgggggcgactcgcaagggcatcatgccacctagaggaaacgcgcagtgagtgagccctgggtcactgaaaatgggagaacgttcgagtcggtcaagatgactccggcctgcgagttgattcagactcctcggaagccagaaattgggtctagagtgggttggtccctccgtcagcagttcgggcccagccgaagcttggggcctagagggcagacgtccggctctgatcagagtacagcgggctgggcctggcagcctgcaggcaacctggaccttgcaataaagctgcattcaacataactcagaccacagtgggagcccccgaagggcttcatgtgagtcccgaaaccagagtttagaggcgcggcctggttctcgtttatcttttccagacagcatttaggccaataaggtctttgaaaggaggcagccccaaagccctcaggccgacacctgagggcagggtctcaaacagctgtgcttgaagaaccggcaatcttgcttttcccacacgcagctgcacctctcctgagcgctgcagctgatggctgtatagtgaccacggactgcgtcctcctcacgaagctgcgcaacttctgctaatgctgcaaccctggcaagacgaaatccccctccctctggctagatcagcccagcctcccctcagggaccttcctgcccctccctacccctccctacccctaggccgctccccccttcctctgttgggatccgatccttgttcgctcttgcaatcccgcagcccggagttttgccccgtgcattaaaacatgttctctttcatttgcagatttaataaacaaacgtaaccatatcacatggagctccacactgtccttctgaaagcaattccgctgttgaaaactgaaaaatgggtgtaatttgctttcagaaagtaatgttagggtcacggcaatttctcgggccgttatttatttttactttaaagtctttaggaaagatttgcttatatgctcggaaaacttccaaatgcgcgaataccagcaatcccgctcgccttcaacgcgtggggtaagggggggtggggaacaattactgagtgacgctaatatggggaaactgaaaagaaatgtcgattgtttttattgtaatagaaggagtgagcaaacagaaaaaccaaccccgggtgatcggaaacaggcaggcggagaattaaaagcgggtttcagacagcaacaggcccctcccctgctctcgcaaggagaaagcgggcgacgagcgctcgcagcccgtggggctgatcccacctcccgcagggctccggtcgtgtcgctgtcggaccgcagaacgccgggacgcatttccgagctcgggcccgacccaacccgctgccattctagctacctagtggagccgcggagagactcttgaaccgtgaggctccttgatgagaaggtggagaggctgccgggctattctccgagcatcttccggccgagctctgctcctctccgtgcgcttgcggcttctccttgggctagcgcgtccgcagtccctccaagtccaggccgcaggtcagtgtgcgggcgcctcctgtcctcctctcccaccagaggacccctgagggctgtctccatccccaatccccgcgactgctccttttagccgccatcccctcccgcagcgcagatcctgcaggggccagcggtccagggcggaccctccctcgccctgggcgcggggcgagcttctggctgtcacttgccgctgcagtttcagggaaactcagctggccgcttaccctgcaagacggggacatagcagagggccgagttctggagaagctgggcttgtttttctctctgcaccgcgcgcaatgccccaactgaccctgaagggtcttgcctttactgcatttgcccccagacggctagaagtcaatgcactatgaaaatcccagtcccggacctcggactctggctgtcttacaatccctgcaactcctccgagtggacaagaggcgggcaccgtcgcctgggcctcgcttggggggggggggtcctgtccccagcagggggcaagttggaggaatcagctccttaatattttctgctagctctgcttgtcataactgcaactcggaatagaacaacctctctcccgtgcatacgcacccccaactctcgccctatcctcccatccacagtcatcactgacctgtagacccggaggagcaggcttccgggggtcggtccaggttggggatacagggaggggtcctcgcgttccgcacaacctgtagtgcttgggtctgacttcggcagccctcgggtacagtctgccgggctagattcccgttgcttctccgcgtctctccgcgggccgctctagcgccgggaagggggcgccgcaggtgactgcccgcggcactgggacggctgacagatttatggagacttcacagcatctggctggggtccgcgagggcagggcggagtccggtcgatccgatgctggggtctaaagcttccccagccacacgcctgggccgagtcgcccccagagtccccggcccctgctccccttgggcgggtgtacacaaggatccggccagcgccgagtgtactggttcctcccacccagtgtagccccctccttcacactctcggcccggcttccagtctcccactgagtaaatatttatccagagagcgagtcgcccctcggaggaggcctcgacgcggctagggaaaggctttttggcttctccctggcctgagccagctgcaggccttactggtgttttggttttggtcttaaatttctccttttagtcgaaattcttccgatctctttgagatcttctgagtccagagtgagcagtgtaaccaggcggcagactccagctgacccttgttcttgttctaggtcctgcatccttacattcatcgggactgaccagaccttagtgttctctcttttccttccttcatcctctttctctgcctcagatccgtcgtctctcggtcggtctcagcgggtcgctgactacctcgccctagcactcggtccgcgccccgggaaggcttcagcatcaccctcttctgctgggatcccgctgaccctgtctgcagcttcggcttctcttgctcccagagcggggtcttcggcttgccgcggatacttagtggcccaaaacacctaaaattgactcttcctgcgctcttgagaatgccaagagcacccagtacctgcaaccgccggcatgagtggggcctgcggggagccgccagagctgcggacaggccccctgcccactgagttgctacccctggcacgcccaggggatccgcagcagctgaggccacatggtcacagccaagttcagcaggtggaaagggagagatggcattgaacctatgtgagaactgggaagatgtgtggcagctgaaggagttggtaggaacaagagggagttgtatgttacctccatataaccagagctctaaaccatatcagtttctgaaccagtctaggctggtgctgcagaactgctaaagctctcgcagagtccccagaccccccgcgggacatgaggtcttgcctgttcgtatgcgaacatccttgtacccgcctagcagccctgcagactgcaaattttccctgggtgcacggaagtagagcagatttggcgcagaggtcgatactcactgcctgtctgtgaggggggaggaggattccttcctggtcaagttgccgggcatgttcattccctcccgcaagcctaaggcagagtcataagctcccctacgatgcggcaaagtctcaactaatttatctaccagccttaagaaggagctgctgcttctaaaatctttagatttccgaaggctctccttgtgtaaaaatgccaggcggtgccacacgatgtcacccaacaggtattggaggtcagaccaggctgaaattgctatcaagtcaaacttcaagaagattttaaacatttgaaaccaaccaacaaaaagggacaagttcaagtatgtctagttgtgggatttcccccaagattccgattctaaagagtggtgttttagttcatctttacctttcacatcctccattctgttaggtaattttgaatttagagtgcatcctaaccatagaagtgggaatctggagatctgttcctttgcagaagcatggctgatcatagttttttcctgggttcttgcaacttactgacctgagatctgggaccagtcagcttctctgggtgtagaccgcagccacacacatccctggggactgttggggaagcagcctgaatcctgtgtgcacaaggagccttctggattgtcaagtagcagtaacagagttcacgaccccaagcagtacaaactcccaaattcacgaaaatatttctcttcttgtttattattttgattcacattctccacaaccctaactgcaaatattaataactttggctgcaaaaaagcatacggtacactttagattagacttacagtgaataaaagtgaaaattcagaagtaggcagaacgacagggcctctggagcttaatggaagacgagggaattacatccatgtgcagcgtcggtgtccactgtcttgggactaagtctctttcatatttaaacaaaactctttcgagagggctcgctggtttggagaaccctcacaaacgctgccaccgagggccaggggcccaggacagtggctgaatgtggcagagccttgggctccgtagtgagtgccccacgcactggcaattggtcgacttggggctgtgcgctagcgttagccgcctgtggttcagttccccgcacacggcgtggcccgtgcctcgagcctgtgtggctagtggttggccggtccacctggactggggggtcgccgcaggaagctggcggtaggaggaacctccctggccttaacagtggcccccacctgtggctcgggctccccgcaacctcctggtttctttggccggcctatcttctcacacctgcgctctcgccacctggcatggactgaagtgaggtagaatcggcaggtgacacagatcctctcctgacagtccccagaggcccgcatgacagattttgctaagacaactccctcacccctgtgcacggtggcgcccgcctcccacctatagccttaacttcaattctgagatgtccatcgttccccggaggaggtctcctcgggccccttctcccttcgagtcattcacattcagaccaaacgctttcatttgtttattcagcattttaccagcctgattttgctgtctttttgtgccgcttctgggaagatgtcaaacacccattttcattgtatttagtcacccaggtggggagctagcctgaaagagacagcggaaggagtttcctggactgcgctgtcgctcaccgacccgcaccaatcaccatgcagcttgccctcggacccgccccttttagggcgtgtccccagtgagtgatagacggagccgccctgccctgctcagcccagcccacgttgctgcttagattgaaatgcagaactcaagcctctttcatcggggcacagacttccttttacttcttccttttgccctctcgcctcctcctcctgggaagaagcggaggcgccggcggtcggccgggatagcaacaggccgggccactgaggcggtgcggaaagtttctgtctgggagtgcggaactggggccgggttggtgtactgctcggagcaatgggtgagtggcggcgggggactctgtcagagccgggaagggagggagggagcgagcgggcaagggagggagggagggagggagcgcgagggcgcgcgccactaggcgctcacattctctattgactttgctcgtgttcctacaagttgtaggaacacgctaagggtcagcggcgcacagcagttcaatgtgaacgctggctactgggtggctgttgatgccattttctgattttaagagaagggagctgtggcatcagcgagcccctcagcccgagtagcaaaccaagttttgtttcacacgcgcgcacacatgcaaacaaaacaggaaaagaaaagaaaagaaaacaaacaacaacaaataaaacaccctctagcttcccctagactttgtttaactggccgggtctccagaaggaacgctggggatgggatgggtggagagagggagcggctcaaggactttagtgaggagcaggcgagaaggagcacgttcaggcgtcaagaccgatttctccccctgcttcgggagacttttgaacgctcggagaggcccggcatctcaccactttacttggccgtaggggcctccggcacggcaggaatgagggagggggtccgattggacagtgacggtttggggccgttcggctatgttcagggaccatatggtttggggacagccccagtagttagtaggggacgggtgcgttcgcccagtccccggatgcgtagggaggcccagtggcaggcagctgtcccaagcagcgggtgcgcgtccctgcgcgctgtgtgttcattttgcagagccagccttcggggaggtgaaccagctgggaggagtgttcgtgaacgggaggccgctgcccaacgccatccggcttcgcatcgtggaactggcccaactgggcatccgaccgtgtgacatcagccgccagctacgggtctcgcacggctgcgtcagcaagatcctggcgcgatacaacgagacgggctcgatcttgccaggagccatcgggggcagcaagccccgggtcactacccccaccgtggtgaaacacatccggacctacaagcagagagaccccggcatcttcgcctgggagatccgggaccgcctgctggcggacggcgtgtgcgacaagtacaatgtgccctccgtgagctccatcagccgcattctgcgcaacaagatcggcaacttggcccagcagggtcattacgactcatacaagcagcaccagccgacgccgcagccagcgctgccctacaaccacatctactcgtaccccagccctatcacggcggcggccgccaaggtgcccacgccacccggggtgcctgccatccccggttcggtggccatgccgcgcacctggccctcctcgcactccgtcaccgacatcctgggcatccgctccatcaccgaccaaggtaggggctcagaggctgggcgtgtggatgtgcagcgtctgcccccgcactctcgcggaggtcccagtatctgcagcctcagggacactgtctttcccaccacctgaggctttttcctttggaaacgtaaggagtcttcctagggggtgttctgatctcattaacttgaaactcatgcccctggtttccttgccacacacagtcttctgcctcatctcaaactaccagacccataacatccccccatccccaacacatggttcgcattttccaccctcccccgcctctcgcgccgaggcagcctcagcccggcttgctcacttgg

SEQ ID NO: 94 - DNA, 6438 bp, Artificial Sequence
  subsequence of HOXD10
ctgaaatggaaaggtccccaaattctgaagacacatcctgcctattctggctcactgcagacctacccgcccgcaaggaattgcttgtttttagcctgtatgttttcttcatgtataaataaagattatgaaagggcatttgatgggcataaaattcccgattagattaaacagtcttgcctttcttagggtagggaaaattgggcaaacaagttcccagcagctcttctggccaagaggcaaaagggcgacactcgatactgctggagcgccaagctagtgtcagggcgcctgcggcaagcgagtgcagcgggcacttcattcagctttgggcacgcttagccctggcttgaaacaagatctctactatgtgagaaagcaaatttcagggcgctggtcagtggctgttagctttgaacagtaccttagcaacctagagtcaaaagttatgttttgtcttgttttgtttttccggccatcctcaacccccacccccaagctcgaaaacagccacccctgtggaaaggtaatgtttaaaccccaggctatgctgggatgctgcagccaaagcagttagaggcgccaggcttttaacctgacaatgatgttgtatttgaacagctgcgtaaaggtttaaaaggtctgtctcattgccaccgcggagttttttaaaagaggttattgtttgggcgagagcaccgagagggaacaggcgaaagggtcccaaggccgaaaataatgttcaacgcttgtttcaaccaggccaggggagattcctttttgggaagagggacaaagggttttttgggaaaggtttttgtaaaggagcgggcgggctactaggtcccccagaatgctgaggcgctttaatgaacctcaggccccaaagaaatgttctattgaggtggaaacccaatttacccacaagttcagtctctgggcctgaaatccaaggccagcccatgccccagcgagtacggggaaaagagaacacagatgccctcccggactgagaactggttcatggtcagagtttctgggaccgctttagtttcttggctggcaggaagggaacctggcaggccgaaggacggctccaagccctggccggcccctcactcagggcaagtctttggaaatcaaggttaaagctccccgctgctgccccgaggcccgggcttccgcagggcggctcacccgcctgcagcaagagagggagggaggaagttgttgaagtttctcataaaactctgagtttccatggtggtagtggtggtagtggtggtagtggtggtagtggtggttctttaatgagccggaccacaaccccagaccatatcctcttcatagaagtttccctcaccaaaggaaacagagaactaagattcccacctccttcccctcccttacccaagcaaagccttgtcaaataatttcagaaattgtccaaaactgtaaaactcaacttctgacaaaagtatagcttttaatatttgacaatttgtattaaaacaaaaatgaccttcttggttagcagcgaagggaaaaaaatcaatagtataattttcaataattcataaagcgaacgccattatggtaaatttaactattaatcttatcctttacaaagtctcgattgatttgttaataaagtatcttcccgtgtgtggcaacagcgggcataactctttaaaaaccttcagaagcaagaaaattaccaagtagcccaagaatgtagatgagaattttattgatcgccctgattcttcttaatatgtattaataaattccacaaccttttgtaccttgcacttgtcagaaaccagtgcttttacaaaatccataaaaggaaaacatttttctttgcagaagaaccaaaatgacacctttgcatttctctctgtccttcttggcccagtcagttttctaaaacttctggattctgagggtcagagtttccccttttcttaaattattttttcctttcttccttcctgtttgccctccattatcagggtgtggggagagaaacctctgtttagttctcctacagggagcccgagggacgctttcgcggatagtgaactgctggcaaagaattgtctctgggctgaaagctcccagaggtccggccccgcagaggtcagggtgtggttcctctgagagccgaagcctcgtgtagccctccggggcgcataggaacccggagccaggcaggaggaccgagcgggaggacaacgcagggaccaacccatccatggggatccgggcccggccagctgcttggcctcccggccctcgcggccctttttgcctccgccctcctagagacaccttggccgcttcttagccccaagggatctttcctttggacccctggggtgggatgtctcagggcccgcggaatccgactcaccttcccgctgggctgcccgggactaaattaaccagcctgcgcccccacccgcttgtcctggacccggccctcttaagcgcgttctgcctggttgtgtgtggggggaatgcttctgtgcgctggcgccagggcactctggcttccctccccgtccgtgcgtgtccacttggaggcccctagagctgagactttccttccgccggttgggcccaggggccgaagcgggggacgcgagtggggcgggctggccgagcgagccctggagaggcggacaggagggcggcggagagcgctgggccggttgtctccagcgcgcactatcgcgggcgcgtagtagatgtcgctgttgtccgtgcttacccggccggccggccaggctctggagcacgtgacccgagaggaggctgcggctcaaggccattttcaaatctcattggcttggttgtcatgtggtcggcagaggcatccacaattacacggggaatgttttcctagagatgtcagcctacaaaggacacaatctctcttcttcaaattcttccccaaaatgtcctttcccaacagctctcctgctgctaatacttttttagtagattccttgatcagtgcctgcaggagtgacagtttttattccagcagcgccagcatgtacatgccaccacctagcgcagacatggggacctatggaatgcaaacctgtggactgctcccgtctctggccaaaagagaagtgaaccaccaaaatatgggtatgaatgtgcatccttatatacctcaagtagacagttggacagatccgaacagatcttgtcgaatagagcaacctgttacacagcaagtccccacttgctccttcaccaccaacattaaggaagaatccaattgctgcatgtattctgataagcgcaacaaactcatttcggccgaggtcccttcgtaccagaggctggtccctgagtcttgtcccgttgagaaccctgaggttcccgtccctggatattttagactgagtcagacctacgccaccgggaaaacccaagagtacaataatagccccgaaggcagctccactgtcatgctccagctcaaccctcgtggcgcggccaagccgcagctctccgctgcccagctgcagatggaaaagaagatgaacgagcccgtgagcggccaggagcccaccaaagtctcccaggtggagagccccgaggccaaaggcggccttcccgaagagaggagctgcctggctgaggtctccgtgtccagtcccgaagtgcaggagaaggaaagcaaaggtcggtatgagcagagttgccaccccagcggggcgcgcagcccgggaacccggcagagagggagtgccggggtgcccagcgccgagccggagcccgacttggcaggtgctgctccgcctggttttagaggggtgatctcagccctgagatagtccccgcttctcccctgctgccctggccctctccgccagtcctggccccacgctgatggcgcccgggcagaggaaaagcttgccggttttatttttcctgagctagacctgaacacaacaaaagagcgcaaaggagacctgcggctcataaacacgaccacagagcctcttttctcctgctcagatttgcagttccagttttgccttgagcccaatgatcatgttaaggtgatccagggcaccgtgttcgtgttcaagtgtatgcaccccgcatcctgcgagcttgggggtggtgaggggaaagagatggctgggctggttggtgcttgagttgggaaacagggcttactgcctttgctgggctaggtaaccttggctttgtttaggaaaagtgctgcagtctttgcaatccgtcggcaaagagggcaaaggcggagggggagagtggaacccgcattgccctccctgcaaggccagccttagggctgggctaaggcaaagagccagggatctggctttttgagaaggaaccctcctcctctcccccagtgcttagaggtgggccacagtagggggctccctttctgggggaatgctttagtgtgggggcaagaagacatgaaaattaaggaaattctggggaatgcaacaatacccaggcaaggtgggggaaggtgtctcgcttccccatttatcttttgaaagagaatgggcacctataaacctgactgtcaggattcctgactgcctaggagaggtggggaagaagtggcagatttggggacctgaggcagcagtggggttggtaggcttgtccaggtcgtggcgtattcccctccgtccctgttaggagctgaacccttagaatgttgctggggagatctggaaagtttactattctactaatgttttgtacaagtgagaaagttgaaagagagagcgagaacccaaatgcagactgtcctgccatcatgtcatttaagtaatgtggcatcaatgtaagattcccttccaaggcccacttcatgtgagtaatgtttaatactagcattttccaaagcggcctggctgccagcagggtcacggccaagggtacatttgaacagtctgaagaaaaaaacaaaaacgaaaaccaaaaccaaaaccaaaacaaaaacaaaaacaaaaacaaacaaacaaaaaacctcttgatttttttcttcttctccctttaattttgttagaggaaatcaagtctgatacaccaaccagcaattggctcactgcaaagagtggcagaaagaagaggtgcccttacactaagcaccaaacgctggaattagaaaaagagttcttgttcaatatgtacctcacccgcgagcgccgcctagagatcagtaagagcgttaacctcaccgacaggcaggtcaagatttggtttcaaaaccgccgaatgaaactcaagaagatgagccgagagaaccggatccgagaactgaccgccaacctcacgttttcttaggtctgaggccggtctgaggccggtcagaggccaggattggagagggggcaccgcgttccagggcccagtgctggaggactgggaaagcggaaacaaaaccttcaccgctctttgtttgttgttttgttgtattttgttttcctgctagaatgtgactttggggtcattatgttcgtgctgcaagtgatctgtaatccctatgagtatatatatatatatatatatatatatataaaaacttagcacgtgtaatttattattttttcatcgtaatgcagggtaactattattgcgcattttcatttgggtcttaacttattggaactgtagagcatccatccatccatccatccagcaatgtgactttttcatgtctttcctaacacaaaaggtctatgtgtgtggttagtccatgaactcatggcattttgaatacatccagtactttaaaaatgacatatatatttaaaaaaaaaagattaagaaaacccacaagttggagggagggggacttaaaaagcacattacaatgtatcttttcacaaatgaatttagcagttgtccttggtgagatgggatattggcgatttatgccttgtagcctttcccttgtggtgcatctgtggtttggtagaagtacaacagcaacctgtcctttctgtgcatgttctggtcgcatgtataatgcaataaactctggaaatgagttcactccctctgctttctgaaatggaaatatgttatggtggaaatgaaagcctatggtgagattatcttctggttacactccctgtttggggcatttgggcaggggagtgatagactagtaggggaagggagatgggggagaaaagctggaggaggcctagggtgttggattttggcagtggttgggggagaggaattataagctagcttgagagtgaagttttcataattgggaggaaggggagtctcctctttcctttcccagtccccagtgatagtaacataattgcgctctcaatgggtgtgagctttcctctggcctgaacctggtaagtaagcctataccccaagccactttctcctcaaagc

SEQ ID NO: 95 - DNA, 4212 bp, Artificial Sequence
  subsequence of TBX20
taactaaaagtagaggaatttgagaaatggttactggttagtgtttttgccaacctcaactgaaaatgacgacagttataaaatgatgtcttggtgataagtctagagcttcagtgaataaaatggttcgtgtagggagaactggcttattctgctttacagtggtttcaaagagcttgctctgttttgtctattcaagtcagaggaaaaccaaaattaaactggtttcttatgaagtcctttagcgtctccgtttcaaacgcacaagcaggtgttacatgcacagtaaatcctgttgagccttaaaaggggttttattattatttttccatgaatgtttttaacccaacaaacatacacacacaccatatgtctgatctgatatatggattctagacaacttaggagaccgacgggcccagatataagatgcacattcacagcattcaacagactgaagcctccaaagaaaacttggacccaaaagaaaaagatctcccacccgcgatgtatggctcatggcttgagcatcagacgttgctgcgagccgcctgcagccaggggcacagacggatgcgcagcatccccagtcctcggcggacagccgggtagcccaacttacccaggggtttgattgtgttctccgtcgcctccttctccttagagccgccgctcgacatgagcgcggcaatggagaaggcgttggcccgagaggagagttggggcttgggggacgccgtgaactccatggtccccagcacgcggtcctggccagggacggggtcgtccgaactgccgtccagattccccaagggagacaaagacccgaaacacagctcaaagtttccgagagcagtcacagcggggccagggactccagaagtgtcagctccaacgactccagagctgcacactggcctctattccccaccgcaaagccccagagccgcagagacttcgaaggcagccggagaggagagggcccaccgagcactacggcgggtgcgcacgccccggggcgctcggcaggacgacagtctgcacagcccgaaggcggaaacgagcatcaactgcacaaagtcctggggtcctggagcatcccctccgcgtccttcctccctctggggctggggacagccgggatgtcccaggctgaggtggccaccagccgagcgcggctgctaggacgctggcgtggggagcgcggcgcggaactacggacagtgagccctggcgctcgctgccctgcgccttaatttgctggcggcggcgatcccggaggcccgcagccagtcagcgccgtctcacgtcaccgcttcctgattccgccgccgggggcggggccgcgggccgggcgcggagggcgcgcccagggtgcggcgcccgcgtggcctgtcgccccggctgttcggtaccccagcacaggttcagggaaaagggtgccaccactaggctgacgcagcagccatggacatccccacctggtctcacagccccgggcgggagggcggtgcgcgctgtcgggagaggacacatcaccgcggcctaacgcccgagatccgggcatggcccagttggggaagctatgaccagaccagtggaagcctcgggagttctcgctttttccattctttttcttacctgcgctcaaagttccaaaagaaagtagggaagcgctcagtagaatccggcgcctctgagccggcgccaactggccaggtccatactcggggcagatcccagggctgggactctcattcgatggtcccaaatttgatctctttgacagtggggctgagcaaatgcagccctgaaaaagataagcttagcgagagctgcgggcatttggcaaaggatgctgctctcagaaccgtggcttctgctcctcaccagtgcgtctctagcctggggctcctattttccctagagggaccctggaaaatgggacggctgccgaagagaaggcgaaggtcagggcgcggagctctacggtaaccacgcgacgcgtagtgggagagtgcgaaccttatggtagcgcccggactaagcggacacagacccctctggttttacaaaggcggctgtgacgaggacttcccacggccttgttggagggcgtcctgttagccctgctcagcctgaataactcaaattctccgaattcgcagggtaatccgcttcgcttggagccgtgtaaactcaggctgacatacacactcaagctgtatatgtaaatatttgttctcttttccagccagtcggtctggctattagaagcctcttcgtgggtcttaaaattaaaataataataataatttttaaaactccaacttttcactgcaccaccccctactcctgaaataaatttgtgagggaaaacggcaagttaaaatagctgtcgaaaacgtttgtgtgaacaaaatgatgacatctggaagagtcgtgatttcagcctgtcttggtcatcgtgtgtcaatagtttttaacaaggtgctggatgaaggtgactaaaaaatgcattctcatatcactatcgaagttcgaagtgtcgatttataaatgtttgaggaaattcgtaaaaccatagactttggtgctggaaggaatttacgctgtcttctagccggtctccatgtgcgtttgagagagaaaaacagggcccgacgtggcagagcgcgcatctccccccagtccacctcagcggctagaagcagacggaagggcaggactcacactccgggccccgaccctcatcagcccctggccctgcagctgtggctgggctgttagctgaagagccagagtagagttaacgacggctggcaagcagagggctttcagattaaaccgcaggatgcatctgaggatccgtttctattcttaaattttttgctggttttggacaacccagacccaatccagaccccttttttggttattaaagtcccccgccccaacctgtctctccccagacacaccttctcaatgtaagtgacatttctttgtattgagtggaaggcacacactgattcctttcctatatcaaatgcttaggatttgtccagaaagaaagatttccctcaagaagtggggattttggaaggtcggggaatgttatggttagtctagaccttgatatatgcttcaacagggtgtggcaaaccttgttagctgaatttagttttcaaaaacttgccgcaaaattatacttcataaaaaaacccttttgattgtttttctacacagaggaaaacaactgtgtcttttcttaattacctcaatgattttgactggaatatgaataatatcatgagaggaacattttcagaaaatttgatgaagcaagggaaaaactgaattgccacaaatctgtttcctcctcctttccaagtgcctacgcggccttagcccagctctgtgtctcttgtgtgcaggaaaccccgactgcgcatcctactacaggtaaaatactatacaagaaaagatgagtgatcgttaggaggcaggcggcaaacgagaggtattgaaaaggccactttaaacaaatccgggtgacccgtttggggatctctaggctatgcatcttgttggtgttgaaacctcggcagtaaaacatatgctgcaacattattatcgggggcacaccgtccctcctcctagatttattgcctttgcaaatcaatatgatgccataaaaacggggaaccggttctgggcagagaggtgctgtcgaaggatgattcaaagccttcccaaagcggagagtggggagttctgaggtcccttcaaaacgccactgatagcgcaggttaacaggtaaaaccacccgggggttggaacaaaacaagcacgttaacacatctctctaggcccttctcaggctgactgctggcagcggaagctgctgaagtcttaatgttaactttaacacacgcattagcccggagactttgtgtaggttactaaacctctcaaagcttcagtttcttcatctgcgttagaatagggccaatagttcattcctacctggaagatagctctttggattaatgccctaatgtgtataaaaagcctaacactcacaataaacccaacataagtaaagcataatacagaattttaatcatatttaattttgaactgcaaattcgcgattggagaaacatgtctctcgggtgctcatatataatgtagatagttttggaatgtaca

SEQ ID NO: 96 - DNA, 7272 bp, Artificial Sequence
  subsequence of CACNA1B
gccctccctgccggggccgctgggccggggatgcacgcggggcccgggagccatggtccgcttcggggacgagctgggcggccgctatgggggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggccatatcctgccgggcggggccgggcggggccgggcggggaccggggtgggggccggggccggggccatcttcccggtggccgggagggcgcgacctggggaggcgtcgggagacgggcgaggggggtacggagcgccccagggcccctcggagcggcgggtgcgggagcatttccgtgagtgcggcgggcgcggggcggagcccctctgctccggactcgtccctccgcacgccgggtcctggggatcctggtcggtgcacagtgcgcggctagcctggagggcaccgagggcgaagccgcgtccgtgcaggtgcgagcgctgtggggacgcggccgtgcgcgcgcctggctgcaggtgcctgcgtgtgcgggctgagctggagcctggcgctgcgcgtgcgtgcgctcgcgatgcgggctacatgcatgttcacgtgcctgtgtaggtacatgtggctgcgtgtctggagggagcagcgtgcgcctgcgcggggtctccctccagcctcgcctgggggcgctcggccccctcccctcggggccctggagtcgggcggagccgggccggaggcgctgtccgcggtgctgacggccgcccgggtgtgccgaggggctggcggcccagactgcggaggccccgcctcgcggccgcccctagggatgggggcagggagtgagggagaccaggccgcttccgccaggagaggggctccggacccagttgtctcagcccctccggagacggcctagcggtggctgctgcactgggctctgctggcgtcggcctcgtgtttccctccgtgcggcgtctgccggccagtccttaactcacgcactccattcgagtatatgatcctggccaccatcatcgccaactgcatcgtgctggccctggagcagcacctccctgatggggacaaaacgcccatgtccgagcggctggtgagtgcccggctgggcctgagggcagggtggtggggaggccgcgactccactttcctttatggtctgtgcctgagaacagcttcctcgggcagggtcgtctgggcagtgcccctcgcgtctgaaggaaaaggtaggcctggcagataacaccttcctcgagggccagggcagctggggtcaccggcttagccctgggcgtggccacgttagccctgggccagtctgccctgtgccacgagctactcctgcacctcaatggctgtcctggctttcggcgctagactttgatccagaaaaccatgttctaaacttggggtggggaaggggcattcatgtcttcgggaaggtcagctctgaatgccatgtgtgccctgctgggctgagatgggctttggcctggatgtgggaaccctgagcctcatcctccaagttgtcttgtgcgggcctgcttggaacctcttctccagtgggcctcaccatcctgtgggaagcattccttctcaaacattctgaatcatttttacctgtccccagattccagccaagaggcccccgtggtagcccccattggagcatagagtctgtgtgagtcagggtgccagggcaaggtctccgtggctcaacaggaggcaaggtggaaggccagccctgtggggtctctggaggggctgggtcagaggaagggcagcattttggaggtgagcagtttctgaggtcagagcatgtcccccagcccccactcttccctgatgcctggccccatgtctgggttccgagtgtgcctaaagactggagcctccaggtgagcagaaggcagagcaagtgggggcccagggggtgcggggccagaggcgtccaggagacggcctctctggggtgtcaggagctgctccctcttcccttaagcctggcttccgcccccactagcatcctgagacaagagctattcagactttagccctgaaactgaactccctttgaacctgggccaccgcaggcccaggagtgcagaaggggagacagggagcctgtcctggaggtgaggcaccaggaggtgagttatgggtggccgaggtgaggaggtcttaatggagcacagtctggaatgcccagtggtctgggttacggccgggtgaggaggaggagcatggacagcccagccacaccatgaggcaggcgtgagtcatggtccctgtgggggacttgggcaggaccctggtggggcccattgctttctggaggctggagaagagggtgagcttgagaggaaacaggcagagagccttgaatgccaagtcacaggaaggagggcagggccagggtcagagggacgcaggcccaggagccaggccggggcgggggcagggaggatcaagctgtcttgccctgccaggcagtgggtgtccagccttgcaagttcctgcctcgcaggtgtgggagggtttactttgtggtcataggttggggaggacacttctaggagtggcgagctaacctgttgggaaaactgcagaacatgcccgccctgagccagcctagaggtctggggagctcttgaccagtcccagtcccatgtgcaggcccagccattgatgtcccctgggatggaggggaggctgtgctgctgcccacagagcaggccgaggctgggtggtgcaggggcagcccctcagttgtctcccagcctggccctgggcgaggcctccctggttccctgcctctcggggctgtttcccacctccccgtcgactctggagctacctcgagccaggccccagcttgcagtgcaacaccaagctgcctgcacacccatccctcccacctaagccagccttcagccatgggctgggcagggcagagctgtgaggagagggctcgggctgggggctccttcccagctgagggtgctggctggtccttcctaggcgtcgggcctgttctctttttcaccaggaagtttcctgtgaagatctctgtctgctgcggccccactgttcccctggggcagatggtcagcaaccccaccccttgggaaaaggggtccctcctaaacaaatcagaccagaaggctcgaggccaggaagagcatgggcgagtcagcacgggtcatgatggacccttgggtgctggtagcttctaccaggtactgccagccaagccttgggccagctccaccacaggacaggaggagcccaggctgagtaaggtgggagtcagatgcggagagccctcgagagcctggcttcctggggaggaaggcacaggctgtggaactggtggcactcacctcacctggcagctctgtggcagcccagcagctgcttcttcctgtgacttccacagtcctgaagacgaaccctcaggggaggcccttgaagctggtctcctctccccaccccttcacctgcttcctaaggcagcagccctggagtgcaggtgtccgacacccccatgccaaccctgcacatgcagctccctcagcccagcttcccgggcttgccttgcacagggcctttccctgccagccccacgcgtgtgctcacgaggagtctttggggtctccctaagctccacacagccccatctccaccctgtttgctgcttccagctcggcactggtagggcctggctctgctctctcatgtccagtaactgttggctggagctcagcctggggtgcaggaaagggatctttttaggatcagctgtcttcattcctttggctgctctgttccctccacctggatccctttctcaaaatcgagctgtggaagccttgcctgcaggcgccacgccctctgggaggctccctgcggtctgagcccagggtggctcccagcttcaggctcagactgtggctggctgcctccagggtcctcacagatgtggcggtcgtcgctcagcacactcagggctgaggttgtgcttctcatttctgttgttttctgccgagatgtgcatgttctggttacccgggtgcatgttgaatgcataaacggggcctggactgagttcaagaaagcctgagctgtatccaggcaaccttgtgcaggttcccacccagaggctgctgtggcctctgcctggctcctgggcatctctgtgcctctgtctcccacatgttagaatggggatgacctcatggggtattaaagggccaccctagttgataaaggaaaaactacctgataagcacccacaactagtactgttacatcactgtcttccccgaagcaaggcccactgcatggtgctagcagggagattggggccccactgtgacgtgggcagaagctgagatgccagggtgggaggcacgaggggcctgcaggcatgactgggatgttgctgctgtaaacagtggtgtccccattaggggacatgtgcagacagcaagcaggctcagtgatgggaatggctccttggagaatctgcagggtgttgggggcaaggtgaggagggtcagaccctcacgatagctgtggcctgcacatggtggggtggggtcctcaccaaccgtctctgcccgctactacaccgggtaggggccaggggtgaccactgttctgcgcttctcctaggacgacacggagccctatttcatcgggatcttttgcttcgaggcagggatcaaaatcatcgctctgggctttgtcttccacaagggctcttacctgcggaacggctggaacgtcatggacttcgtggtcgtcctcacagggtaggcaagctgaggccaggaggcccagcgtgtgaggcccgggcgtgtgctctctgaagctcagttgcgccgtggagctggggcagctgcagtcccctcactggggtcccctcacagccctgctggagatgaacgaatgtgagacagtctgtttgccaccctgtgtgctatgggggtgcttctcccaggggtgccagtgctgtatttctcgctgacggatcacagtgacctgtccccaggaggcgctggcttgcagagcgcacaggccaggggccaccagcttccatggctggcctgagccagcatggtgcctgttggaggtggctttgctgcccaggtgaggggcttcctagcgaggaggagggctggacttctggcctcacccaagcctgtggtctgggaggggcacttggcggtttgaattctgtgcctgcctgagacgctcacctgtggtgtccacctcgctcagagcccttgtgctgcctgggatgaggctgtgtgtgtcgagtgctttcacagttcctagtgccaggaaggtgcttcggcagtttctcctcaaccacttaccctaacaacagcttgtgattgccaaagtaaccagcacatctagaagatggacccaaggggcctggatgggagctcaggtccagtgctctgcaccccaccctccagggtgttaactccccaacctgtgcagtgggcactgtgaggtcccctttcctgcttccctgactggcaggcggaaggctgggctgggctgctgtggctctggtgtggagagtggtggctggggagtgagtgcttggggagttgcccccacagagcctgttagcacttcagtgtcagaggccctgtgctgggccagggaccactgtctgctctttcctgagccttatggttccgaggctcagagcccagatggaagaggagggagcaggccccacagcaggcgagagctctccttccagagccccatcctcacccagtgactgccccaagatgcctggtgcacaggagggtatatggaggggtgtttgggctggtggcagaggaggaaggagatggctgtggagtcctcgagtctggggctcatgccttcaaggttctgtgtgtgtgaggaagaagcagtcggctacatggcctctggtctcttgggtgaggccctcagagggagcctcgcattgcctgcctggatcccggggtgctgcccccatctgctctgcctgctcctgagcctggggctggcccctgctgggtgagagcagcatgtacagaactgggcttgggggtgtcgcatggaaggctcagcccccagagctgtctgggcttgggagtgtcacacagaaggctcagcccccagagctgtctgtgatgtgctgggcacagcagcattgaggtggattctgtgcctttctgcagcacggccctggggtagaatttatggaaggccctgtgggggtctgtggctccacggccctggggtagaatttatggaaggccctgtgggggtctgtggttccgctagcaatgcccgttgggggctggggtgggaggtggctggtggagagtgtggcctctcagaggactgggcgctcccacatccagtgggcactcccagaagcagcccaggagtcatggtagctgtttacccctctctctccctgtggacccaagtccctgaggacaacggtgccttatttttttcaggaggtccctagagcctcacttggagtgggcacccaggaggggcagtgtgcctctgctgaagtaatgaattgttggggtgtaagcagtgctggggggtgcgggccagcgggcagctgggtagaggtggcagcacccggcctcatggggcgctgtccatggtgctgagtgtgtcggcccttgagggccaggagaggaggcaggaggagttgcaccaagccatgggcaggacccctggatctcccagcacacatagactgggtccagggctgctcctccttcccctccccttcttccccctccccctcctcttcccgcctccctcttctgtttctcttcatctctctccctctccctcctctcccctcttccccccccccctcctcccactttgcctgtctagccctttgtctcctctccacgctctctgtctctggccctcttggtgtcttggtgtctctctgcctctccctcttttcttctgcccctctttcccttggcttgggcacgtctctgctcgcctgtgtctgcagcagggcctgggcgttgctggggtggctcccagg

SEQ ID NO: 97 - DNA, 7265 bp, Artificial Sequence
  subsequence of ZIC1
acacacacagggcggacgaggaggagctgggtatatagattttttaaatgagaaacaaaaaaggtcggagaacaggaagccaaacctggtttggcggagaccttggtgctttcgaagccggagaaagtggctggctcccggcagccgcggaggcgccagctgctcaggctgcagacaaatctaaacccttgcttgctgccaacatctctctccgtgcacttcactgaggactttgccaaaggaaagagctgaggaaattgaggaggccgggttaagggcgtagaaagaaacaaaacagagacagaaagaattgagacagagtggggaaaggaaataatttgggattgaggaggcttttggagctttatgcatatttgaagagttcagttttagtagtgactggttttaagagtgggtttgaggggtggagaaagtccgaggaaggcaaggaagaacttgttgtctattagactccatgagggtgttttcaaaacaccagattcccctctttggcctttaaaaataatcaagacacaggtccggaacagttaaaaaaaaaaaaagttcccacgagctcagggttgtagcctggcatcagacaaaacattctgggccccctgcccccaagaggccgggaactgggtctcctttcctattctgattttcccgggtgagatctgagaaccatgcttgagacttcgggtttaacgcccgagtcagggaagaatttgcggcgttgagttctctccggcccagattacgctataaaatgtgagtgtatgtagggagtggggtgggttggaggaggcgaccaggggatgcggagcgtcggggagggggcagggggaggagagaaaagcaatagggcaggagaataagccgcaaacgtgaaaactagaaaccaggagccaggtggaagcaatccgaaatcgtgtgtagctctgggtaatacgagtttccagatctgatgctaaaaaccgactcctcctactattaatttttttaaacataaaaataaaatgtagatccaattgtattagggattacaatagagttaagatatttataattccactgcagatgtaagaatttattgacgttccgggtttttaagcttgcaaagtgctaatcctgggctgctggctttttgttttattttttttctatttgtttagtttttgaaaaacggccgcggtatttttttctaattagacccctctcattctactttggcaccaggggagaagcccttcaagtgcgagtttgagggctgtgaccggcgcttcgctaacagcagcgaccgcaagaagcacatgcacgtgcacacgagcgacaagccctatctttgcaagatgtgcgacaagtcctacacgcatcccagttcgctgcgcaaacacatgaaggtaatcgccgcactctcgtcgccccctttgaggcaggagctctcttggctctcggcttggggtcgggcggcgagtggcagacaggcggtggcgggagccagaggaagcgggaaggcaaaggttccactcagtggaactgggcagagaaggtagggcgggacctggaaaaggggatgggagtgtccaaggccgttttaaatttttggtttaataataaaagttaaaagaggcgagtatgagcaaaggccgccactgaatttggcccagagctagggcttttgcagggggttgacgctgcggaaactgctgcccttgcagctgccagtgagacgcaagaattggactcctgcgcccaggcctgagctctggctcacctaaacatggacaagcaaagttcagggaaagaaagtgcagtctctggcctcatatccagtctccctcttcccagacctatttttcttttgtgagtcttggggaccctgactttcccaggagcacggtttctgcaggcctgtgcagagaaaccaagcccaaagtcccgctgatcgggcctcacctgtgttcagggggctccaaggggtccaggaggaagggcactcgcggccttcgcctctctagtcggccgccgcactggctctttatgtccgtaaaaacgcgactttattcctgcaggtccacgaatcctcctcgcagggctcgcagccttcgccggccgccagctctggctacgaatcctccacgcctcccaccatcgtgtctccctccacagacaacccgaccacaagctccttatcgccctcctcctccgcagtccaccacacagccggccacagtgcgctctcttccaattttaacgaatggtacgtttaaaatcagaaacaaaacatcgaacaaaaccctatttaagagactgatcacacacgtatacacaacattactgaaagaaccctgcgaatcaaaacaacccccacacagaccccgcaatccttttttaaaaaatctgccaatagacccaggacgagtaagagaggaagcatcaaccttttaaaaatttcctttcgctttcattatttttctttttttggcaaaggcttggtacccaaggtgcggtagggggtcgagggggaggaggccacctgaccaaatgccgccaaccccgagggccagtttcttgtcgaattggtacgggctctctggggcttcggcttctttttttctttgttttcttgtaaatacagaattattagcttaaaactgtactgttgaattctgtaaatagttatatctcggttggagcgggtgggtgggattgtggcgttgtggtctttgcattgggggaggggggagggaccggatgggcggggggagggggagggggaggggtgggcggccgaaagccaactgtttgtactgaatggcaagaatgttctagtaaatgtgtaccaaaatgtgaattactttgtacgattacagtctccacgtcgacctaacccaatattattggtattaatgtgctttttttgtataaagtgcaaacatttcgtcccaaagtctaagtactttagtgcagtaaaatgttgtttcatgtcctgtcaagaattcgtatagtacgagcctggatctgcgtgtcaaactgttccatttgtttatgtaaagtgatattaaaaaagatataaactataactgtccgttacttttggcaaaagatacaaccacataatgtatataattcctagtttccatatttatccgcatgtaaagggccggtttatccatgttacagctcttcaatatttatggctagaagaactcgtatgtacactttagtttccagaactgtttggtaacctttcgtaccttattaaagattcttaaatctccgtgattgtggtaggaatttcttcttcacccaccagcaacctgctgcctcttctgccagtcttaaagggtctccagaaaagctctccagtctcccttgccttccctttctgtcactgcaggtcgtataaacgtgataaatgaatgctaccctgctggctctccgagagagtgtaattagtatttatatcaaaatttatgaaacaaattttcggccgcagtataatttaaatgacctttgcagatgtagaataacaaccataaaaataacaggaatagattgcacaggcgacatcaatattaatgtagacgaattgtcagaagctcaggggctcgctctgcaacattgcaaatgaacttgcagccgagggttccgctgccccctagattaaattccccgggctgaaactgagttgcagatttacaatatcatattttaaattgctgtcttcaattaaaccatttatgaccataactaattttcaggatgtcgatgcatgcttttccaggccttccttctttgtacaaaagtaaatgtccataaagcgtttcacttatattcttcaaacatgatgctaatttaaattaattacttcctatgatatgttattattcctatgattttgccactgttattagttctctcaaaaatacatctagggaagaggattattttaagtaatttgattatctttctatctcttttatttatttctcatttacttaagaaattcgttccattggttggcattgatacagtaaatttgtaaatgaggagacaatataaaaaatctaaattacttgtgcttaatgactgtagcagaatgccttttctctaaatcagattgtctttcttgcagtttagtttgatagatttgcaagctatgctgcttccatgaagttagctgcgctggtaggaacgcaggcttctttgtctctggttgtagcttgcatgatcgccccattaggcagacaacgtagccggagatcacaaatcaggcccttggctgtagttgctagtgtgtggaggtgcagagcaggtggcagaaactgacctcactgggcaagggtggccatggacctgattctttaatgcactctatgtgttcaggaagccacaggccatatttgactctgagaaagaaaacaagaggaaaaaccccacaaagtataacaaccccttaagatacatctattttaaagtgaaattaatttttcagtttataccattggccaattacaagataaaaatgttcaatttctttaagaatcctttgttgacttgtcttttcatctcttgctatttatatttgtcactgtttcaacaaagtcttatttgctgaggaaggactttgctgcacttactgtaccacatcaaacactggggagggtggtgtttaactttttaaaaaatgttattctgattataacaataatattggcttttttcatgaaaagagcgccaccttgcaaggtttagtgagatttatggaagttgaatacctaagcaggaattgctgctagctccaaaaatttgcgaagcaaaagctagccccaattggtttggaagtttgaaactgattaacagatttgcatttgaagtgactccagacattaggtccagacattagttaaaaatagaaagaggaataaagacatctcttctctctagaaaagataacaccacaattaataatccttcccactttcattgagatcagcttgtctgataacctgatatgagtgtgataatgataaacatgataatagtggtacttttgtaattttgctggtgcatttaagaagatagtaaatgatgagttcatcttttcttcgaacattcctattcctagatgtagtttacctcaaattgggaattataactgtctaatttttgttgtgtacctgatgccccttttgctttaataccacagtgtaacaattaaatatcacactatgacatatgatttaagtaggatattttaaagataaattttaggggtaaatgtttacttcaaaatgactccatatttcaaatatctgtttagactgtgaaggccaaataatttttaagaaaacatttgaagagtagtgtgtttgcatttgtgaataatcttactcacagcaagtaaacgtaataaaagccaacatttaagccaaccctcctgtgcattctcttttccaacacctatcacaaagaaaaagtcattaatgatgtaggtggcctaaaataatccttcgaagttaaatgtagtctattattttgctggtacctttaatatctgcttcttgagatgtattcttcatactttttttcatgggctattaggttttgaaatacataaatgtgaacagcaaaatatattgttagactctctttagcaggagctgaagatgctgcaaatagctgtttcctgtgtgtagctttagattctgacttaggtagaatgctgtcctggttcattttgcaattgcattgtattctgaatactatcaaatgattagaacagctaacaagttattaaaaagttatgttgtgtcaacagaaaatgcctgagtacaagcaatcatatttgagaaatgaagagaaatgagttgtggtatgtgtgtgtgtgcatttgtagagcaagcatataaatatacaagcaccatttatatgggggctaagtaatttcaaacatttcagatcctttgttttcttcttcaaaagctacccccaactgctttaagcgtttgcagctgatgattttgattttttaaagtcaagcattcaagttaacctttaaaaagcaaaccacctccaaaagaaagatgtaacctttcaattgccccatctgcttatcttttttttttctttctttcttattttttttttttcctgacaatgaaagtagcactaaccttctttgctgataggcctggcatttagatgcttttattgtgaagttttctcagaacttttaaagggctggccagagaattataccctcaagtacctcacctagccccaaaagcttccagaagttttctttttgaggccaacattaaatcagtgtctaggaccccttgtgagtcggtgctgttggagttgttacctgagcatactgcttttcaggaagtgagttggagaaggccctagaccttaaagagaagatttagtttaagatgttatgaggagttactgcaggtggtctggcacactcaaaataaaggaacttgtgcccttggctgtggactagatataaagacatcagctcttacacattcccaagttacatttcttttctttttccaacttataataccgattacaaatttatctcctcctctctaggtacagagttctggcttccttgtagcatcaagtgtagttcttagaatcatgtgttcgtatttgtggggaaaagcattttcataaagatagtcacagttgatatcaactgttttagaattattgaaaatagtgttggcaatggaaagattgatcttgtctgtgccgtcaagccgctaaaggtacaagttagccacgatagtctctctttcggcttttaaccacactgtccatcctctgtaatatgtaccatttaaactagttagatatagaaagtaagattattatgggtaaccatatagcaatttttaaaaaatgataagcactattttggggcaagaaatacaaaaaatgttaacttcttatgtcacattcaatccttttccttaaagtttattgcaaatacaatggtgtaataggtgcaaaatatgaataatgttctccttaaatctatgcaaggctggtttgctccagagtcccttccttagcaagaggaaacacatcaggccaaaccagaggctatttttactgtggaagctgggatctgtcatcagttactaaagttgatatccactttagagtgaagaactaaaacagtgtgaagtttccaactgatgttatataccaaaggtgagtttttaaaaaatcgggtt

SEQ ID NO: 98 - DNA, 6047 bp, Artificial Sequence
  subsequence of DLX1
ccccggcgcagacgccgcagcccattgtgcttcctgcccggcgcgctctgttgcagaggagccccggccgggaagtgtggatgcgtcttacccgaggccgggccgcctcgcccgctctagtccagcggagcccgagcgcctcggaccaatccccagtgattatgcaagacagcggaccaatcagctccgccagctcatgaatatttatgaccttcgctgagtcaaagctttgaaccgagtttggggagctcagcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggtacgtgcgcttgccagggagagggagaggaggaggtacaagggagagagggaaagaaggagcgggggagaagaggagagggagagagagagaaagagaagagaggagagcgaggtggggtgggggtggggagggcgcgggagcagtggaggtttcgaatatcaatctatagatccttgtcacagcaaataaatttttttaaaaattccctcaatttgcaactatccagccaaggataaatcccagagcgtctcctggcactggctgggcctctgggcggctcggtgtgcagagcacacaatgccccgctggaaaacagaaacccacatgtgcacgtattagtctcggtaattatttattgcgtagcgctataaacaatgtatgcaattaagggtaattaaacctcaactaccgcctgcaaaaatagcaaactttccctgcaaaggcaggagctgagctcctgggaacggctctatccctcctgcagcgtcccgggacaggccctcggattttgggggacccttccctggctttcagagtttcttgaacgttctctccctggtgctgcctccgccacccttcggtagccactcgctctcggctgttcgcactaaaggcggcccctcgtattaacaacgggccctacttctgctgtccctccaggggcggactcggagaagagcacggtggtggaaggcggtgaagtgcgcttcaatggcaagggaaaaaagatccgtaaacccaggacgatttattccagtttgcagttgcaggctttgaaccggaggttccagcaaactcagtacctagctctgccggagagggcggagctcgcggcctctttgggactcacacagactcaggtacctcgccgctgccgctccgttctgccacgcaggctttccgcggccggcctgcgcccgggtcttcatttgttgctcgctgggactcagggtcggggtgtcactgtgtgtatgtgtttgtgtccacccctggctctcagcgtgtctccttcctccctcattccctctgccctagctctgtcactgctctcggtatcccgagctgcctgccgtccggccctctgtccctggacaatctgattagggcgcaggaaggatttccccagacgatttgtttgggaactcagaggtcacacgtgcctaaacaactggaacaatagactccgggcttaatccctcctttgcctttaaattgtgtgactaatcactcggggcctgggtccgcgcctcagcctccctcctcctcctccttcttaccggttgggggtggggggagatcctttcctccgccctctcacctctcaagtcccagaccttagagaagaaacgggaccttccttcccggctttctgtaagactccggggagcccgtgagcgttcctgacggcggcgggcgcgggtttccgacgtccggtccgggatttggagcagagctggagaaagcaaacagacactagtgcaagaatggttttgaatccaaagagaagttcagcaaaaccttgaggcctccttagtccgtcccaactcaagggcagaaaagatggcgcatagaaaagttgggtcgcgttgcaaataaatttcctccactccttcctaggttttcgattctttttattgatgttgatattggaattgctggctcggtgttatgcaggcgctgtatcttccgcaggcggtagccacggtcggactgagcccctggcaggctaaggcctggatcccggttctcccctcatcacaactcctccctggctctctgagagctgcccccgtgggctgacggcggcgggaggttcggccttcgagcctttggctcttatgggggaagggaggaaggagggatgtctctgcttctctggcagggagctgccagctggcgcagggttgggaggtcctatctctgctgttctgcggtcccttttttcctccctgtgacctaggtaggctcagtggtcccagcctgagtcacgttgttgtccgctcttgcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgggtcctgccgggctgaccatctccggatcctgggacactctgcctgaccatctgtgtagctggtgtgggaatctgggggcattggagggagggggttttatttattgagaaatggacttcgcctgaggctgtttgccaattcagggttctgctgggcgcaaggaacgcactgttcaaacgcactgtttactttaagcgcacggggagaaacgaataaggaggacgtggtgatttttaatttatacagtaacttttgtacttctctggtatggagagtttggagccgaatgatttgcattttttacatgtccgacattatttaataaataatttttaaaagaaaagaacgataaatgaagccaacatgattttctcatttcgggaggaactctgttgcttcgcctggacaagaaggaaaatgctgatttcctccttgggtagaaagagggagcgagggcaaatggggagtagagagaaaacaggcgagaacaagcactctaattccagtgggctttaaaataagacaaaatcagctttacaacaatccctagaggctcgaccacagaataatgccagtcaccaccctgaacgcacaatctccagtgcaggatctaatgactgtacatattattgttattattattattgttattattgttgttctgtaaacatgttgcacaagcttagcctttttgcgttctgttgtgtgtggctgtaaaaccccatgctttgtgaaatgagaatcttgacatttttcttgtgaaatttggaaaatgtgatcaattgaaatcaactgtgttttgtgttctctatgtcaaagtttagttttatattgagaatgttaacttattgctttgtatcttgggaaaaaaactttgtaaataagttataaagtttctttgagacagtaaaattatgatttcttgaaagaactgctctcttgtgctgtgtgaggctgtgccagggggccaggccaggttcccgcctctggagacagttcatacagggtcagcgacttatcaacttatcggtgatagaatggagaccctgtaccccagaaacaccagggtatcgtcagaggcctgtgaggtgccccattgggctttttcttcctgccttgctccattgacccccaaggtgtctgattcctacctgaattggaagggtaaggagagaatgcctggcgagggttccccaggaagggggctcaggaacaggtgcatcagggcagggctgacgctcaggaaccaaaaggatgttgaagaccggctgagaggagatgaagtcagagttcaaagtcagccggccaggacgatttcagtttttcccacacccagccagctcttgagaggccgggaggttgggaggctgagaggcctgaaagcagagacactgctttgcctctagccttcctccggatcaggcctccctgcttttgcatgcccggaggcgctggattccgtcgcggaaaagacgcagccagacgccaagcaggggcccggaaagacacgtctgtcagactgcccctctatcaatcagtgcgctccaggggacagggcacagtcccaggcagccaccctgggagctccacagtgagaaaggcaggttcctgcctaatgatccggggcctttctctgcacctcacagaaatggaggtgctaaggaaagggtggctgacactttggaaccccaaaatctgaccaaagtcaagcacaccactcccacctcatcccagtccgaaagtcccaaggccaggcccttatgggagcacctctagtgaagcgcttgctcagattagtttcctccagaggaacacgggcttcaagggactagaaccagattggttcccttctcgtggccctgcgtcccagcccagccccaacccccaccccagatctcgcagccgagccgaggcagtcccggagagctgcgggcccagacaggctgccaagcgctgttcctacttctcccgcactcgccttccagcgctgctgcccgcgggagctggccctccacctttggggggtcttttcaggcgcgcggtggggagcagagcccggaaggcagtgatctgtgttgtgacaagcacccatttttcaattggtgctggtgaaagatcagatgcgccaggctcttccgacgccctttgggaaggggaggaggaagtggtggggctgctgggggtggggggaggcggtgtgatggaagcagggaaagtagtcgggggtgggggaatgatgccggggacctccaagccccttccaggcctgagattgggacttctccgcagcacctagaagaaccagcgctgatgggagagatcagacctcagaaaccaggatgtgggcagtcagatgggcctcaaagctgaggaaagaatgaatttcccagctcccgcgcggggtagggggtcttcctaggttcaatttcccctaggagatgtgactttgctagtgcgaagatttctgtccggcatctgactcaggtcccccagacggcagctagggcccaatgcctcaagctacaggcaaaatctgtttggtcaagcggattgtaa

SEQ ID NO: 99 - DNA, 6023 bp, Artificial Sequence
  subsequence of DLX6AS1
caggagattctttgacccaagagcccattcctccaacagtggcctccggcagacccgggaggtcttgtgttggcaatacacaagcatttctctctaaataacattgacactcgcaatacaaattccaacatctcaacaccttaaaaagcaaaaagcagcttaggcctcaaatcctgcctagtgaatatgaccctcacttactacaagccctaaaagagccttagggctcctactccagattgcagggtttacttttattttgaatgataaagacttcgagggccttgcccgccctcctcctcccacccgcaagggttggcccgcgttcccctgcgggcgagacaaagaagcaggagccaggacccggctggcgcctaacccggcgccggggccttgagcccctgaggtgtgcgccttcctgcaaggccccaaagaacccgagtttttgccccttgctgaagggttggaagtgcgggctggactagtgcaaacaagagtgactccaaccgccttacttcagctcattaccgtcacccatgaaaacggaatgaaggactgggaagccaatctgccgcgttcaattggagagaaatgactgtcggaagaggccctgcggtaaatagcctctgagccgcgaactctatgcgccgtggccggtggcagaggccacacctggcagccactcgtggcgcgccaccttggcatgttgtgtgcgggtcacactatctccagttttccattctcgatactggcaggaaagtctgatagtcaggcctgaagtttagattccatcccgcaagcgcagagatatcacaccaagtgggacctctcgctcgttccacttcccagcccctgactgtctctgagctcagactgggtcgcccagcaggtttcagcaggacctttaccatgcaccacaactaccacctcagtgcagctgcttcactggaacagagtgcaaggattttagccactaactattaggggaaggttgagagccctgaagctacaacccggacaatcccagccttcccggcttcctcagcaatcaaacaaatgagaaaaaaaaatccccttgcaagtagaggcagctgcactctggatacagtgctcaattgcagggaccttccgaggccctgccacgacatactctaattggggtgcagccagagttgcaggaagctggcgaggaaagcggtgcacctgtgagcataaagcgaaaaacatagacaagcacagggtgagggtgtagggggtgaaacgccttcacggtgttaacaacaagctgcagggtataaagcaataaaactgccacgtctacctgctggtattgaggacattacaaacagtctcggcgcgcagcccgcgtgtaccgagagcgcactagggttgggagtgggaaggtggggtgggaaatcttctcttttcctgacattcccttctgtcccccaccccccacatctcccccttacttagcccacctttctttttccatgaagcttttctgttgttgttttaattcattgccgatttgtaaacgtgtagatcgtgttactggttcgcgtgttaaagtgcacctcttgttaaaaggtggggaacagggcacgccacttaaaaatgaattcagagttactgaaccggctccccagactcaagaacttaagcgcaacgggctgggaaaaggagggggagaggagcgcaaacgccgctgacctgggaactcggaggaaactcgcgggcagcgagcttcaggtcttctgatacattgaaggaaagaaagaaagacagaaaaagaaaaaaacaaactgtcctggagatagctagacatccagggctgggagctgggaaaatcaattggcataagccgctgccggcgggcttcgcgcaacgtaaaaaataaagtccaagctgctccatctgcagccacccttttctggagttaaaggggcaagcggcaccttttctctatttctcagtttccttcggtcctgtaacttatggagtccagaagaagtaggatatacagaagtcctcagtagttttcccaaagggagacactgtctccttcccgcaacctccagacactgcagtacagggttgttagatattagatagtacgtttctccgtgtgggcaaaggctcgcggcttaggtctctaagcagatcgaaagtcggggtccagaccgagaggatcttcaggtctcgtccacccaatctcaagcctctggaagggcgggctgccggcgacctcctccagtcccgggacccggaacccgggaccctggacgccggtgcgggcgggcctgccctcctctgtgcctggcccccagtctggccctcctacctgttgaccaggcctgaaccacgcaccggggctccgctgtctctgcttggctgccttaacctgcgtgggtctggagaccggagggagaaaggctagatccgcagccttgggctgctccgaacttaatgtctttccccagtccatctttgaaagagaagtggttctaccacctggatgtgcggatataaatccccttgcaaataataaatggattaataataacaaggtatgaagttctttttatagaaaaaaaggagagaattgaaactaaatgcggcagttagacaaccattttgataagaggataattgaggcgacactggtgtataattagaggataatttatgctatatccacttttattccacctcccaaaataaacccagtccataatcattacaggcaaattgttctgaattttatagcagcaatttgaacaaagtactgcagttaatcatgggagatttttgtgtattcctgattagaactctaattgcctccttccagaaagtaaaaataactgtaaaacgactctatttttatcattaacaatgttgacaataaaataaaatcaattggaattgtaatcgagagacaaatttaggacgaaggcacttttacttgggtagtttatattgtaatcaagatttgccaaaccactaaccgcatgtatcatttgcatatatttgaaagcattacagtagcttctgttttcaataggaaaaaatgcattactgtcagtcctccaacgattcgttttcagaacacgctgctgcgttctgaaaagaaaaaaaagacgccccccccccccacaaccttttgtacaatatataagaaatacggcctgaaacggcagggaccgagccagaacagggtctgatttttctttttctgtaccaccccccagtggggtggagcccaggtgtgagggttcgtcttccttctgtggtttcaataaaatacgtctagaaaaatccacggtgacaaggagctcccacaagagtgtagtaggaccagagtggcagggacgtcaagaaaggggggcctggatttaatgcgggggtggggtcactgcttctaagcacaggcaacctacaagggtgcaaagggattgaaagagagcagcccccagttggctgtttcaaggctgagatattggcaactggggctccgggatttggggcacgagctgcggcgggaagcatcggcggccggccgggcagaggcgcctccctccccaacggccccgggcgccacacagcgcggcccccggctgcagctccggctgggattggaactgcggaaggtaggcgaagaactctgcaagtcgcgcacacgccagtagaagccgtgcgcctgtgacgtcagggggcgactgaccaatggggaggcactgccctttggagacaaaccattcgactcgtggcgtctgcatcaagtctgaaagcagacgcgcaactttcgcagaatccaccttaaaatctctgccttaaactgcaccagcccccaaaaaatccaaggggggaaagcaggcggggggagagcagattcccccctccccctctcctctcccatccctcctccttcctcctccctttgagttaacaaggccccgctcactatatctctttatattaaatatatatatatattagagaagagcgagggagagggagaaccacctccacccccctctttaaattcttttttttttttttttttttttttgcaaggatccaaagagctaaggtggctgcagaggggagagcggcgcgagccaagtgggggagggtggaggaaacccgggagaaggctttctccagcccccaaagtttttgatgatgaccatgactacgatggctgacggcttggaaggccaggactcgtccaaatccgccttcatggagttcgggcagcagcagcagcagcagcagcaacagcagcagcagcagcagcagcaacagcaacagccgccgccgccgccgccgccgccgccgcagccgcactcgcagcagagctccccggccatggcaggcgcgcactaccctctgcactgcctgcactcggcggcggcggcggcagcggccggctcgcaccaccaccaccaccaccagcaccaccaccacggctcgccctacgcgtcgggcggagggaactcctacaaccaccgctcgctcgccgcctacccctacatgagccactcgcagcacagcccttacctccagtcctaccacaacagcagcgcagccgcccagacgcgaggggacgacacaggtgagaggccgctggggcagctcgcttctcccgcctcccgactgccccctaccccgcccgcccgctcacttcctcgacgcccgggcctccgccggccccctcccccaggcggccccgcgcgcccttggccgggccccgtgcgcgcccgctcgcctggcgcctgcctgccggcctctcccagccgggccccttcccgccgccgcggaccctcagcttccgacgcgcagttcgggctgagtcccgggcccggcgcaggcttcgttcgacgagttagtccacactgcgctctccagcctcctgggtctgtgcgcccttttccttgctccttccctccaggctgtaacccacgtttgacccggctggggacatttctgggccggaactggggagcgtgttttgcttcccagcccacgctgggcaggcagtggcctactttgcgtggggcgccccgtgctcctcagtgactcggagggaagctggcctggctttcgctctggttaaactagtaacagtcaggctctttccagtgggctttaaagtgtccctcaccttcatccctgaccgggccccctatctagcttttcgctacagtgttggctgctcctttgttcgtggaggggaagcaaagaacctcgggctgatggttctcagcacccgagggggacaaagttgccgtgagaggcgcagcctgctcgtgggctggtctgagctgttggttgtgttttgtttgaggatttggtcctcctcaggagagtcatttcaaaacatgccgaggatgcttgatgcactgagcttggagctggtgacatcaagtcaactggccgcgtaaacacctgggtggctcaagcacagcttcaggtactgttaagccgggcacaggggccccgctccgcccggacagctgccgccttgctgcgctgcgcaccccagagagcctccggcaccgcctccgctgcggcaaagcgggctgtttgtctgagggcctctggcgctcccttggcccagacgctgcacattgttcgggccagggactgggtcaaggagattacgcagacgctgggagcacacgggcagagtggaccaagagaggtcggggcatcatagggatttttcgaggtaacccttattgggctttggggagactcgttgcatgccacgccaggagcagggagatggcagcagtagcctcccgggtgaccgctcctctgtattatttgcttacagatcaacaaaaaactacagtgattgaaaacggggaaatcaggttcaatggaaaagggaaaaagattcggaagcctcggaccatttattccagcctgcagctccaggctttaaaccatcgctttcagcagacacagtatctggcccttccagagagagccgaactggcagcttccttaggact

SEQ ID NO: 100 - DNA, 4118 bp, Artificial Sequence
  subsequence of MIR196A1
ccctggtttcatcccttgtcactgcatcagtcggggttgaattgtccaacaacctgggctccccattagcttgcaagtcaggcccctctccaaggaggtggcactcaggacctatgtgtacccccagcccagcccaaggccagtgggaagcctttggccactggggtgggtggggaggaaaaagtcaactgagggattaggaaaggaaggacttttcggacctacttctgtaggtctggtcccatagggggccttcgacaggaccctgagggctttaggctgagattgcaagagctctcagtgtttgcctttcagaatgagacctactgagccctcttcaaggacctgaagcttcctgctgaggtggagcaccagcccgcaaagagttacaaaaacctcaagaaccccctgcatttcttttgaaatcaaatatttgtgaagttggggagagtttctctggaaagttctggatgtagttgagagctcagagaccctaaggctggaagttggagctctgactgccatctttgggttgggctgaacagcccaacagggccagggaggtgcctcgggagagttgactgctcctcatcccccttcccctcctaacacacacacaaaaaaacattcagaagtgggaagacacttcccagatctcttctctgggggaacatccttgttgctcctggatgaaggacctcaaacctcttccccattttgcaaggttgatcttccagtcctgattctgctcccctcccccaactcctccagctaagcaaggagcagtaactgccgtgaatcgggtggtttaatgttgttgtgttcagaaacccaggcccaacaacatgaaactacctaattcactcagcagttccaattcaagctctaggtcaagagctcactgggggcaaaccctgtggccatttttttgtgggccaaaaagcggcttaacttcattttgctccacctcttccccaacagaagaacccagttgctactaaacaacacatcggaagaactgaacccaggactctaggtaaaaccattttggagccggtcctgatagctcagattccagagacatttgctgccttccaggagccctggccagagaagaagaccctagacacaggcacaagcctgggcagggcctctttgatatacctgctcagtctctgcctctcagcccagctggaggtctccacagataaaaagaattctcaacttcaaacagatacgaaggtgcatctatactcagcaccctgctagcccctgtattccaggtcacccagcctcccagtgtagctctgagaggccagatcttcaagtcctacacaccccccagctttgagattggggctcagacaggagagtgagacaaaagagtgctgagaaagaaagtgacagagagaaaggtggaaaagcagagtgtcagaaagtttcgggaattctagggaacaaacagaggaagaatgggatttggaggggaaagccggttgatctgaacagttgctgtgaaggcaccctgcccaaaatgtcccagggccctggctctgcagaggccgagctgccccaggtaagacaattcattccgaatcttcgcagaataaggactgagaggctaaggactgggattgggacacagagaacaaatttgcttcctccttcctgaccttcggtctgggtccttggccccctaacgtgcccgctcctcacctctgcctctccagacacccgcctgctcacaggtgctcacgctcacacccgcacccatcgcaaaaacgcagctgtgcaggccgagggcggcggcggacttacctgggcggcggcggtgccggcagtgcgcggccgtcggaaggtgcggagccagcggccgcggcccagtccaggctccctcccgccctcgcccagaagcgagggtccgagtggccacgtgtccggcagggaagtccgaggtagcgagggagtccacctggccgcccgctccccctcccgggtataaatcttggggaccggaaatacaataaaacttcaccgtgttgatgaacttcaaacggtttacatcccctcctgtcctgaaaagaaagacgtccgggggcggattaaaaggaaggggaaacaaatttacgagcggcagaagcacccgcctcggcccctttctttgcccgcacactttgtccgcgtggagagaagcagccccagcccccgggacttggcctccgccgaaccggccgccaggcgcgctcttctctcggaggagtcggcgctgacccagcccgcgcccggcagagactggaagccgccgcctacacggaaaatccacagagccccgcgcagaccctatgatcggctcggcccgccgggcttgggatgaggctccctgcttctcgctagagcagaactgggctccctggcgcttccttgcgaaagcccaggctctcccaaccacgcccggtggcctccattttttgcatctttccgctgaaagcgggttttttcttttttctttttttttgacagagtcctgctgtgtcacccaggctggagtgcagtggcgcgatctcggctcactgcaacctccgcctcccggttcaagcgattctcctgcctcagcctcccgagtagctggaactacaggcgcccgccaccacgcccggctaatttttgtatttttagtagagacggggtttcacaatattggccaagctgatcttgaactcctgacctcgtgatccaccctcctcggcctcccaaagtgctgggattacaggcgtaagccaccgcgcccgacccaagggttttttgtttttgttttttgtttaactttacttttctctttccttttccgtttctagcggccagacctttgcccagccctgcccgtttattgccgggtacttgctggaaggggacagaagataagactcggggtgactcccaaggcctgaagggggctgattgggccgggggctcccagaggataaacctaggggccggtcggtcttcctcggcgtggaagtgagagccacgcggcgtggcggagctccaccaccgtctccttatctctttaaagcctcaaaatcaaacaaaaccactactacccaggcctcaagtttccacagtttttgtctgctctggaccgtttactttttttgaaaaaagagaacttggtggaatccaaattctctactaggcctccgaaccaaaacgaaggaaaatccgttttcttcttgggcttcagagaactgggatcgaagtgattgacttcctgctccttttgtctgctccaggggcctggttaggtgcccttctggatgggagagcaaaaggttctccatcctgcctcacagttattcacctccaaaacagttttgagacacaatggatggctaagattagctcaggctcaggcagagtgttcaggtacaggagtgaggtccagctgcaggcccaagagggggtttagtgcactgaaggcacccaccttcccatagatacacacatagataaccccactccccaaccccaagaaaaggcaccaaagtgctgctggatattttaaaaatctgattaaaaaataaacgggcagggaaaatactaggcagtataggttttgaattttctgaagggttaactctgctgcaggcagtcccactcggccccagctctccagaggtgggtggtggtctgggggcagtgccctctggccccttcctagcaccaaaagggaggaattggggagctctttaacagctggagggcaggaagtatcttatttctggccaacattcatattccagtatttgcatggggaggcccctccagcctccctcatttgctctttatcatgttatattttcattcaaaatactgagatggcagctactggctgtcacttcttctattaggatttccctctgggaggacggcaggactgaggggccagcagtttccagaccattcaagagtgttattacccatcccctaccgggcacctggtgggggccggtgtgctggctcccagcctactgctggcctgggtctctgtggctccctggctgagaaaaggagatggaaaccttttatagtcttagactctttggaaaaactgaagaggc

SEQ ID NO: 101 - DNA, 5393 bp, Artificial Sequence
  subsequence of FEZF2
ccgcaagtggcgcacgtgaaaggcttcttgtcgttgtgggtgtgcatatggaaggttaggttgtagacctggtggaaggccttgttgcagatggtacatttgtactgcttctcgccgctgtgggtcagcttgtggttcttgtagttccctacaacagatcaagaacaaaaaacgtgggggtatggagtagaatggtggggaggaagaggcgggcgtcccaggggcagcccaggtgcctgctcaaaaaaggcgacgcttggctaggcgggcgcgacctcttcgagtgaagaagttgtcaaacttcgtaagcgtcaagccgggtgctctcccgacaagaccgagactgagtcccgcggagccgctctgcgctcctgctctgcccgccacagaggctggtgcagcttccctcccgccgcgctccgcgggccgggaaacttttgcgtagcccagagacgcaccgagtccttctcctggctgatgcctcgctagaagaaattcgcacgaacccgggcttcatcctttttcttttcccccttctcatttttaaagttacttctttctcgccccccaactaccaactgaagatccaaaagtgccttggaaaatccgatccaggggccaaggatcataccggtttcccccaatcttagattgttcccgggagggttattttgcctccaatatttttcatttgaaagggaagttccggatacaaaaggaacctttttctatagggcaataccctctccttttcctacatcctttccttctgtccccgtgcttccttcgacctctcgaattcggagtctctcagtgcacccagcctgcatacagcgtggtctactaaccccgctaggagagcttccccctacaccaggcacaacccgattctaaagaaatgctgcgcccgcgcctgcagatttcgccttctctctccagatccctctttgccttcctgccagccagccgcgacccgagtcctgaggtgaggtgagaagagaaggcctcgcctggcacgttacctttttggtgaaagcctttgccgcaaaattcgcagacgaagggcttgtagcccgcgtggatgcggatatgcgtgttgagcgtggagctgcggttgaacgctttgccgcactggttgcatttatgtggcttttcctgaggaaaggggcgagtgcacagtaaggagaggccacctcgggaacacctggaagggacatcccccccacccccactcaaccctagagggtaagggataatctttggccatcgtatccccggtgttatcactaagattctgggggttgcgcgcgggctaggcccgacccgggggccacgtacctgggtgtggataattttgtgcctgcagagcgtgctggcctggcgaaagcctttgccgcagactttgcacacgaacggtctggctccggtgtggaccggcatgtggcgggtgagattatagtgagcgttaaacacctatggaaagacatggggggccttgtggtgcgtctgtccgagggcctacaatcagccccgggggggccacagcctacctcccccaaccaacacccccttcagaaaccagagcctttttcagtttgtaaagtgctgcccaaactcctgcttacaccaatactgtgtggccgctgacaagggcatccccattttacagaataggaaactgaggcccaacgaggctcccagtttgggtagtcaactactggggagctcctcagcttgaaaaaggggcattccaggctgccccaaggaagccgccgccgccgatcgcccctcccggcccccctcgccgaaccctgggcctcaccttgccgcacacctcgcaggtgaagtttttgggcttgccatctgcggagcctcctggcagcttgctgtggcccttgacgcctccgcgctcggcagtcagggccgagttttccttcagtacctgctccagcggcgccggcaagcgctccttatggggatagggagccgggtgggggaacttgtccgcagccaggccggccagcttggcgttctccagcagaaagagcttggggtgagcagccagggcggcgggggcctgcgcattgaggaggccagacgggaagaggtggccgctgaggagctcagacggcgggtacgcggtcgagtccaggtagttgaagtagtagagcgagccgctggccggcagccccacagcctggttgatgacctgcggcttgatgaccctgcccgcgggcagcgcggacggcgccaggcccagctcggccttgcagcacacgccacagttggttttgcacaagccgctggcgccgcacactggggcccccccgccgccgccgccgccaccgccgccgccgcctccgccgccgcccgcccggaggctgcttttccagagctccgagtaactgagcagtgtctttgacggcacctcgtagcctaggggctggagggggatcatacagggcagcggcgagcagaggttgagcagtttcttgccctggctgccgtccgcctctagcgctccaggccggggctcaaagggcgcacggggctccgacgtcttggccatgatgcgctcgatggaaaaggccagtgtcttggaagtggccggcgacgctccggcgcgcgggcaggccgggggcaccatggtctccagggaagccgagcttgccatggcgcgcggagctgagccgagccaggctgggccagggcgcagcctctctcctctaagtctgcattccggaaaaggcaggggggaaaactgcaatttaataagcacctagtcgggcccgggtcacccatttgcattcaaatgaacaggggcaaaacaaagtgcacccaagggtaccaaattaccgcccattaacccggcgcgcaaaggaacccaccagcccctgccctgggactttgaaagggggaagaagggggagggtttacaaaagaaaagggggggggcggtgagaaaagagtctcaaattaacccccctgagcccttccctggacccgggcctctgccacgcgtgctgggagctggacagtgaaggggcaaagttaaagaggaccggagagccacctcgcatttacctctttcccccaccaccaaggagatgcgttccgagccatgcagcgtgtctcttctgtcacattttgatggcaaacatcttcccctccgcgtgagatcactgtcttttacattcagctgacatcacatgaagtcacttgaagtggaatgacatgggcggcggcgcggctcccgtagcggcccctgtgttcccccgcgcctgccggccgccgccaccaccgcccctcaaactttaaaaggaggcaactggcgcccgcgctcctctcgggaaagtgcgagcggtttgcgaatagggaaagagggaagagacgggagggggagggggaggaatcgtgatggttttgccggtggaaggaaagggggggggggacccaaacccaaacctaggcagagttccgtgcgtgattcacaactttggaggcctgcaggtttgggggacgcggggagggcggagggtcccggacgcggcgcccccaaagggcccctgggtatcagtctagtagccgaagtatcccttagccctccgcagccgagctgggcatcgtgctaataaactgccatttacccacggaaccggcgccagccccgaacacgcgcaaatgataattacctcattaggatgtggcgcatggacgcgggcgccgcgtttggaggggggaacttgttaatggggaaatattaatttatgcaaaagcaatgagctcgccttggctggcgtcggaggtcgggagaaagctcccactgactaaggacgaccccgaagcgtcacggagagcaaggccgctcgccggcatctcgggggcctcggcgccgcgcaggtgcgcctccagctttatggccagctctgggtctgaggctgggggagactaaaccgagagctggaagactttcggagatcctaatagatcagaattgttgatttatgatcgggtttctgaggcaaagccgagttctgcttctggtttagggctgcgtgcggggaggattcggagggcagctctcccagctgatgggtacttactgtttgtataattgagaaggtcccatctccacctgtttaaggagcagattgaaacagcagaacctccccctggatcctattaaaacgtttttcctcctaaggccattcagtcgaaccaattttccgaagtgattcatagagctgaattctgccaaggcagctctttcacttttagccactaaagcaccgcgtggaaccgggttttgtgtttttctaaaataaagagaaggattatcagagaggagagggagggggcctgaagggggacgggcagagacttagtgtggccagaggtacaagcagagggaagaaaagacccttttctagacaggccccaagtaagttcggaggaattcagcgttttgtgcctggcgcacaaaagcagcgcgctccgcgcttggtttggaggggctgaattcatctgtttgcagaccattccacctgcgtatcacaacaaactgctcaaagacgctccagcactttcaacaggccgggtgctcttggcgacaacaaaagtggtcgaggctgagggttcttacggaggggggtggtggtggctagcaagagagcgagcgagagaattaggtttaggaaagggggaggactgggtgcaagggttttttgaaaagttttggctggttttgttcgcgggacaattatatcttgctagccagcttttgcaagaccacgagctttgtagccgcaatcacagtttaagaggattaccgtggatataatatgagcaaacagggtttgatagtgaattccatctccaaagacacacgatcatgtgaagccagagaaagtggtggtagaggtaaaccctctgtttcttcacccaggaacaagtcttgagagggctgagtatcctgtcctaggcccctccatgctgcgaaacatccctggagaacgttaagggacgggggagaaaagctctagttttaacttcaggcaaatcgctccctttttcccttcaataaaagtctctttccttctgcccacccatccagttgcggctcctttgaggtgcaattttaggcaggatttggacatagcaggtaaaggaaggaaaagtgtacttggaaagaaaagagagggaacagagagaggtccctgagggaaagtggcttggaggtggactgcaataggctaatgctctcaccctgccaaactctgaaactgcccctaaattccgcaatcataagtctgttcccataacgtcaccctcccaccccctccccaaactaccagatttctaagccaagaggcttgctcatctggagaagaaca

SEQ ID NO: 102 - DNA, 5442 bp, Artificial Sequence
  subsequence of STAG3
ctcccaaagtgctgggattacaggcatgagtcactgtgcccagccatttgtcttatttatcattactatttttttgaggggggacagggtcttcttctgtcacccaagctggatggagtacagtggtgcgatcgtagctcactgcagccttgaactcctggactcaagcaatcctcccacctcagcctcctgaatagctgaatacaggcatgctctaccacacctgactaatgattattattattttttttttagaaatggggtcttgctgtattgcccaggctggtcttgaactcctgggctcaagccatcctcctgccttggcctcccaaagtgctgggattataggcatgagccacggtgcccagcctttgtcttagtttttggtccccctcacctgaccccagagttctagaaccattcccttggaaagaaagggactggtcagggctggggatggagaagtgctgtggtaggagccctttcttctctttcttccccagctggatcgccatacctaccctgtggtcctcatcttcctggcctcatagctcctcctctccaagcatgtcttccccgttgcaaagagctgtgggagataccaagagggccttgtctgcatcttctagttcctctgccagtctaccctttgatgacagggactcaaaccatacctcagaggggtaagtagatgttgcattgtgtggccacttcctgtgtccctggcagccttccccctcgttttcccacattgatgataatatgcgtgggactgtgtgaaaacaccagaagtgctatactcgttcagcacagtgacaccctccctccagctccaggtgttttttttggcggtcctagatgttgtagacatcaaaatagtagtacatcctgtttttttttttttttttgagacggagtctcgccctgtcgcccaggctggagtgcagtggcgcgatctcggcttactgcaacctccgcctcctgggttcaagcacatcctgtttttaatccatcatccatcttgtctaaacctctcttaaacacatttctatgttcagcatgtaccaaccctggtgggggctgagagctggcagatgggggagtaaagagttctttatttgttgcctggtaaattgtatatactttttggctagtgtcccctatttcttctacttggggattttgtaaataggtctgtgtatccttatttattaccatatatgagtttattctgtgtaggttataataatatatcttggttttttacctttccagaaggaaaaaagcgaatttcatacagatctgctttatcccctggctgttttaattggctttctctgaatcatttctgtttctgtcagtgctgctgtgggggaccagattatatcaaagttataaacttgaattgataagacatgtggtttatgtttcaagggggtgaaatttaactctagatacatattactctcccatttatggatgaatgatagatgacttcctgagccatgattatctttctctacaaagagcagtgatctcgctacctgctaagtgagtatcatttgattctcttcttcggagagaatactaggaagagttgtccttctcctgcctcatttcctcattcttcatctccattgctttcatgtgaaagttttcctttaaggctttgtcccggccgggtgtggtggctcatgcctataatcccaacactttgggaggccgaggcgggtggatcacctgaagtcaggagtttgagaccaccttggccaacacggtgaaacacctgtaatcccagctactcgggaggctgaggtaggagaatcgcttgaacctgggaggcggaggttgcagtgagctgagatcatgcgattgcactccagcctgggtgacaagggtgaaactccgtctcaaaaaaaaaaaaaaaaaagactttgtcccaacttcctactaacaataagctcattttttcttaaaatctggatatgtttttctcatggagatggggtgcggtgagagacagagaaaccataattaattagccttttatatggagggaatagggtggttatatttaaagagaaccatactttctcacaggaatggcgactctttgttagctgatgaagacactgactttgaagacagcttgaatcgcaatgtgaagaagagagcagcaaaacgaccaccgaaaacaacaccggtgagtcagccagttttcttttgtttttgaatcttgtgggggaagcagcccagctacttgggaggctgaggcaggagaatggcgtgaacccagggggcagagcttgcagtgagccgagattgcgcccctgcactctagcctgggcgacagagcgagactccgtctcaaaaaaaaaaaaagaaaagaaaacctaaaagaggttcaggtgatacggttcataaaaggacaagatttacaaagtattaagggatggaaataggaagaagagaaattttgaagagaaaagaagagaaaaggacgttaggatgtagagatacaggaaggggactcaaggactttttttttaagggaaaaaaataaacttcataacaatataaaaatcaattatttcaggactgagtctaactcagactctgctgtggaattggcagcttaagcctacttgtgtgataatgatgaaaccaagcgttaatgtcactgttaccttttttgtttttttttcatatttctgatctttttatacatattaggtggcaaaacatccaaagaaagggtcccgagtggtacatcgtcatagccggaaacagtcagagccaccagccaatgatcttttcaatgctgtgaaagccgccaaaagtgacatgcaggtaaagcagtgtctcacctctgttgataccatctcactttttgtaaggtggtaaggacaagtgtctacaaacttgatttgacgtgaacattttagtgagtgtaacatgtcaagaaagataaggttatggggtgaatccttggagtgatagaagggtaaccttgaatgagagagcatgttctttttttttttttagacagagtctctttctgtcgcccaggctggagtgcagtggcacagtcttggctcactgcagcctctgcctcctgggttcaagcgattcttgtgcctcagcctcccaagtagctgggattacaggcgtgtgccaccacgcccagctactttttgtatttttagtagagatggggtttcgccatgttggccaggctggtctccaacttctgacctcaggtgatccgcccaccttggcctcccaaagtgctgggattacaggcgtgagtcactgcacctggctttgagagagcatgttcttttcttaaaggtatcacaggagtgattatgcctttattgattgatggagcagatgaggagaggcattgcatgacacaggaaggaagcttggatgtgggtggcaccttgttcacccatgttattttttagctccatcttgacaattatacttaattattttttccttgtttctttttttataattttattgaagttctaatacccgaaagtgtacaaagcttatatgtagtttaaaaaataaaaatgaaatgattactcagatctctagccccagctcaagaaatgaaacattaatagtgctgtagagatcctttgtgtaccttctttctactccatttcacagtctccttattacccacaggtaaacactagagtgaattttgtgttaatctttctcatctctcattatagtgttaccactatgaatatgttctttaattatgtaaatgaatccattattcaggcttctatgatttaattatttgagttaacattgtttgagatttacccacattaatgtgtaattttatagtttctgggaaaatatttaaaattttcaaacatatagaattttgctagttatattttcgttatttgatttctaacttcattgtgttgtagtcagggaatgtggtgtttgaaatttatgtaggggccgggcacagtggctcacacctgtgatcccagcacttggggaggctgaggcgggcagatcactcgaggccaggagtttgagaccagcctggccaacatggtgaaaccccatctctactaaaaatacaaaaattagctgcatgtggtggcgtttgtctgtaatcccagccactttggaggctgaggcatgagaattacttgaactcgggaggcggaggttgcagtgagctgagattgtgccacggcactccagcctggatgacagagtgagactctgtctcaaaaaaaaaaaaagaaagaaatttatggagacttagccttatggcctagtattgagttggtacattcgttttatcttttctgtttctttttccacctttgcttgccttcctttggatttcttatcttctgcaaggacctgagcacattttaattccaaatgtcttttccaccttatatgcagttgtacagcgtgttagtttttttttttttttttttttttgagacggagtcttgttctgtcgccaggctggagtgcagtgacgtgatcttggctcactgcaacctccacctcccgggttcacatgattctcctgcctcagcctcccgagtagctgggattacagatgcatgccaccacgcccaactaatttttgtatttttagcagagatggggtttcaccgtgttggccgggatggtctcgatcttttgacctcgtgatccgccggcctcggccttccaaagtgctgggattacaggtgtgagccaccatgcctggccttttatttcttttttttagcccattctcccccttttatttttaaaattttattaaacatagtcattttataatctatgataaattattatctaaagtctttgtgagtataattctgtcagtgccttgtttctttatatgttttactaggttttattgtgagccactcattttccctggagttcttatgtgttggaattctttggggcctaggccaaagctgggtttttaagaaaggaacctgcttttgctttgacagttgtctggggacactccagaatcacttttttttttttaagacagggtctcattctttctaggctggagtgcagtggtgctatcacggctccctgctggtttgacctccctcagtggcgaggtggtgatcttcccacctcagcccctctcatagctgggactataggcatgtgccaccatgcctgactaattattctgtttttggtagaccagcctgggcaacatgagtttcactttacttgggttactgctcccagcctgaatcgcttttaattatctgaatgattattctgggctgggtgtggtggctcatgcctgtaatcccagcactttgggaggctaaggtggatctcacttgaggtcaggagttcgagatcagcctggccaatatggtgaaaccctgtctctac

SEQ ID NO: 103 - DNA, 5140 bp, Artificial Sequence
  subsequence of PTPRN2
agagaggttaaaagctctgtccaaagtgataaacctcattgctggcagagccagacccgcgcacagatctgcttttccacgctgctgctggacccatcctcgcaccctgcgcctcatgtctaagactccccacgccgtctgcgcatgctgccgctctgccaaacctgtccttctcccaccacaggcctcaagtctaagactccccacaccgtctctgagaggaacgcctcccacagcacgcacagagattccctagataagaaggacattcacaaagagggctgaaaggctcatttgagagggggtgattgacatttgatggcaattactaacggatatttttagtgtgcctctagtgtgaataccgttttgacaacagtggagttacaccaaatactggagcggcattctgcactgcaacaacactgaaggacccgagggtggcttctccgtggctctgaggttaacgctgcagcgtatgcagcaaacacctgtcaaggacccactgtgtgctcggcacaccaaggcaggtaacaaaggacccagtgcatgctcggcacgcagaggcaggtaacaaaggacccactgcaggctcggcacgcagaggcaggtaacaaacgaccagcctcagaagcagatgtgcttgctgcaaaccaccttcctacggcccagcagaggcacaacaagcagcgaattttttccagggcctcccgtgaacacggcaccctacgttgccatagcgacgccatcccagctctgagccgcaacaaagcggcagtgtctgtgtctcacctttggccatcttgttgagaaccatgtcgatcaggacgtaggtgccgctccggcctgcaccgtcactgccaacagaaggagagaaatgaaagtgctgccgtccacacggaagcccgacccacaacaaagctagtgacagttcatttccttcctgcttacgggggccggcgagaggaaaggatggcggatgtgagagggggaggaaggacgcgggctgggagaatccaagtgaggggagaagggaggcccctgggggcttcgagcctctgagcagcttgtagcttgtgggatgagctgctgtgctcagatgtgactccctcttgcttgttcccccctctttggccaaagatgtggtaaattttgccagaacaaaatgaactaagcgtttgactgtgtcagagttgatggcatttgtattgaaaatatctgcaaggtgccttctctagtgttgccgggcaacctcccgagcggagaggggagggcgctgggcggctgggccccgctggctcatgcacgcagggacacttcaggcacatccccctaggctgcctgccaccccttctaaacgcaggtcctccgtctcagaaggagaaattgacataacagtcgttactaaatcttactctttaaaaagcactctcatttcatctctttctcatcccaccagctataattggaatatttaccatctgggctagagttgtaaaaatgttttgaataatacttcaacagtagcaaaggaaaaaaaatttccctttttttctggaagaagagtagcctcaggatcatccttttcttatgaggaatgtgtgaagccatttcctgcccccgtgcagacgctgtcactgggttgcatgagtttgagagaccttcttaaaagtacatagggctcgtgttcacacccatgactggcttttaaacagaggaccttttacatttataatcctagcaaaatcaattgtgactttccggaaaacgaataaatgtttccatttcaagacttcaagaaagatgtgactacccagcagatatcacactttcggttctgctttctatgccgtctgtgatcccaagaaatgactggaatcctataggtgatcttttgatggtaaagaatcagcgcattttggctttaacatgtttcctcaaacaaatggttgaaaaaagctgcagttgggatggtaaatgtttaaaaaacggcacgttcacatcctcacaccctcactgcatcaccaaggaggagaggcggtcggcctgggccgccacgtaaccctcactgctcatttcaaagggccacggtgagcattctggttgctgagtgcctttctcacttgtctcctatgcacagcatatattcacatgaatattttaagatatacttcaaaaatcttccaactaagtgttgcttgtcttgatacatttgtgggtaaaaaagatgttctactctactctgtaaggataacaaaccgtaatctattttcatcctcctctctcatagacaggaatgcacacacaaaacacacatgcttcttgattggatttgacagatttggtgtttgttctttggagacaggtttcctttttattccaggcaggaagaacattctataagacagttctagaaaaatggaatatgagttcttcaccaaaaggagttgggtatgaagtccagcagagacagtactattaggttgagttttctttgcgtaaaatctattcttactggtgtggttttaacgtacttagggatatgagtcactgaatttttacccagaggcctgaacccacatgataaactggaggtggctgtacctcctgtaatgacagatttcgtccctgtacataactggccccaagcatttctcaccatctgcagagggttctgagcctctgctggacgtcctggtggttgagacaatgcaacaggctgaaaccattgtctcgatgtttctgtgtcatgggaccacagggtttaagaacaagcaaggtggcaggacatgctcacgggtgagtagaaggcaggacactgcaccgtgggctgctagaaaatattccatgtgaggcctcgcgcggtggctcacgcctgtaatcccagcacgttgggaggccgaggtcggtggatcatttgaggtcaggagttcaagaccagcctgaccaaatggtgaaacctcgtctctactaaaaatacaaaaaaaattagccgggcgtggtggcgggcgcaatcgcttgatcccgggaggtggaggttgcagtgagcctgacgagaccacgccattgcactgcagcgtgggcaacagagttaaaaaaaaaaaaaaaaaaaagcaagccatgtgcagtttatacagacataatacaagcaacgcccaattgtatttacccaaatggcatagaaaagtaagtttatatttttggtttcctttagaagtgccagttagttatatttattaagcaattagaaagttaagcttctgtatttaattgcatagattagtttttgaggtagccaaaacttcttaatccattaaaagttgtacttgcctgcaatgaacaattattggacaagaacggcccctgtagcacttgtttacttttctgaaataaaaagagatataaaaattatcgttgtgtactaagactttgcgttatccaaaacaactattaaaacaaagcgcaaggtctgtgtgtttgaaatcattttgcaatcgcttgttttgacggagaaaataaaaatcatacgcataatatcaaacatttattcttcttctcaacatgacgttattaatcggagctttaaaaccaatgttacacctctacactgctttcttcttccaaggaggggtgccaggctttgaaccagtggactcccggcctgtcctgcccgtcccacccctgcaggctctcctctttctgtttctgcttatgttgacttgaccttttgttacactacatcttgattcttcactaaaatttttgagttttgcaatttatgctcaaaatttcttagtccagacatcatattgaaaactgttatttccatactgtctaaaaacagcatcttcaagggcatttgtgacccgtcagcttttggtaaggatatctaaattacggagaaacctctatgaagtccaagacaagtcccttccatgcttataactttgagtatacaaacaacttcattgtgcagagtttagatcagaagttgtaaaaattagaaatgagcatatatttaagatttcataaccaacccctaaatcaaaacagaggactgatttaaaattcactgtgtaaatccaggtgtttttttttaattcagtgaaacaatgaaacatgtttatgaagtccattaaagttgtcagtgaactatttctgaataagtaaaattaaaggcggaacctcgggttattggaataaagcatgtgtgaaggagagtgttctacaatgtgctgcgtgaactcctacaagaaacaaattaagaaaaatggaagtatggaccatcaatgggaaaatgtcacgtttctttgcaaagccttgcctatccagcaccaaagagtccgtggaaagtaaacattccatcaaccggtgctttgctacaaacctttgaagccaggaggggcagggtggctgtggaaggagaggggaggtgagtgtccagtgagcatgaggggagggatgcttgaatacggcggagcccaaaccacagagctttctcctcctctcttcctattccctgaggagttaccagaacaaggcccttctgatgggagtgtgaatgcccagtctggagggcaccacgggagctgaaggagcagttgcagggactggactcggcgtgcccacctgcagatgggccgcggccacagcctagccaggacgcacttgatctgtgctgccctctggtctgtgaaacacgaggctgatgcccaaaaatgggcgggctcagacacaggcacaaccagcaacggcctccagggccgtgtcaccgtttctgttagaggtgtttgttgttgtttttccggggcacacagtggtgcgaaggctttggggagaggcggggaaggcaatggtgaactccttagaggcaacagcacttgtatgccagagtgccttgaacattcttggtccctggagaataaagaggttggaaaacagacccacttatgtctacttctacaattttccattcctgactcaggctgtaaagcatcgagggctcctgatgacagaatggataccaggcctcaggggtgcaccccagcatgggggtgggaagccctccagctcagtaaagaccatgtgtgtgtgcatgagactgcacgcatgtgtgtgacgtgagcacgtatgtgcacac

SEQ ID NO: 104 - DNA, 4606 bp, Artificial Sequence
  subsequence of BARHL1
cctccccagcctttctggtcccaaaacccgaggggggaactggccaagcccttgttccttggggacagggtgactgggaagtgggtgttggggcaccggacttgaactcgtttcctaatttccctcccgaacacactcaccagacgtagatgcaaatgtacaaacacagaggcatccacacactcaacccaaatagccacacaccgacacacacatcaatacaacttgcagtcacacacgcacgcacgctcagccccacagtcactcgcacgctcttagcagaggcaggaatggtgcttttcataaacaagcctcagaacacatcgccccattaataataaatactgattactgctccgggcatcaataattaaggcctgttacagtaattacacaattatgatgatgatgaataattcagggagggaaacagggctggtgtggctgagcccaccttagtgggaccctggctggtccgcctagcctcacctgcaggtgagcctagggtccaggcctgacttctccaaaccccaggccgttggctggccaaggcctctggactttggggccagacagtgcgcagggctcgcagtgcgagccctggaggatggcaggtgcagcctgctccagccggcagggcgtgcgagccgggagcgggtcctctccggttgccatttcaggcctgctcgcttctgctagcttgcagcccggccagcctgcagaggaatcaatcgcattcccttccctgaagagcacccccccatcccctcaggcctctccagggtgatcctaccttcataaagaggcacgcgcagggaggagcaccccctacccagctccaagggttcgttctgcgatctcctgggcagggtaaggcgcagttagagccgggctttggggtcagacagcagggcttaaaacccaggctcctttttgaaaaccggcccagacctcagttttcctctctgtaaagtggggtaagagccacggaaggccggtggaagataaaaggagaggatgcacgtaaggcgtgcgatggtgcctggcactcggttatttattaacaaatgagtacgatccctcttggcctccctacaggctctctgggccaagcagtggggaggggctggggtcgctggatgccctagccctgatcccgccatacgcgtttatttcggcccccaggactaaatggaagcgacagacggccgtcgggttggagctgctggcggaggcaggcaattactcagcgctccagcggatgttcccgtcgccttatttctacccgcagagtctggtttccaacctggaccccggcgcggcgctctacctgtaccgcggccccagcgcgccgccgcctgctctccagagacctctggtgccccgcatcctcatccacggactccagggcgccagcgagccgcccccgccgctgccccccctggccggcgtcctcccacgcgccgcgcagcctcggtgaggcgcccgtcggctccggggcctcctcccgcgggctcggcgtggccccttccgcccgcctttctgagggcgcaggttcgacgccctttcccgggagggggccctgcccggccctccctggcgccccagcccagtgccccccgaagggccaaatgccaagtccactgaggcccggaccccggactgcgtctccccagcccccctcggcgtcctctctcgcggccgctctgtccgggagccatccccacccgccgggtgtacatacgcgtctctgccactccccacccccagcctctgccggctcccctaggcaacccctttctccccaggagcgggtgcggctgattcccaggcttcgctctctcccacgccccttctacgctccaggtggagaacagcccctctccccgcgcccccgccagggagagaaggggagtgcggagccccgtctccctacccctcgagcacctgggccagcggctgagctgtacataccgtgtgcaaagtgtatatgaagttatttattcgtgacccatgagcccgtgaccgtgtccgtggattagtgagtctgtggcctgtgccctccccactcccaggcggggcaggaaggggccaagggggcttgcccacccaccccgaccccagcccccagcctcagccccggtccgggggcagccaggcctctcgggttctctcttttttaaatgtcgaaataaacttcttacaaatgaccaggcgcctgtccgcgctcgcgtcgctgggtccgcactcaacccctctcccatccctctcctggggactcggtttccttccatcctcaataacgaaacatttttgtcccccttctcttccccggtgctgtggacatgcgcgggggccccgacgggactcgctggggcccgggtctcccctccctggcctcagtcttcccatctctacagcgaggccagctcgttgttcccctcctcctcctctgtctccatctggttcctagcggctcccagacggtcccgagggccggggcgggggtccgtgccggaacaggcgggggtcagcgcggggcctcggtcggctgcgggccgggctgcggggccctcgccgagccgactcatctggccgcgacaagggccccggcagctggtgaccggcccgccgggcgcgggccgacagatgggctccgaggctgctccccccatcactcccaccctccgcccagcgtccggccgcgctcgggctcccgacgctcgcgggcgccaaggcggcccgggcctcccggcctcccgcaccaggcaggactccgcgcgccttcgctgcgaaccagacgccacatcgggtaaacccccactcccacatcttctcacctcccctttaccccgggaggaggttggaaatgagacccagaggggagccaaatcccagcgtctgccccatctcttctggctgccccagaccgcagctctagcatctcaggggagaggggtttaagtcccgagtcagactcgaggcttccagttcacgtccaaagggccacggtcacctgagactgctccctctctggggaggtgggcgcaggtgctcccttcccacacaccacctgcagctcgctaaccctcagcgctgggaagttttgggggccggcaagccgtggcccctctagtttgggggcagggtttctctgaagacgggggctgtgttcagactataacccctctcgaaaaccatacccacagccaggtccgctactgtttcctccctgccccgaaacaccctcgcacagcagctttctttcctgggatggcgagggtattattctaattttgcaaaatgcacagcagctaattaagtcaaggagcccctcggctttcgagttcttttgcatgtgaatgggggttggtaaatccccagctctggctctggtggggggtgaggagagactgtccccaactcagggttaatgaagtgggaaaggagtctctcatagcctcctgcaaacaaaaccccccatgcaacaaaccctgcccaaatcctacatggattgacttaaacccaggggataagtgctttaaattaatctcattgaataagaatgagaccaaacaaaactctttaaaatcatattaaaaatctatcaaaggacaacttgcactggtgtgcttttcattttgtgaaagtgaaggagatagaccgtagcaaccaccctcacatttacacagtggcagcgcaaaccacagacacattctcaactatgtaattgtccttgtacctcagaggcgatttcatatcctacattaatgaagaacaaggcttccctacaagtttataattagaatattttaaattgtcctgcacatcccatctgtaataataacttcacaacctttgcagtgaggtgggctcctgccttccatccacttagaaggctaaattggtttctgttcccaaccctgaggttgggcgagctctcgtgtaagaaaaaaagaatgaatgtgggaccccaacaggcctgacaataggagagggctgagaagccatcttcccccgctctgccactgacccatcccccctcataaacgccgactcaacagggagggggcaacaaagttgaaaagaagtttagagctccctgctggccctcggtcgcaagggaaattgaggactggggcaagttgagaacctgagctgattttcagggtgccccactccccccacagcgacaggcctggctctggattcttcctagtgtggagcagattaagagataatgtctaatgatcaagggggaggtttaacaagaccagttaagaagccttttcaagcagggcaacttctcccgtggaaaagcctccaacttgcgtctctacaaagcaacgcccggattttaatggcttccactgactgtggcctgaagggctaagcggggaggcccctggcagtttgggagatacctgctccgaaaggtctcatttactcccctcttccctaaagttctcctcccttcaccccagggggaaaagaaaaaaatcaaaataacccactgggcttcgttaaaaaaaaaaaaaaagtcctttactgagatttggttggttcgttgcttctctagagtgcaaggggctgtctgcacatcagc

SEQ ID NO: 105 - DNA, 3540 bp, Artificial Sequence
  subsequence of CCDC140
tcttcccaaaggttaagcagagtcgacagagcactttctggggtcagcggtaattgctgtgtgaaggaggtaccagaagtgtaatttcaagaaggcaaaagtaggctcaactctcagctaaaataaactcttggaccccgcgagcagttaaagaacatctagagctgagtctcatttgtttttgagccggaggcttggtctccaagccctcccagcgtccacccgtctctctcctgccgggagttttctctcctaagagccggcagatgctggagggaagcgcttgctgcaagattccttctcccggtcccgtagattgcagagtccgcgggaccgttgaacggaggtactgccggctgagctgggtcttaagtgcccatgggttcataaactagctgtcacttgctataaacatgtacaaatggaggccacaaggatgtgggggtgctggggacctaaagctccggaatcaggaggcgactcattttctctcctttccggcctctttcttttctgccgagcgctctgagattcatatgtacccagatgatcagcccggcccttctcaccggccagtggccagaggtccctggacacccggagaggggtgggagggcacccttaggctctggacagctgggccacatccaaagacttaatgaatctaaatttcttgtggcctgtgtccccgaagtccgacctgggatggccaggacagcaaagttggctgaggctgcctggcctagggcgcccgcacgccgccaaacctaacccggctcagagccgtgactccgcagaacagggcgcaacaggtctcaggaatgccgggtgattccagccactctttctctgtctccgagtcttgggcctcccctttatttctttctgaagtctctccggagcccaagccaccccacacccaaaccccgcagctggatgggagtccaggccacttccctatccgcagccggtgggccaaggagtcctggtgccaaatttggccgcggtacaggcgctaattggtaaagggatgacccagcggtggggcttggaaggcgagggcccgcgccctgcatccttgaaccctggtttgggaggcagtgacttttacctgggctaacaaattacccagaaagcatcagtgggactctccacaacggtgttgtgaacgagtgtagggtccactcacgctctgtgcacgttcgaaatgaaacgggagccttgaagacgatatttggataatttgacaaaaaaaaaaaaaaggtgggcgggggagatagcttctattgtcccctctgcgcctctgcccccagtttcagagacactacaggtgaaaataacagcgggctggctgtccaggcctgagtggagcgtgcccttgttagcttgaaagttctccctcgcagcccgtttggatgcgtgcgtctacagcccagtcgcactttggtgaccggcctgggctgtgaagcaccctttagcgaacagcctccgcacttggggacactggcacaagggtttcatggcccgtggacttctcagggcctctgagggttgacttcatactcagccagacacaggtcttaagaggaaattatgatctatgcaaaataatgtccctgacctagtggggagcgggggtggtgtctgccacccagacctttttcctcccgtctgaagggccgagctctggcaggctgacacggtcgagcagagaagttcctggccacccgcccggccccaggcgtgattctcaggatgcagtaaaagttgggcaaacgccgagaaaccctcaatcccacagaaaggctgccgggggctcacggggaggacatcaaaacgccaggttcgtgatcatgagcttccggtccggatgtgatgatacaggattggtgaatgcccgggttaaacccgtggtccttcgcagccgctgcctcagtgacagtcattaggccccccgcagttatttgtcaaaagcattggcgccgctcttctccccatcgaaaaatcagtcgggatgagttcggattaagggactagatgaggatgtggacatcccattgaaacatggtgtaggccagccgaagtgggtttgctccttcagctgggggagccccagggaacgtgtgagcgggtttacaataaggaacatatggtttgtgggaagataggaagagagaaggaaaataggaaaggaaaaagaaaagaggaaaagaagagagaggtacttttttttttaaaggcagtgagggagggagagaagaataaatctcacagcagctggtgtcctagttcatttgggtctgagaaaattatttttaggcaaacaaggaagacaggattccgccgcccgattctccagggcgggaagaataggatgtcttgtaaaaaataagtaagtcctgtctaatcttccaaaaaatgttctgctcaattaggtaaaagttcatggtggggtgggggtgatgtttagaagacagattcttcggtaactgctctttggtgtgtgtattcggggtgtgtgtattcggggtgtgtgtattcggggtgtgtgtatgtgtgtgtgtgaaagaatctggtgtgtgtgaaaggggtaagggttgaaggaccttatttagctgggggaccgtcttgtattgggagcgggcagcctaggccttggaaagaccagaaaatctgatctgtgcggaggcaggtgaaggctgtgagtgaggtgggggcgggaacagagctggaggaagtgaataaaagaaaccttggggtccgttggtttccttgactcgtggagcaaaaaagtaggcgagtgaccactccaggaactcctgcctccgggcgccggtacataaagagctggggctgtgtgtcctgctcttgggggaggagggtactgggaagggggaactaacaggtctttctttcgctcggatcaggactggactccttctgactccacgcggccccagctcctgaaccagcgcagtgagcctcagccgcttctcaaactgggtctcagaaaatttctcctcgagatggatttcagcggcaggaaagaggggccagtgacgcgaggagcaagaaaagagtaacatgggagatgaatgttcaaacccagatctcctcgctgagcctggaagctcgccaccgtgggatcacgggaaccagaggcaagaggctgcgaatgagtccaacacccgcgttcctcgagttttaaaagcacacctggggccagagactgcacagccaactaaacggagtaaacgcaacaggtggaggaggcagagttgtcaggggccaagcccggcgcgatctggccagttcttggggagcgcggacctgggactgcagagaggcgttttgaagagtgctgcgcgcacctgcctctcggagatatccaactccacccgggcgtctcctgagagcgcacagtccacagaccccgggagggctgcccgccctaggacacgtactctcccgacccctcactctttcaaaattggggaagaggcggaggagatgaaaaagaagaaagagagaaagagaaga

SEQ ID NO: 106 - DNA, 2301 bp, Artificial Sequence
  subsequence
atcataaaagaacctcattttacggctagggcaatgaggcccagggagagcctgcctaagggcacgcagagcaaagaatacaacattacacccaactacggagggccctgtgctccggacgctgcccggcgtcgctcagagtgggtgctcacaaattatttgtcaattgattggaggttgccaggcccatcttgcaagacacctcccttctccctggaaggagaaacccttgggagtctgttggttaacgggaaaagtgctttctctgagaggaaggcggggcaagccggaacaagctctcgctgggaatccgcacactccttctcccggtgtttcttaacccctttcttctcccgggagtggagagggggctcaaggatactagaaacctgtccccttggcggagacacgtagtcgcctctggccgccgcggccgactgatcttgcggagtggagctccccccaccccgcccgcgtttcgctggggctccctccgctctgcaaagggagcggggagctgggggagccttgcacaccagtcttcctggagctgcagacgaggagccccgcgccttcgggagcctctcggcggcttcccctctaacgcgcgactgtgcagcctctccccgccgcccccacccgcggctcgccctcgctccctccctccagccccgccagccggagcgcgcacaccgggactccggcacgtgggcgcgcgggaggtggcggccgctgctgccgctgctgccgctgccggagcccgaacccaagccccagctggagccggagtcggagccggcgagatcgccctccatgctgcgggagctgcggccgccttccgctcagcctctggtcccaggcgagcctggcgagccggcgaagcaccggcggggaggaggactagaacaggaggaggggcacggcggattgaagcgagctgggctgtgagcaagggacacccacagcctggagaaacagccccgctctcttgcgcgctgtctgctccagccgctactgggggctctaagcagcgcgatgctgcttcgcttcttctaggcggcggccggcggaggctttccgcagccgcttggccggcgccggcccctattccgttggcaagtcccttgtctatcccggagggcgcacccggacgctcgagccggagcgagcgcgaagtccgaagtccgcccccagagccgccaacttccctgtgagcccctctccccgccgcagcctgcgccagacctgggagcgatgcgcccccacccgggcggcgcgccctctgccaactcgccacccatgggatcgactgccctcagctgcccggcttggagtgcgtagccttcgccgcgtggcccaggagaggagcgagacaccaaggcgtttcgcggactccgcgcgcaccagtcggcttggtggccctgcgggatagcgtttatacccaggggtgagtaggccgctaatggaggccccctttacccccagctagcggcttgcatggacggcaaagagacgggagaaagttgggtctccttgcgcttaatctggcattggcagaggaatgcaacactgtttcctccaactcgattgccgcgggggtttccccagttccctccatctctaagccaaggagggagtccccttccgcttcctaaggagaaaaggggctccctagagcagatcgctgacttcctcaccaacgtgccatttggggcttttcagccgcccgcgcgttaagctagctcccagcataagcgcttagagatccgcagccgcctgggcgcgctaagagatcgtgggcagacctagcccttagggcagcctaagcgcgcttcactaaagaaaaaagactgttggtatattaaagaagtgtgtgtgtgagagagaaagagacagagaaagagacagagagacatgggtgggggcaccaggaaaggtagcccgagtaggaggtgctagtgaaatttatcttttgctgacggatccagcctccggaatgcccgattgtcttgatccgcggtgcggtgcgggttttaggggcgcacaatacagggtgtccagacagagtccaacaggtggcctgcgcgggcatcctgtggctaggcgcgggggaggggaacggctccctccccgagccccgggctgcctctgctgcttctaggggtgcatctgaacatggataaggaaatgcgctccctgctctcaggccctatttggggtgagtgtgtgaggcatccaaatgcctcctgtcggaaaacagcggcctcattgtgtgcccgattgaggtttcaacttgcaggc

SEQ ID NO: 107 - DNA, 5043 bp, Artificial Sequence
  subsequence of GATA2
aagcatttccctctttctccccaacttttctcctcttccaccaaagctctagaatttttctgagtcctggaaataatggatcgaagttgttgttctttaaaataaaataaataataagctctttcttttttagataagtggcctgggttttttttgtttgtattttttttttcaattaaaagaccatctggaattcaaataaaatgcaaaacaaacaggagaaaggaccaagaaaaggcaagctgacgggtctgagcagaactaggaccctgagccggggctcagcgggtgctggggccagcgggggctggggctggggaccaagttgagcaggatcactggctggcatcgtgttccccaggaagttattcttgatttagtttaaactaaatgaggagtacagcctggcaagagaaggtggttcctgcactcaggagtaactgttttaaaaaccttccccctctgtggctcaaagggattcgggaaagagacatggacagcaagaactccacactagccacgagtttctgggtttatgctctgtggccagattcttggacaaacccagtttggccccagccagctgcgggaaaaggggcgggggcactgcagcttcatcccctcactccagccctcagggagggagagggaagggcctggcgagggcagggagcaggagcaggtcctgggaattttgctctggggaaaatctcccgaggagatcagggagccatcgaaatcccaagatcagactgattgagttagagacccagattccttaatggtctggaacctccggagtgcctgaaacatgcacacacaacatgcacacagacacacgtatacacatgcacacacgctcccaaacacatgcacatacagagtcacttccctcgcttcacatactaagtcctgaggtggcttgaattttcacttaagattcagggagggaaagggggaattcctgttctatgttctggtcaggcagtgaccaaccctggggcaaggaactgaactttgggggtacactggaagcacttaagaaaatggcaaaagttttagagtctcctctccctgacccgggggtctcaaacatctgctgggggctattagagcgagacatcacccatccccagatctgggaaaccaacactgccacctctcccaagtcacagctccccaccacaaaaacgcaaatgctcccctcttccacgaagtccccagcacctgcctttacctgaacaggaacgagccttgctgcgctgcttaggggtgaagctggaggccggtccccccaggaagcctccggggtggaagagtccgctgctgtagtcgtgggcagccgccggcacataggaggggtaggtggggatggggtggtgtgtagcaggctgggtgcccatagtagctaggcctgggcgcaggggactgccactttccatcttcatgctctccgtcagtgacacctggtacttgacgccgtccttgtcctctcctcgggctgcactaccccccgcggaagatgaggctggagacgcagcccccgtggtgctagggtcaggagacacttctttgggtggcgtgggtgggaagccgaaaaggtgggagccagagtgggctgctgtaggggtgagggaggccactgagctcccgctgcctcccccgctcccacccccagcccctgggtacacagagagtgggcctccagggcctccagcagctgaggggtgcagtggcgtcttggagaaggggctcacggtccaggggttgtggtggtgggccgcagcggcagagagggctgctttgcccccgtccagccagggcaaacccgggctgtgcaacaagtgtgggcggcacatctggcctccggtcaggcgggctgcgggcaaagagagagaggatcagggtgggcagaaagatcagggtaggcagagctagggacgcccctgacagacattgagatcacgactcccagaaccagcagtcatcccctccccaaagaaagccagaaacataataccccaccggtaataatcaggaatgtcagtccaagctgaaggacaagtggcatagaaggaaccccaccggacagaccctacagggaaccctcacaggccagctggaagtgggcagaaaccctgtgggtcccagaccctccccaatcggccgctgctcccacctctcccgccccaatttttcagcagctcgattcctgcggatcctacatccgggaagcaagcagacgggccctcctcccctccctcgcctggcgcgcggcgcctgggttctcatcaccacgggcccagtgctcaccgtgcgcggggctgtaggagacgcgcgcccgcgcgtgagcggggttggcatagtaggggttgccctgcgagtcgaggtgattgaagaagacgtccacctcgtctggaggcagcagctgcgcgggttccatgtagttgtgcgccaggcccgggtggtgtgagtcggggtgctgcgcattcagcacggccgggtgcgccatccagcgcggctgctcgggcgccacctccatggccggcggcggcggctcagggtctgggtgcagacggcaacggccctgcgcgaggaagggggagtgaggcgtgccgccagcgcctgacaccccccaaagtcccaccacgaggtgtcccgcacgccacggagccccagcccagatccggcgagaaagagcaccagtcccgggtgggaggaaagcccaaggctcaaaacgaaaggaaggcgggggagggggttcagccacgcacactcacgtggtgacccgcggctccagaatcacacacccgtgcacatggggtcacgcccggggacgggtcccgacaccagtgaccccaacaaacgcacagagcagcacttcagtcagacactcacactgagcccccccgcccggtagacaaacacatgaacacagactcaaaagttggagacaggcgcccgggcacccagtgtggcacttgatcccagcgacacgcacacacccacacttggcgccagatacacatactgatctcaaccccgaaaacatgcacacgcagccccctgagcgcagtactaagcggcacaatcaggacctctcaacaaagcacaccaaagcagtcgcccgcagcctggccccccgccctaagtccccccagagtcccctcaaagctaggagcgccccaggcccccagccggctctcaaaccccaaacttacacacgcagccgtggggaggggagggactcggcctctgagagtgaaggagttccggcgggagccccgagggcgacgggcccagggacagcacgtccggaggctggcggggcttacagggtaggagctgggggtagagtgcgcctcggcctcgggccctcccggctccggcccctcggcatcctccggccgccctggcgccagctgccgactcctgcacagacatgaagcgggggccgcgcacgcctaagaagcccaagccagccaatcaacgccgcgcgcctcccagcctccgcctcccattggctgcgtccgcaattcccgaaccgctggactccgggactgacccgcgggccggcccccctcagccggcgggccccctccctgcgcgctcctggcggcccctgctgccaacgcgccccaccactaagggaccctcaccccaaggcccgacccctgcagcccctcttgcgaacactccgggatccctcgatgctctgggcctccctagcagtaactaaccaccaactgccccctccccaggagcccgggccgcacaccctctggatggcctctccccgagaccctaaccccgccacgctccttcaccgctgaacgtggtcagtccgagcgcagttacctacccagaccgttccctcccctagctcgggcgctgtctgggtggactgggggttgggatggttcggacgacagcggcggaacagcaggagccgagaggggcagagaggggcaggaactacagggctctagagggcacctgggcctcggacacgcggccgctcagggctgtgcttgacccagtggaggtggcccggccggtggctccagaaagggggagggggcacccctccccgggcgtggccttcgcaggccaacgggcccaattgcctggggcgaggccgtggagacccggactggcgccggcgccagctggagggagacgcccccgggcaagaggtggcattttttttcctccggggggggtccccgaggtggcctctggtgagggggaggcggtggtctgaggtctccgtcttctaagtcacctggaagtgctccccactcacttctagcccggagatcggctctatggttcgtgtgggccgggcgcacaagtcttcctccctcccattgtacagactggggagacggaggctcgaagggagccccgggccgaggagggaggtccaggacaaaggcattttgggggacctagacaggtgccgagtactggggggactcaacagcgtcctccagccctcttccctgttgggcccgaggctcccggggtagatgtccagagggcccccaggtgggtcgggggcaggaggagggctggggtcccggccacccccgggagagtgggggaggggcggcgggattgacagtcggtaattgggtaactggaggcggcttctccggccgggcggccccgccaccgcgacgccgagcccctgacgtcacccgattcgccaggaaatgaactttttaataatccgaggagggaggaaagccctcggtcccctcggctccgggggcgcccggctgggcccagctccgcgggcgcgcccccgtcaccccctcccaggccagttcagggcagcgccaaggggccgcgggctcaggctgaggcctcctcctgcctaggcgtctggcgtccgtttgtctgtccgaggcctcagcgcaggtaaagcgcggggctggggggcgcagagctccattgggctgggaaccagcgcgtctcgtgcccttgggggacccgcctgcccgagccgcctgctaagcctcccgctcccctccacgc

SEQ ID NO: 108 - DNA, 7737 bp, Artificial Sequence
  subsequence of ADCYAP1
ggttcccggaggcagccccagagtcgcggaccgatgtgccaggctgtggatgagccctgggtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgcgaagggggggtgggcgggccgcccggcggggtaggagtggttgggtgtcgttgcctcctccttacctctgctcccacccccagtcctgggagaagagacaattctcagcggaggacttttatcacctgtgaaaatccgcgcgagccccttactttggatcctcgccgagctggggaggaacttgcactgaccacaccttctgtccccggccaccccgcaggccagaggaagaggcgtacggcgaggacggaaacccgctgccagacttcgatggctcggagccgccgggcgcagggagccccgcctccgcgccgcgcgccgccgccgcctggtaccgcccggccgggagaaggtgagattcgcgcggcctcgcgcacacccgcggctgggagctcgggactgcggtgacgggaggggcagtgtggtgacccacccaggatttttttttttttcccgtgaaagtcctcaagcctgtcctctccctggcccgatcctattgcagcgacagaaaatcagcagcgggcgggtctgtgtggacctgagggccgcgtggggaccgaggggggctgtggcccaaagagtggcagtgagtggcgtcaaggaacccacactccgcatctgccactcctagagccgggactagctcccgatcctagcagttgctctcgagatcatcccgggagttattggcgagttctgggcctctggaggtttccctgtcagcctccccggccgccgagggggcgcgcgcccaacaagggggtctctagcggccacctggggacagaaacagtgaccctgggcgcgcactttgcctccccgttagagatgtcgcccacgggatccttaacgaggcctaccgcaaagtgctggaccagctgtccgccgggaagcacctgcagtcgctcgtggcccggggcgtggggtaagagtttgtggaaggattaacctgcgcgcgccggggtgggtgcctgtgcggggcgcgcggggcgggcggcggtgggtgcccgtgggggccagggtgagtctgcgcccctgggtctggggtgggcatccgccacgggtcgcagttggagattttgaagtggcactttaaatttgcccagagagctctggaagaggcaaaaagggaacgcgagccagggagtttgatccgttttgaatgaaaagaaagagaaaccaaaccaaacctctcagtcatccaaaaccttcaggcttccagggaggttttgctataattttctctaagcatgactgtttctgggggaggggaaaggggtggttgtatttactgaaaattcaaatcgaaataataaatggccaaatgtggacacttatggacccaaacagttttgctcacgccagagaaactgagagcacagggcttgtgtgaagcctatctcggcagaaggcaacattctaataaagcccgtgggaaaacagattacattttcgccatgaataagtcatgcagtgaaaaatattgcctacagcctgtcgacttatattattatcacgtttttcaactcggcgtgaggagggagaggagtgttcatatttgactaggaattgcaggatcgatgcaaactccagggcagcagccagactggcatatgtagggctctccggttactttctctgtatgtcgcgggtgagaggaacagcgaggacaatttagcgcaaacacacgaagggtcggatctcaagggggcagcgctgggagaaaggttaggcttgaagcgcgcgtcgcctgcccggatcttatcccgggccccctccgcagggtttggtgccaggagatcctgcgtggggaggggggcatcgaggggctgccgtctcggccctccccacggctgcttccaggcagaggcgggcgacgcggtgggcagtgcgagccccgggccctccccgaaggctcccgcgtggggtggggcccgcctgctccccgcggcgattgaacctgtgtctcccgccccgccaccctcttcccgacccctttgcttgcagtgggagcctcggcggcggcgcgggggacgacgcggagccgctctccaagcgccactcggacgggatcttcacggacagctacagccgctaccggaaacaaatggctgtcaagaaatacttggcggccgtcctagggaagaggtataaacaaagggttaaaaacaaaggacgccgaatagcttatttgtagcgatgggttaccagctaccctgtgtatacagccctgacgcaatgaaaagtcgttttccaaactgactcaacagtcatcgctcgtgtgttctatccaaacatgtatttatgtaatgaagtaaagccattaaatgaatattttgataataatattgtttttctttctacaaagcactagagaatgcacagatatactttgtggaccaattattgatatatattataaatatatataaagaatatatatatatatatatatatatatatatataaagtatagagagaagttcatacaaagcgtgcacaaggattgaaaattcgcccgagctgtttatgtttttataaaaataaatagaaaagtagacaatcattgttttgaatattactcctatttttgtaaactggaattaaaaggatagtatttttatccatgacaggcctgaagatattactacttaccatttgctactgtacataaacaatgatgccctgctccagggagattttgaggtaaagatatggagaattgctgaagggcattctttcccagtgagtctctggggcaggctgcttcaatcccagcctaactcaactgggctctgtccccctggttgggtggcaattccaatatttctgctttctttgattctccttttatgtgtagttgtctctcttcagactctcagcccagaagaaaattctcctgataaaacaacagctcgatccaaattgtgcttctccccagaattcacgcctctccctaggagaagagttgaggaactgtacagaaaagggcggcttcgttagaccgctctcttttctgtacttcctgagtggccagggaatctaatatccccaaattagggcaattggaacaaagtgaaggacatagaggtatattggaagaggcagagcctgaggtggtaggaggaggaccctggaaatggactggtttgagattgccccaggtctgggaagctgagggcaaatccagtcccaatggtcctgactttgggcgctgggtattggaaatggatgcaaagtacaatgtgtttttctccagtgctgtccatgcttctcatcttgtgaaatggccaggatcctctcctttgaaacctgctctgtaggagctacccttttcctttgtggttttatggagacctctccttcctaccctcctgcactgtttaagtactgtttaccatttttcattcacttctcttaaacttgtgaatgcttctcactttttttttttgtttgatgcaggcacttattgtaaattttagaaacccctctgtagccactagtaagtaattatgcactaaatatgaaccctttgtttcttgtttattgagtttgtaggtaaaatgtatttttctacattattgcttattgcttagtaaaatttatttcataaaaccaacctttgtcatattagaatgtgtagtgttcacatgttgctcagttttgctaactgataaatcatttaatcctcttcttcatatgtatgagtactatcttatatctgtggtcaagagtgaggtaagcaagctccaacagaccctgagaacctacgcttgtatcctttctttggctaaagaaagcatgtctgtttcctgtcaattctttgaacatacagagtaatctttataaacaaaagaaccttcacccagcaatcagatcgagcagcaacagacaaaccagccagccaatctcccaaatttcaggcacaagtttatttatttatttttttatgttttgaaaaaagaagatgaagaagaagaaaaaaaaaagaacaaggaaagattaaacgttagcttgtaaagtttaaaggacctttccttttcctttacggatttgatcagtatgaagtcataaatcaaagaaaacagaattggatttgcattcccaggcgggatggatgctgccaggagatcacattgcaaatagtgaaaacagaggcattcggtctatgcctgagtcctgtgtataggatcaatcttcctttaattccgcagtctcctcaggcaatgtgacacgggatgcagtttgcagctttagtgcctttcttcgccttttaaattgccacgaatcacagatggctatttagtggccctacaatgctgcaacacatcagcttgcattttagtcttaattatttgtttcttggataatgggcagagttttctgtatttgtatcagctgttagtggtgaaatagggctctagttaaccttttatttatgaagtctaatttagtgttcccgtggctagttgcaagcattttacagtgatcacccagtttaatcttttgtatactttttagaaatgccaagagccttactaaactgaagcagatttatgatatagtgataatttaggtagatgttagtcttgaagctcttattttgtgtgcaactgattataaaaacaccttaaccaagtattattacacacatgatatctataactaggactttgataactgttatataaagtgtgtaaaatttgtatgaataaatttttgtaaacaatgcaacttggtctaatgtttgggaaaaaagacattcaggaaataattactttaaaatctcttaaagtattatatttctttagcaaccataagatttttttacgtctggaatatatatctatctaagcacccttgtattttcatgaactgcactttaataattgatgggcaactggattctgctaaaaatttaaagtagctactcagatggagatgcctaagaaggttttaagctcataaacaggcatgatgttgcaacattataagacacacaatttagattaatttccatcccctagtgtgtatatactttgctcaatattcagaaagttactaggtagtagtgggagacaatgctggagcattagttacacatctaaaatagcaatctaacattgttcttttattttttattttagtggccaggtctcactatgttgcccaggctggtcttgctcaagcgatcctcccacctcagcctcccaaagtgctgggattacaggcgtgagccaccacacccagcctaaaatagggatctaacattgttcttatacaagtaactctgcagactaaacttgtcttgataaaattttgtataaaatgatctaaataatcagtttttggaggttttaaaatgtgtttagagacatacaaactactgtctctgattaaaatgctttaggtagaaggaacgtgaacatgagtaagtaaagagttaattagatgcctttaaaagaaaatgtactttgaagtccaggaagaaacacaagaagtcatttgtggattgtatgctttcttagttcatatttacaaactttagggcaaagctttcatacgaaattccttcaaattccgtagtggtgtgtgttttggcacctttgactatttctggcttagaaaatgtatagaaagtcacacaataattgacataccatttaatttaaaatgccagggtttcatcctaaaaattaatggtctcaattagtaaatcaataaatatgttacgatagaattaaaggatgagtgaggattctaaaattatcttcagaatttagctcagtatttaaggcctagaatcaaatagggaggagcccacagtctagaaatcccgtttgtagtcaatgaaaaaatgaatccagtacagttcatatttgcatttgattttattggataaggaatttttcttctccatctttaactgcccctctttgtccttgaagacatagtgtggtagatgaaaaaatgaagaagactttattcggttggggctaggctaacgacttgtcaagaacataaagataaaccccagacttggctgacttcaagtgaatttcatgtatttagcaacttgccatattatcttcggtgataactcaaattacatctttttaaaggcagacttgatacatatgggtattcaagaagctgtaataggtgccttaatgctgttagggcggagaacacacttattcaatacaatgcacacttattgaacacctgaaatgggccaccgcactggtcgagagagcttgactaacacctgggagctcaggtagtatttttttcagaatgtttttctgaaatgtgatcatctttggggcggggggagcttaaaaatgcaaaattgtagggctctgccagatccagtgaatctgcattttaaataaaaaccctagattactgtgcactaaaatttgagactgcctagatttagaattggtgacatatgtaggcatacatatgtgctggtctgaatgtttttttcatatgaaataagcaaaaggtcatgttacctgcatacagtaataaatacataactgtgccatattcttccaagatatctggtcattaagct

SEQ ID NO: 109 - DNA, 8315 bp, Artificial Sequence
  subsequence of TBX5
ttatgcggggtttactgcttacccagaatagcggctactgctgcctactagggcgcacctaccgctggagcctccgcggcgactgcccacctccaacacacacctcctctgctgtgcgcttgctctccctaaatacttcccagttggcaagcgccaaagaacacaaaatagcctccaagagcaccggcaaccatataatctcagtgccccgctcctcctttacacccccagggagggaaagttggaagcccagtgaatggtcgaagtcctttctgagcgcagctttcaggattaaagttcccggattcgaggtaagagtcagtctctctcactctctctccctctctctctctctctgaaatacaagccaactcagctgagcacagtgacgttgggttgcctcgatgctcacaaagtactgaaatcgcctatccaactagctcccactgcactctgtcgccctcccccttctctctccctcccaccctcccgtcctcccttccccccaccccaaatttccactagatttcccgaacactcaaatcacaatacgactctcaacacagacctgcaataactccctcctctctacccctttcccaacctccatccatctctgcttctctgagcaagcggtggctgcaaaaactcctgcacaaatcatttcaaacgcggtcggcttctaatcgggaagtaatctcagtgacgctggcggtgcagagaaccgagtctggacgcacacacacaaacacaccgcgggcctccgcagccttgcagacattctctccaacgctgacctcgagaaggagagctgcctgaccccgcctggccggcgctcgccttcctgacatctcccctccaccccctactcatcacccctactctcaacgactctggggtgaaggcagggtgaaggtgcattgggatgggggccgagatcaaagatgctaagaaatcgctgcgggccctcaccccccacccagtggggccaccagacgtcccctgctccaggtgtacacaaggcagtgcccagttttattaaaataatcaaacagtcactggggactggctagggttttcatctccactggtctcctctccttgccctttccctctctgagatgtattttttttttttagggtgagttgaagaggtcaataaaagcacaaacacagtcagtaggtctcagtctgagcagacccagattgggacaccgaatattggtgcttggagtaaatgtcaggagtcctgttctgtttcccatccttacctcctgtacccccatcatactgatttttctagggttctgataagagacttgggcctgcaagtcaaggtctggagaaagggatggagggaggagaaatgccccgaggtcagcagctgtgtaaccctctgtgactgctctgctaaaatcccaggccaggccattgctgcaaaatgccaaagtggagaatctccaaccctgaatgtggggcaaccggggagaaagccagccctcctttgctatggtgctcactccaccttccaggtttgtttgcaaacagatattttccctttgcccaggcgatgtatttatttgtgaaaacaccctcaccctccaagtcagcttggaggcctttaggcttgctggcaaggcgaccctaggccgaagacacgaaaaacaaacatggcatcggaagaaggctgaaccgctacatctccagtagagggaaagggaaatccagaaagaccttcccaatccctttaagttctcagagatttggggataaaattaattcccataaataatcttcaggcccctccagagaggaaaaatctggactctcccaggtaatagcccagccagatttgcttagctcattgagcacagatggtctgggccaccaagcaaaatagggattttcaaacctttcgagagaatccattcttgccaggtgacacacgaagccattcttatttcgttcatgagaagtagcagcacagcaagagaagattcagcaagactaagacggggtgaaaagccaaggagggccaagtgcaaagagaaacctatttcccccctcagtcctccagggtgaaactcacctccaactatcccacctcttctttggagcgcaaaagccaggagtgcgcctcgctggagagcagggctcaggtgaagactttgaaaaggaccaccaaaagagacctctttaggccaggtcttcttccaacgtctgtcaagaagagcacgtacctcccagctcaaggttggtttgtggtctccgacaaattaaatattactgtttattaccaggcataccccagtaaaataaagaggcaaccaggcgatagcgactatctcaccagccgctgcacctataggacttggagacgtcacgagtcacgcaaccggcccgcgcgctgtcacgtttggctggggggcagcggcgggaggtaaatttctctccgtcttcgcctatcagtgccgggtctgcgcagccacaggttccggacgtcttggccccgggaataaataaagacataaaccaacccggctttctccggaggaatgagggtgatgaacataagacacaaatagagccaagatcgactttcttaggaagggggagaggagggaactcttcacgaagggaggtgggagtccacctcagacctctattggaaggaaatcgagttgttccgggggactgaggtctcttgcataaggcatgggatccttattattattattattatttttaaatcccccgcggaggagctctgggcaaatgaataccgaggcgccgctctagctggttaggcttgggatgcgataactcagtgccctcttgcagacttgcatagaaataattactgggttgtcgtggaggggacacgagacagagggagttctccgtaatgtgccttgcggagagaaaggtccaagaatgcaattcgtcccagagtggcccggcaggggcggggtgcgagtgggtggtggagtaggggtgggagtggagagaggtggtttctgtagagaataattattgtaccagggcccgccgaggcacgaggcactctattttgttttgtaatcacgacgactattatttttagtctgatcaatgggcacaatttctaagcagcgcagtggtggatgctcgcaaacttttgcgcaccgctggaaacccactaggttgagttgcaaaacgtaccgcgtagacgcccctggtggcgccgagagaagagctaggcctgcccagcacagagccggagagcgtcgggccttccggaagggtaagttctccgccaaggggtcccgagggagctggacgtctgaatctggacttgcccccagcttcggggttcgattctgggttttgcgcgtccccaacccccagggctttccgaagcatggcctggctccaggcccggtcctgtaaggactggaacggcagcaaaatgtgcagggaggcagtcggccggcagagctgcggcgggagccaaggtcaggcccgcggggagagcgggcagcttccagcgccggccacaagctcccaggccagctgggccgcagacccctttgcttccagagagcacaacccgcgtcctttctctcagccaggctgcagtggctgccccgagcttcgctttcgtttcccaagctgttaataacgatatgtccccaaatccgaggctcgtgtttgctcccagatgccaagaacgcaacccgaaatccttctcccaaaccctaggtcgacgagatgagttcctacttgacctctgagccgaggtgggccggaaaccgaggcctaggccccgccggggctgcaaggaaaaggggaaactccgagcgtagcgtcttttccttgtggttcctttctccggcatcccggactgcgggccctgcagccacctggaccggcattcaaaggattctgcaagtccagcttcacagactggctttcccagacgctccgaagcccgcaccacgaacagaataaaggagagacgagagatcgcaactagatttgagaatcctcgttcttttccccaatcgttcgggcagtaaactccggagccggctacagcgcgcatcctcccttcctcctttcggagaactgtcgctttcccgaagctgctcccagctgggttttctcccgaagtttatttttctcattttgcaaaattcctgccctgattttaatactgcaacaggacagcagaggttcctctttaaaaagtcaaaacccaaaccaaaggggcccattgctgttgataatttttttcttcaccttctccttccgtttttaagaaagacacttccaaaactacccattgttttcctctcaaaagagaactcgctttaaaatgcatccctttctatttctctccggatatgtccccacctgacccggtggcaacttgtggggcgtgggggtagtgaaaaatagaagtgtggacggggctgggaaaatgaataagaattataacaatagaaatgatgacaaaactaaactttgaccaattcaatttgcaaaagaaagggttggtctgagggtgagacattccctggtttctcaagggggtcactttccctctcaccgagttaaatctggctctgttatattttttccaggaacacagtatgtctcttcccaatgttttattcggtttctggtttccccacttttgaccggaagtatgtgggaagaatactggctgttattattatgattattgagtgtccaatactctcctccaatatttccaccaagaattattatcgctgctctcctcattcatgttagtgttgctttgtgtgctttagatgacaatcgtcctctttactctccaagtttggagggtgagggttttttgttgtggtggtggtggttgggttcttctttgtttttgtttttttgtttttcctgttcggggtaatgaaatgagaactggaagagaggatatcatttcgaggcccagctgagaaggatcccaatgcgaaaaatttacacacttcaaagagcagaaaggtggaagcgagtggtgcttggacacaaggatgccactgttcttatctcctccaatttgggaagtgcccagcctagaaaggtgagggtcccatctgggagaccatggagtacccccagggtcacactgagcctaagaacttcaaccatcctgcaggtcaaaacccactgataggtctcccaaaatgtttggggtagggggaagaaacatagggcattccaggtctgtaaccctgcaagagagagactcaaactaatatgtctaggtctggggtgaaggtggggggcctgtctggatacctcctccctcaattgaagggctcgttggaaacataggaacccactgctctctgggactctggaaagacaaattttctttggcaaacggaacagtgtcgcgctgtcgacagagcagactatttttcttgttccctcacaaggtgggggtggggggaaagaaaaggaagcaagaaagaaagggaaattaaatccacacgtgttaagctcgctcaaagggccagagtcaaaaccagagatgaaacttcgactccagttctggctgcccgcgggcctctttccaaccagccaggcctcacagagcaaggcatccccactggagagaattcgatctgatgtttcataaagcgctttatttaaggagaaggggcatgggtctgtctccaagtctgtgctgcagaaatccagaatttgtagcggcaaagaaaacaaaggagataacaattgtaatcataatgaccctctttatgaaaactcatcttgatgtcctccaaatcaaaacaacaattcaggcaatattcaagcaatggccaaaatcatccaggccattaaatcatccaggccattgttttacaaaattcctaaggtctgatcaggctttcggacttgggcctagctgggtcaagagaacctgaaatgctgttgtcaagagactctgaaatcaccagtttaatcaggacagctaatctctacatccttgaggcctctcttttggggagagactttcttgagagaggacactacaggactcagctacagagctctgctatacatgggtgggagtggaggccgccaccccacctggaactgtgagtgaaaactctagtaatgatcttgaggaggtgaagggaggtaggtgggggtgtgggtgatgagggaggccaaaccccaatctggaaggatctgtcactgaaacaattgcttggtcgagacctctaatttttcttcctctccagccagtctcctgtggctccctggtgctgggtttctggcctttggtgacatcctaacagaagggtccggaagcagggaccccagacaccttgctccctctcccaaatcacacccaaaacatactggggtgaaacaactgacagagggtctctgtggtgagcaactgactgaagtttccagtggaaaagttcctgacgattgtttccgcagggaccttgaatttcctggtcaccatggcgacctggaagtcccaccgaatgctctcaacaatggggtaccccgaggacattcgtgcaattggctgcgcagtctgaggcctgaaaagcgtttctgccaatcgttcttgactttctggagcctccttagtgggttcaacggagcagggatttttaaaactgcccccttttttaaacaggggagaaaattgagatttagtaccaggtgggttcaggtcctgagcaaaagccccatctttgaattcagaatgctgaccccagggccgatcgtgtagagcctatggtgtatcttaatcgaaacccgtggcccaaacatctcagccaggaacctgggactttaggtgtcacttaatctcttcacacctgttaactacacctatctgcataggaggctccccacatcagtccaggcaacttttttctttgcctggggccatttcttctaaatggaaggagaaaagtctgagcattagaaattgtggcattaatgtctgcaagggctaatggagtgaaaagaatacattattctccttttcttcttagaaaaaaatttcaattgtggatgtaatagaaataccattaaaataatatttctgattgcattgctttcttcttgaatgtttatcagaggggagggggaagtggatcttttcattttggggtggacagtggataatacaggctgattttcttgtgtccggttcggaggcactgcgcctgcagggtttggcagcgatgcaggcagcaaggagagaaagggtgacagagagaaggcgtccaggcctcaggtgccgacagaggtctttggcggtgggacgggagctactcctcctctgggtcctctctcagctttgttccattctgctgccaaccccatctttctcctgactgctcaggaaagacaaaatttaacttctctccccctgtttccaacaggacatctaatgtccctcaaagctgctggctggctctgtagtgcctccacccattcctttctcttctgagagcccaggcagcctacagaggctcccgcttctaacccaacgcctcctgtttccttccctccccagcattttgcattaatattttagaagagtcctacttatgtctacacaaccaccaagagagcaatgtgagtattttaggctcacaaatctgttagcatttgaggtgcaataatagccaacatttatggcaggccaggaaagagccattgtaacctggttttgctcacacatttagctctgaatcgctctatgagaagagagctcaattctcctcaaccttacagaagaggtttctggcgctctggccaccgccccactctggtgggattcgaacctatcagaaggcagggttcctctatcaaggcaggaaggggaaccggggagaggtctgttggattgggggagtgcaaagggcactttttcagcgtcgaaaagcacacttagttgaaaaccgggggggaattggtccttctgtcacggagcgaaggctcacatcatttctgatgtctcagtcttttctccccattcccactgaggccccagaagagagcctgggggcaaacagcccagattcactggcattaagcaagatgggagggaggacggaaagatgctgatgttctcctttccaaacctcactgttctattcccaaatcctggcccacattcatcatagcctcaaggccagtacacactgtcccctggctttgagggaggaagatgggtgggtgggatggagccgggcagagatgtgcccggctccatttttttttttttttttgaaacaaataattaggctgtg

SEQ ID NO: 110 - DNA, 837 bp, Artificial Sequence; identical to SEQ ID NO: 66
  subsequence

SEQ ID NO: 111 - DNA, 156 bp, Artificial Sequence
  subsequence of NECAB1
tttacctatgtaacaaccctgcacatcctgtacatgtaccccggaacttaaaaaatttttaaaaaagtatttaaaaatacacttagtcttactgttatcatacattaaaaattgttttcccacactgggcctaccctaggcaaaattgagaagatg

SEQ ID NO: 112 - DNA, 3718 bp, Artificial Sequence
  subsequence of ADRB1
gggaacttgggcaaaggagaaaacaagcacgcagaccaaggaatctgaaatcgcagttcattacgtcagcataaactgacagtacctttttgtttgtattgacccagctcaaattataaaatcacatgagtaataaaacacaaaatacaggtgttatcattgaaaagtctaaaatgtaattaaacgcgttttttccccctcgcggtggtatcgtttactctgtatctcaacgtttctgtctccctaagcctctcctttcatatcatgagcatacatttttgcatcatgctcacacgttcattagctaggactgaggagtgtgtacgattccaaagggccctcagcgttagctgttagatgcacaaaccttcgcttcctttccacatctactgcactcgaggttcaacagaggatccttgcaagaacagcgcccagagagcattcttgacagatgcgcgcttggctccagcaacccgctctgctgggaagtttcttctaaccactaacacccacctccaatcccccaagctgtcacgacgcatgctggctgggtcccgtcttgacgggggaagggttttacacacaccctgctaggctgccccacatcacaaccaagctcgcagggcaaactcctccaagcctggcggacaagctgtcccaggcgctctggcgcttcctgaacaccaaggtcccctccccgctcaagggagctagcgctctgttccgcagagaaccccggaactgcaggtcgaggggatgcggggggagcgggggcgcaggagggagccgagtgctggaggcaaacggggcgcaggagcgggtgcgggaggcaaacggggcgcaggagcgggtgcgggaggcaaacggggcgcaggagtgggtgcgggagcgagtgggggctgagggaggggacggggccccggggggagccaggcgcggaagggggcgcgggggaacagggaccaggaaccagcgggcgcaggaaggggtgcgtccgcaggaacccgcgggcgcacgggaggcactagctacgcgatcagctcgggactctcaggagccgctcaattgccaacgggaggggggtgcggggagttggaggtgggggtgcagaccagacgggggcgtgcctttgcccggattggctgcaggagcctgacgcgaggccccgggggttggcttggggagtgggagcggggtggggtgggtgctgggtgccggagctgcgggcccggcgcgctcagaaacatgctgaagtcccggcggctcttccagcagcggcagcggctccagcagcagcggcggcggcggcggcggcggcagcggcagcgacagcgctcggctcctgcgggaaaggcgcccggcgcccatgcctccggccccgcgccgcggctgccctgacccggccgcgacctccctctgcgcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaagaccgatagcaggtgaactcgaagcccacaatcctcgtctgaatcatccgaggcaaagagaaaagccacggaccgttgcacaaaaaggaaagtttgggaagggatgggagagtggcttgctgatgttccttgttgttttttttttcttttcttttctttcttcttcttttttttttttttttttttttctgtttgtggtccggccttcttttgtgtgtgcgtgtgatgcatctttagatttttttcccccaccaggtggtttttgacactctctgagaggaccggagtggaagatgggtgggttaggggaagggagaagcattaggaggggattaaaatcgatcatcgtggctcccatccctttcccgggaacaggaacacactaccagccagagagaggagaatgacagtttgtcaagacatatttccttttgctttccagagaaatttcattttaatttctaagtaatgatttctgctgttatgaaagcaaagagaaaggatggaggcaaaataaaaaaaaatcacgtttcaagaaatgttaagctcttcttggaacaagccccaccttgctttccttgtgtagggcaaacccgctgtcccccgcgcgcctgggtggtcaggctgagggatttctacctcacactgtgcatttgcacagcagatagaaagacttgtttatattaaacagcttatttatgtatcaatat

SEQ ID NO: 113 - DNA, 5583 bp, Artificial Sequence
  subsequence of OTX2
aaaccaaaacccaaaactaagagattctctccacaaccggcctaggtatgcactctgcatagaaggcatgggcccttcaatctaactttcaaagctcttttcctctcgaagcagggtccagaacggggaaaagaaataacttttgaaaaatccacgcctcactatgactgctcacgaggtggcctctggagaactcatacctagaagctgaccgcggtcgcgcggcgagttccggcagccggtcggcgaccgcacctccgggcgcgagtgcctgggtcccgcgttcctgcccggcagccccgcagccccgcagccccgcagccccgcagccccgcaggcctggcgcccgaggtcccgctccactgcccgcgccccccgcgcagccttatatctaacggtcaattcgtgcaatctgtcgcttctccctcccccacgccttgttttttttttcttccaagaagcccatctaccagttgctgtgtcctcgctcaacaataattacctcgtccgagaattaattataataaatgttttcttgataaactaacgagataatccgaggggcacacgtcccttaattacaggccgccatgctcctctctgcttctcgtccgggctgattaattttctgcatgatggaaaggaaacaaaactacgcggactggcgactggcctgcggctgggaagacgacgaagaggaggaaagaaagaaaaaggagacgtgtgggcaccgcggaaaacggccggcgctggcctctctccggcgaactcgagtgaaagtttctggcctcggggaatcaaataactctgccacccgcgagggagggaggaagaaacgtgccaaaagggttggccttgactattaattatcgttgggagaaagccccggtgttagcgctgaggtctgggtgtctaccttactctggggggaggagttccctctcctactcccctctgttgctaataacttttggtgcctgtaaaaagtcccgagctgagcaggagaaatcctgaccccgaagctctaggatggaggggagaatttctaggagcgacttccccgtcccctccccaagcaatccaccaccgcagggtcggcgccgctcggccttcgctccccgcgcaccagttctatctgtgaaggaagcaaaagccaactcggtggaatcctgacaggggacttgggtttccaaaaatatgtcccgaaatcgggcatcgatttcaagagtcacttgaacgcaacaacgcgaagacttcttgggagtttgcagagcgacccgtcgcccgcgcccggcgctggcagggaccttcggatggttcttactgggccgatccatggcacaggctgggcctcggcgaacccctcggcccccgcccggccccgagccacgacacctcattgtcctggagcctgggaagggggtgcgcgagcgcgcgggcgagccctgcctctccccgccagagaacagctgaggggccgcggtcccagcgggaggattccggtccctggcccggccgcggccttgggcggagcaggggccactagctgccacttctgcccgccccaggtgcgcgcggagggctacgtggggcgggccgcgacccggcaaagtcatgttgaaaaaacactcttcacgttcgctcggcctggtgaccagggtcggggaccacgacaaccgggggttgggaggctgcgtaattacaacccagggtggtttggattttggggggtggtggatatttaaaaacaaaaaggagatctggaagcttttgggagaaacagacaaccgagctgtgctaggctgagggagaggaggccaaagagagcgagcagtgagcgccggggcggacgagaagccagcgctcccagcctcctcggttatccgctccggtttccgctcacgttcaacaggggccgacaggggctcgagcggcggcccccggcccaggccgacccgcaagcgaaccgagcttccggcgcgcgggcccaaggaggcgcctggctttttattattgttgtttcaatccatccatctagttacatctgcatctttttgtctcggactctaaaaaggtccctgggatccatccaaacgaccccaaccaaatctgggggccaaaacgcaaagatcgcgggagaagcccagaacggcgttgacataaaaacaaaaccacaaacaaaaccttccaaaacaccccagattacattcgcagcgtttcgacgacgttttgcagaagcgacgacccccaggagcacgctctctgcctctctcccactaccgctctcatctctagatcacatttttctttttgcaacgatcgttattacacctcaaaatttgtaagagaaaaaatacatccgcctacagaactccacgttcgcagaggggcatcggtcggtctattggaggtactggggtgatggccgacagacacggggacacgtagatatggatatagacaagacagacggacacttaccttacagcctcatgggaggttagaaaaagtcaaatatcctttaaattccaaatagccagctatcctaaaagaaatcaagagtattgagttagaaaccttgcagtcttcacccttccatgaaaagtcaaacacaaccaaaaaaaattttttttaaagaacgaaaatgaggaaggcggctagagttctaaacacgcgtgggcccctctgcaatacatacaaagtagatgtggcgagtgagggctgctctagcaatttttatttttaatcacagtggaggctgtgttggagctgaggccggtcccgctctcagggagatttgctgagaaagcgcctctctggcaactttttgacgcaaactctccaaccaatggcagggagagaatgattgacacatctaagccagagggaaaataataaaaacacacaacagggggaggaaaacaggccgctgctgcacggggggacgagcaggcaacaaatccaggcctccgacattaccagaatgttctttaaagacactgggctttgcttaaaatatacattatgggccggagcatgtgaactaattgtataattaaaatgatggacagggagttcttctcttaccctcttttcccctgcttcctttactccgaaatttcctttcctctcttttttttttttttttttttgttgttgttgtttttagggaagggaagaaaggaaacccatgcaatgtaagatggaagataaaaatatttaagtaaatgctggagaatggcgggttagggagtgactgggttatcacattcaaccccagcaataagtgtgtggatgacaggcagaaagcaagtcagactgaaactcaaaaacaccactatgccgtctcagacagggccagagcacgctgacggccagaaagggtaaaggagggaggcctgggagtgcgatgtgggcatgtagggaaaacagagagagaaagatacacaagtccaagaacaaactattttctaaaactcagtgtcttctctattccctacgcccttataaatatccttacacatataaataaatgatccccgagtgagagaccccggcttctggtaggttcctggggttttttggaattgagaagcccacgacagactgcagggaacacgaaccccggaatctaatctcagggcccatttaggagaagtagctgggaccacatcgggcctcgaggatgcgagtgtgaggtcgacaagatatcgtggaagagagcgaacgagggaggcccccgttggaaacagtttctgtgttagaacaaacgccaccaaacgaccgacactgcgaggcctggagctacctgggctggcgccgcccgagccccgggctgcgagcccagaggctgggaagtgatgggacctgggggcccgagccggagagtgcgacccccagcccaacttttttgcctggcatagaaagcagggaccggacaggtttcaaacggatccgagtccctagaaaagcaaaaacagactgggcggagtgtggggtggggggtggctagtggaactggtgagtgtgtgtgaataagctgggtgggggtgggagcagaagacagctcttgcttcacaacacccagtgcaggaaaagaaaaataaaatctcttcctaaataccccaccccccgccacccacccaccaccaactaccatcaccaccacggccagtgggcccaaagaaacagctcgctgcctttaatttggggacctatgttaatgaagttggtaattaattcgtcgctttgttgtcggttcttatactgtatgtgtactcaaatgtggccgtccttgggatttacagcaattaacgaattacgggagtgcgcacgaggcagcaagctctcggcagccgggctccggctcgggctcggcctggggctggggactccgcggcagcggcccctgtcacaggtgtccctagagctttgggccctggcggcccctcgagcccccgaggggcccggagcccgcccagctccttcccggccctctgcagcagctccagtccggagacccgctggctgtcgcgttcccaggagtcaagagtttctaggggcccgggcgggggggagggtgagtaccgaccttttttctcaccccacccccactgtactgaaacacctggcggccgcccccgaagcctttaatggggatggtggtgaggagggggtaacaaatgccccgggcgttatgaccctcgtggcgctggctgtcggaggcactggttgggtgcgcttggtcagagtcatgatgctttcgatgaaggaaaggcaaatccacctcgatcacctgaagcagagtctccttcctacccctagaaaagacaacgtttcccccagtttgatcacaatgatgtttaaagcataaaagagccgaaaaagaaaaatggacgttgtgaatctggcagaggggaaggccggaggaaggccttggggacagtgggcatggggtgggatggagaatgatcagaacatgcgcttgtgagctaaaccaagtaaaccattttggactgggagatccatctgccgtagggtccagtgtccagggtaacctttcaagcccgatgtagaggtttgcgatgtcctcccagatggcgggggcagctgaatctccctaaaaatccccctgttctattcgacaggggcagatttatgcatggtggggcgtgagagggttttgcccttccagcggccggcagcagacagctcccgcttctcctttgccctctagtctaataacttcgcagaaatacagggccaataaactacgagttggaaatggacagtgttaaataaacaagggtctctttaaaattctcatccacttcagatgtctaaagtaagattgtgatgattttgtcacggtttggtaaatttacccctttaagaggctttcataaatcccagaacacaccttcagccagatttgaatatagatttaggttttaaaacccagaagctggaaacaccgctttatcaaagagaatttccctttagcttagcaatgtgctgaagctcttaaaagattggatctgagagagagagacagagaggagaaagagaactagagacagagag

SEQ ID NO: 114 - DNA, 7286 bp, Artificial Sequence
  subsequence of NKX2-6
actccgtctcaaaaaacaaacaaacaaaaaacaaacaaacaaaaaaaaaactgtgtgtgcatggtaaaccttcgctgtgaggaacaagtgtgagtggaggtgtctatcagattgaggagactttggaccctccttggaagtagagaaagaaggagaagaaagaggaagaggaagaggaggaggaagaggaggaggagaagaagaagaaggaaaggaaggaaaaacagagagaagcccgggtgtcaagaaggcgcaaactcggtgacaggctctcaggaggctcctgtctgcctcattttccttcgcagccccaagcgcactgcagggaacttggctgtaagtccaggagcgggagccttgagtgtctcctaccttcacccaacttttgtgcacactcttgtctttttgacgcagcgctccaccaacaccagggatgcatcccagctttcttggatgtgatgctttgtaatccaaattaacaatatatgatgaaaacaaaatatttattggctcctgacttttggctcagaaactgaggttctctttgagagatacataaagttgaggtccacatttggcttcttaaaaagtagcctctccctcctttggggtctttctggcaagcaggtggtgtgggcttcaccctgaagtcttcctttcacccagggcgacccctacagctgagactgtgcccaccaagatgcagcgttttcctcccttctcacttctctcctgtctgggcttaaaccccgttttaccccaagggtggatagaagggaaatgctgagttttcatgggatctcacgccaggagaaaatcaggagcatggaaggggtgcaagttcatggcaagacgggacaggactctaaatgagccagtgcgattggaaccagactcctaatacccaaggcactggcacaatctcctggagggggatggagctgtttggtatgtaaagtttgaggacagaacaacttccttcagagacgtctgtgctcagataaactgaggccatctccacttgcaaagaggaaaaattctgaggagggtttgttttcttagcgcatcgaaaggtaacaactgaccttcagccaggctgcagagcaaggaggaccccatttcaaagtcacagatcatgcaggccctggggctgcacagcaccgcagggtgggccagcaccctgcgcccttctgttcacaatcctggagttcttgagggcaaggtggttagctgcgatggaggaaggcatgaagcccaccgaaggtgtcttccctgtccggatcgctaccgcggccttgggctcagttcctggagccggcctcgggtccggcctcgggtccctcctcggcggcgtccactgctgccgcagaagagacgctcccaggatttggtttccagtgggagaaggaaggcgccgcaaataccatcggctgcgaggcgactgtggtggtcgaaggaacgtgatcctttgtcagctcagcggagccgaagtgatcgccgagttggctgagcagcgccgagggagtttagaatccgctgcagaaaacctaccagcttcttcgcggacacagcaggtaatgttcttaggcttctggagttagtgagtagccggcctgcaggccggtaaataactccgatctagccccttggtttatttctttcgttcgcgccttgaatggaggcgccatggcacagtctggcagaggaggcctgccctggcgggcgctactcagggcccctgcgttgagaatgccgagtgaggtgctgaggtttggagagaaaaaggtggcgttttcatcccttggaatatcgggcctctccagctgaagcctccggatcgacacaacacaactcccggatcgacgcaacacaactccccgccagccccttcccttccagcgggctggacacaggtgcgtgggcctcgcagcccggcgcgcacggttgcgcctttggatacctgcgacaggactcggccagaaccgtgtcccagggcagagaaaaccctggcaacctttaacgttctccctgacgcggagaatcctgcgccaacggggccttccacttcgggagactcaatcgccacccggtccccaactttcgtcttccagaagaaaaatccatgaggaggaatgggggggtcccccgaccagaccagcaagaaagggcccagttaggagtgacctcagcgccacgagggttccactgctttgatggccacacgcttttgcaaacggtttgggggtggagagacaccaggtgatggtagggaagggcggtggtgactggcacggctaagacactgcggagggtttcgctgtggagatgagaaggtggaggttctgggcactgtggctgcgagtgtgagcgatgcgtttgtgggaatcagaggagcggatttgggaaaagtgaatccttgagattcagggccttactttcgttaggggtgtgtgaagcacactgggtgtcaggagaggatgggaatgggattcgagaggcctttttttggactcctcgagagaaaatggagagagtctcgaacccaggagataggaggcgtattttccccatgcacccatggatcacgcccccgcccccacattccccccgtaggaggcaagacctgagcgcttactcacgtggctcccccatccgttccgcgtccatctccaagactgcctcacagggacccccaggaggctccgaaccatccagctttctgtcaccgccgccgccaccagcgttgtgaacctctgaccctcgcggctctgcgtccattctcaggtactgaaagttttccgggctcttccgcacccgcggatgtggcgaagccgcggggcagctccgctcgcgctccagtcgcaggatgtccttgaccgagaagggggtggaggtgacggggctcagcagcatcccgaaggcggatggggcggggccgaggaggtccgggtgaggagcggcaccctgaacttcccgtcttgtcgctgcaggccccgcagacagacccaagctctgggacagacgcccagcgtcccagacagcgccttcctctgggccatgctggtaggcccgggtccagggccgggtgacgagaccgtagccccccattggttctcgcagaaaccacgtgctagctctgcaccttcctcccccagcgctttcttcccgcgcccgtcgaatcctctccagtccccagtcgccttcctgggaggtttggttcgtattgacggttctcaaagcaaagaaaacgtggagagacagtgctgcgcgcggcttggatctccgtcaatacggtactagcgggagcttccgaggtcaacctagggtccacggggaccctcctagtggacatgcggggattggaggtggagtccttgctgtgcaacccgatccggtccatccagcctggtcttagctcaactctgcattcttatggcggaggaaagctaagatagagacagctgggacccgcggaatttgaggcgcgcgctgggatcttacgtgcgcagaggccggaccgcagagtagaagtgagtcttgacccaccagggcctatattgagatggaaacctctctctttgaacagaatccaggtctgcggagtcccgacccgccacttcctaggagcagcttctcagccccaggaccgaggaatgggtgtgggggctacatgtggggagccctcgacctcttaaagggctccgcgaatggtgggggagactggaaggcaatctctgcggcccgcgcaaagtggtcagaggcgggagtgggggtgggggggggtgggcttccgtcgcccttggcgtccccacgttctttttcttcctctcctttatcctccttaaagcggattgcctggagcgtgccagcttgtgtgcgcgtataaatgcggcgcccgccgaagggctgggaagatttgactcgcattagatctccagaaaaggcccttggttacggcgggcaccgagtaaatggcgcagtgtggcacaaaggttaccaacacggagatagcgccaacagatccagcccctgctgttactactcctggatcagggaaacgtgatttggtgtcagctgttccagcactgaatcgaagagggtttaaaaattgtgttttaatcacaatgggatctcattagagttcttttggggatttcagatcaaatgtttctttttgtttttctttccttcctttccctccctccctccctcctttttctttctttcttttctttctttctctttcttcctttttttttttttttttggttacagggtctctctctgtctcccaggctggaatgcagtggccggatcatggctcgctgcagcctcgacccggactcagtgatcctcccacctcagcctcctgagtagctgggaccacagatgcagccactaagcctggctatttatttatttatttattgcttgtagagccggggtttcgccatgttgcttaggctggtcttgaactcctgggctcaagtaatgtgcccacctcggcctcccaaagtgctgggattacaggtgtgagccaccgcgcaggcttcaaatgcttcttaaacctacttacgtgaattcaatgttacaacagctgtttgaatatgattatctcctgaaattcagataaacccaactcatctccccaggctaatgggtgcaaaattgcacacaaatatttatgcaaaatttcagcacatttgatcattcagctacaaagtaatgatttcctatacatggtaacatttaatcttcacatcaaagctatacccaaatatttccagctgctgctccttcttcttcttcctcctcctcttcttcttcttcttcttcttcttcttcttcttcttcttcttcttcttcttcttcttcttcttcttcttcttcccctcctcctcctcctcctccttctccttctccttcttcttcttttgttcagcgaggagtccagatcagagaggtggcccagatagttcaccaaatggcaagaccagactagcctctaaaccaagacaatttaccctgtctttctttctttttgaaatatacatcctttgctcttagagatctcaaaatcttctgatgacccatacatttatttgaatttttgtttcctgataaaagtcatcatgcgcaccttggaaaaagtgaagaaaagttaaaagaaaatatccacccagagacaacattttagtggaccttcttgtctttatttctgtatatatgagctcatagggtatatatgtttgtatatgtttaaatttacatcataagcatgcccagttcatctttagaattttttaaacaaatataatttaaaataggaatctaatatttcatgttattgatagaccgtagtagactgtgtttgtatttttagataatatctttttttatcactgtaaataagactttattttgtgccttcaattttcatccttgtcaatattttctgttattttctcaggatacatatgtagaagaaacatcaatcagtcaaatattttaatgctcataagacatgccattccagggcttcctagaaaaacagtattatcgtattttgttcccggaggtgcaggcgagtgcctggtctacgggtgtttacaatcatcaccgggcacccacaagagaccctgaacttagaatatttccgcggtaggagttccgcctcgtccttccaggggatactgacgactcgctgggttctcgccttccaagtgctggaggggggaagtagggccacccggggtagaaacctaacgccagaaaccttccagcgccttatcagttggtggaattttcgtggcagacagtcgcgctctacctaagtcgtagaagaaaagacaccttgtgggggacttggcgtcggaagccttgagttcaaatcgtagccctaggtccccagccttgcccttcctggcttgctgcgaggatgaaatgagatttttgtacgtgaaaatgctttctaacccctctcgaggggtggaaacgtctccattcgaccagagtccgggtctgcggagcaatttacccgccgctttccaagagcaggctccccagcgtagcgagtccttgttatggaaagggtcgtttcggctcaggatgcgcgctcccggcgtagacctggggataggggtccctgtcgcgctcgccccacccctgcagggagcaaggccgtccttctgcagcaccgcgccacgcggcgggaatttgcagtgtcctgctttctggtttcagcttcttcgctcgcttgggtctctctcggtcacctctcggaggggcctggagtccctcctccctcctcctacttcagcgccagaagcattgaaagccgcccgcccgccgagagtggaaccggtacctcgaatctcgctcattccgggtcaattggaggcgcattagcggctgccttgcagtgcaacagaggctcgagtgacaggccacctgcctaagcgctggcgcccaggaaagccggggaggaaactaggaagggaggcgacagcagaaaagccagagagtccagagactcctaggcaggcctccgtgcagccccaacagcgtggtgccgggaggtgtgtgtgtgtgtgtgtgtgtgtgtgtgtgtgtgtgtgtgtgtgtcccttactcaggaatggagacatcgtgatcacctctgttcttaggtgagatggagacggccgtgctaggggtgtgattgatccgctttgcttcactcaggtccgcagagtgttttaagcgcctaccgtgggccagctctgcccaagctctgggaatactgagaaaatgagaggtctcagccttcttatccgaacttgcattcttgcatagaagacagacttttaaataattcccatacattgtgttaccttgcacaatcagacggaatttgtacactatatttattgattttttttttttgagacggagtttcgctcttttgcccaggctggagtgcagtggcatgatctcggctcactgcaacctccgccttttcggtttcaagcaattctcctgcctcagcctcccgagtagctgggattacaggtgcccgccaccacacccggctaatttttgtatttttagtagagaccgggtttcaccatgttggccaggttagtctcgaactcctgacctcgtgatctacccgcctcggcctcccaaagtgctgggattacaggcgtgaaccaccgcgcccggccctgaacctctacttctccgtggttaacaaaagtcattatttcctgtttctttcactctcctgaaacgaagggattccctcc

SEQ ID NO: 115 - DNA, 7007 bp, Artificial Sequence
  subsequence of SIM1
caaactttctctggtgatgcccaaaaaaataaaacgctccggactcttgtggccgagttttaaaaatttaatatggagtcggctagagtacccttttgcggaaagaaaaaacaaagaaggcaaaaaaactttcgaagacctgtgttaagctatttatgagtattatctttttaatagtgctgctgctaaaattattttaactaggtagttaaatccttttatccttacaccattaaacaaagtttaacatgacctcccccatcccacccctcagaaaggtgatttactgcgttaaggagcaaatcaccaagaagtacacaatgatgaacactcgccttgtaatatgaggttgggggcagatctttcaaaataaaacatggagttggattttttaaaaggtgttggctaggagctgcttcctaagcaagcaatcaaagccttggtaaggaggaaccgtgagcgccccttaagctcaaatccggtctttctggttgtttgcacatctaaataaatctaacgatttccccaccactctgcaaacactgatgctccccgcccgcactcgggactcctctacctctctggctcccggtcaactcatcccgactctttagcccgcccgaggtttcctccccggagtgactccactgccctggtccctggggtcctcctggaccgttctggaagggcccccgccacagccacctgtacacaccccttggccttatcacatttatcgctcttcccctttcagccaacctattctccttcgtttctgtcacgacctgctcttctctttctaaatcccggctccgacatacccactcagaaggagtgacaggagggattggctgcggaggccacacgggggaaaggagaaaggaccctattgggaaagccgaggacgccggcgacgctggcccttgggcgctctggggcctacggaggccttaaaccccaaagattggcggggacgaagggccggggcactcgagcgagcctggagcgggcaaagactgcgttgcgactggggacaggtctcgcgcggttcccgaagggtgcgaagactgcgctgagcattttgaaggcctcccaaagcggctggaagacaccccgaagcggcctcccaaccagaatgatctgaaaatcccccaggctgggctcgggataaatactagttctgaactcccggcgccagcctaggccatttgttgtttcgggtttcggcctcattagtatgcgcctacgagggaaaatgagacttccccacaggttaataaacaacctgtccggcacaggttaacaggagcctctctcgccctctcctgccccacccccgccaccttttaaggaagcttttgccttcccggaggtttgccttctctttctgttccccgtagaaatcttacgattgtgtctgggttaatgttttataagggactggctactctcgtctacctgccgggctttcccgatctaatatcgaaggaacctccccaagtcctagtaaggggtcccccctccctaaagtctcgaagtggggtagagaccttctccctggagcccctcgcctcctgccttcggtgacgcgctctagcacacccaatttcagttggcgagggggtaagctgagatttcgtttctgcacttgacctaccgcagaaaattcgcaaatcctttgattgtctgtttaaaatgaggaggaaaccagctccctttgcggcgggattacagtgggaacaagatctaaagattctcttgcaattcggcgaggccgagggaacggggttttgcaaaagagaaaaggagctctgtgagggaggcgataactaaatcgggttttagacaaaaggattttcctcagctaaagtggccgaagacaaaggaaaacagaatctattttggaagatttccaagaaatgccacattaaccttagctgataaattttgcttctcccaggcccccctcttcccttaaagtctgctttatcatatttacataagtagcattagaagggatgtatctattatggatctaaagggctagctcacatttcctgtgaaactcaggcaagtttcactgtggctttaccctccgtgcttagttgagccatttccatttaaataccccctgacccaaggattacctcaatggactgactgcacgggttttgtaatttcctgaaaatgagatttcgatttataaaacaagaaaccaattagtaaccaaatgaggcgaagtagatccactaaaattgacctaatcccacaggcaccatttaggcccgttcttggcgaatgaatgatgagccccagtctagtgtgagctcaaagcatcatccagctgtgttattgcaggcagcaaaaataaattgatagagcttttccttcggaatgaatttaatacccttgacaaaatatatattacagacccgacatcatcaccgccctcactctgacaggaagaggaggaagataagcgtgtgttctgttcttttaaaatttttctttattttaataaataaacgctacatctcaatagaaatgtatcctccttgcagtccttcccgggaaccttcacctccccaactaattgaggtgccttgtaggcaatgggggagggtggcggaggccctgttccatgacctgactacgacttggtgactgaggaggggaaagccctcctaggccgaggccactgcggttcctgaacagcttgtctcaaccaactgtgtatcccttgagagagtgtaggagagtctggcccagggggtaatcctgaaaaccccaaatcccatgtatcctgccgggcctgcacatcctcgctaaatgtgtactgtgcaagtcttccccgggctgaggcgtcgagtggccttcgtgttcagcctccgggcgcctccgcagccgagctctttagagaaaaaaagagagggaaattggtgcaaccagcccgaatcaagctcctagagcagtcactggtaggaggtcccttcacgcaccaaggcgcacccgaccccacctctggcactgctgcccgccctgcctgcggccctctccggctaaggagcctgtgtggttgacctgagggcatcggcgagcgcgccgggccaggtcttgttccacgcagcagcctggaccccacagtgtgtgcctgcactcggcagtgccctgatctctcctgcctgctgatcccgggccagagacaccgcggccgcacccagggaatccactaatgttactgtaaatgtggggtggaggccgaaggtcacaccgctgtctgtctctctctttctctctctctctctctctctctctctctctctctctctctctctctctctctctctctctctctgccttcgcggagctccagccctcgagtttgggcttcccgtagggatagagcctgcgggccaacctggctgcccgagttggagccagccgcgaaaagacggcagaaacccaaggagggcggggttcggccagactaaaactgggggcgaaattcatgcagcgttcatgagacagtcctgcgtgacggccgaaaaacgtcaatcgatgtcgccctgggactccgggtcttcgaatgcggcccagctttggctatgccacctccctagaggcccgcgctggcgaggccgagcccgaacgcgcgggcgaatcccacttaggttctacttggcgcagggactcggacgatggcacctaacccgccttcccgggaactgcccaaacagactggatctggacctaaggtttagacgactgcttgcccttcagggacagtggccgggagggcaggaggggctgctggcacacacctgcacctgcgggacctctgatgagggcagggggcggctcctgggccggcctggggagtaaggagactctgtgctgaggccacgagctaggcccttggctggggccaccgcggacagaggatgggacacaccgcgcagacctggctgactgcgtgcttcgggcggtgcgggaggcgcgacggggacctcgacatgattgtcccctgtcggccctgcgccgccgaggccgagtgacaaggcgcaggaaccgcctattcggtggggctaccagcggaaggaagtttactctgcagtggacactgttttagcactttaagagtagcaggaccattcctaaataattgaggttccccctcctccacacaaaattttaaaatttcacggatcagacactggcaatgggtggcccattaaaatcattaggtgttagcaaggtacaagcctcattttcctgagaaatacctggcgggacggagaagtgcggtgagaatctatgggctctcattctgtgtccacatttgggtctgtaaaagctggaagctcccaagatttcttagaactggacaacaactcccctagaaattagatttgttctttagaaattcagccccgttttatcattagacgggtgattgcaccctgcttgaaagcatcccaccatagcttataatacctgtttttgttgttgtttgttgtttttattagtgatccctcccattaaagaaaaaagtattttttttattgatctgatctacaatttctagcatataaatccgccttgcaaatgctctgcacggagtttagttctgcactccacattttaagtcaacatcaaaactggaattaaacacagaaaactagcatggagggtaaactgtgcttaaaggcctttaaatgagaaaaacttctgtaagaaaataaacatggaaacctaatgctcaatcatttccccaagaagcaagaaaaacagccttaaatcaccgacccacgtgggacatggggcctctttttctttttcagttctctgcctcaaaaaaatataacaccattaagaatttcaatcttgaggtgaatattatagaaaccatctgtggaagtttgtttaatcacccttgtagagatgatctttcctgagtagttactaatttcaactctgtgattatttttatttttccaagacatcttgtcaatgaaatggcaatgtgatgttaccaagattctgaatagctatagatcagaaaatatatctgatcctttcaaagttgattatgaaagcctccacacccccagccaaaattgctttagagctggataacaaatatgtatgtctttctagttgaatttgtcctattttgcacacaaataaagtcatgctattttttagatcctgcccctctggctttgataataccaaagatttatacatttccatgtgcctcaattttttctttgcaaggctactttggaatagaatatgtcactaattggcagatatgaagtgttagtgatttctcagttatctttcctaattttcttttttctctgaaatctccaatctaatgctttattcttaattattccaggtacccatattccatttctctgttctcccactcccccattttcacctaaacagtttaatttcatctagtcaggcaaataaaagctgaaacttgcccccagacaaggaagctactagacttgggagaaaaaaaaaagtgtattcggcaaatgagaagggaaggaaattgcctgtatttaaggtgtatttaaggatatttcttctttcaaatataattatgaaatgattttgtttgttcatttaacttatagccttttggcattacagtgtgttagagatgaaatagtgtatgaacaaaactgaagttttacagcatttttatacctatagctatgtcaccagattaataattatttagattatataccttataaaatccatatattacctgtgataagacacttatacctatctttatttcatccttaaagctgtattgaggacctactatgtactagggtggcttcatgataaatgtaaagatgaatttcatcttccaacaaattccatttaacgccactagaaaagattacatccatataaagtgatcagtttccccaactgccttggaccccaatcttgtttgctctctgccttcagtcaacactgaacttaaatttggcaccccctataggttgtgcctggtcttgcgattgtgactattttattcctttgaaagctgttgttgttgttgttgttgttctttaatattttaaagagctgtgtttgattttgggtttttgaagcttgtccaccgtcaaaaattaaaaactcatccctaggagccagctgttctgatgaccctgtctagctcactgggtggaggctgagggtgagttatgagctcaacagtagctagagaaacaaatgatgtgcccagagttcacacatcgtccatcccctcctccccataaactcacactcacccacagaagggagagaaaggaggggtccggggctcgggggaaggtgagggggctgcagaaagcacagtcactgttaaagtagccagaggggaccgttctggccaccaagaggactccagcgggagccttcattggcgctctctttaaaaataacgagaggttggctgtaagagtttattttgttcctatttatttataacgagacttataatgtgtagagttagcacttaattatgcaaaaaggcttgctcttttaaaaacaaaaacaaaacaacagcagaaaacagcccgatttcctcccagctgcgcctgcccccgggtctacaccaagccgactagcttccctggccggccgactccccgggctctcccggccggctcggtgcccgccccagaacgccgccctgctattcttctcctcccgcgattactcgcagccccacaagcaacaccatatgctgcttctgatcaaacagcgagcgacatcggcaccaataattgacgagaccggctggaaatcacgcagaaagggagcccactgagaccgcaggggctgacaaatcactgctaataatcctgttaggaacagtgcggccagcggagttcgcaatagctttatttttataaagtaggcgctgacagtataaaagacaacgagatctcttttttaaatagaag

SEQ ID NO: 116 - DNA, 6193 bp, Artificial Sequence
  subsequence of DRD4
tgcggccgcacctgatgcctgtgcacacctgtccctggtggtctctcctgtggtgacttcttttgctcaagggttcacaagggcagcagactgatctgcacaagagggactgagcctggctgcggccgcacctgatgcctgtgcacacctgtccctggtgcaggcccttcccccgcagccccgtgaccttgtcctgtgaggctcacggtgcctgctccttctcagagtcctcatttctccctttaacgcacgccctgtcagcgcctccgtctagcccgtgtcctccagaccttcgaggtcaggggcctcaattcctggcacagcccctccactccaggcctcccagaaacagactagagcatcaggatgtgcgccgcctggggtcccacagagtggtgcccccttttagtgtcttctaggccccttagtgacagactacagaaaatacctctcaggtcacaggtcacccctctttggtgaagagtccatagaattctctgctgcgctttgcaagcactttctcttctgcacgtttggaacctaccccggcctgtcgtgtctttctcctggcctcctcgcgagccgaacctactgtccggtcccgggaccccctgcccagggtcagaggggcgcctacctagctcacggtcttgggccggagggaatggaggagggagcggggtcgaccgctcagctgtccgcccagtttcggaggcggccacgcgaggatcaactgtgcaacgggtggggccgcggctgaccgtggtggtcgcgggggctgagggccagaggctgcggggggggggcggcgggatgagctaggcgtcggcggttgagtcgggcgcggagtcgggggcagggggagcgggcgtggagggtgcgcacgaggtcgaggcgagtccgcgggggaggcgggcagagcctgagctcaggtctttctgcgtctggcggaacgggcctgggagggaggttttgccagataccaggtggactagggtgagcgcccgagggccgggacgcacgcacgggccgggtaggatggcgctggcgtcgatgcccgcgcgcttcagggcctggtctggccgcccctccatccttgtcggtttctcgggtcgcggaccccgcgcggcgccgggcgatgctggcctgcccgtggccaccacctcgcttcattcccgtctctttgggccgccgcattcgtccacgtgcccgtctctccctgcgcaaaattccaagatgagcaaatactgggctcacggtggagcgccgcgggggcccccctgagccggggcgggtcgggggcgggaccagggtccggccggggcgtgcccgaggggagggactccccggcttgcgacccggcgttgtccgcggtgctcagcgcccgcccgggcgcgccatggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtgagccgcgtccggccgcacgagcatcctcacctgctcctcggttccccgtccctgtccctacggaggacccggcgcgacccggcccctttctggtgcggagcttccagctggggcggcggcaggggcgctgcgccttgtccctcggcgatacacccaccgccgccacctcgcgaccttccacccgctgcgctgtctgtcccccgaccctcgttcctcttctccttccccgtctgtcttggcgtctgttatccaggagatgcccgtccttctatccagggaccccggaaacaggcgactttgtcaagcccagtcccctccgtagctggatttcacctccagggcagccagctggacagacaggcagatgcaggctcagccccctggctgccgtgggacacacacacacacacactgccacagccactgcccaccacacacacctagtgcagatgctggcacacccccagaaggaggctcacagctcgcaggggagacctgggctggacaaaacccaggggaggggagggtgtgtggggaccaggcccctgctgagaaccctggggggaagcctgagggggaattgggggatggagcccacactccacaccaggtctggccctcgagtgggtcggccttggtgccagcccctctgcggccagagaaaagcagcttagggctgagctggagacgcggtgtccccgactgtgggggagggggactcgaggtttccccttgatggacacagtgaatccaggcggctggggcagagaccagcagcacgggacacgcgtgacctgtgctcctttcgagccgcagacgtcacagtgacgacgtttaagctcctaatctccccaaatcggcgggaaggattagaggggctgcctgctcctttgcccttggagagagtcactccacgtggagtcctacgctgggcactgggcacggtcttcatatttttaattataatttatcttcaagacaattatgatgtggatactatcatcacccttgtcttacagatgaagaaacagaaatgaattaacgtgcccgagtctcccacagagaacttagccaacagagcagccagcttcgcttctaggcccagcggggaccctgcctaccccaggctggggaggggctccctttgagcccgatgatatcaggcccgcccccagggaattccccaggccgttctcatgtttgtaacaaaagggagaccaaggccagaagccgtgattcacgcctgtaatcccaggactttgggaggcaaaggcaggaggatagtttgagaccagcctgggaaacataccaggcctggtctctacaaaaaattttaaaatatcagccgggggtggtggctgcggctgttgtcccagctattcgggggagctgaggcggcagaatcgcttaaacctgggaggcggaggttgcagtgagttgagatctcaccactgcactccagcctgggcaacaagagcgaaactccgtctcaaaaaacaaaaagaaaaacaaatcagccgggggtggtggcgcgcggctgtaatcccagctactctggagactgaggtgggaggatcgcttgagctcaggaattccaggctacagtgagccatgatggagccacagcactccagccgcggtgacacagcgagaccctaactcaaaacaaagggagatctgcgtggggaaggggtgtttccctgcccggtcctctggcctctggctcacagccgggcccccttctccgtattcagccctggaactacccataagagtgggggcgggtcacaagggcccgcggtggctgggaaacctcagggcctgtggtgtcgccgcgcaggtccagggtggcgcgtggctgctgagcccccgcctgtgcgacgccctcatggccatggacgtcatgctgtgcaccgcctccatcttcaacctgtgcgccatcagcgtggacaggtgcgccgccctccccgcccgcgccccggcgcccccgcgccccgcccgccgccctcaccgcggcctgtgcgctgtccggcgccccctcggcgctccccgcaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactgggccacgttccgcggcctgcagcgctgggaggtggcacgtcgcgccaagctgcacggccgcgcgccccgccgacccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgcggccccgactgtgcgccccccgcgcccggcctccccccggacccctgcggctccaactgtgctccccccgacgccgtcagagccgccgcgctcccaccccagactccaccgcagacccgcaggaggcggcgtgccaagatcaccggccgggagcgcaaggccatgagggtcctgccggtggtggtcggtgggttcctgtcctgaggggcggggaggagaggagggggggggtacgaggccggctgggcggggggcgctaacgcggctctcggcgcccccaggggccttcctgctgtgctggacgcccttcttcgtggtgcacatcacgcaggcgctgtgtcctgcctgctccgtgcccccgcggctggtcagcgccgtcacctggctgggctacgtcaacagcgccctcaaccccgtcatctacactgtcttcaacgccgagttccgcaacgtcttccgcaaggccctgcgtgcctgctgctgagccgggcacccccggacgccccccggcctgatggccaggcctcagggaccaaggagatggggagggcgcttttgtacgttaattaaacaaattccttcccaaactcagctgtgaaggctcctgggggctgatggggagtggggaagaggggtttctgcctcagtggccccaggccccccagccagttaacctctttctccccgccaaggaagcccacagagcagaccccaccaagccggccgcctgctcagggtgaggggggaaggggcccccgagagccactcagttccgcagcgctggggcccagcttcccctctctgcaggggagaggaagcagcacccagcagggcaaggtgtctcctggggaagcccagttccgatgggaagaaacaaatggggagaggcggggtggggggctgtcagccgccaggccctgcccgggccaactcccacctctgagaaccaggcacagggggctccaggctcatgctgccccagcagaccccggcaggggagcccaacctgtcattttagaagctgccttgaggccagagcctagcttcctgccagtccctggccagaggatgccccaagccccacagcaaggggcctgtggtgatgggtggggccccaccaagtggcttccctggagggagctccgtgctcagtcacccctggtgcccagccaacctctgcccagcgtccaccaatgtggctgggccaaggggtgggagcagggaggcggcagcggggccaggggctgggcagagcgacccaagctgcctgtctgcaccctggctccatcaaccagtgcagcccctgaccctgggggaagaggggcggctcccagggccctccatgcaggaacagcctaccctggactggatccagctctctcccaggcccaggttgtgggagaaatgggggaccctccgcctcccaatcgtgctggctggaactttcctgtgctggggattcggcgtttgcagccagggtggccagtcagggtgccaggctcccatctgaacactgacagactgtgggctgtgcagtctacagcattgggcacaacctcagcttgctaaaatactcagtgcaggctgggtgtggtggtcacgcctgtaatcccagctactcgggaggctgaggcaggaggatcccttaaagctagggagtcaaagctgcagtgagccgagatcgtgccactgcactccggcctgggtgacggagaccctgtctcaaaaaagaaaagaaaacgagtattgggtggagagaggaccaagcccaattactactttagtgcggctgccccgtcccgtctggtgaccacgcacttctgtgatggcctcagcggctgcctcctccaggacaccttctccgactacctgcttcccaggaccagcctgtcccagtcacccccggaacagactgagtcacagtggattcctctcagacacccgtgagagtgaaggaggggctcccgccagctttgtcc

SEQ ID NO: 117 - DNA, 8064 bp, Artificial Sequence
  subsequence of TBX15
agggcttttcctatctcatttcactagcagttgagctgacgggtgcgtgcgagaatgcgagacctgccacgtgtacacgtgtacgcggagtgtcgatctgcctgcgtgtccttggatgtgcctgtctaggtgcccacccgcgcgcgccaccacatccccaggcctctggaacgcagctgctgtgtggcccggcccgaagtttctggaggaggaaatccctccacagaaggtttacattatttcgctccgcgggagacccggcttcggcagcacttagcagaagattttggcgggaaaggcccaagccctagctgaggactccgggtggagcaggggctgaggtccgagcgcagatggcgccgccgagcgcctgaaatatacttgcaaggccgcagcaatatacttgcaaggccgcagccggagcagctgttccagccgatcctagctcgaaagttcctctgttgctctgggagagggcgggggagagcaggctcgagagccaggctcctccgaggctggtcttgaggcacttctctagtagcttctccaaaagactgagagtgccggcgtaggtatgacagtgagggtacctcacagacccttctccaaagtctggcgggccttggggtttttcggggccaccaggctcggtggaatttttgaaacgctttcgaaatacatagtttcctctgtggagtgagtgcctacaacgcgcaggccggactgatcccccgttgctgcaggttggtgccccaagctgcgggtgctcgggcgccaactaaagccagctctgtccagacgcggaaagaaaaatgggctgtgaaaaagcaaaaggcctcgtctttgaatgaaagttaaacattaaaatctgaccctagagttgtctaaagatcgcggaattttgaagctccggcagagcggactaaaaaacggtgctatgagagatggtgagaatactctaggcatgaacgtgtgcgtgtgtgtttgtgtgtgtgtgtgtgtttcattcttcccgcaaaacaattttttgtttttttcctattcccggtttgttatcggcctagggcgggagaaccacgcagcggcttctgggccctaaggacaaaagagttaaaacaatgaggctcacccgggaagagacgctgccctgggcacaatagggtcgcctgcattactcctccatacacacatctttaaatgtgtccctgtgtgtgttcgttagggtgctgtattacagaaaaagaaaggcctaaaaacacccccagccctggtcgcgcctttcgctaccgcctgagtctggagccgacagctccacctcttctgctccctggaccgccgcgtctccacgccacggcgccctttttactaaaagatcttttctcatcctatcagcaaatcgttaagaaaggcttagccattgcgggggctccaacttaaggattcccccggcccactaaaaggctaggcccggcctgtagcccagctccgcagaaagccagagggtgctgggctttcagcttcttcctcctagacacttgccccacaaatatatttcgttttctctaatccaaatacccatctttttcttttttaaaaaatgataacgtaatgggaaatgaccaaccgaactctgttacataaagttagttctgttagatcttccaccccacccccatcccgcgggagcgagtaaatagaattcatgagcttagctccccaggttcacgctctggaatggtttctttttgcctcattccctaagttttctctcttctgcctcctgaatggagctcaggctaaggagaacggcagaaagagcaaactctgatctgaatctctaattatgaccccatgtattacccatttgaacataaggccctagacgggctccgtgcgatctggggcctcccaagagaaaacttccccgggacaggacgtctgccacgcgcagctaaacaacttctgttttttccgccgtggggaaaataaaagaaccttacaaattctaaggcgtcataacccctgcaagaacttctaactgtatgaaggcccacgcgagattttgacaatagataaatgagctgaggaaatagggtctggccagcgaagggaaacacacagtagccctgggtgcctttctggaatgcccacgcaggggtccgcgtggacaagcacttgcattcaaatacaggaaaaggcttggacggtcgaaataaatctccttttaattttcttttcatcgactaataaaaataattccccagcactaaactcaaataccgtaacgggccacaaaaacacggagaattcataaaactctatctctgcaggtcacccgctaatcgcattattattagcctcgggagcatggaaattgaactgtcactgcctaaagagaaaatgtaagcgacagctgtccctcctctgagttggacagctttgtggctgagatccccaagctcctgagccccagaccgtctctatcccagtgcaggccgctgccccctccccgggcttgtcagcgtgtgaagcggggtctactccccagctctggctgttcagcaggagcaccttccagcatggttttagaagtccgggctggaaagcttctgaaaccgctaaaccaagttgcgagcgttgtatgtgcgcgcgcgcacactcaagattcttccgacaacagctgttcccccgggctgtttttgcacagttgaaatcttggcacttaacacaaaacgttaaagttggcttctcccctcctagggttctgttaaaatacagacatgacatcccccctgctttttacatgttccgtgcacataatatacatgggttactgtaggatctgagacacaaatcaatcgtacccacaccctaaatacacagagatgtgggccctgaagagtgacccaaggagtaaggggtctaacatccattcttgtttccgtaaacgccaaaggagtagaaatataattatgttccaccatcgaaaagttggtgggagaaaggcaaggacgaggttgaaggctaaatgagacaactggtcattccccgaagactgaggtattggattggagaacgccgcgccagtcctcccgagttctcagaagcgagtcctagatctcggctcccgccgttgccttcggcccgcgcccctcccaatcccagggaccggcagcccttcatccacggacacccagaaagctgcgctctccacccaaaagaaaaagaaatgcaaacaaacaggactccttgcttaggctggcgacgaaaatgcaaggctcagtccagccccaaaaattggttccggtgaatttagggaggctcagggcatttgcgcgaagaaacagaaacctatgttggggcaaggcagggcgtcaatggcagggcctagctcctcacccgcgaccggcgactggcccttcggcgtcccctctccgcccgcctcccgcggtcggctgcgacgcactcacccgggtgaggctccaggccgtgtgccgccgcgtcctccgtgtctccgagtgggcccgcggggctcagcgcctccatagacaggtccagccccttctcctcccagtctcgcagtttccgttttttatttgagccgatcaaggcttcaacggagaaggcatgtgctcgcgagctcagggcgactgcagatcttctcctttcactcattttagccgcccacacccctgcctccgcttgcccccgctaccgagggagcagccggcgccctcaagctctgagcgcccaccgggcccggcccgggagaggcggaggcgcgtcggacgaggctgagactgcggctcgcgggtctctccaccctccccctgcgtcctcctccgccctcctctgccggatccgacctgcgcccctacgctggcccagctgctaggaactagcgccccgagcgccgcccgctcgctgcatgagcgcccgagtcctgcttcccacccaccggggcaggcgctcacagactgtgtccactgaacttcatcttgtttttgttattattattattctctctcctctctctctctctctctctctcccctccctctctttttctctcctccccctctctctctcttcctctctgccttcccgtcctccctgtgatccaaacttctgggaaacttttttttccgctaaattcctcccttcccgaggaacagggggcaggcggtgcgctcctgtccgaggggtgagggagagggagggggaggaagaaaaagactgagggggaaaaaagccagcccccaaccaatagcgaggagggaacaagagaaactccgagagcgcgaaccaatgagaggaagggagagactgaggcccgggccaaagcgcagcgccagtcgtcccctggatgctcctgggccgactttaacccctggcttcccggtttccccatgggttactccaagagggaggtaggacagttgaaagttgaggtctgatctgcgcctttacaaactcctgcaaatcccaaatccatacgtttttcttccagatctgctggtctcatagacgctgcaagtccagagtcactttagttgcagatctggaattttggggctctgacaaaatgttaacctgtccaaaatgccagaaaacgattctgataatttcgcgtcagcccctaggtgctttagaatattctggttcctcctggatttcgccacacaggttttaatgttcctactgaacgttttctaagcaaatctgggtaaggcaaaaggaaacaaacaaacaagcaaacagaaagccctgttactaaccagggtcttgtaactgaaaattcaaagaccgcttcctccttccagctctacctggtggagaaggagacacaggcgccttgagagcaaggggcgggcggcggagcgcggggtttagggcgagctcctcaggcgtattccagctgggccccctcacgccgcaaatactctctttccctacggcccgaacgctgggctgcgcttcgcctgttccgggctcttgttgcagagaacctatggagaggtgacaggcaagaacagctaggaactttaacagaagagaaggagctttttcaaaattccctgcaaatcccgttacctcaagaccatggctttcccggccagctgatctcccccctggccgccccctgccttttgcgatctctaaggactgcattcaacatggatttctaaggaattaaaaaatccaacgctcctggccattcttaattcccacacctcttcccaagttacgccaccggtcgaggacggcaggagacccccgagtgcagagaaagctcaaaccggcagcgaagtcggtcctagccaagctgaaaaaacgtctcggatttcgcggacagcggcctagacacagcccgatcttccagtcctagtgccctggtcgagacggttctatccttttgcaaagaagccggaaagagctgggtcccgggggcgggggacagactgagaggccaagcaggccagtcgtgacgacagccaggcccttagaagtctccaaacctgaaagtttaggttagggggcccacagcctgttaagatttcttgttgttgttttcgcgaggcccgggagaatcagctctcccaccttgcggcaagaaaagatgacttgtcaggcctctgaaacgccgaattctctgccggactgcggaggggcccagggaaatctagtctttattattacagttggaagtgttctggtgctgaaggcaaccacagtgttgtgaattcaccttgggcctctttggacaagaggctacttttgcccccttccttcacccccgccccaaattaggatccaaccaggatgtcaggaagctagcatcccaggcgaatgcaaaaagaacaatggaaaagccaccggatgagtgtgattcagccaagcctgacctccccggagcctgtgtctcaggcttggaggtccccagggagcacgaacatgaactctggggagctggaagcagggtactggtccccgcctcctgcagctctgcccagaggacttggggagcccggatggagaggcgcaggatctcccacttcagtcagcatttggcgttgcttccaggagtcgtcgctgaaagtcagcgcgcattcactgctaccgggcttcagcagagaagctggagacaaggcagacgggaacccgcaatttccttccccagcggctggggcctctctctcacctcccaactctggtgtcgcccggcgttttccgcctgcggctggagcctgcccctaccaagcttggtggggtagcaatggggccggcctctagactttcccagctcttttctcatccccatagccggtatctcccatctctctctggcagccttttgcctttcacagtttctcacaggcctggtttggactgagaagcgggtacaccatatgcctcaccctctctgagaccatggaattttctcaaaaaggaggacttagcctgggcaacatggagaaaccccatttctacaaaaaatacaaaaattagccaggtgtggtggtgtgggcctgtagtcccagctactcaggaggctgacaggggaggatcacctgagcccaggaggtccaggctgaagtgagccgagattgcaccactacactccagactcggtgacagagtgagaacctgtcttctcaaaaatagaaaaaaaggaaaaaaaaaaaaaaaaggaggactttttggttatctaggaacataattgcaagtccagaggcccgattgttcctgtgtgtccaaagacgcagcatctgagaggcatacatagaggaatgagcaatggtttcaacagcatgcatctgcatcctctccgaagcttgcactccgcagagtcaggagtcagccagaagagtttccacacccctccttcagattgcacataggctgctcccacccccattccacagacacacatatcctggaattgtaggttaggccactaaaaccaaccgaacccaaatgaaagcaggatggtttttaacagtcttgaaaataaggggtttcctctaagtaaatgggaggagcagtgaaaactcccgtgactctataggagttaatgtaaggtttttcccagaagcatgcactccttggtgggtgactgctgatgggaaggaaaatctggagaaagagtaagcaggatggcttaatatgtgtgcttaggtgtacactcgtgtgcagttgagggaggctttgggagattacatgtaagaatctgcaagtgtgatacaaattttccttctactgattacatcaacgggctttaaaagtctctcctcattaccccagcttatctctcttttaacctttatgggtaagggtggggtccgcagtttatgaggctaattccctaggcactacctaggtttccctgctacaggcttcaacttttcgccctgagtgcatcctacaggcgatccaccctgaaatgactccatttcagggctgtttttggcctgaaactgattgacaaagaccaagcaagatgtctgcctcccacttaccccctgcaggtcccccaaaaggaagcgaaggttattggatacctgggaagtctgaggagccctggggtttcacaggctacctgcaggggcttacagaggggtggaggtccaggcctagtcttggaaggggaaaaattagaaagagaaaggcaatacagtaactgttcacctacaactgaccaggtccagatattttgatttttcaaaggaagagcagagtcatgcgacaacagatcagagtcataaacacaaataggcattccaagaaacggagcgcccacatcctccacatacctgtcttttcctcccattaccccacactgaatagcaccttaaataacaccactgctaatgcctcaataccaaggtgaaccaccagccggctttgatttcagagttttgagtgagtaattgaaaacaaagttaaaaatagttggccagccggacatattaggctgcccttaaacacgtgggtaaggcaccctgtgggggtgagagaattacaatgaattcgcccatcaaggtaggtataaaacattatgag

SEQ ID NO: 118 - DNA, 5519 bp, Artificial Sequence
  subsequence of TRH
tcaaatcctggagtctagttgggagaccaagcctgaggtttcatgtttgcagtgtctccctcactgcctctgaactttttgagggctggggctgagcctcattcacttctaggccttcaggacctaccaaggagtctgacaccatagtgggtgcttgttaaatgtttagagatgggccgggtgtggcggctcatgcctgtaatcccaggactttgggaggccaaggaggaggattgctagagtctaggagttcgagaccagcctgggcagcataagaagactccatctctacaagaaataataataaaaaatagccataaaaattatggtgcctggctgtagtcccagctacttcggaggctgaggtgggaggatcccttgaactggggaggtcaaggctacagtgaggtcagattgcaccactgcactccagcctgggtgacagaatgagaccctgttttaaaaaaaaagtttcaagatggaggcagtccccagggctagatgtggcagtcccacatctgggactttgggaactgactcggggggcaagctccttggcacccatgtgtttctcctccctgcttcccctcccctcctttgctagagttgggtgaactccaatagattcctgtgcaggggctgcagactcgaatgtccctcctcagtgacaggggcaacacattggaagacattagggagtggtggagactgtggtggccgagagcagccaggctcagctccacctggctgttgacaggaggaaatgggggcccattctttttcaagagaagccataaatccaaaattttctaagttatcttggttttaaaatgttggttattcagatttttttcaaaacactgtggaagtcaatcaaaattgtccatgaactggttgtagcctacaggccgccacttttggaattctgaaagctttcctctggctcctattacacagaggagaaaactgaggcccagagaaacggaggcactagccaggaatgacacagttaagattagcaccctggagcctgcggggtggctcacgcctgtaatcccagagacgcaggagtctgaggcaggaggatgacttgagcccaggagttcaaggctgcagtgagccgtgatcgcgccaccgcactccagtctgggggacaaagcgagatcccatctctttaaaaaaaaaaaaaaaaaaagtagagctccgctcccacttgccccccattctctctctctctctctttctctctctctctctcttgctctactctcgcccgtctgcgttctacattggttctgcggccgtgtccctttcgtgtgtgtctgccctgtgtttctccgtcgtctccgtgtggatctcggtctttgtctcgccctctagctctctcgtgcgcctgtgtttaatcgtgcgcttctctcaatctctctgtccctccctttgccccctacacgcgtgttctcgtcaacagatctccagaggtggtgcagaaacgaccccgcgccggcgccccatcctgcggccagtgcctccgcgccccggctccggtccccaccgtccccgccccagatttccggaggagcaggcgggcggggtcccgcggggccggctgccgtcagcgccccttcccggcggccgcgacccctccccgctgacctcactcgagccgccgcctggcgcagatataagcggcggcccatctgaagagggctcggcaggcgcccggggtcctcagcgctgcagactcctgacctgccgactgcggatcccgagtccccggatcccggacccatcctgtggagcccactcctggcaggtaaccgccccaacccctctccttccgcagacggtgtccgggagcactggaaagggagccctctcggcccgccctcaccttcggttctctgagagcttcctggagaacagggccggtgcacaggcgggcgctgtgctcgcacccccgggtcctccccgagcgcaggccggggacccgacgggaggcacggcgcgggcacatcggaggctctcgctgctgggcactcaggtcgccagagtccgcagcgcggggtgggagctccgcggcaccccctgcctgggtgccgccggacagagctgggtattcagggcgtcggcctgagaacctgggagttcgaatcccaatctcaacgctagaaatagaattttagcgttgaagttaggattttagcgttagaatcagattcctggcttagtcatgaaagcttagcattagaaatagaatcttagctttgcaaatagactttgagctttagaacctgaatcttagcgatagaaatgggatcttcgcgttggaatgaggacgtcggccacggagcttggagttagaacgtggcgatggggacctttgggggagtcttggcggggacgcaaccgttagcgtcgcggctgtgcctggcgctgcaatctcaatggttgcactcccggcgaggcagtcccagcgttggcttccccagttccgcgtcacattgaagggtggcggtggcgctggcgatggggttcctacggcctcctgccttgcgcaagcagaactgggcgcaggcactacaggcacccgtgggggcgaggacgcctagaggtgacaggtgtggacgcagagccggggcgaacccgccgctgctcccgcccctgcacgaggggcagtggtcctgcttctgggagggcggttgaactgtgcgtggtttgtttctggcgggcgtcgggggagatggagcgggatgtgctgtgattcggtgcgctcaggctccctctgtcccctaacccagacgccgcgatgcccggcccttggttgctgctcgctctggctttgaccctgaacctgaccggtgtccccggcggccgtgctcagccagaggcggcccagcaggaggcagtgacggccgcggagcatccgggcctggatgacttcctgcgccaggtggagcgcctcctcttcctccgggaaaacatccagcggctgcaaggggaccagggtgagcactccggtgagtggatggggctgttgggggctgtggggctaggctattgggagagccgtggaaacctgccggcaaaggggaggggaggcttggtcagctagccttctgcctttcggaagctcagaaaccctccctggctgcgcctaggcgggatctggagttgaccattcctcggggtcagattcctcccctgtctcaatgctgggaatatttcacagagagcaggccctgggtggggaagcttctgtgtgctagggagagagtttctttgtgtgtgacagtttgaattactactgtctaggcacttctgtgccagacactattccaagcacttcaaacaccttatcacactgtttgatcttcctaacaacctcttagttaggtactgttattacgattttacacagaagaaaactgaggctcccagagatcaattgagtcgcttatggtcagacaactagggagcgccagagctgggattggaacccagaggcaatgcctttgactattgtgtgaatgtgtgtatctgtgaggcctgaggggcaggtgcaagaccatgcctctgagtgggagctgcaggaaatgtaccctgcctgggagaaaagcaggagtgggcactcaggcgcctgcctattacatgggtcaggggcccctcactgacctctttccttccccagcgtcccagatctttcaatctgactggctctccaaacgtcagcatccaggcaaaagagaggaggaggaggaagagggagttgaagaagaggaagaggaagaagggggggctgtgggaccccacaaacggcagcaccctggccgacgagaagatgaggcttcatggtcagtcgatgtaacccagcacaagcggcagcatcctggccggcgctccccctggcttgcatatgctgtcccgaagcggcagcacccaggcagaaggctggcagatcccaaggctcaaaggagctgggaagaagaggaggaggaggaagagagagaggaagacctgatgcctgaaaaacgccagcatccgggcaagagggccctgggaggcccctgtgggccccagggagcctatggtcaagcgggccttctgctggggctcctggatgacctgagtaggagccagggagctgaggaaaagcggcagcaccctggtcggcgggcagcctgggtcagagagcccctggaggagtgaacccagttttccctgaagtcgagtttgtggtctaaggatgtcttgagccctgtgtgccccaccattcatgacctctgtattctctagttagatccctgaccataagcctgagcccctccctcccagccccatattcacacacatcccagcccctggccttgccctcttcctttaggcatgtgagaaaatcagcctagcagtttaaaccccactttcctccacttagcaccataggcaagggggcagatcccagagcccctctcaccccccccaccacaggcctgctccttccttagccttggctaagatggtccttctgtgtcttgcaaagactccccaagtgggacagggagcccctgggagggcagccagtgagggtggggtgggactgaagcgttgtgtgcaaatccagcttccatcccctccccaacctggcaggattctccatgtgtaaacttcacccccaggacccaggatcttctcctttctgggcatccctttgtgggtgggcagagccctgacccacagctgtgttactgcttggagaagcatatgtaggggcataccctgtggtgttgtgctgtgtctggctgtgggataaatgtgtgtgggaatattgaaacatcgcctaggaattgtggtttgtatataaccctctaagcccctatcccttgtcgatgacagtcatcctaatgataataaaacctgcatccagataatcacggagctctgtggttgctgttctgtcgcagctggcatgggattcttctttcgcattgagagggatatttaaaaggggatgggactagagttctcagctctgctgcagagggaatcagggaccctgggaggatgacaagtcctctctactgtccccatttcagaacacacttgggaggtggatggggttctggagcccatttgactcctggggagactaaggcctggagagaaggaagggcttgcccaaagacacacagctggctgaggtggagccatgggtctgagaggccagctcagggatttgccttgagtttgcctgatttgggaggcgggggtggtggtgagaggggaggtttgaggggaggactagtctggctcaggtctgtaatcttcatggttctgttgacagcttgtgtcaccaaacagcctgttaatgaacaggaacaaggagggcagaggaccaggctgcagtcaggagacacaaaccaggggtcttctggggctcggtgcaggacaggagggagtggtgtgaactagagttaactgaagaagtcatgtgaagacatttgaattcaaaaactttaagaaat

SEQ ID NO: 119 - DNA, 10262 bp, Artificial Sequence
  subsequence of FBP1
caagatcacctcagtgtccagcatctagtcctgtcagctaccccaacaaccaggcatgcacaccagttccaagggtacagcactgtaatacccaaaggctagtaaaaaccaaagtgccccaaaatagagaaaaacttcaatacctcatgatgtgtgcaggggagcagcacacagcagttaaaaggaggtagatgtagatgtcccgatattaatggatgtttcttgaataatgttaaataacaaagcaaactacagaaatgtgtattgtgatcccattttgtctgctattataggcatatgtaaatatgtgttcatgtgtaaacttatatgactgtaaaagagtatccggaaaaataagcagtaaattatttgcagtggataccccatgggatatatatggggtgggtttcactcttcccttgaattcttttgaatggtttaaatttttgtcaatgagtgcaacatagtatttctttaaaacctggtattcacaactatctagtataaaagtacacatattggccaggcacagtggctcatacctgtaatcccagcattttgggaggccaaggtgggcagatcgcttgagcccaggaatttgagaccagcctgggcaacataacaaaaccccatctctacaaaaacacaaaaattatccggcatggtggcacatgcctgtagtcccagctacaggggaggctgaggtgggagcaccaattcagcctgggaggttgaggctgcagtgagccattatcacaccactgcactccagcctgggcaacagagtgcgactctatctcaaaataaataaataaataaaacgcataccttacgtagccaaaattaacatgcttcatccaggccccaccatcaaaatctcactatccaaggtcccactgtctaaggttacttagacagtgggacacctcaaaacacatcttacatttctcagtctctctaggggtgtgaaaggtactgacacattttttaaacaatatcagacatgacaagcccttttttttctttttttgagacagagtcttactcggtcgccaggctggagtagagtggcacgatctgggctcactgcaacctccgcctcccgggttcaagcaattctcctgcctcagcctcccaagtagctgggattacaggcgcctgccaccatgcctggctaatttttgtgtttttagtagagacagggtttcaccatgttggccaggctggtctcaaacacctggactttgagtgatctacccgtctcagcctcccaaagtgctgggattacaggtgtgagccaccacacccagtcgacaagccctctttttaaaatatatccctggtcctcaaaggcagttaaagctgtgagccacctatactagctgtaaattaaagcttaagatttcacctgggtatgtttctaaaataagaatccttaggaatgtattattaagaggggccagaggcaacagtaatggagtcatcaccttccttaacatgtctgcttaatattcaaaaataaatgtgatcataccactcctttacatgaattacctccaccagtccatactgtgggaccaaagccagacagcctgccagtaagaaaaggaatctagattgcaggatgcggggcggagaagagtttttaagaatggagaaatgcattccaaacccaagtagcagggttgtatttgttcaggtcagctctgagcatcatgcgtcttttttttttttttttaagacaaagtcacgctcttgtcgcgcaggctggagtgcaatgatgcgatctcggctcactgcaacctctgcctcctgggttcaagcaattctccttcctcagcctcccgagtagctgggattacaggcgcctgccaccacacccagctaatttttggtagagacggggtttcaccatgttgatcaggttggtcttgaactcctgacctcaggcaatccacctgtttcggcctcccaaagtgctgggatgacaggctagagcctacgcgcccagcccatcatgtgtcatttttatagtgtgcataagtgcggggaatgttagtaaagtgtatggacaatctgaaaacccagttacacttcagttaaaagtgtggatcagttagctagaaaatacctgcgcacggcggctcatgcctgtaatcccagcactttgggaggccaaggcatcaccggaggtcgggagttcgagaccagcctgaccaacatggagaaaccccgtttctactaaaaaaaattacaaaattagctgggcatggtggcacatgcctgtaatcccagctactcgggagactgaggcaggagaatcgcttgaacctgggaggcagaggttgcggtgagccaagatcgcaccattgcactccagcctgggcaacaagagcaaaattccatctcaaaaaaaaaaaaaaaaagaaaagaaaaagaaaatacctgtgcacaccaacacaaacgaaaggatgttgtgtgacttacaaaaaacctcacaagaaggggtcctttgagtctccactccacttgaccccttgctactcacaatgtgatgagtagacaaacagtatctgatgggcagcacccaccacaggagaacttcttagaaatgcagaatctcaggacctgtcccagagctatggaaggagggcacacatcttaccaagatccccaaatgattcctgtgcccagtgacgttcgagatgcactccatagcgcattcagaaccattctacaaaccacttttgagctttccactaattgggcagaggctgcacacaccatcattgcaaatcgataggggttttgaagccctgggtgtttcctgtttctggcacagcatggcaactgtcccagcaaggggcagcccacgggagctcctttgcacacaagccctgctgctcctaaaggctctggacacacaggagacctcccagcctgcttgcggggggggcgggggggacactcgctcctaacaggtcttgtttcaggtcagagataactggattagaaacctgacttgggtccctaattgcagaaagagtgaaggaggacaaagaagaaatggggaaggaaggcacctgagctagcactttcctgtctgaggctagggcgggtatgacgggcaggccaggtgcccacacttggccggctgagaggggtacgaggcaggctgctcagggcagagaagaggaatgcaagatggaatcaggtgtacacctgtgagacggacccccaggtgggcccctccaaggtcaagggaggagatgaggacttgagtgaggctgagccagcttctggagagaggggctaccagacagagagcgagagaggcgctcagacatcagacggacagacagaggctgacggcaggaggctcagagggcccaacgtcagcccgccgggcaggctccccaggcagacagacaggacggggcccaccgcccaaggcccgactcacaggtgcgcgatgcccgccttgcgcaccgccgaagagatggctttgactgctgtgcagagcgagttgagcagctgggtcaactcgcccgtgccgcgggccttcctgccctcctccatgacgaagcgggtcagggtgttgacgtccgtgtcgaagggcgcctggtcagccatgcttgaaccgggtagagcgcggggctgcaggtgcaagcggcaggtgcggggctgcaggtgcgggcggcaagagagggcagtaggcactggccgcaggtgcggagctgcaggtgcgggcggcaggtgcgggccgcgggacctggcgggaggactgacagaccgactgccgccccgagtgtccgcagcgctcgtggtgcaactgggccaggccaggcgccggcgggtgaatcgcggaaacctttagacgcgggtcggccccccgcccccgggaacactcttgcgccccgcctaccccgcggaccagaccgcggctccgcctgcttggatcttcagacaaggtcggggatcctccctgcggtctcccaacccccgcccccacgcgccctgccgccccctagtcttcctgggctgtcgccccccacacacacccctccgtgccctccacccgcactgtggagcgggactcgggccatcggactctgccagagagaaagctatgactgtttcctccaaaatcataaaacggaagttcagaaaggttaagtaacttgctcaatgccaccagctaggcagcgaaacccgcagccagtccacatggcctggctttagctctgcagctcctgccccgtgtggctgcctgcccccgccccctgcccaagccccgtccactctgcctggcctgtcacctggcccttctgtccccgctaggagacacagagtccccgtttctcacccatctgagggcccacgtctgaatccttaatccaggcctcttctcaagattcctagaattcgagagctattgagtatttgaatttccaagatgtgagtctcgcccggaagtttcccggaaaacggtcgctgacacagagtcctcggctaccacacaaaggccagagggctccaccgcccgcatcctgaccccctggtagccacaatgctgcaattgatgtgctgattcacgaaacaacacctattgagcacctgctgtgctgaacactgtcctaccttccaggggtggagcctgatccaaacagacaaaactcctgcccttttcgagtgtacgttttcacagagaaacagtaaaaataaatatataaacaatagattagtttgttacataagccataagtaccatagagagaaacgaaatagaaaaggaagacacggaatgtggaggggagaagagggtgagcagaggcctcagtaaaagcttctctgagaaagggatatgtgagcaaagaggtgaaggatgcaacgcgccggtggagatgggaaggagagcggtttcctcaccgggagagccagtgcacaggccctggggtaggagaccatgtttcaggttcagggaatggccaggagaccggtggagctgaaatagagctagcaagggtcagaggtggaggagataaggccagcgggaggatgcagaggtgaggtctagagggccaagtgaggtttgactttgactaggaggaacccactggagagtttggagaagaagagaggcatggtctaccctgtgtcttcatactgcggctactgtgtgaagatcagactgaagacagtgccctccaggttactaggagatagtgagccaggtgttagcagtggaggtggtggattctggacatattttgaaggtagagctgatgggattcgttgacagatgagatatgtgagagacaggagtctcggatgattctaaggtttgaggcctgagaaactagaagaatgcagttgtcctcgactgcgatggggaacaccgagaggggcgtgtgctggaggcaggactgacaattaggagctaagtgtgggacctgctaagtttgaaatgccttctagacatccaaggcaatgtcaaatagacagatttatgtatctggagttcagaggagagatccatgctgcaggtataaattggaaaagccaactggaaaaaccaaccaaaaaccaagagatgtgacatccgggaagccagatgaagaaggtaagtaaaggcgggctgacccatgtgccaagcggtgtggatggggcaggcaggacaagctctgaaaattgactgctggattcaccgtggagacctggggtgcagattcctgtgcccagggttgtcagatgcggcggcgttgaatgaaggaaggtggtggtaccaccaggaaagacagcgactgtctcctcaaagccccagaggggacttagatgcctggcaaagagagttgatcctttaagggtcttatttaactctttgttcctcaggcaacttttcaggaaaaaccctgtgttaaaaaaaatagtaagagaaatacatccttaaacataaaaactcaaataggcccggctcactggctcacacctgtaatcccagcacttcactttgggaggccgaggcgggcagatcacctgaggttgggagctcgagaccagcctgaccaacatggagaaaccccgtctctactaaaagtacaaaaattagctgggcatggtggtgcatgcctgtaattccagctactcaggaggctgaggcaggagaatcgcttgaacctgggaggcggagattgcagtgagcagagatggcaccattgcagtccagcctgggcatcaagaacaaaactccatctcaaaaaaaaaaaaaaaaaagaaaaacaaacaaactggaggctgaggcgggcagatcttgaggtcaggagttcaagaccagcctggccaacatggtgaaacccatctctactaaaataaaaaattagccgggcatggtggtgtgcacctgtaattccagctactcaggaggctgaggcaggagaatcgcttgaacccgggagacagaagttgcagtgagtcaagatcctgccgctgcactccagcctgggcaacagagcgagattcaatctcaaaaaaaaaaaaaagaactgaaatgtgatgactagtatttttggcatagtaactagttactaggggagagctagagctagtaactaggggagtgtgcccctttacttgccagactctcagtccctcaggatcttggattggctaggaccgtaaaagtcatctagtccagccaaccctcattggacaccatccctgataagaggtgaagagcttgggatctgcattaagcaaacttgtttgagtcccagctccagccctcagcagccagagatctcggaccagttacctaatctctgtaaacccagtttgttcatccatatagtggaataatggttccatctggcagtggtgtcatcgggctgaaatgagatggcattggcagagtgcttagctcagagctacacacaggggacacactgaatacgtggaggagacccctgtctagctcagtcacttctgcttgtcacccacgatgatgggcagctcattgcctacaaggcaagccattccccttgggactaggccacgtcgcattgtggtgaagtgcacagactctggggctgcatggcttgggtccaaaccctggcctggccacttactggctgtgcaagcctgtgcaattggctaaacctcaccaagcaaccattggctcatctgtaaactggggctaatgattgtagccacctcatagggttgctgggagaattatacaggcagtgctggtagtgtttaagaacagtgcccagcatgtagtaagtacacagtatgtaagtgtttgcttcgttacatgggtagggccaactgttgttttcatgtgaacagaagcaagaaagttccctggccatcaaaaggggctagagggatgtgggaattggcagctgagcctgtatttgcctggtgtgcagtgatatcccctccatcatattggtcgattgtgactcgctcttttctgggtcaagatgcataaagcccatcacccaggtattagcaagagacagaggaaggcggccacctcacatgggttcctggcctgtgggttgggcccctccctcctccacccaccaccctcgcagagttgcttcttgccacatactccaacttcacatgggattgcagattcaagtgatgtgagcagtattaccacatgtgtaacactcgaaatcctgcgaagaatcagtgctcatgcatttcctcacccagctcccacgatggggttatgttggaggcgggcagtggagaagtaacagtgtggacagtccctgcccctacaacagccgccctcaacccagggctccactgcttactttcccctctgtgggggcggttagtgccacaggaaagaaaaacaggccaggcacagtgcacacacctgtaatcccaatgctctgggttgccgaggcagacagaccacttgagcccaggaattttaaaagcggcctggacaacataaggaaaacactgtctctaaaaaaatttaaaaattacccaggagtggtggcgcatgcctgtagtctcagctactggagaggctgaaggaggaggatcacttgagcccaagaggtcgaggctgcggtgagccgtgatcacagcactgcactccagcctaggcaatagagtgagagtgtgtctctaacaaaaacacaaaaagaaaaacaaaagccaagtggtgtatgtgagagggattgcagtagcatggtccttccttaaacacctcccccagctcctgtggccccgggggcctggtgtggaaactaaacagcaaacatctatttagcacttgctaagggtgtggcacttgcccagggcttcagaagcagcctttaatcaatccagaaccactctggggtggcttctaatcaaacccctgtctttacagatgaagggactgaggcttttctttcttttccttttcttttcttttcttttttttttttttgagatagagtctcactctgtcgtccaggctggagtgcagtggcatgatctcggctcactgcaagctccgcctcccaggttcacaccattctcctgcctcagcctcctgagtagctgggactacaggcccccgccacttcgcccagctaattttttgtatttttagtagagatggggtttcactgtgttagccaggatggtctcaatctcctgacctcgtgatccacccacctcagcctcccaaagtgctgagattacaggcgtgagccactgtgcttggcccaatttttgtatttttagtagagatgggatttcaccatgttggtcaggctggtctcaaactcccaacctcaggtaatctgcccgcctcggcctcccaaagggctggaattacagatgtgagccaccacgcccggccaacaaggtcaaataacctgtccaaggcctgcagccaggcaggaagtgacagaacaaagactcaaaccctcatctgtctgattccaaaccccatattcccactgacttgctggttctttcctaagtggcagtttccctccacgtcatgcctccagggacaggcaaggctctgagcccgcctacggcagctctctggactctagagctatttctgggctcatgttcattcattctttcactcaacacatagtttttgaacacctgctgtatgccaaaaccataccacaccctgaggttgcagtggtgaacaggacagacacagctcagtccactttaggtttatattctttgtgggggagataagaaaaaaaatcatttaaccaacaaagacacaaaactgtttcagggagcagagtgctatgaagactgtaaagcaggatgaggtaaaaactcaaagcctttcttatgaaatgacttttgatcagagactgaacaacagaaaggaatggggcaaatgaagagctcatagaggaacatacagaagaacaagcaaggctacggcaggtgcaaagaccctggtgcattcaaggatcagctgggagaaggccagtgtggcctgaagggcaagtgagggaagaaccacaggagataaagttggaaagagagacgagggcagattatgaaaggcctcacagcaagtcgctcaggttctattcatgcagccagaagacaggggaggccaggtgcagtggctcacgcctgtaatcccagcactttgggaggccgaggcaggcggatcacgaggtcaggagattgagactatcctggctaacgtggtgaaaccccgtctctactaaaaatacaaaaaaattagcctggcgtggtggcgggcacctgtagtcccagctacttgggaagctgaggcaggagaatggcatgaatccgggaggcggagcttgcagtgagctgagatcgcgccactgcactccagcctgggtgacagagcgagactctgtctcaagaaaaaaagaagatgggagggttttaagctggggagcgacatgacttactaaacggtgtgagaatggaccgtaggtggtcaagagcagaagccaggagacccagcagaaggagtctgacctaacctctacttgatttacagtcaaatatgtgaagcccgctgtcatattccccatacatctttacttctccagttaaatattttcagctcctccagctgctcctcaggtggcaagattttgaagttcttcccttcctgttctggacctggtggccccttgaggctccttataaccccatgctctatttccacgattccacccatctgctccacctcatgttccaggggtcctctctcctaggaa

SEQ ID NO: 120 - DNA, 8338 bp, Artificial Sequence
  subsequence
tgtctctaataaaggccttttttgtttcaaattacatacgctttttactgcacaatttttgtattgaccttatttcaactgaagcgattatttccagcaggtttaatttcgacaataagatatattttaatagttttacctttcgctagaattttgtattgtttaaaacagtaaatgaataaatttgctgatgattcaagaatatttgtttggtttcgttttgtatccctcaaaaggtttatttccaaactttttaaaacaaaatcacgagaaaagagttgttttaattacgtttgatttttaaaaatacttttatctaaaacccattttgtgcattaggagatagtttgcttccatgctcaaaacatggagttcacatctgggggacatttttatccctcagtttccttctaactgacgtttttgtcgggaatctgctcagacttttccccttttcttcacccacctcccaagttttccttatgtaatttagtaaaaggccgcctgtttttcacaaacacggatttgttattttaaaaatcaatagatctttatatgctagtgttaaaggtgtatggtttttcttatttccatcttctctccttttctctctgtcaggccttcttttatggcctgcactctaatgacgtttcttgatttctctttgtcgacaccaagtttttgttgtgcgtgctttcgaccctggtcaaccttccttcctagaggcccgctccggacagcgtaggcctgggcgcctgggtggagggtgcagggtgccgggcgcgggacttcggctctcgcctgggccatcccgggctctgagggcccggcatccatccccaaggtccatggctttcaagttcggtcacccaggcagagagaccctgccttttaaagagccctggaaagttgcgccgcgttttccctcaaccctggaacacgcagggaaaactatgggaatcgcccgcgtcaaaagctttcgacggcttctaggaacccaagcgggcctgcctcttcccctggagcttgccctcctgggggcgggaggagggcgggagggaatcatttctttcaaatgaagcccacgccagctctgaagccaatttccgccgcggactgtccgtggagagttggcgaccaacactttctcggacggcataaccgcgaggggcttagactggaaatcagatgcgccaagaggacaagagccaggggccagaataccccagtgggggacgcgccttggaggacggttcgcccagggtgggcttcagccctttcccagagtgctccgggcgtctggggactgaccggagcgcagggcgggggacacttacttcgttatccgcacgccttcctcgttggcatcagagccggacaaatcccaacccgcgccaggcaaagataaatgactcctggctcccccggcagctcagggggtctgggcgggggcggagagggaaggaagccgtcggccgccggaggtagagactgtggaggtcctcctcccggccccaagcgaatgagcccctgcggcccgagcagagagatacgcctcagagggacgactgatggggaaggatggtgggcaacgagtctgcgctaggcggtgagcctagcaccccagaggcccgagggtctggggctagtggggccgaccgttcaggagccgggtgggcgggaagggaattgccaaggtcggcccaggagctgcctcaggcgaggacggggagctttctgcactgggcttcccggggtaggagatgggcgtcgggtgtgggcactgcctgccctgggggaagaaagcgaggagataggtttcagagctgggggagtcgttggtgggagatctggtgtcccttcggcggccgcgggccgggggaccggggaagacggagggtcctgcggcctggagcaccaccctaccacaccaggcaacgccccccggggcgcccttcattcttttgcagagcccagaacttaataggtgggtccccttctgcaaccacgctgcgagagaatgactgccctgctaatctagtttacttgtagaaatcgactgtaaaccgggacaacaaggcaagggtgggggaagaaaaggaagcgccgaaggagccgccgctgcgtccccgcccccagctcggagctgccggggttggggccccgggtggcgctcccgacccgccgcgttttccttagggaaacaccgagtgaggcagaggatctcaggcccaaggtggctgcgaagagccccgaggcctcggcttggcgtcaatgtcccgccaccctcgagcccgggtcactgcccgggtcccggcccggagccccagcgtcctctcccgacagcgccaccgcgtgggctgaggccgaaaccttcggcgccggctctgccggtcgcgtcccggagccaacccttccgcgtcgcctgcagtccacgggttaaggatgtggcgggggatgggcaggtccctggcccagggtaccaccgggccactcaacacaaaaacgccctggcgcgtgcaaaatacgaacgcccacagtttgtcccaaccccttgaaatgaaaagtacacctggaaagcccaagatagcacccggtccgcaccccaaatttcaggcaggccagcaggcgtccagaaatccgctcccacccctcgtaggggcaggcaagctcaggcagccgggtgcaagggggcccaaaccgcagttgcctcccgaacgctttgaggctatgggcccacgccggtcttgaaccaagcggagagggcgaaaaagcccgctgcgggacccacggtcccagacacctactggagccagggacgaggggggcagtgtatccggcggcccaaactcgtaaggagtgggggccaacttagagggcttcccgagctggcgcctcgcctgttttctcggtatcacgaatcccctcggacaccgtttcagtccgtcaagaccttggcctccgggaaaggcccaagtgactgtgacggcggcgactcgggctcagttctcccccaccccgtggtgccttccctccccgccctacagccaagcccagaggaacagggcatcgcctcgcgcggtcccggagccacatccggacttgggaaccccgtgtcgggcttagaggacttcactgggcgggttggggtttattttcaataaattcagctgttcacatacacccccaccaccatttccacgcgcgcgcgcgcacacacacacacacacacacagagggacttgtcaactattgagacagcgacccagaaaggtgtaaattcacctggtgcccacccaattgcaaagctcccagacccacacaaaacactagcgagccctggaggtctgagacgccccgcgcccgactcacggcgcgaacaccagcgctccggccgggtcgcaggggccggtgcccagcgggccggggtctggcgagccaggcagcgggccggctgagctcctgcgccccgcggtcgcgtggcctactcgcctactcccttattattggctgggacctgttcctttccaaagtgtgatgcccggtgtcctaccctaggccacgcggagctctctcccggcgtgtcgggccctgcgcccctgcccagtggctcacctcggctcgctgcgcccgccttcgccccaagtgcccaggacggagcgaggggctgtgggtcggcgcagggacgctggctgctcacctccaggagccgacagaaggtgccttcggcgtcacgcgtccaaccctggggctgatttaaataactaataagctcagagaaattttacgaggggaacttttccttttagtatcaataaagcctagtaattgggtttgttggttacctaagctcgagttcgactttctcagtctctctctctctctctctttctcattcagaggcaacgttttcaaaaaccggcaattgcgtgtgcgtttggggtgtgcaagtgtgagtgtagatgtgtgaaagttatttgtttcagcagtccttgtctcgagagcaatcatcattgtagctttgagtgaagagttttcaaaggtgaactgttgttatttctgtcaacagaaaactgttgacaaccattcaagtctcacaatacaagtataatataagggagggtggaatatctaaagactgtcccgttggatatgacagaaagacatctgcagtgtttccctaatagtttgcattttaattaactttcattacattctgacttccactcaatatccgttgttttcagtaaagacttctatatgataaaacgttgtaatttttgttctggttttccaatcttcttgaaaaccagttcttaattgtaaggaatttttccacctgggaaccatgccgtaatttaaaaatataaaagacctttcatgaacaataactgcaaataacccatttgttagtgtctgcggagttgctggcacctatacaaagtctctgaaatataaaggcaatctaagaatgtttctctccaaccatagtgatatatacttttttcgtttttgttgtgtggaagttaaaaacacttttaaagttactttttagaaaagtgttgtgctaaaatttacctgaacttaaattccttatcaatgttagcctaacgaaatgaagagtatttctgaatcagattattgttagagtcatatgctaaatagtaaaggaaaagaaatgggaattaaagtttgcaaaccaagaccagggaaaagtcggctccagaagttaccaggaacccacccaaacctgtgcactcatatctcctgtatttacatattatagacaacttttcctttccacttttagctagaaaagtggtgggctccccttagttcgtctcgttttatgctgtaagacccaaagtctgctgagggctgcccctgccaggtaacttccggagacccggagttacttggagacagacccggccctttgcacattccaagtgaaggacgtctcgcgcattcttcccagagctaccacacacctaacctcttctggacacccaacagctggggcactggagcacctgaccctactcaagatcatcccccagcccttgccggcacttagggacagcagaatctgagctgtgggtgtacatactccccattttctgctttcccagaggatcacagacaacttttcttcccatctgctcccgctggactcctatgatcacccatcatccccagttgttgctatctctgagctcttggtgaccagcccttgtcccccaagctgaccaagttgagagagtacttgtcctcccagcactgtctctcagtatcaggaagttgagatcagtggcagggaaagatggaaggattggcaaggaaagtgcagggccaagtagagtagctcagagcctcagcaatgggcacctgtcctcccccaaacaacctgtggtctctccggctccctgcttttccaccattggtcccatcctgggctaccagcctccactgagctgctctgggaacttagggtgtctcacattacttccctgctatggcatattttggtttcagttgagctattttctgaagatgtggatactccagttttattctttttctgtctgtgtctcagtactcttttccttttttaagaatgttccccaagcctggcttttgccagaccctgtgccatctccccaagctctatttacaagaacaagaagtgatttgtctgtttatagcaggtttaattaagacagttatgcgccgtgaactctttgggcctcaatcaatgcacttagatttggaaactcagatagaatcacatcttacacgtgcggccccggctggccgcccaggctgaggacagagctattgttttctacatcagcctcactggaactgggcaagtcccctgcagaactggcaccccctgctcctcccaagagacctcaagtcaggtctcccggccagactcaggcctttctaatacaggcgtgaagggcgggacgcccagagccctcttgttggccggtggctaaggagccaggtggataacttctgcggattcggctttcaggctgctggagctctccccagcccgaggccttagcccagcgcaatcccttgtccagaccggccagtttccgcgtggcgaaaaagggggaggcaagtgtgtcttttccaaggagcttttatccaacagcctcccctccccccaactctcagccttaggtgccgggcttcagccgaagcgtttttcaaatttgtcatcattggaaacctcggacaagtggagtgtaaaattttgagggtttccaggtaggctctgacttcaggcattcaatgaaaggggaagagggctgagggcgggggagagagacaagcacagagggctccctgtagtcggcaggaattccctccctctgcagcccagctgcacccactagcacacactagtcttgccttctcccggctttggaagaatccccaccacattctttagctctcagaaaggagagggcagtcaggtcaaggtctttcagcatccctaccgcaatatacccctcccagccccctcaccaccaccacgttggcctaggacacaggaccccggggccagggcctcagtgactgcagcttatgccacctaatgcagaatggggtgtccctgcctccactgctcctctcggtctcagcatccaggtcctgaaacccctgcgattctggcctgggaagccagacttgccaccaacatcatggtatcacccataattgatccactcagttttcagtagatgtggaatccaacaagagtaacaccaccctccacgccccactcttttttctttctttctttctttctttttctttctctctctctctctcttttttttttttaagaatccaaggcacttgaaacattctgagtgtgcctgcagtagggtctcagaggagttgctctgaaagctgttgagagcagccacctttgctgtcactgagagcttggcagggtcagctctggtgggccgtttgcacccaggctctgatggcaccaggatggcttcctgcaacccggtaggaggactggggtgggtgggtccagtatttcgtttcttcctcttcctttttttttttttaatgtccctctttatttctattgaagactaacctcagtatcaaagggtgcacttgtttgtcctaaatggcttgtagctctttcctactctgccacaggacattagctctgggagcaccgcctggcagggccttcgctttgggcctaagcatccaactgaggcccagcaattccacctgtggggaatcttgcagtgaggtatttgtgcatcaggagtggggagtgagggagagagagctgggctggtgcagtatccactctcgcacctcctccctgagcggagctccagtatagactaagtgaatgaatccatgaatcagctccatagccgaggggtagaagaagcagccgtcttcgttaaatgggctgttggcagttgcggctgactgctctacccctcgcaggcccgcctctgggcaggcctgaggtggccctggagccccgccagcagcccccgcccctcaggccaagctctggccctggcaatctctgcactttgtcatcacgcccgagtatttgacattcgcaattatctgcaaataccctgatcagtttgtatatgaaacactttttttcctgccttaaggttggaatctggtaagtttcagacaagtggttgcagggggagatgacccaggggaccgtgcgggacagggccggtgaacccgattccgcgaggcgggcgtctcgaagggactggtctgcgtgcctcaccgggttccccagcctggctccgaggaaaggccgcgcggaggcgagaaagggcccacatctcgagtctccagtctctccttgaaaagagcaccccctaatcccggaggaagggagggtgaaggaggaggggagagcccactacaggcaaggcaggaaggttaatcattttgttaaccggaaagggtgctgccccgcctggccgtcgtcaccgagcgtgtcagtcacccgctctcgatttcgccgcagaattacctgctcaaagagccaccccaagaagccctcagctccgaggtcttcccgcccgtcgtctgcgggaagtaaacggcgtcctccctccaaagaagtggatgccccttcgactctcctccgccccccggcggttcctaaatctaggaaagccagccccggcatcgctcactactcctgcacctcagctctgggatacccgctcagtacagacagaccatcaaccaaaaaagaaaagaagaaaagaaaagtggtggtgggggtgggggtgggcag

SEQ ID NO: 121 - DNA, 8717 bp, Artificial Sequence
  subsequence of BCAN
ggtcggggacagggagaagggagtgcctgcctggtctgcgccccccgcctgtcagcccttgcctcgaggctctggggcacccaactcgtcgactcctgacaccgcagcggggtaggctgctggacagccccgagcgcctgcagctgctgctgccatctctgatctacatgcttccagctctgccagtggcagcccccctgctgctcgcagtctgatcagcaacccctcgggtcctcgcatcttccctgctgcgcgctcctgtcccagcgcggccgccgagatttccgaccttgtcccaggcagggcggtagcgttccggatcagtccttgcctccgtgcccccacgcacaggcagctccaggagcagcggccagcaacccttctggggacaccttactgtagtccggcaggacagccgatcagagccgctctagggggtggtcggagtgcccatttcggctggaatcccaactcggctccttgctctgtgaccttggggtagttagttgacttctctgaacctcgggttcctcatcggtgtaatgagcaaaacataaacaatctttttctcacagttgaggttatccacaggaagcatttaggagaactggctcaggtagacatcagtaactattagctggaattcggttaccactattgcattccttcctagcactgccaggggcctgggaattccctctcccaggtatttccatatcctgtcaggtattcccccacctattggaatagctctgcctacatggttctaataggaccaagagattggcgtcaagtccaaggggcaccattattatctgcattacttagattgcagatgatgacagtaagatcagggaacagtgttacccttagacccacccacagtattaccagtgcctatgttgctatggctaactttgctatttataacagcatcatccacatttcagattctatacgagagggatctgcaactgctcggctggtgtcattgctaatagtattattagttttgcttccaataaaaaggactatttatatgatgattgcaattacttctacaggccctaaggccctgcttatattgtgtaggctcatgttgctcctcatgaagccgttaagggggtagcagagagcttccctcctgctatagccaaagctctggcttcttgtgcctccctgctgaccctgctgagtgctggagctcagttcctatgggacagaccttggctgccggctgatgggcattcagcatggaggtggtgggtgttgttagatgtgtgtacacacgtgcatgcctacacacacacacacacacacacacacagagagagagagagagagagagagagagagagacctggtcttaactctctgggttgggtctatgaagactgtgagggagatggacatcccgggtcagacagaaatgatgtgacaagaggaaagtttcctgaacagctcaagaaattcaaaacaagggcagagatacagacgctggagctgaaacagagagggggatgttgtggcgtcaagggaacggaccgcatgactgctgagctctcagggttagagttggagaggtgtggcctgctctggaggaagagaagaggaagtggagatgagtggtgtgtgggatagagaaggccacagaaccctggacccctgggttctgaggctggtaccggatgtggggaagtctgagtttcctcagggctcctagctgcaattgggaggctgaggcccgagactggagagacagagctctctcagaactgcacggggctaccctcttggcctcactggtcagtgactcccatttttggggtcaagggtaggttgccctccatgttccagagcagttaggtcctcattggccttaggccctcttccctcactctcccactgtcagacagaggtgaaagggtggtccagggtgttggccccaaaatgactccaactatatgggtctctgggctgcccgtcagagagatgggagccaggtctgtgagcagctccagctccagcccagagagaagggagggagggcaccagaagggggccaggcccaggccctggcacagctcagatgcatggcaaacagtcttcccagcacccggtgggcactggctgctcctgcccaatggtccccactccagccccagaacagtcctcccaagcaccccaatgcctggagctggtctccgcacctctaatctggccccatcaagaacctcctccctctgaaaggtcagagagaggccctaagcatctggttgtgccactcctctcccaactctgaccctagggaatgatatccagacacagtgggggagatgggggttatgatggcagggaggtgggaggatctgggaaaagtaggaaatttcccatcatctcaggcttaggaaagtgcagggacaggtatgaaggcagatgctgagagggcagggaagatgggggataaacagtgggcctggaatgggagtgcacagccagtggaataaacctgatctctaggaagtcctgcctgtagtctaaccttcaattctcttgctgcatcattggcctattccatggcatacctggatctccatgctcttctaagtttagcccaacccttaaagatgatgtagctctgagtcttctctcctgggagcacattgccctcgtgcctgacttggctttactagggatccagggtcagccctatccttctctgagcctcagtctccatttctcaatgtcacaacacatcctttttatgagagtgtaagtgcttcgagatctttggaggaatattctgggttctagagcttctgatagccaggggtaggaaggggtagcccctcccatttattctccccccccttagtccaccctccagcccaaggctttcacaaaaggtagggtgggggaagggaggggctggatagaggccagactggcagagacaggggctttgtcccaactgatttctgagccaagagttccggtgggaaagcccgtgccaggcagaaaggagaggctgggaagaagaagggaggaggcagccaaggaagggggatggagaatgttggggagtaaagagaggagtttccttacaaataatggtggggaggaagtagaggagagggtgggctcaggagaagctgtgcttacttgcgttcggtctaggaggccctcctggaaaaggccaggtgagtatggtgtccctggaggcagggacagtctagaaggcagaagaggctaaagagatactatgggagaaggctctgggttcgaggaaggagtctggcactgggaggagcagatggaattctgagtataagagcacccaccattcatgctgtaaactgagctcctactgtctgcctggtgctatgcatttgtacaggagttttaagatactaaacactctcaaagcacatggtcctatctcaactcatgagatagtaatgatgatgcttttttataagtaagaaaataaaggctcagagaaggtaaggagcttggcttaggtcacacagtgagttgactaggactcgggttttggttctgagaagaaatcacttgctgggaagatggggatcagaaacagagagtccatggtgatgtgaaggtaggggacttctggagccaggatatgtgtgaacccacatgggtgtggtagggagcaggggggaagtccatcctgaagtctaaccccatctttccttctcatgtccctctgtcagcctgcagcatggcccagctgttcctgcccctgctggcagccctggtcctggcccaggctcctgcagctttagcagatgttctggaaggagacagctcaggtaagcaaccccacttggggtcaccgtctctgtcttgttcagccctaaccaactgcttccaggcttaggggccccaggaagagagagaattggagggctgtggggaagcgtccaggctggaacacttggaaataagggctgagctgtgagcatctggtgggttggagaaatcatggggcaggactagggaagggcactgggtgcaaggctttgggcccctaaggtggaggataggggatcctgaagctaggaggtgggaaaggggtggaactgggagaaaccctgaaggaggccctaaagggagcagactgggaaactgggctggaggactcagggggcgaggttggaggaccctggggtgtggtctaggaattttggagaggaagtggaagactctgagagcagggcaaggaatcctgggggctgaattgactgggagactttgggggatgggtctggagaccgtcggggtggtcctggaggacctagaggtagggctgcaggaccctggcccctggcccctggcccctggtcctaggggggccggggaatcctggggccggaaggagggatcctggagcggggcttggaggccaccgggtgggactctgagggtcgacagcgttaagttccagccggctccacccgttcacagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaaggtgagagggcagggaggttccagagggagggagggagggagggaagggaggactcttgccttcggggatcccacagtgtgagagggaagcaaaggtagctggaaggcgcagcctgggttggaaaaagagtgaggagacacgggcctttgttgtctcctttctctcttcaggtggaggacattctacccacaatctaacttaagtccctcatgctgtagagtgagcacaattgaactttatttacccttgtgtacagaggagtgacaaggagggtgaggggaggccagcgtgctgggtgctcacctggctcaggggtcctctctgccccacaggggtcgtctttctctaccgagagggctctgcccgctatgctttctccttttctggggcccaggaggcctgtgcccgcattggagcccacatcgccaccccggagcagctctatgccgcctaccttgggggctatgagcaatgtgatgctggctggctgtcggatcagaccgtgaggtgggcaggggctgtggattggggcttctattggcccctgaggtggccatggccccccttctgctgggtgctgcctgctgtgtcaggctggacatgcagggctttttgcctctgggggatgaggctggtctgaggaggggaggtgaggaccctgagcatgtgcatccctgcagtgctagaaggacagccagctgtcagcaagtgtctgcactgtggtggcagtggggttcaatagaatcaatatgggctggctccctggtgaaagcatctgtactaagtgattctgtccttcctccctaggtatcccatccagaccccacgagaggcctgttacggagacatggatggcttccccggggtccggaactatggtgtggtggacccggatgacctctatgatgtgtactgttatgctgaagacctaaatggtgattaggagtgagaggttcccaggggacaatttcctactcccatgctgcccataggtcctcccggggcctctgcaggaccttactatccctcctgagtttaagggggcaagcaggagtaaggagacaattggtgtccctctctccaaacactgtcacagaggcaagctcatgaccaaagtctcagccaggcagcaggataccgagtttaactcatctaccacctgccaggtggactctgtgcatgactctgcagaacaagaagtttgggccccatctctttgagctctaatatccatcttgaggttctattcagactatgatcctatgaagtagagtagttgactccctgccacagatgagggaactggagaagcttgcccagggttcccatgggagaccagagtggaaggagctaccagctgcctgataacccagccttctcttctccacccaggagaactgttcctgggtgaccctccagagaagctgacattggaggaagcacgggcgtactgccaggagcggggtgcagagattgccaccacgggccaactgtatgcagcctgggatggtggcctggaccactgcagcccagggtggctagctgatggcagtgtgcgctaccccatcgtcacacccagccagcgctgtggtgggggcttgcctggtgtcaagactctcttcctcttccccaaccagactggcttccccaataagcacagccgcttcaacgtctactgcttccgaggtgagcccacctccctgcagaagctgagaccaatctcagaaaggcagagttgagtcctaaaagtcctaccccagtctgatcagtttagctcagggctttgccatttgcctgaagtggtcgtgggtgaagtccagcttgtcatctagggttctaattctgggcatgccctctggtgtggtgtctgtcagctgtttggcccaaggagtagtcaccttttggcctctccttatctgccttcccaccagtagtccatgccttgtttgctccagctgggctgctgaataagctcccatctcctgtcagcttattggcagagcaaagacgaccaacattctgtgaattcaatgagtaatctgcccaagttcttccagtcaacaaccaaatgccctcaggagccctctggaggatttccagaaactacagcttaagaaggtgtcagatctttaactaccaaatactggtggatggatacaattcccccaaaagggagcaagaccttaagtacagttgggaatagagatagctttctgcccacatcttatggcttgtttctttttttgagacagggtctcactccgtcccccaggctggagtgcagtggtgtgatcatggttcattgtagcctcaacctccctgggctcaggggatccttccacttcaacctccggagtaggtgggaccacaggcttatgccaccatgtctagcaaatttttgtaatttttgtagagatggggtttcaccacatgcccaggctggtcttgaactcctgagctcaagcaatctacctatcttggactcccgaagtgctgggatttataggtgtgagccactgcacccaaacctgcccatatcttatgttgggagctggagcgggaggcagtttctgggactctcagatgcaaaaggcccaggccaagggtgaccgacaataagagaggcattgccatgcggtgtctcggtccttccccagttttaagcctgaaatactgttacaagagctggaagcagaaatggaaggagcagacacagccttgaacctcagggagcacttggtctgaaggatgaagacacagcttttgccatgggaacccccagcaaagggaagacaagatgcctcactcccaggatgaggagagactgagagaaaagcattacttccatttggacagatctgtggacatatttggctggggttggggaggggtggggaagtaaaagtccataggaagacaggtccatctgaaaagacctcctggaacaggggtagtagtagtagtagtagtagtagtaataataataatagtaatagctaaaacttctgtgtgctggctatgattgtgtgccaagccaagcactgttttggaaagaaatggcctggatcatgaggaggggcaggggagggggaatatgactacagttgggactacgattggtggaggacacaggcagcaaggacacaaggtctggtgaggagtaaggctcactggagcaggtgatgagggcagggactcactgtcctgtcttgcctggctgagcacagacagccctcaggacttttcttagcccaattccaccttcacttcattggttttctgctcttggctcccttccccagaggacaagtaaaccattctaaacttctggttatttgaaaggagtaggtcccagaaggaggtggccttttcccttgcctacttgccccaccactgagatcctcaggtgatgcctgaagttattcctgcactctgccctccagctatcctgaggggatgacagcctctccccttctatttcaagaattttggaggctcggtgcagtggctcatgcctgtaatcccagcactttgggaggccaagaccacttgaggccaggagttcgagatcagcctggcaaacacggggaaaccctgtctctactaaaaatacaaaaattagccagttacggtggcacatgcctgtagtcccagctacttgggaggctgagacgggagaaccgcttgaatccaggaggcgaaggttgcaatgagctgggatcgtgccactgcactccagcctgggtgacagaaggagactctgtctaaaaaataaaagaatttggggatacctgggacacctcacaccttgccccaagcccaagatcacaaggtggtaaagctatttcatc

SEQ ID NO: 122 - DNA, 8797 bp, Artificial Sequence
  subsequence of TP73
acatgcatgcactgcctcttggcaggcgagggagcttggggaaggctcttaacattacacagcctgtttccccatcagcaaatggccacagaagtaccaggctcactccttgtgaggaataaatggtgcaagacctcatcaggggccccagcacaagccgggggctccattggatagagtttgggcccccttcccctggaaggtcctcatgtgggcaggaccaagcatgttcccagtgccgtgagcaagcacccccaggtgtttcctgaggccctgggatgtggccagcactccagtgccagctgcgattgcccttacaacccagtgccccgtacgcacggtcacccccgtccctactaggcggggcgaggagacagggccccaggtgttcagcccttgctcaagcctctagctggcaggggtgatgcccagcgtcacatcgccaggccttgggatggctccctggtgtccagaggtgtctggagcctgggtggaggctgcacctggatgcccagcctggctgccctgatggccccacctgcctctcacccaggccccgggatgctcaacaaccatggccacgcagtgccagccaacggcgagatgagcagcagccacagcgcccagtccatggtctcggggtcccactgcactccgccacccccctaccacgccgaccccagcctcgtcaggtgcgtgggctgccgagggcctgagcatgtgctgtcaccctgtctgttcacctctgtccttctggccatgtcagctgccctgccccaccctgtgtgctcaccactcgcaaccctggatcagacaggcgggcgggggcagtcaggccaggagcatctgcagatgctggggaaatggtccacttagaggaaaagcacaaaaagccggggtcctccactgacctgtccccagctgagcacgtcccctccctgagggatgccgtggccacctgtgggctggagccacccttcggagacagcggcagtctccgccccagccaggccactctcagagatgggggctcgcgcagccctgtgctcggaagctaatgctgcttcctttctcaaattctctctgcagttttttaacaggattggggtgtccaaactgcatcgagtatttcacctcccaagggttacagagcatttaccacctgcagaacctgaccattgaggtaacgcccgggtggaccccgctctgcagaggcagtagctggaggggcccctgtccggagggcaaagagccttctcttccttgctctcgtggctgggaaacttggaaaccctttcccacgggcaagcagatgcgatgatttgactcttgagagcttaggcagatgcagccaggcacgtggctggtggcgcgggacacaggcccaggcctccggatgctgaactggtcatttgagcctttttggactcccagcagccagtgtgcctttgattttaggggatgggctgtttccaaggcacaaagaacagaagaatgtaaggccccgagggagtcagtccagttcagtgccatttacagagaggtcagcgccatttacacagacaggaaaggggacctagggaaggcaagcacctggcccgaggccacacagctcttggcacccatggcagctgactgcaggcaccatgattaaacagccacggcttgtctttgggttagagactagtggagtacaaagcctgctggtagcctaggacggtgtgaaccacagggcaccgtcagtcaaggaagagcaagccctctggtgggaacctgccccccagccagtgccgcggcccaggtccaaggcccgtcccagaccatccccagcctggggctgcagacagagatgagggtcagtgcgagctagggccagctgctcagcctaactgtccctcgtgcagagagtggccgcgtctgtgtccactaaccgccctccccgcaacctgtccccagtgacccacgctgagccagctccaggtcacgttaacccttgcctcccctgagtgatgtgtgtgcttggtgtggtgcccagagggtgttgggagctcagggatgagctgggggtccactccagggggcagggacatggagaccaaggagggccctgccctgaggctgggaggcagttcctcccccagggccaggtagatgctcagggggctccatgtctaacactcccaggtcagggcccaggccccgcacaggccaggagtgactctggtgggctctccccctcccccgtctcctgcctactctggttgggggtgtaggggccagggtgtggtgtggccagacctccaggcccagggcgaccccccctgctctccctgctccactgccccctgcccctaatgcgccggcctctcgcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccggggaaaggcaaggtccggcccatccccaggcacctcacaggccccaggaaaggcccagccaccgaagccgcctgtggacagcctgagtcacctgcagaaccttctggagctgccctagtgctgggcttgtggggcgggggctggcccactctcagccctgccactgccccggcgtgctccatggcaggcgtgggtggggaccgcagcgtcggctccgacttccaggcttcatcctagagactgtcatctcccaaccaggcgaggtccttccaaaggaaaggatcctctttgctgatggactgccaaaaagtattttgcgacatcttttggttctggatagtagtgagcagccaagtgactgtgtctgaaacaccagtgtattttcagggaatgtccctaactgcgtcttgcccgcgccgggggctggggactctctctgctggacttgggactggcctctgcccccagcacgctgtattctgcaggaccgcctccttcctgcccctaacaacaaccacagtgttgctgaaattggagaaaactggggagggcgcaaccccccccaggcgcggggaagcatgtggtaccgcctcagccagtgcccctcagcctggccacagtcgcctctcctcggggacccctcagcagaaagggacagcctgtccttagaggactggaaattgtcaatatttgataaaatgatacccttttctacatggtgggtcagcttttttttttttttttttaactttctttctcagcattctctttggagttcaacctagcgcccatgagccaggctgaggaagctgagtgagaagccaggtgggcgggacttgttcccaggaaggccgggtggggaggaagcctagagggaaccccaggaagggcaaatccaggcaaatctgcaggaatgctctgccatgggagcagctcctcccttgccacggccaccttctctagcactgcaaggtccacagggcattgctttcctttctaggcggtggcagtcagggaacagactgaggtaggtgtaggggggtctaggccttcgtggagcaccccagggagttagtaggccccggggagacagagtctgcacaggccctttctggggccacctccatccacgaggagcagcctgagccttggtggccgaaccttgaccgtcccggagcacagcttcagggcagggaaccggagcccctggggggcctcacgggtgtgacgaggcccttcattgcaggcaggtgggccaatgggagccctcacccacgcaagccgagacaccacccagagtgcaggctgcctggccccttctggcacggccagctccacaccccctgcctagggtatgtgtggtcctaagggctaggagcttcccctactaacatctcccagaaaaagcagttaagcccctcagggcacagcaaggttagacacagcccccatccccagatcaggactccatcttgctaagtggcatcaccgtcaccagcctccccttatttaaaagcagcgactggtgttgccgcaggtacctggtctacgaagacgcaggcatccctctcccaccgtccacctccccgggggccgctgacagcacagtcgcctgggtgcacgcttgtgggggcagcaggaacggggctgtcggctctcaggggatctggctgcagccagggcgagggcctggcccttccttccagctccttccggctccttccagctgaagggcaggaagctctggccgcttagcttctagggttccatctccctagaaaggtgcccacgcccagggcatcagtcagtagcggcagcagcagcagactcggggctttcccagggtggcgcagccaccccagctgcatgtcacctcagctctccatcttattgccattttgtagatgaggaagctgagaccagaaaggctaagacccatgccccaggcaccacacccatctcttgggggctgggcacctgctacccgaggccacctcctgaagcccccactcttcccccatgttccacttcaggagccgcgggggcccatcctgacacccggggttcctcagcccagcgcagatgtgcttcagttccagagggcttgttgatttgtttcttaggtacgttacctgtccaccctgagtccagtgaggctgtcccaagagcccctgtagtgtgctcctgggaagggctgggggggctgggggggctgggagaggcccaggggcagctgtcactggaaccccagccagatgtccaaggaagccggccagaacacggagcagccagatggccccagctgcacctgtctagggagcccatgcagcctccttgcactggagaagcagctgtgaaagtagacagagttgagacttcgccgtggtcaggagaaaatgcaaattcccaggaacaagaatcctttaagtgatatgtttttataaaactaaacaaatcaacaaataaatcttgaaggcggatggttttcccagcagtgcaggggttggagggaggctgctggcactcctggggccaagggggacaggcagtggtcctgagtctgctcagagaggcaaggcagaaggagctcgccaggcaggtcagctcacatctgtccaagtcgctctggtcagaaacagcgactctcccccattcccccagcgttcccaccaggcctgggctgctgggaagcccttgctgtacccaggagcccgacccgcagtatcctggcacagagccacttgtcactcagaacagtcagtgtctccaacgcacaaacatccactcctctgttaccagttaaagcactttaatgctttaaggtgaaaacgaaatcccatccgtgtttttcgtgtaagatcgtgcttctccgagcagtattaatggacgccctccaatgacataacaactgtttttggtaatgtaatcttgggaaaatgtgttatttttttagctgtgtttcagtggggatttttgtttttgtaacataataaagtgtatgttccaatgaccggttctcatgactcaacctaaatctccaaagggctcagacaaacagggcggctcctcacaggcaggaagcccgtggcctctgtggccggccctggcaccccacttgagtttctctgtgtcgtgagtctgatgggtccctggggtgtccctctgccctgtgcctcggaggacctgcgggccaagccgagtagcctcatgggactgaccgggcctctcaggcctcctgaagtacaggcccatgagccctcatccaagccctgtggggcccaggccttctggactttagctaggccaagcagggtctcagggcgtcacaaacaccgacacagaacgcactcccgtttctcagcaaaacctaagaaaacaaaaggcagcagagagcctcatgttggctcaagccaggctgagaccaacgggcttcatttcagttctcttggttctcaggtttcttggtttcagagttgtgaacagggactagggacttgtcacagcaaaaacaaggctctagaaatctgttcaaccttgaaggaggcctcagtctttttagaaattggcctaaaacaaagatagaaaggaccaaggcaagagggggagatgccgccaggaagctccgccgagttcccccacaggtgaggtgccatgccaagccctggccccatcttcctggctagcgctcacgccgtcacaccagagcccgcccacccctttccctaggctcctacctgtccccacgtccaccaaggagggctgggcggtggtgggcccaccccctcatagccgcaggactctggatcatgaaggtgttgaggcctcaaactagacaggaagtggagggattctggatgttccctcggcaagagggtgccacatagaacgtagaaacggggactctaaaacgccctttcaggaatgaacacagaaatgtgagtgctttcagcacaagggtgagtttgaatatctttgtttaaaaggttagttattttttttaagtatagcgtgagaaatacaggccattattttcaaatatataaaaacatctccagtaagttccacacagaattttggaaagctaagctggactcagagcaggtctgctgctccccaaggcactacccttcgaaggaaggtttccacccgcccccggctgcaaaatggaaaggagaaagcccgtttccaaaaggtctccacgcccctttgggtggaaaacagccactgttcattcccgcgttcacatccaaagaggaagcctagattcaggtgacaagaacagaatatgggcttttcataccacacacatatatttcctatttctcaggaatggtgctgtgtcattactatgcacaaaatcggattcttcaagaaaattgttttttaggccgagcatggtggctcacgcctgtaatcccagcactttgggaggccatggcaggtggatcacgaggtcagtagttcgagaccagcctggccaacatggtgaaatcccatctctactaaaaatacaaaaattagccgagcgtggtggcacacgcgtgtaatcccagctactcagaaggctgaggcagaagaatcgcttgaacctgggagacggaggttgcagtgagccgagatcgcaccattgcactccagcctgggtgacagggcaagactccatctcaaaacaaacaaaaaaaaatgtttttaaatactggtgtaattaccccaaaaaaggcctaaggaggcctgcagcccgcctcatgcatgcgacatttagccttaacagcttcaccagccctcggccctgtttgccacagaaaagctgaaaccgcaggtttctgagtcaatttacaccgcccaatccctgacctgccctcttcctgagagagccaaccagaggcctccaaggccctgccaagggaactctgaccggaagagcatctctgaggcctgcgcacaacacaaaacacctaagcaagcccaacttgtgtccttggtctgaagtcccatcctgaaaagcctggttaaaccagaaatgaaacatacatggtttcccaatgcttccccttctgagtctccagtgaggacaagattgccatgaggttttgtctccctctcccagtaacagtaacaagtgaaacaggcaactggatatcaggcaggatcttggctccagcctcgagttggttcacggagcttctgtcttcaccacggaggcctctgccgattcgcagggcaggtccacagctgaccggtttcaccacgactgctccacggccacggtcacccggaaggccctgaggaactgctctttggtgatttcaggaggttatttccctgcacatttgcacagattcaggacaaaacccaacactgcggagccagacagtcttcctcttgttggggcatgtgaaggggccactggcctctgggtgggacactgatcctagagaggcccacctgcccctcttggacaagccccacccaggtccaggaattctgctcaacctctctggttcccagtcacccccaggactgacagagggtagaaagtgtcctatcttccaggatacatggtatccactagatctcaggagttcagaagtagttattttataaactcagagtttaagaagatttctcaggacgtgtcaggcaacatggtacacgcacagaaccccaaaggagcatctctcagaatctccctcaagtacacgcaacgtaaatgatcactgagatggacaatttcaaatctgaacccggtatctggaatgcaattttccacctgaaattggcatatcatctgcactgactgcagcctggaaggcatctacccattgagcatcaggacatcccaggttcctcctgaaaacgtcacccacctttaagggttctgacaaagtgtctgaagagctctcgataggagtcctggtagcggctagcctgggtctgggagcgaagggacagggacatgccccgcccggtgatgcccaa

SEQ ID NO: 123 - DNA, 8476 bp, Artificial Sequence
  subsequence of MIIP
gtatgcgccaccatgcccggctaattttgtatttttagtagagacggggtttctccatgttggtcaggctggtctggaactcccgacctcaggtgatccgcctgcctggacctcccaaagtgctgggattacagacgtaagccaccgcgcccagccaatttttgtatttttagtagagacggggtttcgccatgttgcccaagctgatcttgaactcctgacctcaggtgatccacccgcctcggcctcccaaagtgctgggattacaggcgtgggccactgcgcccggccccagtttttctaagttatatagtttaggtttcacaactagatctatgatccattctaggttaatttttgcatatgggtttcttttttttttttttaggatgagtgtgtggatatccaattatacctacaacatttgttgaagacaccatctcttcttgttttcccaagtttccacacacattattgataaaataaatttttttggggggggtgctgggtgtcgctgggctgtaactccttacctcaagccatctgcccacctcggcatcccaaagcgctgggattacaggcgtgagccaccgccccgggccgaaggcattatcctttctccactgaattgcactttcatttttgtcaaaaattaattgctcacatatcggaactctattctgttgcattgatccatttgtctacctggacgccaataccacactttctttcttttttaaattaatttatttattgagacagggtctctcagtcagggtggagtgcagtggtgcaatcaaagctcaccacagcctcagcctcttggactcaggtgatcctcccatctcagcctcccgagtagctggactacaggtacgtgcctggctcattttttgtagagacggggttttgccatgttgcccaggctgggtatcttttttttttttttcttttttgagatggagtcttgcgtcttgctctgtcatccaggctggagtgcagtggcgcgatctcggttcactgcaatctctgcctccctagtagctgggttcaagcgattctcctgcctcagcctccctagtagctgggattacaggcgccggccacctcgcccggctaatttttgtatttttaggtttactggcgtgagcaaccgcggccggccccaatactacactttcttgtttactgtgtagtccttatcttctggcgcaaagcgggcactcgctaaaaacctgaatgaaagtataaagcaggccgggcgcagtggctcatgcctgtaatcccagcattttgggaggccggggcgggcggatcacgaggtcaggagttcgagaccagcctggccaacatagtgaaaccctgtctctactaaaaatacaaaaaaaaaaaaaaaaaaaaaaaaaaaaaaaaaaaaagccgggcatggtggcgctcgcctgtagtcccagctactcgggaggctgaggcaggagaatcaatcgcttgaactcgggaggcagagattgtggtgagccgagatcgcgccactgcactccagcctgggcaacagagtgagactccgtctccaaaaaaaaaaaaaaaaagaaaagtatgaaacgatgagtacaatgagtagccagccgcctgccccacctaccccgccgttccggactgccgcctgcgccgagcctaggccgcccagcctgactgaaactgccgctgctgaactcagcgtatccagtcccgcagctgaccaatcggagctcgcccttccggggcccgccccattccaggaatcgacctccgcaccggcctcgaaaattgaccaatcggttgtgtagctccgctcttagtcccgcccccacgctgaaactgacacttcggatcgccgagtaacgctcaccagacgtcccggccctgcccctcacctgaccaatcaagacggccggccgaggcccctggaacggctcaggcggctgcggctgctacggcggcgcatgctaggggattctgccgggtgagtgctgggcggcaggactggggtggggcggaagggctcggcaggaggccccacgtgcattcccgcgaggggctcgggggccgcagtggaaaagaagggtaggctgcatctgcctctgcgggggtcccgcctcggcgtccgcccgctcgccctctgttacttctgcggccccggtgtttctggagcgtcgctttcccggtgtctccgctcagcttttccctttccaaagcgcggccggggccaggggagggcaaggcctgctgtgccctttgaccccgtgcgaaaacggcgaccctctgactataggggtgtggcccagagccacttggccagttaggggtgggggccgggatctttgtcctgcggatcaaacagttggcccctctggactgcatctccgtgccccccggccctgcggggtaatatacgtgtgtgggggaggttccggggtagtcggctactctgtgctccagaccttgcacccgatcctgaggatccttggaggtggggggcaagggctgggaggaggaggcggagttttcccaggctcccccacacttccagggaaaggctttccaagaaccctgaactcatggaccctggagtcaaattttggttcagatctagcctcttgctagctgtgtgacttggggcaagttgcttaacttccctgagctttggtttcttcatatgtaaaaagaaagtgaaggaggaagggagggcaggaaaggaggaagaatcccttttgaagcctgatttgtgttggactcaccagttataaagcaagcttgtccaaaccactgcccaggaggcggaggttgcagtgagccaagaaaaaaaggaagcatgcccttgccccagccattttggtaataatagacgtttattgaatgcttactgtataccactgtaagcactgtattaagtaagcactgtactgagtgctttgttttttgtattttgtgtgtgtgtgtgacagtcttgctctgtcacccaggctggagtgcagtagtgtgatctccactcactacaacctccgcctcccaggttcaagcgattctcctgcctcagcagcctcctgagtagctgggattactggcgcccaccaccacacccggctaattgtttgttgttgttgctgtttttgagacggagtctcaccctgtcgcccaggctggagcgcaatggtgcgatctcggctcaccgcaacctctgcctcccgggttcaggtgattctcctgcctcagcctcccgagtagctgggattacaggcgcgtgccaccacgcccggctaatttttgcatttttagtagcgacgaggtctctcgccacgttggctaggctcatctcaaactcctgacctcaggtgatcctcctgcctcagcctcccaaagtgctgggattacagacgtgagccacttggcctgttttttgttttttttacagtgatgcaaccatggctcactgcagcctgggtctcctgggctaaagctatcctcctgagtagctggggccacagacaagtgccaccatggccagctaattaaatttttttttggccaggcactgtagctcatgcctgtaatcccagcactttgggagaccgaggcaggcggatcacaaggtcatgagatcgagaccatcctggctaacacggtgaaaccccgtctctactaaaaatacaaaaaattagctgggcgtggtggcaggcgcctgtagtcccagctactcaggaggctgaggcaggagaatggcgtgaacccgggaggcggagcttgcagtgagccgagattgcgccactgcactccagcctgggcgacagagcaagactccatctcaaaaaaaaaaaacaaaaaaacaaaacaaaaaaaaatgtttctacaaatgttgtccaggcttaacatgctttctttaattctaacaacaatcgtatgaggtagatactgttgtcgtccagcagatgaggaaactgaggcacagagaagttaaataacttgcctgaggcagtacagttaaagagtgtcgaatgggggtctctggcaccacagggcaccctactgagccccgtgtcctgctgtcttgacttcaggtagaagagctgggcctggaacccagccctgaggacatcctgcggcccaggggcaagtgacacctgctgagagaggcccaggatggtggaggctgaggaactggcacagctgcggctgctcaatctggagctcctgaggcagctgtgggtggggcaggatgctgtgcggcggtcagtggccagggcagcctcggaggtgcgtgcccgccttgggaaccccagaggaaggggctacctgaccttcaggccagcatccaggcttctgggctcatcttgttcaatacacccattttactgatgatgggacattgagggctggggagggaagagactggccttggtcactgctgaggccggagcaggatgaggatgcttgccttggtgcctggcgtaggcccggtgggtaggttctggcagccactccctgggtgacccggcccctctctccagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaaatgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaaggtaacgtgggagagcgggacatctgctgatgggaggagcttccttgggcctcagtttccctatctgacacatgtgcttgtgggtagcctcccttgcagaataagaggctcctgggagatactgtgagttgggtcttgctcagcctaaatgctcgctaggtggtagctgtgagtatctcatacagtgctttgagaagaggccaggatttgttcacagccacacataaagtggggccaaacgtttcccttcttgctcttgtgcccttggctgagccctgggctgcaagtcagggtgtaagttaagacacagtctctctgtccctcgaattgcggcttcctctgggccaggcctcttggcttagtccttgtccctctgtgcttccttcctcagcccagggtgaccttctctgaggagtctgcagttcctaagaggagctggcgcctcaggccatacctgggctatgactggattgcaggtaaggcgtgctgttgccctgcacacactttgcctgggagtgggaggtggaggcctgggatcctccatgctcctgtctgagcctccacccggtgctgttgcttgtttctctgaccgtgccatcctccgtggggagcaccctcttttttttttttttttttttttgagacggagtctcactctgtcccccaggctggagtgcagtagtgtgatgttggctcacagcaacctccacctcccaggttcaagtgattctcatcccttggcctcccgagtagctgggattacaggcgagcaccaccacgcccggctaatttttgtattttaagtagagatggggtttccctgtattggccaggctggtctcaagctactgagctcaagatatccacctaccttggtctcccaaagtgctaggattacgggagtgagccaccgccccccacagccaccttctttgttttcttatttatttatttatttatttatttatttatttatttttgagatagtctctcgctctgtcgcccaggctggagtgaagtggcgcaatctcggctcactgcaagccccgcctcccaggttcataccattctcctgcctcagcctcccgagtagctgggactacaggtgcctgccaccacgcctggctaatttgtttgtatttttgttttttttagtagagacgggatttcaccgtgttagctcgcctcagccttccaaagtgctgggattacaggcatgagccaccatgcccggctttttttttttttttctttgctttttaagagaagacaggccgggcacggtgactcacacctgtaatcccagcactttgggaggatgaggtgggcggatcacgaggtcaggagtttgagaccagcttgaccaacatggtgaaaccccgtatctactaaaaatacaataattagccgggtgtggtggcacgggcttataatcccagctacttgggaggctgaggcaagagaattgcttgaacccgggaggcggaggttgcagtgagccgatattgcaccactgcactccagcctgggtgacagagtgagactccgtctccaaaaaataagcaagcaaaaaaaaccagatgagcagcagtgagaagaagggggaaagagtagaacaaagagtttggtctgtcactgactgtgagtatacaattgcgataactcactaccttcacgccagcctctgagcaccttctgagccgtatctgctctctcaacctcaggcactgagacggtgcatctgttgaatgaatgaatgagtcccagctgccgtatgtttgaacatttgttatgtgccaggcccctgcacaacttcataccaatgccagctcttttcacgttattgtccagctctccccgacagcaacctaacctcagctgccttctggctggtttccccctggcctatgtcccattgctgtcctttgcatgtgtatgtgtgtttgtgtaaatacataacataaaatttaccactttagcattttttttgagatgaagtctcactcttgtcatccaggctagagtgcaatggcgtgatctccgctcactgcaacctccgcctcctgggttcaagcgattcacctgcctcagcgtcccgagtagctgggattacaggtgcctgccaccacacccggctaatttttgtatttttagtagagaaggggtttcaccatgttggccaggctggtctggaactgacctcaggcgatccgcccgcctcagcctcccaaagtgctgggattacaggcgtgagccactgctcccagccacatgtagcgttttttaagtggactgttaagcggcgtcaactatgtctacactgttgagctatcattactaccatccacctccagaactttttcatcttcccaaaaacttctctccccattaaataattactcccattccccctccctccagccctggacagtcaccattctactttctgtccctatgaatttgactcctctagggacctcatataagtgaaatcatatagcctttgttcttttgtactggcttattttatttaacataacggcctcaagatttattcatgttgtactgtgtgtcagaattcccttcctttttttttttgttttttgttttttgttgttgttttttttgtttgttttgagagagagtcgcactgtgtctcccaggctggagtgcagtggtgtgatctcggctcactgcaaccttcacctcccgggttcgagtggttcttgtgcctcagcctcccgagtagcaggtattacaggtgtgcgccaccacacccagctaatttttgcatttttagtagagatggggtttcactgtgttggccaggctggtctcgaactcctgcactcaagtgattctcctgcctcggcctcccaaagtgctgggattacaggtgtgagccgctgtgcctggctggaattcccttcctttttaaggctgagtgacattctgttgtgtgtagatgccacatgttgtttatctgttcattcagggatggacacttgggtgctttccatctcttgactgttgggaataatgctgctggtgacatggttgtgcactgtcacttttgacatagcagccagagggaatttttaaacatgggaatcagatcatgtcactcccctactcaaacctccccatgggcttttcccagcacttactgtaaaatccaaaatccctatctagacttacgaggcatgatctggcctctactggccccatgatgtgacctcccgtctttcttccagcctcgcggcctcctccttttttatttttatttgttttctctgttgcccagagtcaagtccagtggtgttatttctgctcattgcaacctccgcttccttggttcaagcaattcttcctcagcctcccaggtagctgggattacagacgtccactaccatgcttggcttatttttgtatttttagtagagacggggtttcaccatgttggccaggctggtctcgaactcctgacctcaggtgatctgcccgccttggcctcccaaagtgctgggattacacacataatggggtggccacaggcatggcaggaccagaaaggaggccacgaccagg

SEQ ID NO: 124 - DNA, 7991 bp, Artificial Sequence
  subsequence of HOXC4
attgaatcaggaaatttttctgagaaaggcaggtgggtatgggcaatctgacacgatccaaaaggcttactgccttgcacccctccccgcaaaaaggaagtagagaccaagatcttgggctttaattttatttttaattgtaaaaagatttcaaataagccaccatttgaactgggaagggaaactagggatggtcttgggggaaaatgtcattctctctaagaaagggaccccggggatgtgggtcgttttgagggtcctagaagttgaccccaattctcacctctcctgctgcactgcaggattgcccagggtaggtctagtcccctaggttcttcagaccactccaggtcaggtttggttgggtggctgccagtggtcttccactactgttcctgtctagggtggagtaggagctttcaggggtacggcatagaggggatgttgttctcttactaaaaaaaaaaaaaaaaaaaaaaggtaggggtagggaggcaccctttcaatccctgcaattcaccagccatcactgcctcaaggccacaaatctttgcaaagctttgaattccgcctgatccaaccttaatgcccccatttattaaggacaatatttaaaactcggcaagccaggagaggcaaccgaccctcagtgcttgaagctttctttcgcctgggcgtccgccctcagctctttctgcgctcgcctgcagcagcctggcccgtggggagtgggcggtgggtgggggtaggggtggggtgggggtccggggatgctccctctatgagagaattgggtaaacatggcgaccgcggcgcccatttgcacacgctacacaaatgcatcgcattcccggttgtttgcagaaaatttacagctgagtaataaaagtttacgatcgactcacaagttggattggccacaagaagtcatgtggattccatccatgaacgtgaactttttattgtggtttgtccgttccgagcgctccgcagaacagtcctccctgtaagagcctaaccattgccagggaaacctgccctgggcgctcccttcattagcagtattttttttaaattaatctgattaataattatttttcccccatttaattttttttcctcccaggtggagttgccgaagctgggggcagctggggagggtggggatgggaggggagagacagaagttgagggcatctctctcttccttcccgaccctctggcccccaaggggcaggaggaatgcaggagcaggagttgagcttgggagctgcagatgcctccgcccctcctctctcccaggctcttcctcctgcccccttcttgcaactctccttaattttgtttggcttttggatgattataattatttttatttttgaatttatataaagtatatgtgtgtgtgtgtggagctgagacaggctcggcagcggcacagaatgagggaagacgagaaagagagtgggagagagagaggcagagagggagagagggagagtgacagcagcgctcggtaagtgtttccttattggttcaaatatgtaactaatggtccgtggcggtttcaagaataggacagcagcggcttctcttggttagggagaagagggggctggcgagagggtgggcgcgtagttaattatggggaggaaaattggcggggggagagaaacggggttaaactggaataccggtaactggtagtggggcgattgtgttgtcctgatgtcactgtgtgccaagttgggggtggggaggcaagggaacagcacctgtggggctcaagccggcggtctagggcgccagctctctgctgcccactgtgtcagtacctgcttattatagatcttcggtctcttggctaatggaactgggtcggagcggaactggaagctcaagctccaggcaggagagagaacttagaggctgtgcccattgccccctaagcaaagttagccccccaacccccaaacaaactttccagcttcccctccctgtctggcagccccctccctcccagagagcgcgactgctagagctcacacatgcgcagtgtgggcccagggccgggccgccgagcaggaagccggcgcagctaggcggccggcggggcctgttaattggcaattaggggggaggctggtggctggtgcgcgtcagccgagaggagagcgtctgcccaccccctgctcccgcccccactcgggcggatggaagggtgggaggtgccctgcgttgggtggagggtggaggttgtagggtgggggtgggggatgctgtactcaaaagccatcttgtgctcagagaaaagaggcctaccggctttcccttccggggtccggcgcccctcacccccagccgcggccatcccagccgggatgcccactggaccgggatgcccgctcgccacgcatggctgctctgggctaggacctgcctcgcctcggggggaggtggaaatggggaaggccctaggctgggcggggacaggcccagtcctcctggtagagtctcctaggtgctgtggaggcagtgaggattccgggggctgctggcttatggggtagaggcgtcagcctggggcggcgggagggtcctgggcactcgggatctctctaataattggtgctaatggcctgttgtatagtcttaacccgacgcatatggtttcataacacagtggcttaatttcttccaaatgtacgtggccctgaatgtgttgcagtttgatatatgaccccgccctgctccccggcctgggtccgaggctccgaggcggccccataagtgtgaatttctgtttataaacggcgacgcacacgcaaacgcctacacacccaggcagacacacccggaggcgtgggggaacatattccagacgaggtccgagagaacctgggggccagacttaccttaatctaagccaagacaccccctctacccccaccctctcccagaggcgcgactgggattctgcgccaagtcccagtgggggaggggatcgctccacgatttcctgtttaaaccctgacccgtcgcctgtgggggggcggggatgggggaagaaacctgtttttatcaaatttttaaaaatacagtttgcattcagatcaaaccgtttgggccagagctgacggtgaattttatctcccctccccccacaacccccttcccttccttttctgcccccgctaaattcccctttcgcagccatcataaaggcctctgaggtattctcccgcgggaagaaatggcattttctctccccctcccccacccccccacccccagtaggacttgtgtgtcggcagaggcgtctgacgaggggctaatttcgctttccttgtttacgatcgagaaaagcgcttggctctccccaaatgggcccagcccgccgcctgctcaggctgccgcgcgctctcccgccgctcaggcacggcccagtggctgatgccccgcggatccagcctgcggcaggccggaacccggagtctgtgggacctgaaaagggcaaagggagtgcggtgggacacaagaggggcccgagaggcccgaaaaagggagcctttctcctggagtaatgtgggaatcgccgaccggtgaggcctctatttctctctttcgcctgcattttgtccgcccccgcccctgtggatggccgctgatgggggggaacacaagtgtcctttgtccgtccccgaaagaaaaccttttccagagaaggccattcaggccagtggagattcacctagaatattagaagggccccctcccacccgaggggcgggcctgggcgaggctgaggtatttctcttaatcctagaacgacctcactgagtgtacccacagcgagatttggaggtgctagagttcgaggttggccgcttaacagcaccatcctgaggtgtctttatataatattttaattgattttctgtcccttcctggctacagccctgcagctcccccacccccatcctatgcccttcacccctagacccatccttacaagacagaggcaagcagagaaggcatagcagcagcgaccggcgctctgttttcattttccactctggccaggggataaactggaccccagtggactccaacaccaactctctgcttcccagttactttcgattttctttcccttctccttctttctctccctctctggcattcaaacagaagcacaagaggacccaccagtggcaactctaagcctccttgcaggccacattccctgctgacgtggtccttcctgcaggagagcccttctgtgattatctaataatagccagggtggctgagaatagaatctggctcaaggactctgcacacccggggcagagacactgaatctgtgggtcagggcctggcaacattccttggagcctccccctctgagcccactctttgggcacccttaggcttgcctgccccccttataaggggctttgtcagccctgccatccgctgtatttcatgtgctggttcctagctaaggagaattgggcagagcaaaccctttctcaccctggtctgacagttgtgatcccgacagaatgcgaccttgtttggagaaggcgaccttgttgggttggctgtgttctgaaaccaggcttgggagtgttatatatttattatatacaggcttaaatatacagactcaggcttatgcatgaatatatacagactcatatttccttggtggttaattgtatattaccatgcctatattcgctcaaacaccattttatgttcatgcaaaattttgttagtcctcactagatgagagtgggctcatggaggcaccgggtgcactgagcaggccctctgtgcatgctctgctggaaatatgtgctcactggaatcggagtatgcgggttccagtaaccaatggatgccataaaggctttctcagaccttattgcttttgttgcccaagcagctggactggggaaatactaaatcccttctgctttctctccacacctcctggcaaactctcccaacttagaaattattttacaagggagaccagttgtcttgaaattgagttctgcgggattcttttcacgggcatggtggggctgcaagcagttcggagtagtcagggccctggcgctggaagcctcactggagcgatgaagggatcagagtggttaataaggtggcctctggacagtgatctggggcagctgctggccagggtgcttggagaagcttgtggaaaaggcaagccaaaaggggtagggatgcagtgaatatctgtttctccccctgcttgccccagccggccgctcaccccgccccagcccgatttgcctgcatccgtggcggcagattgggagttctctaaggtcccccaaatacccagatctggttactggatgaactgcgttttaatcctcgtcgctatctgagccaggacaagacctggtcagggagacggctggtccgggcagcacgggagaggacgctggacccgctgcctgtggcctggagcactgagtatgggctaccaaggctgagaattgatgggcctcctttcccagcctttttcccttccagagacacaaaaagacagagcagaggggaaatatcaatgtcagtggaaaaccccataaaagaaagcagggaccagagcggggctggaggggttttccgctactctctctaggtgatcgggttcctcccaagactcttgtccagacaggctgcaccaagaggtgtgcagctttcttccaccttcctcccgcccttcaaggagggcagccttaggctttctgggccttggtcaccccaggcactggggtgacattactccaggattgtgcccctcgcccagcccacggcctctgccacggcgccgcagccgctcctgtttattgtgtctgtgagttgtttacttgttttgtctgctgccaccctgcgtcagaaacccgccaaaccagcgaactcgcccaggagaacaaagtgctgtgctatccccggactccctcccctttccagcaaccccctcctgccctcacaagctcctgcattctccctttcggaggggaggaagcagaaatagtcttaaaacagggcagctgtttatagggtcaggggaattatggggacaccataaacctctgggtgtccctgtggtggacagacttctgagcagcccttcccttgcctaggcttttcttccttcccctggtgaccacttggttaagggggccacactcattgagaaagttgagcccacccccatcaacccagtcccccactaccccactcctttctgcaggagtttccctgagacacaggttgttgggttttaaatcagggtaatgttggtggattcataaagctagatatagcatgtcattcttctcccacactccctctcctcagccccccaaaattgcagtgaatttaagcaaatcctacaaaattaaattgcccctttgctaaagatcctatatgtctccaatcctgccaggtgtagagtcagatagttggcttttggcctttaattttgctgctagctattaaatttaaaagagtatcattgaagtaagtaatattctcatttctgaaattaattttcccctcccagtctctctctctctctttttgccagcacagattaaatggttctttcagatctgcaaacaataaaggataaaattatttatagggaaatgaggatgctgttttattgttggcatattgttccagaacataacaacattttattttcaagcaggagaggttagaccagggggagaaagagggggaaaggaaaaaacatgaaaggagggcaggaacccacacacaaaactcctgccatgtataactggtaggacatttacaatttctatatgacaactcactgtgatgtcataataccgagttcttgaattttataattctcatgaacctctcaaatgggtccaagcagtgcttcatgtggaggggtatagaagaagccaggaggctggggatcctttgacccagtgcagagcagcacaccccctattgcggcttcttcctcatgtgacatcctcttgaggccctattctgggggctgaaaatattctggatattctgcattttaatttttctcctgggccaagcagggccacttgctttgcccccctcaagtgtccctaggcatctaagctctctccccattcccagatcacagagagctgtgtccccaggctctccccaagccccctcctcagctaatactcagctgcttggaatagaggtgggaggcttgggcaccagcccccaaaaagccagaaatgattttttccagatggagctggaagggggctcagggctgagcgtgatggggtagagatgtttgggacctattttacgttttaaccttaagtcagagaagtgaattgagaagccaaacaaatgccaatgtgagagtttgtatctgctgatggtagcagcaataagtgaagtgctggtaaggaaaggtcaggcagaaatctggaaagagagagggaggagaggaaatgtgacatgagttagcagagcccttccatatacctttcttctccgtttaattgtaataaagcaaaagcagctcgggtagcctctcaacctcttagctcaaaagaggaatcaccagcgcaggccttggaggtgtttgggggcagttctcagcagcttaacatgcccaatctgccctcttcctcctttccccctccctgctgatccccagctcccttcaactggtcagcccgggccctgtccccaaaatattagcacccccaaatgatatccatttctgtgttaggtgtgtcaaatttcacttctctccatgcctaaactttgaaaggaggagtgtacctttttggaatgcggtgtgtgtagcgggggcaggtatgtgctcagacagcttcccccatcatatctatgggaaactggaagccccttaggtgagccatgctttggtgttcccaaacagcatt

SEQ ID NO: 125 - DNA, 8141 bp, Artificial Sequence
  subsequence of HOXA7
ggagctgagcagggtacaggacaacacaggagatgaagggcattgcggagggcattggacctccccacccactacagttaactcaagacaacataccatgctacaaagtcaccccattaacacatcctttccaagtcaagacactgccttacaaatgaactccaagactatagaaatgataaaaaaaaatcttgttcaaatatacagtatctgctattataggaaacatcagggcgtacatatttaacacagctgaacagtaagatacaggagccagaggaaaggacagcgaagctggaagcatctccacagtcctgctaagcagaagctaacccacagatctgcagccagctcaggaacattcccctccagaagtgggggttgatgggcctgagctgtgggtgccaagccagagaaggagggattgattctagggtgcaagcacttaggatgctttttggaataaatatattatttttcgatttaaatagatgccaataccctgatcctggacctcagcacattctcagggcagcctcagggaccccaaaagctgcgggctgtaagcagcaggggacttgcctgggagcagtcggcactaggtagcaggcaagccagccagcacaaaataggtagttttaggggagtaggtagtagtgagattcactttcttgcgggtctgggagggtggtgctgggtgtctgccagtgttgggatacatagggacttcctgggaatggaggccctctggggctggatacataggtagtttgggggtgcctcgagcagaggcctgtgctaggtagtattttggacgcgccagagcagggccggctggcctggggttgggggtgtcttttggggtcctcggaggcagagggaatccaaggcgacccagtctctgcggccgctcagtccacaaaagttgggagctggagtaggtgatgggggtgggtagagtgcaggttggggactgggttgcttttttgtttttgtttttgttttttacattttcttttatttttcccatttttgtaagtaaaaccagtgagtctcttaaagacgcttttccgactgtccggtgcagagagggccccggatcggcccctcattcctcctcgtcttcctcttcttcatcatcgtcctcctcgtcggccttgtccgcggcagcagtggcggcggcagagggcacggcgccctcgggagctgcggcggcagtcggaccttcgtccttatgctctttcttccacttcatgcggcggttctggaaccagatcttaatctggcgctcggtgaggcagagcgcgtgggcgatttcaatgcggcggcgccgcgtcaggtagcggttgaagtggaactccttctccagctccagcgtctggtagcgcgtgtaggtctggcggccccgcttcctgtcaggtcctgagaacagacatgcagacacatgaacacaaggacagacaagtagacagggcactcgttaggctgctgtcccagagcccgcaccttcctcctggcctagtccccagcgagcatccccctctgccccaggccccgaactgagctaggggaggagggggagtgttagggaaagaccccaactgcagtgccagacgcgcaggcagctctgtaatgagcaaaggcacagaatctcaactttacaaccgacctttccagccggctaagcttccacaatgtcctgcttcctctgacaaaggaaaactgtaaatatagagtgtgagcaagtgggaaacgctgcacttttgccattcaaagatgagcccggccattcccctgccttgctaggcaagtgggcgactcttcccagcagcctgagccctcatccccaggaccttcctagggcaccccgaccctctgtcctcattccctcgcccccatcttgaaatggaccctggcacagggtcgggtgagaggccctggagggcttggctctcctagcttttgagaaagaaatgtcaggcagcaaggaaaatgaggagagagagaagaagaaagggagggagggtgacagaggagggagaaagagagacagaatagcgaacaaacttaatgttaaaattccaagacaaatggagttaaataaatttacgaggatcgaacccattaattgggccataaaaagttttatgagcctcatttacatacaatgctatgggctccacgcaatggcgcctccgctccaattaaaaccagaaaggctgcgccgggagtcacggggctaccggctcgcaacagcctggctccgctcttccggccccgcgccccgcgctccgcgctccccagcgctgcgctccccgctcccggtcccgctccgccagcctggcccgcctagcgactgcgcctacctgaagaccgcatccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccgactcctgctagtgagccctggaccaagcttgggatcctccccatccctctcctgtccgcctgcccagaccctggaagggtctctgtcccccgcaacagcctgccccgcggtggccttgtgggcaggactcagctatgagcagatcgactctgcccaagtcttctctcacccaggtccagtgggcgacaggccggacttagactcggatccagacggggaaggcgcagcatctcttgcagctgcagagagattgccaccgcaaactggagccatgtggttcgaataaagtcaacgtctcccagcttcctttccttaatcggaggcacactgtttatccgccctaaaggaagcagtgaaatatttatctattaatgagactcatttgccaacagatttattaacgtggggttcccctccctcctcccggacgctgtagtgctgcaggctctgtgccttcgctcctgggcacctggctggctccagcagtccgataaattgctaaagattcctttgtcctttccacaacttctggttcccctctggcgcatggggagccagggctgtttcccccagcttggaaaaatctcgggcctgcacccttccaggcactcccaatactggaaggtttctggggtaggccggggtgcctgggaacaatacatgctttagagcggatttggagagggggctctggcgtctagggactgcaacccactgtggacttccttttcttttgaagacaccgaaaacaaataggggaaaccaccccctaaaggccacccagctgtggaggcttcacctatccaagtaccagctcacatggagctgagcgcacagtggtctcacttcctcccatccaggcctccaattcctaccttccaggcctccgacccctgcaccatgttgcaccagctgaagcccctgccgcccaccgctcggtggaggatggtggggagaacccttcccctaaacgcctcataaactgcccggctgcgctggaagcccggccaccttttactgctcagctcgattacacaccataaacccgacctcacaatggaattgccccggaaattctcctgtaaattgtagagtttagttccttgtgtactgagcacttcccatcactttcagagggtccaggccgtccccgtttaccgcaccctctcagggggccccaggaatacaaaaggtggagggagggttcagatcgggacgccacggagccccagttcctgcgcagtgaaccaccggggcttgggtaggaaggcgggggctggtggggcaggtgggcgctgggctgtctcctcccgggccacggaagcctgggggtaggggtgtggagtgagggacaacatccggggacgcctttatggcggatcagatgataccttgtctccgatggagaatagagggagtaagacaaaaagaaaagacctaaataatcccagtcgcatcgccttctggaaggaaataaaaacccaagaaagttaaaggaagatgaagcaaacaagaaggctaggagatcaaagaggcagaattagaccgttttagaccaataaatttttcctggggtgacacacagcaagacgcaaagagaaaagaccaaggcccccgccgccgccgtctgtctagactcaagcgactgaaggggccaacagagctggtgtttaaagtagaacctgcccagtccaacagcccgagcagggagcgatttcggggatcgcgggaaggaacgcacttcgccaagggagggccgggtgccctcgccaccggctcattcctgctccggttttgcccgatgcgcgtccaggaggttctggcaggacgcactgcccctctgccccggccaaggaggatgcggatactgcccgcaaggcttcggcctttatggacccaagtcagccaactgggccgagtcctgcggacaccgaaacctccctttcgtttccaggcttccttctcccctcttgccctctgtggtctgatttaaaacgaaaaggtcggataaaatcaggctttcaataaggcttctttaactgtgtgttctctattcattggttctctacttatttgactgaaaagacacaaatgcactaggttatgtgagataattttcacagaaatactcattgaccctcagcctgaagcaggcacatgtaggcgggtttctaatcagtagagctatgtttagatagacattttccactgccgtccctgagcttccttcctacctactggcagagcgtgttcactctgcttcttgttaccaaataccaatatttaaattcaattatgaaggtaaagccagctctaggcagggaacagcgccttccagagatttgggtggcaggcaaattgcttcctaaaggtttccacccttgcctctctcaccagctgcatctgttccccatccagtgagtgagtgatccctagcctgggtgcaaagacaaactcgtacctgtcgtgtacaaatgagattgggttggtgcggggtcttgcctgggtgaaggtccgcagcggggaagccactcccaggcttcccagaggaaaagcagactcccagccaccagcctgccaagggcaagggaggagcctttggacagtccacccaggcctgggagaccctgggcggcctgcacctacctgaggcctccgtgatcagtggagagacgcagtcgccaccgatctgtgacttcatttatttgtagttacacaaatcgagctttctctctgcatccacggatctgcgacttcactatttatttgcagttaacataaattgagctctctatctgcggcttgaatacaaagccacggttgactcccagggaagctataaaacccttcttacaatcagacgttgtgaaattatatacggaaatgtaatgaaggtgtgtgtctgtaatatctgtatctatctatctatctatccatatatatatgtatatgtatatatgagatgagagacagagagggagagagagaaaggagagatattgtgtgtgtgtgaaaggaaagagagaacaaacacccgggagagacatcaaccaaaatccagtccccagttttacagcgtgaaagcactgggatgcgggtcgtaaacattttgtgggcttggcggagactattacgacccaaataaatgcactgtgtagcgtgttcacagggctccggggcctttcgaaaggttctctgtttgcttttgcgttttgcctctggaaccattcgacatccgtggctgatgcgcgcacccgagaggaggccgaagcgtgttccccgcctagggtctgggagaggctgggcctggattggggtcccctcccttctggcctcctgatgggtgaacgccagagcagcctcggttcctgtacagcggagggcatgccgcggccagagacagcccgggcggcttccacactgtgtgcgacacttttggtgctacggtgtgattgttatattaaacatcacattaaaaaaataaccaaagcagtccctggtttgtcccccaggattctcctcccagccaactcggcccggcccctatacctacagtggtcagacaccgagattgcctttacgcccaattttcaccgcctaattttattgtctcgattcaagccagccgagtcccggggtcctggctctgtcttgggttctggctcgccgggccctgacccaggtcgcaggtttcgggggcctcctgggcggccgcggaccccgcggtcacggtgtgagccctcggcacgcaccgtgcacaccctctgggcggtcatcaagttctggggctgcaggcgttcctcctgtgtccgccagtcagccggggctctctggcgcctggctgtgtcaggcctgtcccagctgaggacggtgaccctggagccgcgcccgcgcctggagctgcagccccgcggccgcccggacagctgcagcccggcagcactgggcccccgggatggggtcagggaggcgcgagcggtgagggtcgggcaagcccccgccgattccttgtttcccagcgaggcttctagccaggcttgagcagcccagaaaatatagggcggctgttcactaaaatctggggctcccattccagaaaggctcgtgtcaagtcgacatcttaggaacttcacaagggtcgcggaggcgggagatggcggcgcggaagcctcttgcatggagccacactgccatctgctggccgccgtttggtactgcagcctcagctacctccgccggcctgagctttgggagccgccggctagcccaccgcaccccactcccaaacgaggggcgtgggcctaggtcccggggagtctgcgtggagcccggaatctactgcagaggaggcaatgccaataaaagaggtgtttccgcagccgctttatcggcggcagacagaacaaatgaacaaatgaaagggctttggtggaatatcctaattggggctggacagttgtaaactcattaagggacgaattccagacagttcgcagacccggcctttatggcacactcttactgcctattctttgaagttccttctgtctgcaacaacagagggcgtcccaacctggcggcggggtgggggtgggggttaggggacgcctcatccacccgcgccctggttccccttttaagcttagaacacatccaactttgcagaagtcgtggagtgggggaagggggtcgggcgagggaagcaagggggtggaagcgaggccgaaccctccggccacatctggaaagcgctcaggcctccgaagttgtggccgttcccccccccccgcccctcgtagccctttacaccccataaacggtaacagccctcattttcttttatggcgcttaggggctcgtcaattgcctcagccctgcctctgttaggccttgtgctcgagaggtgcttgcagtgcaggacccagattctcttgggccaggctggcaggactccgcgggggtggcgggcggtgcgaggatgcagggctttgtt

SEQ ID NO: 126 - DNA, 7667 bp, Artificial Sequence
  subsequence of BST1
aagtagttgttacattggattatttagggaatgatgacaaggaaaaaaaagtccgtacatgttccgtacagatgcaagcattctttttttcctcaaatgtttctcatccgtggttagctgaatttggatgatgaatccgcaaatctggagggccgaacatgcttaacttccctgagtttatccattgctaaagcgagagaaattacaaacttagctaggaagggtgttgcggaaagtaattaatattatacttgcaaagtgcttggcacgttgttgtggttattatttcctgaaaacaaacaaacaccacttattggttggtgtgaaagagggcatctgtcaaagttgtcgtcctttggagccagaaaagtatttcttctgatgatgactgacctctcctcacctctccttcaaccttgcggcttctttggggctccctccccgtggacagcggtcaagattagcctgggccacagtgcactggtcctgtcctgggtgatgttccaagctcactcttttttaattttatttatttattaattttttttttagacagagtctcactctgtcgcccagggctggagtgcagtggtgcaatcactgctcgctgcagcctcgatcccctggggtcaagcctcctcacatctcacctcgcaggtagctgagactacagatgcgcaccaccaggaccagctaagttttgtatttttttgtagagaggaggtttctcacctgtatgttactcaggtttgtctcgaactcctatactcaagcgatccgcccgtctaggcctctcaaagtgctgggattacaggcatgagccgccgcgccccgccccacgctcagtcttgaaattgtctggaacgggaaacggcaaacagcgagatatccgagcgagagtcccgccctgcatcagtttgcggaaccgccttggtagaaggagagaaggggagtggaggaagcacgggactggagggaccaaagttccccgatggcggcccaggggtgcgcggcatcgcggctgctccagctgctgctgcagcttctgcttctactgttgctgctggcggcgggcggggcgcgcgcgcggtggcgcggggagggcaccagcgcacacttgcgggacatcttcctgggccgctgcgccgagtaccgcgcactgctgagtcccgagcagcggtgaggcagtcggccgggtggaaggggagccggaaagaggcaacggtggggagggcctggggaggggaaaactggcgctaaagttcggggtgagggggcaatgagagaggccttgaggggagaggtgagtgtggagacagcgatggtcctgaacgatgggggcgagtgaggagggggctgtgaagtgtgtctgcaggtgaggggtgcgctgtgcggggtctagagatgagtgtgtgtgtgtgcgcgcacacacacactgtagaggtgaggggtgtgtgcgtgtgttctagaggtgaggggggtgtgtgtgtgcgcgcgctctagaggtgggaggtgtgtgtgtgctctagaggtggatgtgtgtgtgtgtgctctagaggtgggggtgtgtgtgtgtgtgtgtgctctagaggtgaggggtgtgtgtgtgtgttctaaaggtgagatgtgtgtgtgtgttctagaggtaaggggtgtgtgtgttccagaggtgaggtgtctgtatgtgtgttctagaggtgaggggtgtgtgcatgtgtgttctagaggtgaagggggtgtgtgtgcgtgcgctctagaggtgaggggtgtgtgtattctagaggtgaggggtgtgtgtgttctagaagtgaggggtgtgtgtgttctggaggtgagatgtgtgtgtgtgtgttctagaggtgaggggtgcgtgcatgtgttctagaggtgtgtgtgtatctgtgttctagaggtgaggggtgtgtgtgtgtagtctagaggtgacaggtgtgtgtgtgtggtctataggtgagggatgtgtgtgtggtctagaggtgagggatgtgtgtgtggtctagaggtgaggggtgtgtgtgttctagaggtgagggggtgtatgtgtattctagagctgaggagagtgtgtgttctagagatgaggtgtgtatgtgtgttctagaggtgaggggtgtgtgtgtgttctggagttgaggggtgtgtgttctagaggtgagatgtgtgtgttctagagatgatgggtgtgtgtgttctagaagtgagaggtgtgtgtgttctagaggtaagggatgtgtgtgtgtgtgtgttctagaggtgagttgtgtgtgtgttctagaggtgagatgtgtgtgtgtttgttctagaggtgaggggtgtgtgtgtgtagtctagaggtgacaggtgtgtgtgtgtggtctataggtgagggatgtgtgtgtggtctagaggtgagggatgtgtgtgtggtctagaggtgagaggtgtgtgtgagtgtgaaggagggtggagaggtatccctctagaagtgagggtggggtagaggtgaactgggtaacagcccattgccacccagcagatgtttagtctgaatactcagaacccagaatgtgatctcaggttggaattccttgagtccagtccttccagaattcaaatttcagatcccctaaatctggaggatttctgaagtttgggtctttcttgccagtgctgcatttcttactgatttttgttccttctgtggccccccttttcttatgtttgtgatgtctttcttgtgctgatcatttttattgtcgcacatttcaggcaaataagaaacagcagagaaccacctgtgttacacaccacccagtatgagaaacaaagttacaaatgctatgggaaatattttttgaaaactagcatggacctcaagaggggcttgcactggagggtgcacctgctttgccagaggtggaagccttttgcagcattgggcctccctcctaacactacacatcctgctggctggcctggcctcacctggtccccgagctccacactggaactggactatgagactggccccaccgttaggtctaaatgaagaggcacctgctgtatagttgactgaatttgtcacctcctgctcggtgccatttctgcttgtctccgtgagcctggggcaaggctgtattccttagctctgagaaatctgtgcagtgccttcagagagacaattccagtgtcagatttctgaatctgtcacctaagccatgacaattcgttgtcttttttgtaagcctacttctgcagttactatatgaaaaatctcttgtaaagctcttagacaaatgggcatacttcacaacacattatgatgtgcatgtgtgtgttcttcccaacttgtacttttgcacaggaacaagaactgcacagccatctgggaagcctttaaagtggcgctggacaaggatccctgctccgtgctgccctcagactatgacctttttattaacttgtccaggcactctattcccagagataaggtaacaccacaaccatcttgggtaaaactgtgttctctgtctctacagaaatggatggtgcatgggccaggttgacattagctgtcccctgcatcctgcaatgtcacagaaacatcaggcagcacacataatgtgtgtgctgagctctctgtgggaagctagctttgtttctgtattagtccgttgtcacattgctataaagaaatgcctgagactgggtaatttacaaagaaaagaggtttaactggttcacagttccacaggctgtacaggaagaatggcaacatctacttctgggaggcctctgggagcttttacttcagcagaaggcaaagtgggagcaggtgtcttacatggcacgagcaggaccaagggcagagggaggtgccacaggcttgtaaacaactggatcttctgagaactccctcaggagaacagcagcaaagggggaaatccacccccataatccaatcacctcacaccaggccccacctccaacataggggattacaattttacaagagacacaaatccaaatcatatcagtttccaatagtgattgtagatctcccgctggttgtgggcagaagaaaatttcctaagttagcaagttagtgtgagaatcaacatgaggacatcaggacatggccatacctgtcacactgggcctccacatctggccagttctaaataaccttcatcctaactggcccaatggcctgaaccttgagtaattcagaaaactctcaatctcagtatcctcccgggagaccagtctctccatttaacgggtcttaagattatatttatttagtaccagtttatttaagaaaaccacatatctatattctgtatacactctcaggtctaataaaatgaaaataatgagaaaatttccataaaacccttgcttaaaacaatacctcaagtttaaggaatgctagacaccatttttgtttctgtgacataaacattgttaacctgattttttccattgtgtgttatttgtaaaggcaagtaaatggaactgtgaggttcaaagatagaatagactgaaaacatgatttttgagtatatgctgtcttttgaaccttgctgccctcctgtcactaaaagtggggaagctgactttttaaccttctgtttcatagattcggtgctagttgatgaactcagtaaatacttccggggctatttgtttgccagtcattggtgctaagggttaaggatgtaacagtgaaaaacagccctcacccttggggaccttctagtctaaatataaactgattggactactgagggtcagatgaaatagtgactgggggatttgtgttattgttatataaaaacactatggtggaaactataaagtataatataaaagcaatgtgtttttgaggcacttgacactttaggaagtgctttttgtgactgcttattatcctggccacagctctggaagaagagaaagggctcatgctctgcattttccagaagtagaaactgagaaacagggagattgcatgccttgctcaagattacatatctaatacatgacagagccaggattccagcccagttcccttgatcatttttaatgtaggggatcttaccacatgcacgtatatttagtactagtgagaaaatattccattaacatttgcatgataccctgggaataaagggaggagagatgctagcagtctttgaatgaataaataaagacaacgaaaaagaacgttcagttgttgtgcagcaggtcagagttgaatgagaactggattgcccaacttgccttgatgatattgtgcctgaggagtcggttttgattctgttttgtattttgatgttttgtttgtctttccttagtccctgttctgggaaaatagccacctccttgttaacagctttgcagacaacacccgtcgttttatgcccctgagcgatgttctgtatggcagggttgcagatttcttgagctggtgtcgacagaaaaatgactctggtaagactcctgcacaaatcacaggagacttaagaactcctatttactatgcattaccatttgtgctaaatgctttcatatgcatttatttcacttgatcctctccaagtcctctgagataagtggcaagaatagttaataataacaatactagagaatacttaactttgcaggtgccagtctaagcactctctctctctctctctctctatatatatatatatatatacacatatatatacacatatatatacatatctatacatatctatatctatatatacacacatactcatttgatgcccacaacaacctcatgggatcctatcataatccccattttgataaatgaggaaactgaggcacagcaagattaagaaatctgctcagggggcatagctagtaagcagcagacagtctgactccagagcccattttctcaactttgcacaactctgccttactatcctcatttgtccagagggggaagctgaggcttagggaggttaaatgactggaagtggtaaggctgggtctcagtcctgtcctgatggattctgaagcctgtgctctgtaacaccactccaggccgccggcagatatttcgagatccaagtcacaaggaaagttagagtcacaagagctggcctgaatgttctgcaaacactcctcattcatccaactgtggtgctaggagctggagacacagcagtccccatcttcagggaggttatagcctcatgggggatatcagctcgagtgatatgggctataatgcaggaaaaatggtgttcgaggagtagcagagtgagacaccagcctccaccagatcatcagggaagcttcagaagggaggctagcaaggaaaggaggttagggagtgtttccattatgaagactggagatccttcctgggtatcagagttacttcaaaagttaacctaatatcaggccgggcatggtagctcacgcttgtaatcctagcactttgggaggccgaggtgggtggatcatttgaggtcacgagttcgagattagcctggccaacatgaagaaatccaaactctactaaaaatacaaaaaaattagccaggtatggtggtgcatgcctgtaatcacagctactcgggaggctgaggcaggagaatccctcgaaccctggagaaggaggttgcactgaggcgagatcataccactgcactccagcctggatgacagagtgagactcggcacacacaaaaaaaagagttaacctaatatcagttctgtttatttctcctccattcatttgtccatcaaactaaaatttatagtgtctactatgtgccagaccctcatcttctagacagtaggaaaacagcagagaataaaacatgaaattaataccccatgcttacattataggagggcagataagacaataaacaggcaagtgaaatatacagactgccttaagattataagggctatggagaaaattaaaacaggtaataaggacaaaaaagattgagggtggagggccagggaaaggaaagtgttgcatttttacctagatgccgagggaaaacctcactgataagctgatatttgaacaaggacctgaaggagatgagaaagagcattttctgatgggagaacagcaattgcaaaggccctgaggtgggagcgtgtgtgacatatttaacagcaaagaggacagtgggcaaagaggaaagtgtgagctatgaggctagaggaataaaggagggccaggtggcacaggatgttgtgaggcctctgcttttcactgtgagtgagatgggcagcattgcaggttccacgtcaaatggacacagccattctttcaatggagaacttccaggaagcttttgtttgatgatgctgcagagggtggggagaaggaggggaggaatgtgatgaaaaacacataaagaaagggagagccttccagaaaaccaccagttatttcttcttcacctcgctgcccttttctatctatgccctcctatcagggctctttgtttcagaaagaagtgaagtccagcagttcaggtcagctaaataaccatatgtgtatgtcatggaatcctgtggggccagcagcaatggtgatggggttttttatttattgacccagaaaggggcatgtttttaagcaaaaaataaatttacat

SEQ ID NO: 127 - DNA, 7594 bp, Artificial Sequence
  subsequence of C1orf114
ataaatcatgattttactatttatttatattatttaaattataattttataatggtcactttagtttattaaatgctagcgtaataactgttctctcactaacctgccctatgccttaggatacaacatttaagcaatctacgcctcatttttcttatctttaagatgaggataatagtaatttgcccaatgtagatcaaagaaattttgtgatgtttagatgagataataatgtgaaaaccttaaagaattgcaaagcactttgtaatataattattatgaatgctgttggcaatatatacctctattatgctggcatcacttttttcattttcattaattaaccactccaggtccttttcaaagtcatcttcgtattcttcacttttctttgaatcagtatctttattttcattcattttctgtttgtgaaggaaatatgctgtaagattttaaaagataaggtatatgttatgcccactctagaggaggaatggagtatggagattaatgctgtaggtaagctaattaacacagagaaacacaaaatagaatgttcaaaatgaacatgcacttgaatacaccatgaaagagagatctgcaatggcaagacatttccataggtaaacattcctaaaaactctcccaccttccccctgttccccaaatagttattgatgttggttccttttccctctcaagataaagttaatttttggtacactttaggtatgctgaattaataaataatccaccagctgttttagccattagcttcgctttgttctccccaaacctttggccagactaagttattactgaataataactgttattcaacttaattattattcaaaaataactgttaattgttcaactggataattattgtttttttctaaactattcattggactataattctttctagatacttatttattttaaactaaaaacacaaaatagatttgctaaaggactcaaaagtaagcccttcaataatttctaaatcaggttgagcacaaattttgaagtgatctggagaaatcaagtcaacacttcagagtgagtttcacttcctaactgcaagacaagctaaattgcttaagctattatatctaactacctaccaattaaaagcaagggcaccatgaatgatacagaataaaactcactgtaatccgaaatacttacagtaaaaatagtacttttcacagtcacaatgccttggaataatcagttttcttttcagagcagggatcttgaaatttattattttaagatgtttgagttacataaatattataagaatatttctttggaactcttcagtgctttcccttattttatgataaacatagttctgaacttataaataaatggttgcctgaagactttagtagatatgttttattgtttacagagaatcacacacacatgaaaactaaaatttaaaatgaaaacataaaatctgtataataaaagcacagtaattaaaaatttaacaggttaggggtctttaagtcatactattatgccataattcaaaggaaaaaaaagccatagttaaggtaactctgagaagtaaattaagtcacatcaaatctgaagtgcaattcttttgttgtaacttaaaaacaaaaaaacaccctcatggccggttctcaagtaaacaaagaacataaattacaaactctggttatcaaatcccttaagcctttttcaaatgcgtcacaggatattcaaccccttcaattcttttaatcttcatcaccagggtactgtccatatggtacagaatcaacaagctagaacaaaaatacatcttatacaaacaaaatcacttaacacttcctactttatgcttgtgagtttcacacatttaaatttcaacatttatcatcagtaaaaatctaggtaaacggcttagaccagctctaccctcttaatacggaactgtagtaagagacggaaataaattccttcctccctgagtgtctggtaaatcccatgtggataggtgaactgccgatctttcagtaggagtcttaaaccaagtccacatgtatacataattttgataactttcttccagaattcaggatattcaaaaatagtttaaataattgggtcccatcttacagcgttttaaacatatgcttgcttaaaaatctaagaactgaaagcatgtcaaagagaatgccttttaatataaagtaagtacttaagcaaaaactaatccatacatcatgaaaccatctggcccattattcctgtgtatcctgccaagactgtgatcctataagtaagtttttaaaaaatcataaactgtcacaaagtatgatgataaatattttatggactttaggtgataattttaatctgtattcaggatcccaaggtaccaccaacgaaatgaataccattgcctcctcatggaagctacaatttcgggagtgactttttctcccagggacaaaattattactttccacgttcaagaactttgagaggggaaagaatatgacaaaaatttagtctaagctactctcagacgagtatccttcatacatagtgaagaaaggtgttaccagacttccccaccagtgccagactcggtggacctcggactccagaggataaggcgccaccagaaagcttcttccctcactacctaccctccctcccagctataccagacgtttatacctttaccctctatcccaccattaacatctccgcatcgtccttcccttctctccctctatatccctcttgcctgtgtaaaatgcggcggctgggatcacagagatggccgggggagttctcctctcctcctctttctaagctcttcacattgaggcaaaggagaccccggcacctgcctccgcggcagccaggaccacactgccatggcaacagcgtgcctctgcgtcctccatccgggcctctctaactacgagagcgacaaaaatctgcgcaggcgcatgcaatatcctcagaccaccgacccctcccggcacccatcccggtgcttgcgaagggccagtgaagctgaggtttctaaggggacgactaaggaatccacgcatcactaacgttatagagactcgcgaagtgccgctgtgttggttgtgaaatttccctcttgtagctgtctctcgtcagatttagaatttcttaggacaacttctaaaactatatttctatatgttatgtgcttcacagttaacgtagaaaagtagtttttagttttcattagtagataagcaaattgctaatgagttaccatatttttgaccctcagcttctttttttcctaatcaaaaccaaaactttacccaagagagttgccaaagaaaataggatttttgctctagctttgaggcatgtcaaaatggtcaactagaattttctcaaattctgcttatctttatgtataaaggcaaagcaagatgaatttagattgtggtgaaagttgagatgattttaacaactatccattcccagccagaaataaataagaactgggagaagtaaaaatatttcctcagtacaatccagtgtgttcggattatttttaaattgtttattattaggatttctatcacttgatgtgatggagagcaccttttctccaagagaggcaatgtaaaattaaatttttagatattcttcaagttatagacttaaggtcttttaaaatggaaggttaaaatgaattgtaatattgagtgcacttcctagatgtggtgtagttgtaatgacctaatgttgctactgcttcagcgcttgaaaaaagacgctcccaaaaaaggttattcactgctaaaggttgaaggaattttagtcaaatctcctttaatggatgtagagttgtagagttacttaccaaagctggaagtagaccctaggtttcagaactcctaggttaatagttttttcctctgaccatcctcctaaatttgtatttttcaaataatgggtgatagcagctttttttttaagtatcaggttttgggggaacaggtggtgtttggttacatggataagttctttagcggtgatttctgagattttggtgcacccatcacctgagcagtgtacactgtacccaatatgtagagtttttttgtttgtttctttgtttgtttgttttgtttttgagacagagtctcgctctgtcacccaggctggagtgaagtggcacgatctgggctcacttcaacctccatctcccgggttcaagcgattcttgtgccttagcccccagagtagctgaaactacaggcacgccaccacacctggctaagtttttgtatttttagtagagatgggttttcaccatgttgcccaagctgttctccaacaactgagctcaggcagtctgcccacctgggcctcttaaccaatatgtagtttttttatgcctcatcctgctcccaccctttctcccaagtccccaaagtccactgtatcatctttacgcctttgcatcctcatagcttagctcccacttacaagtgagaacatatgatgtttggttttccattcctgagttacttcacttagaataatgatgaaataacctccatttccatccaggtagctacgatgccacaatttcattccttcatatgactgagtagtattccatggtatgtatatgtaccacattttctttatccactcgttggttgatgggcatttggtttggttctttatttttgtaattgcgaattgtgctgcgataaacatgtgcaagtgtctttttcacataatgacttcttttcctctgggtagatacacggtagtgggattgctgcatcaaatggtagatctacttttagttctttaaagaatctccacactgttttccatagtgattgtactagtttacattcccatcaccagtataaaagtgttccctgttcaccacatccatgccaacatctgttgttttttttattttttgcttatgaccattcttgcaggagtaaggtggtatcgtattgaggttttgatttgcatttccctgatcattagtgatgttgagcattttttcatctctttattggccatttgaatatcttattttgagaattgtctattcatgtccttagcccactttttgatgagcttattcgttttttccttgctgatttgtttgagttccttgaagattgtggatattagtccttagttagatgcatagtttgtgaagattttctcccactctgtgggttgtctgtttactctgctgattatttcttttgctatgcagaagctttttagtttaattaagtcccatctacttacctttgtttttgttgcgtttgcttttagattcttggtcatgaagtctttgccaacgccaatgtctagaagggtttttccaatgctatcttctagaatttttatggtttcaggtcttagatttaagtatttgatccatcttgagttgatttttgtataaggtgagagatgaggatccagtttcatttttctacatgttgcttgccaattatcccagcaccatttgttgaatatggtgtcctttcctcactttatatttgtgtgtgctttgtcagagatcagttagcttcatttcttggttctctattctgtttcattagtctatgtgcctgtttttatgccagtaccatgatgttttagtgactatagccttgtagtatagtttgaagttgggtaatgtaatgcctccagatttgttcttcttgcttagttttcctttggctctgcaggctcctttttggttttatatgaatttgagggttgttttttctagttctgtgaagaatgataatggtattttgatgggaattgcattgagtttgtagactgcttttggcagtatagtcattttcacaatagtgattctacccatctatgagcatgggatttgcttccatttgtttgtgtcatctatgatttctttcatcagtgttttttagttttccttgtagagatctttcacctccttggttaggtatattcctaagtattttattttttgcagctattgtaatggggttgagttcttgatttgattctcatcttggttgatgttggtatacagcagtgctactgttaccggtggagggtgtccatgttcttggtattttgaacaaagaattggaaaaaaaaaaacacacaaataaagcaaggaaagaatgaagcaacaaaagcagagatttttattgaaaatgaaagtacactccacaatgtggcaacaggcatgagcagtggctcaagggctccagatacagaatcttcttgggtccaaatacccactagaggtttcccattggacactttacattggttcaccccatgtaaatgaagtggtggcccgcaatcagtctgattggttgtggaaagcaaccaatcagaggttgaagtgaagttacaaagttacactcctatgcaaacattacactcctatgctctttgcaaccaatcaggggtactttcaatttcccatctgcaggaaagaggatttgccaaggaagtagcctctggtccttttgttacttaaacatggcaagttagggttttcctttcaactgcattctaggaagtcagtgtgaaatggccttaggttcactgactccagaccctattctcctgcctcactactgatttgtgtacattgattttatatcctgaaactttactgaattaatttatcagttctagaatctttttggatggttttctagtatatgattatgtcactgacaaacagcaacagtttgatttcctctttaccaatgtggatgccctttatttctttctcttgcctggttgggatatagcagcatgattggtagccaaaagtggaaatgactcaaatgtccatcaaatgatgaatggataaatgttatatatctatacaatggaatattattctgcaataaaaataaatgaagtactgctggaggattctaaaaagatggtggaataggaagcaccaggaatctatctccccacctggaaaaacaattgcactgtcaaaacctatctcttgtaactattttggaactgtggagtctattaaaggcttgcaactttcaagggaaggcttggacagaaaactgaacttattttggtgaatttcagctcttagttcagtatcatctacccaaccccaaaccccagctccctagcaggcagtctgtattacttgcaggagcagctttaacatagttcatgagagtcaatgagcagtaaggaccctgtcctcctattgatgagggtctatgttctgataattgattactgcttctgatcaaggagatgcaaacacagaggtagccgccattgttgttgcaccttcccccaccattgtaagcttttactcctccagctgaagcaacttccagggaattttccttgttttcttccttttggaagacaaacattaaagactaagacattaaaaagcaactacatttacagagaaaagtataaagttaaattgaacacccagagaaaggcataaactcagaaaaatacctgagaagaccttaaatcataaagatgctcaaataactaaggaaagatgtggaga

SEQ ID NO: 128 - DNA, 5644 bp, Artificial Sequence
  subsequence of FOXG1
ttccaattcggattagaagctgaggctggagcggcgaggagaaaacgacatcgagtcaccccgcgcgtgagcgacgctctggagcaggagagaaggaaaaggaagagacacacgtatatagcatgtgttacctgatttatttcgagattgtttggtactgttttctctctgggcacctctcatttcggaaggccatcagaggcgcccactactgagcggccccggccgccgcagcagcacccggagccccagtcccggtttccccgcggtgccggagcccggagctcgccgccgcccaggcctcaggaatcgagtaagtaacccctgtttggccccgcgctcctctgcccacttcctcctcctcatctcatcgctcccaaagctctcccacaccctcctcccccgaagtaaaggacacccccgcacacacgccagcggtgggccttatctccccccttatccaaagctgcgctagactattacctacaaagggcacaattttcctgctggctcagaaatgccagacactggcctgcaaggccgactcggaaaagtagtgcctcgcctctacctcctccatttcctgccctctcgcatcctctccctgcactcccactcttcccactaccaccaccacccccgctttcaaaaataaaattggatacaaactttaatcactaaggacaaatattgacgcttaaacgaaaatgacccagtacaatgaggaggaagccggaaatgtgagctattggccctaggaggggaatttcggtggagcggagccggcggggtggagggggtggcgggggcgcagggaagggctgcggcaccgcgtgctcccgccggcgtatccctacgcggctccgcgcggcctcggggtccgaggcccgcggagagggggaggcgagcgcccgagggggcggcagccggcgggcggggcgggggtgggtgggcccggcccctccgattggtcgacggcgagagagacgctcccgcacgccgccagctctgattggcccagcggtaggaaaggttaaaccaaaaatttttttacagccctagtgtgcgcctgtagctcggaaaattaattgtggctatagccgcctcgatcgctgtctccccagcctcgccgcggccgctccgggacgcgcccgcccgccgcccggctctccccccctttgggctgctgctgctgctgctgtgactgctgctgcgagaggaggaggaggaggaggaagcagcgggggggggagcggggggtggggggggagaccaagaagtacagttgggagcgagggagcttcacccccggggcggtggttgtttcttttttctttctttcttttttcttttcctttttttttttttttctaattcctgaggggtggttgctgcttttgctacatgacttgccagcgcccgagcctgcggtccaactgcgctgctgccggagcgctcagtgccgccgctgccgcccgcgccccccgcgccccgttcggcacccaccggtcgccgccgcccgccgcgccgctgtcccgctcccgcgccgccgccgccgtttccccccgacgactgggtgatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaacatccctgggaccagactgtaagtgaacgttttacacacatttgcattgtaaatgataattaaaaaaataagtccaggtattttttattaagcccccccctcccatttctgtacgtttgttcagtctctagggttgtttattattctaacaaggtgtggagtgtcagcgaggtgcaatgtggggagaatacattgtagaatataaggtttggaagtcaaattatagtagaatgtgtatctaaatagtgactgctttgccatttcattcaaacctgacaagtctatctctaagagccgccagatttccatgtgtgcagtattataagttatcatggaactatatggtggacgcagaccttgagaacaacctaaattatggggagaattttaaaatgttaaactgtaatttgtatttaaaaagcattcgtagtaaaggtgcccaagaaattattttggccatttattgttttgtccttttctttaaagaactgtttttttttcttttgtttacttttagaccaaagattgggttctagaaaatgcacttggtatactaagtattaaaacaaacaaaaaggaaagttgtttcagttggcaacactgcccattcaattgaatcagaaggggacaaaattaacgattgccttcagtttgtgttgtgtatattttgatgtatgtggtcactaacaggtcacttttattttttctaaatgtagtgaaatgttaatacctattgtacttataggtaaaccttgcaaatatgtaacctgtgttgcgcaaatgccgcataaatttgagtgattgttaatgttgtcttaaaatttcttgattgtgatactgtggtcatatgcccgtgtttgtcacttacaaaaatgtttactatgaacacacagaaataaaaaataggctaaattcatatatatcttgatacttttgtctcttttattaagtagagctaattttttaaagaccaatcaacttatagggaattcaaaggctttttcagccaaactaaaatttaaactgctcctttaatttgaactgactctaaaaatgaaaatagtatttttccctttgtgaacaaattttacaaggagcagcctatttaataaacactagctttaaacaaagtataggcttttcagctgatacctgtaagtttctgtggatatacagcaaaaagagatataatttaattttctgtgcatagctctttaccctgtgtttatttccaaatccattaatagaatgccatttatatattttgtttcaggtatattgttaatagagcttggcaaattataaataaatatatgtatatggttagatagaagtgactataatgcacacatatgtaatatatatagacacacagagcccttcagttcaggtacaatttgcgctatgaatgctgcaaacatttttgtttaaatatttgtatttatactttctaagtcagcatttatttttgtggctgtttacccacaatgaaagagttctaataaagatgtgctgaagttgcaatatagattttgctgaagtgatcacctgtttaatgacttgcagatcaatgtttatattttattttaaaattataacaatttagaattctagatattcagaagtaaaatgcctctttttttaatttttacaatgtagaggtatttatttgattcccttttaaatgtctgtgtaattcaatctagagatttgttccttttgtttagtcataattttaactttccatatgcatatgtatgcatatactgtatatgcgtatatatgtgagacaaaatgaaaacattttttgaattgtttcaggcagcccaatcgctataattgcagaaaataattagaaaaccttagtaaaaggtttagtaactaattggatcctgtggtgaatctgtggaggacaagttgttcatttgttacaattgtatagtctgaaatgtgttctgcagtattggaataagctggttacttcttacaatgggcccagtgaaaaataatctggccctaagatgttatcagcaaacacttagacagttgctcagggggaggaaaaaaaaaaaaagcctaagggagaagcaagcttactggaatagatttgaaactgaaatgctgaatacttaatattggaaaatagaagggggtggactgtggagtggagagagggaaagagagagagagaggatctttatggtgaaaaaataattaccaccaaaaagaatgcatgagagagagagacataaggtgaatgatagagagagagaaaggcctaagaaaaatatataagggccaggaaaattaaaagagctggaaccaaatatacacaaatggggaatggacaaaaataagccaagacagctagtttaattatctcctttaagcaattagacatgattttattttcttagtataaaaacagtttcaccagaggtccaaattttcttgaaatttaagttcttcagaatgaagcataaaaatatttcacaaaaaattattgtgttaaaagtcaaatttatggctgatattaaaaatagtaataaagtccaagtgaatggttttatgcaaaggaaatccccattacacatt

SEQ ID NO: 129 - DNA, 7260 bp, Artificial Sequence
  subsequence of ATP4A
tgtctcgctgccttccgagatgatgcccacactggctgcaatggccttggcggtgatggggtggtcacccgttaccatgatcacctgtagggggaaccagtggatcactgaccccttcagatcagcccaatctccctgtcctccctgggagacatctgctgatacacgtgttcatttacttgaccaagcgtgaccacctcctacgtgcctgggatatagcagagacaaaagagacaaaaatccctgtccttgaggagctctagtgagggtgacagaaaataaagaaacgaattatatagtgtgttagaaagtggaaaaaaacatattaagcttggtgaaggaaacggtgactcgattttaagtgtacccagggaagggcttattgagaaggtgatatctgagcagagacctgtaagaggtgagagagtcagccacgtggaaagctgggagaagagtgctcctggcagggagaacagcaggtgcaaaggccctgaggtgaggacaggcccgaactgtcggaggaacagcagggaggacaccataactgcagaggagtgaacaaggaaagagggctaacgaacaagcttagaaaggtaacaaggcccgatgtaacagggccgtagagacacgggaagggcttgggcattgcctgcctggccttggctttcccacccttgttttgtgtttggggaaccactcctctcccctacaccatcagttgcagtgaggtccctggctaagattgggtaagtaagcccagacctggccaatcaaggtcttccatcccccagccccagtgactggctcagggataaatgagtgacccaatccagtgctactgcaaaagaggcactctctttttgtggggtggctaagcaggtgggagttaagtttgagctgctggagcaggggtgtgtgtgtccccaagtgaagggagtgcctgcaaataaagccagcaaagagaacagcagagctgagacatggagaaaggaaaattcctaatgacactgagctcctggatctggctagacctgaagcttaattccccagaatttttcagtatcataaaccaatgcattttggttcaagccagtttcaggtgggtttgtcatggggttcttcaacccaaatcctgctgccatcctctgtcctcactccacttgttcctccccacagtggcatacccggatgcctgcggtgcgacacttgagcacagcatcagggacggtggcccggggtgggtcaatcatggatacaagtcccgcaaagcagaggccgctagatggaaagttcatggcctctacgtcgaaggcatagccaggcgggtagtccttctcattcaggtagagctggcagaagcctgaccggaaacggggaagtcagggaagagccctgggcacaccctttcttagcagggccaggaaatgggtaaaataaccaggccccttgcaccaaacacctatggatgcctgaccttgtgctgaccccttcactcacattatctgaatctaccctcacaaccaccctgtgaggcccattctcatcttacagatgaggaaactgaggctcagagaggggacatttcttgccagaggtcacacagcaagtaaaaggcccagaattggaccacagatctgctttctagggtagaggcagcgaagtttaaggcgtcaggacaaaaattgggagaggttagaggtcaggatacggataaaagtccaggtgaggactggggtcaaggtagaaagtgaggacagacaggggtcaggactggtacagggaaagtcaagggtgaggctgtggactgcgacaaatcagccagcagccagggatgaggacggtcagggctgggccgggagtggtgggcagggtctgtgctagctcctcctcgcacctggagtctcctcccctgcccaggggtctcaccgagcacgcgttcgcccaggcctcccaggctgaggtaggcggtctggaaggcctcgcgccactgctcgtccagcggcagctcctggcccttgataaggatggagctgcagcgctccagcacgcgctcgggggcgcccttcatcaccagcaagtgtcgcgggtcccgcgggtcctccagcgtatggatggacagctgtgggcgggggggagaggcgaggctgtggacgggggaacggggcggggctgtggacgagggaacggggcggggctgaggagaggggcggggccgagagctcggtgcgggctctgagagctgcggggaagggtgaaggtggaagatggaggccttgtgtggaggggtccttggtagaaggtaagcgttaaggcggggctaggtgcagatttggagtcctggacgcagggaatgaacagaattagggtgcggagttgggctgggggcggatttggagagcgaggtgctccccatggacagtcccgccgaggagaagctgtgggcggggctgggtggtgggcggggccttgcctctggtggacggggccataggcggagcgggagatggggtggggtttggctgcggagagaaggggcaaggagcgaagcccctcgtggcccgctgatgtgggtgtggcctggggcggggcccgaggtgggcgggcccaggccgtgggcggggccggctgcgcacctggaacttgttggtggagttgaagggtatctcgcagacttttgggaagcggtcccggtagcccatggcgttgcccagcgtcagctccgagaacttgagcagcgccgtctccgatgcgtctccaatcacgatgcgctgggagcggggaccggtgtcaggggcgaagccggctacaccagcctcccgggattccctggaggccccctggctctcaccttgggcacaggcactgcatcctggccggacttgaaggcggcgcggttgcacagggtgagcacccggcacagcgcccgccacgtctccgaggactggtcaaacgtctgccctgcagaccaggcgtccaggctgggtcccgcacggcggctctcccggaccagaaccgagccccctcctcctaggctcatatcgcgggccccctccccagacctgggtggaattgggttccacccaaccctgagggacccagcccccggatgacccttccctctagacccggtagcgagtctcctttgagacctggagccgagccgccccgccttcgtacaaagccctccctacctccctatccctcttcaggtctccaccatccaccagatcctgccctggcgcctgtgccctccctcccccacacctgactggtcttccgtggtgtcagctgtgtggatgtggttgtcaaaccacagatgggacacagtcatgcggttctgagtgagagtccctgtcttgtccgagcagatcaccgaagtggagcccaatgtctccaccgcctccaggttcttgaccacgcagttcttactggccaggcgcttggctgtcagggacaggcagacctggggaaggggtgagcaccgcaggctggggacccaccctggcttccagtcctcttccccgcgtcaaagaacggggaaggctttaccccagccgcggggctgcgtgtgcaacgtgcttctgcaaacaccaggtgttttcttggccccagcttttgttcagccagtgctttgtttgggactccttccccagtctgcccagatacagtagcgaggcccctctctgagctgtcccagccgaggttctgaaaattctcttcacacaatcaaaatgagaagaggaaccttccccgcacctccctggggactggtctggagctgggtgctgactgtggaaggtctggtgacatgctggctgcctgcacaggacatcagcctcttccagttaaggacccggcctcgtcatctctgcgccctcaatgtgtggcacaggacttgccccagggttgcctcgggaaggacttgctgaatgagtggatgatgggaaggcaggagaatggatgggagctaagtggacagatagacaggcagggaggtgatgggggaaatgtggaggaaagaacagatggttgagcaggcccctcagctccctgcatccccgcctgcccccactcactgtgacagtggccagcagcccctcaggcacataggccaccacgatggccatgaagaagaccatggcccgcaggaaggtgtagccaatgcacatggccacaataaaaaatgtggcaccgaagagaatggccaggcccgcgatgatgtccacaaaatgctcgatctcgatagcgatgggtgtcttctcgttttccacccccgacgccagcgatgcgatgcgcccaatgatggtgcggtcgcccgtgttcaccaccaggccctgcacggtgcctgcaggggggccaaggcgcgactcagggatagggggcggcagtggggtgtgcactgccgtgtgagctgaaggacagccagtcactgccagtggaggatgtgacctgggagagggtagtgacagtgggagacagagaagcagtgtgccctggggaggtggcagtcacggggaggtggcagtcatgcaggagagagacagggaggctgaagccccctgtcctagaagatagcaggagagaggccagtggaggcagcagttactgggcaggcaggcgggggcttcacagaccctcaaggcacatggtggagaagaaggcgatgttgcgggtctccagagggctctcgtgcgtgcactcgggtgagcgggtctgtggctcagactcccctgtcagcgaggagttgtccaccttgcagccctgggccgccaggatgcggatgtcggcgggcactctgtccccacctttcatctccaccaggtcgcccaccaccagttggtcagcgttgatctggaatttgtctccatcgcggatgacagtggcttgctgcggggcaggggcaccaaagttgaggtggacgggggtgggggtgggagctgctgcatgtggggaggtaaaggatgaggagagctgggacccatggggagagatggaggccacagatgagggacaggggcctgatggaaggaggctacaggagcagtttggagtctctgggatctggagtggctgggtgctggggaacccacctgtggcacaaggttcttaaagctggcgatgatgttggtgctcttgaattcctggtagtagccaaagcagccggtgacgacaaccacagcaatgagagcgattgccaggtacagctggggacagggaaggggtggggttattcagaggggccggaagctgcctgcctgaggccaccgacctgctccctggtgccctggttttcttggttttccccatgcttttccccacctactaggaagctccatgcaccattccctgtagcctttagccccctcccatgtccctcatgtcccacactcgcctgctctgtcttctcagtgtcttgtgtttctgtgtatatttcatatcctctggccctgaatcctcaatcttccatgtccccatatctgtgacttgtgccccaaatcactgtgtgccttggtctgacgtgttcccttctcctgtgtccccaagccttgcattccccatgtccactgtcccatgtctcctcctgtccccatggcctgtattccccgtgtctccagtttcacatcctgggttttccataatctgctgccaccctctgtgtgtcccctgggcccccttgtccctgcatcctcacatccccgtccctgcgtgtgctgcagaccttgtacgtcatggtctgtgtcctctgtggccacggcccccatgttctagctccccagtcactgtgtcccgtgtctcccatgtctgtgtatctccgagttcccatcctgtgtcccatggccatgacttttcgagcctctgtcccatgtcacatgtccctgagtccccatgtcccttgccctcatctctgccatatgtccaatgtcccttgccctgcacctccatttcctctctgtctctgtgcttctatgtctcatgttttgtgtctctctatgtgctgtgtcctgaggcttgggtccctgccctggtgtcctatgtctgagtcccagggtctcctattccccgtgcctcagtctgtcctgtgtctgcactgcatgtcctctgcttccaagtcccatctcttttttttttttttttttttttttttaagatgcagtctcgctctgtcgcccaggctggagtgcagtggcatggtcttggctcactgcaacatccacctcctgggttcaaatgattctcctgcctcagcctcccaagtaactgggattacaggtgcccgccaccatgtctggctaatttttgtaattttagtagagatggggttttgccatgttggccaggctggtctcgaactcctgacctcaggtgatctgcccgcctcggcctcccaaagtgctgggattacaggcatgggattacacgcccggcctccaaatcccatctcttacgtatcacattgccatactctttatgcccccacctatgtccccatacccaagattcaccccagtgtccctgtgtcctgtgtccttgggtctgccccatccatatcccaatgtccttcaccctccatgacccctgtcttgagactgcctttcgtgttcgtctatccccatccttctctgcccctcttgtcccaagtcccaggctcagtgtcttccatccccaggtccccatgtcctgagcctgtccccacaacatggctcacattgtcgtcggtggtgaggtccccctcactagcctggatggcaaaggcgatgaggcagatggcggcggcaacccacatgaggcactgcaggcccccggccagctgcctcgcgaacttgacgtactctggggtgccccgtggtggccgcagtgcgttgggcccatcccgcagcagcagctcagcagccaggctcgcagagaggccctgggacagaggggcagggcgaggcggtcctgggggctttcctccacatgcacaccccgtggaaagccccctgcttcagtttcccttctccagcttggtctgtcctgctccttctcatctctttgtctcttggttcctgcccccaccccatctctccctccctccttcatctctccctttgtctctctctcctcgctcccctttctatgtgtctctctcctgctgctctctccttatttgtctctgccttctctgtcttgctgtctcttttcagctactcctctctccgtctctgtgtgtcaccctttctctctaatgctttctcccttctctccctctccctctctctc

SEQ ID NO: 130 - DNA, 7809 bp, Artificial Sequence
  subsequence of NOTCH1
taaaaatcaacatcttgggacgcatctggtcatgccccctggggatggcaccacgcggcccccgtagagccgggggaggctgccctgaggagtgcaggcggcacggcagcggagcgtccccagtgcatgggcggctaaggctccccgagctgagccaagtctgacgtccctcactggcatgacacacaacagactcattcattaagattttttaaacaaaatccacctttaaaaacatatttacagacattttttctgccataggctatacttggtattgcaaaaatctgctcctcccaaactaggaggggtggcccaagggcagcccccgcctccctccagcccctgcccgaccgcatgccccctgccagggctgcggggacaggaccaaaggacgcagcccacggcgttgcacagctcaatgtgcccggctctggcaagtctcctacaaaacacgggagcccacggggggtcgggtcccgcgagctggggcccaggctgtgttgctggagcatcttcttcggaacctgggggacactgtgcaggctgaggtgctggggccgccaccggggtcagcccaggcagtgtctttccccagaaaagggtaggatgcctccgtgtgtgacccaggcaagtgccacagtccacacatctcatgtttgacatgcatgatgcctacatttcaagaacgggcagggggccggggtggttctggagggaccaagaacttgtataaccaacgaacaactacataatactgaacctgaaacaaagattcatgattggtaccatgggtgcactcttggcatacacactccgagaacacattttcacaagcatgcttgcaagaaaccatctaaaacacatggcaacatctaacccatatgctttcacttgtttcctatttcagatgcaaattaatccgcgtgcggaaggtgagccagctttgcctccgtttgcctctggatgcagcttctcctaacaggcaggtgatgctggtggagcggccgggctggcttggggttgggtgggcgtcggggaaagggcgcggcctggacgccccaggagctttttggactatgctcgttcaacttcccttctccaacatcatttctttttggattttgaaaaaaggaatcaataaataaatggcatttataaatcatgaatctttgtttatattttataaacacagaagaatcttttcatcctacgtaggaaaaccctggctctcagaacttgcttgttttctcagaatagataaaagtttctacctggggccagataaaacagtacatataaataaaaaggcagtgtttctgtgtaaaataaaagtacataaataaatactaaaaaaaattaaaatcctcgttcttattttgtataaaaacatgtgttttaaaaaggctcctctggtcggccctggcatccacagagcgcacacagacgcccgaaggcttgggaaaggaagccggggtctcgtggggcgcgccgtttacttgaaggcctccggaatgcgggcgatctgggactgcatgctggtgggagggctggagacgccctcggaccagtcggagacgttggaatgcggggacgagctggaccactggtcaggggactcaggggacggggtgaggaaggggtgctcaggcacctgtagctggtggctgggggtgttgtccacaggcgaggagtagctgtgctgcgaggggggcgtcaggaactgggctgcggtcacgggtgggaccagcgaggatggcagcgacgtgggcagggcggggctctcctggggcagaatagtgtgcaccgccaggctgctggggcccagtggctgcacgtctgcctggctcggctctccactcaggaagctccggcccaggtggccgctggctgctgagctcacgccaaggtgcggctgtggtggtggtggtggcggctgcaggctttgctgctgctggatgtttgctggctgcaggttctgctgctgcatctgtaagttttgtggctgcacctgctgggtctgcaccaggtgaggctgggtggccagccgggtgctgggcaggccctggtagctcatcatctgggacagggcgctggcagcaaggctactgtgcagcgggcctaccatgccatgctgcagggagggggcctgtgtgctcagggggcctggtgccacactcccccgcagagggttgtattggttcggcaccatgccgctctgcagccgggacagccactcgcattgaccattcaaactggtggacccgcccacagtgaaattcagggcccctccgctgctggagcccaggacggtgctggtgccagaggccacaggcaggtgggagagacgaggtgggccagtctcaaaggccagccggccgcccccacccagcgccgccatctcgggcttggccgccacgttcaggtgcccgatgcccaggtgggtgtcgggcatcccaggcaggtggttgaggggcacggacggagactgctggaacggggagggcagcagtggcggcgaggccacgtctgacaggtagccatggggtgactccagggagtccacgggcgagagcatgccggagctgtccagcaggcagcccttgccgtcctgggacttcttcctccgtgccttgaggtccttggcctccttgcttccacaggccaggcctttgctgctgggcttgcggaccttcttgccctgcacgccgggcttgaggctgcccaggtagccgttgggcgagcagagcgggggcgacagggtgggcgtgccccccagcggggctccgtgcagctgcgggctgcgcaccaggttgtactcgtccagcagcctcacgatgtcgtgatgcatgcgctcctgtgcgatgtcgcgcggcaggcggtccatatgatccgtgatgtcccggttggcaaagtggtccagcagcaccttggcggtctcgtagctgccctcccgggcggccagaaacaggggtgtctcctcctgggggatgagggcgggggccggtgaggggggccaggccaggcgtggggaccctccccaaggttccatcaccagaggaagcagcagtacaacctcctccgcggggtgggggcaggcaggctgctcctcaggaccccaccccagcgtcctgcaaccacctagcctgactcttgttaaatgttaaggctttgatacatcttctgaaacccacccagagaggggatctgtgcagtcctctaccctagatcagcggggctccacctctatgggggggtcacagaccccactgaaaatctaaccaactgcaggccctcttccctgcccccaataaggcacaaaaggaaattcctggagcggttcagaggcaccggcgagggcaccagggagggagctcgtcatctggacggccatcaagccaccctggtggccaccaagctccttcctggggtctcctctgtgcggtggacacgtcacaggtctgggtgaggctgctgtgtgggagtgggaaggggcttcccaggccttgatgccctctggggcccagtctctgtggggccatgctgccacggactggggagcgcctgggccaacatgccccggtgcccaggcccgctgtgtgccctcacccaggagggagagacgcctgagcctgacatgccaagggggcagcaggagttgggggcgggggtgggggttctggcggcagggaggtgggggtgtctgcacctgtgccctgagcacccaggggaaaccaacagcagctgcagggccaggagctgcagggccaggatgagggctgggggtggctccccttggatatagtcctgagacttcttcctctgagccttgaggtccttggccttggtgaacacaccaagaggccccgccccgcagggagctgccgccacgcgtgttctggctcaatctgcaccacctgcgtcaccacattcccatccccacccccgcctggtgacagctggggacagaaggtgaggtccaggtgacggtgacacagcagtggcagcagcagcgttccccacgccaggctgtgtgccccgccatgactgccgcccaggagggtagggctgggtctgaggggcgagccgccgtgtcctgacagctggacctgcacttggaagggctccataaacgggggctgaaggaaggcaggagcactaacgaggtgcccgcatcatgcgggtggggcccacatgcgggcccagcgaccctcgagaccctgtgggtcaggcccttgtgtccctgcgccccgtgggtttggccctcacttctctgtggattcagccctcacgtctcccctggcatcccagcctcgcgctcaccctgttgttctgcatatctttgttagccccgttcttcaggagcacaactgcggcatccacattgttcacggcggcggcccagtgcagggcggacttgcctgcgtgaaagaagcagatggggtaggttggagaccagctggaggcaacccagtcccacccgtccctgtggcggtcccgccccacgacagagcagccgtgcccccgtgggctcacccaggtcatctacggcgttgacgtcggcgtgtgagttgatgaggtcctccagcatgccctccacggccaggcgggcagccaggatcagtggcgtcgtgccatcatgcatgcgggcatccaggtctgtggctcggttccggatcaggatctgggcaacagggagaggctcaggcgggtgctgggcagacgtacaccgatgcctcctgcccagaacgaacgcctggacgggaagccccactgtcttccggacacagacgagaccctcctgagatcggcacggccgggcaagacgaaacgccatggggctgcccctgaggaggggcccggatgggggcaggggctgtgctgcccacagacaggccccttggtgacagtgggatccatcgtgaccacgtgtggcctcatttaatcctcactgtggtcctagctgccatgaccacctacaaaacagagaggtcaagtccctgcccccaacggcggttacagctggcactcaggaagccgggctcacggccctacagccgtctcagaaccgccgagctagtgtccggctctgcccagagagggggtgggcagcccttgtaatgcagccgggtcttggcatctggcaggaggaatggcccctgctcactggcgggtgcaaaaggcaacttggcctgattcctgcaaagcacctgggaacttccacatctgtgcaaagaaggcagctgagagaggcacacaccaaacccgcacggggctcccttccacgctctttctgctcacctacatttatttctgcgagatgacaatgtgtcacctgagctaagaaatcaggaaatgagaagctctgtggtaactccccgggctgtgcccatctgaacctgcttgctccagggccgcctggagcctggggactcgcagcaggggtgactgcaaaggcagcccctgccagggacactccctggagacccaggcatccaaatccctaaatccattcttgccacgagctcaccggcaagctgagccctaggtctgcaggcaccgagcggcagcctctaaaccctgcttctcactgccacaggctacgccggcagccaaggttgagggaggggcggggctgcgaggggctgcagcagtggaacctggaccttgagcaaggcccacaaccctgatggagcctgttttgctgggggtggggaggtgctgcctcactcagcagatcccccaggcctcctgccagacccgcctctgcctgacttccctggagaacacagatgggcccgacgccctccccctcgcacctcgctgactgcgaaggtcccaggtggaggcaccagttgtcccggactcagctgtgctcggggtcaggctcccagggccacgtaagcctggccactgccccagaccaccaggcggccctgaggagggcaggtgggcacacaggcagccactgcctacctggaagacaccttgtgcgtcggcagacacagccgcatgcagcggggtgcggcccatgttgtcctggatgttggcatctgcgctggcctccagcaggcgcttggcggcatcagagcgtgagtagcgggcggccaggtgcaaggcggtctcgcccgtgcggtctgtctggttgtgcaggctggcgccctggtagatgaagtcggagatgacggccggcgcgtcctcctcttcctcgctgttgcccgtctccaggccgcccccgctgcaggaggcgatcatgagcggggtgaagccatctgcagaggcagagacgggtgctcagtctggagggccggtccccagctgcagcccagggggaggggggcagcagccccaagctcaaggggccacggtgtggatgcaccacccagcacttggtggccccgtgcacacagccaccaaggggcagctgaagtccgcctgggctgctcagtggacagagccgaatccagcttaaactcctggcgggcaactgcttcctgacctgcccaagacctggcacccaaaatccacccaggaaaaagaggaccccagaagcatgcagttagaaaattaatgcttcagcaggatgcggaggctcacgctgtcatcctagcactgggggaggccgaggtgggcggatcacctgaggtcaggagttcgagaccagcctggccaacatggtgaaaccccatcgctgctaaaaatacaaaaattagccaggtgtggtggcaggcgcctgtagtcccagctactggggaggctgaggcaggagaattgcttgaacccgagaggtggaggttgcagggagccgagatcgtgccactgcactccagcctgggtgacggagcgagactccatctcaaaaacaaaaaaaaaagaaaaaaaaaaagaaaaagaaaatcaacgctttccatgtctcaagaaggtttcagaaaagagtaatttacagggacagagtggccctgcggtatcaccccaggaaggggttggtggaagaacaggaggactccaggacccccccacgtctactctgaatgggaaacaccccaaggatgaaagctctcacccccaattctaagtttccagagtatcaactgtaccccagcctcggggcttaggggagagaggcaggtgggccacggggctagggaagccctggctgctggcacccttaccaggcccgcggacattgacgtccatgcagtcggcgtcaacctcaccctggggcggtgtgggggccatggcagacatgcgcaggtcagcggcatccaggtgctgctgagtccactgccggtggtctgtctggtcgtccaggtcaggcagaaccacgggctcctcgaactacatagagggagtgagcagagcctgtcagggcagcccggcagcaggtgcccgggagcccaggagcccgggagcctcgcgactcacccggaacttcttggtctccaggtcctcgtccccccactcattctggttgtcgtccatgagggcaccgtctgaagcgttcttcaggggcctggggggtgaggggtcgagaagtgaggctgagcgagctccctaggaagcccccagagacccctggcccgggcctggcgtgggaggtgggccctgggtcgggaggggcagactcccggtgaggatgctcggccaggtcccacctcccaccggggacccagaagcaggggcggcgtccgctcacttgaggcccacggagtcctcgccgaggggctcccgccgcttcttcttgctggcctcagacactttgaagccctcagggaaccagagctggccatgctgccgccggcgcttgcg

SEQ ID NO: 131 - DNA, 6917 bp, Artificial Sequence
  subsequence
agttaataaatgaaaagagctgtaaatgttcatagcagttgtatcactgcctagcataagaaccccttaaaaacctgtttcttaatttgggaaacagatatgacgatagttagcatttattaaaggatgacagttaacaactgctatgtgccaggccttgttctaacagcttttcatatttagcccacttaacatatttctattttcatatgaggaaactgaggcagagagaggctaagtaacatacccaaggttttccagctagaaaatggcagagccaggactcaaacccaggcagtctggctgctgagccctggttcttaattatgacattaatgcttattctgcccagtgaggataaaatgagtgaaacataaaatcaaacaggatgttttggtagggagcagtgttttttccctctgaaaaatgaaaaattaggttattgtgattttgtaatttacagcagtgaatatgatgtgaaaaataagttatccatataataatttatgtcaggagtcatgcagcagaaagatttctgtccatcacgtaaactttcatccattacataacccatatgtttctgtaccattaagacacttggttcaacaagacccttggagaatgaggttccttttgttccctggggttctctttttattttatttttggattaatatttgatagtaaagccaaggatttgggacaggaaacttagatgacatctagttcaaactccttgatttacatatgaaaaaattgagacagagggaaatgaagatttccccatatcatataactggctaaagggagctatgtaggtaaaaccaagatgtcctgatattctagtctaccagaaagtgttctttttttctacccaacttattcctgatttaaaggctagtatacgtgtgctgatctcccctcagtgggaggggcatggacgttgggagtagtctctattcacaacaaattaaaaatcagtaatcagccgtataatgggttgtgttagaaagtaaactaaggcccaataaaatatttaagagtttatttgagcagtgatccagaagtggctagggagctccccagagagaacatgaggaggaggctttttaggacaaatagataaaagcaaagataatatttcattggttacagttatacagttacacagttatacagttgccttatttggtctatcccatgaggaagtcctagttactaattacgtttttgttggctgcttctgattggttgagcttaagttctgtgtttctttaacataggcatttacaagaaataccacaaataaagtttcagacatgcttgcaaatcaagcaaggttaaggtcacttaggaggcccaactggctctgtctgctcaaggattcttctggcctcgtctccattttacatgaactgttgcataaataaacacagagtacctgaaacaacggaggtgatcattctgcctaccgagtgttggccaggccaagcttggagtgttgctcttattcttagggagtttatttttaagtaatctcatctgtaaatgggattacaatccacaaactgaccttgtatatgattccattccttctcccagcccagccccacactccaaggttttccctttgcttataaggggtagtcacccttttttatttcgaccttccaaacattctgggagttttcctcctttaggccaactacagcgcagaggagcgctttctcctgctgggtttctccgactggccttccctgcagccggtcctcttcgcccttgtcctcctgtgctacctcctgaccttgacgggcaactcggcgctggtgctgctggcggtgcgcgacccgcgcctgcacacgcccatgtactacttcctctgccacctggccttggtagacgcgggcttcactactagcgtggtgccgccgctgctggccaacctgcgcggaccagcgctctggctgccgcgcagccactgcacggcccagctgtgcgcatcgctggctctgggttccgccgaatgcgtcctcctggcggtgatggctctggaccgcgcggccgcagtgtgccgcccgctgcgctatgcggggctcgtctccccgcgcctatgtcgcacgctggccagcgcctcctggctaagcggcctcaccaactcggttgcgcaaaccgcgctcctggctgagcggccgctgtgcgcgccccgcctgctggaccacttcatctgtgagctgccggcgttgctcaagctggcctgcggaggcgacggagacactaccgagaaccagatgttcgccgcccgcgtggtcatcctgctgctgccgtttgccgtcatcctggcctcctacggtgccgtggcccgagctgtctgttgcatgcggttcagcggaggccggaggagggcggtgggcacgtgtgggtcccacctgacagccgtctgcctgttctacggctcggccatctacacctacctgcagcccgcgcagcgctacaaccaggcacggggcaagttcgtatcgctcttctacaccgtggtcacacctgctctcaacccgctcatctacaccctcaggaataagaaagtgaagggggcagcgaggaggctgctgcggagtctggggagaggccaggctgggcagtgagtagttggggaggggagaaagtattaagccagaacccaaggatggaaataccccttagtgagtcagtttagacttcaggctgttcatttttgtatgataatctgcaagatttgtcctaaggagtccaatgggggatatgttttcctcccgtgaggaaatgtttagttcttgagggaaaatccctaaatcctctatatactcaggtttagggaaggaaaacctacccctcacaactccacgcgcagggaaaatgatggacgtgatgctcgcctttagcttcctccctatctgatggaagaccatggaagacctcttggtctctgcaatcagaagtctcaagttgacaagaaaatcatagtccctaccctgcaggagagggtacatccagaaaaagcgaccatggactctattctcagaaatcagtccaacttagtgcagacctggccagatgaccagtgccctccccggggcatttcacccataaatgtgatgaggaaagcccataaatggtggtgaattttgctgagtggggttaagactggaaacccccctgcaggagttggttcttgagcaagttttaaagaaacaaggaactaggatgagtgtggaagaaggcgggcacgtctcagcccgtgaaaaaaactcacaggtgatcagtagtgagtcatgagagagagagcaagagagagagtcagagagaggagtaaatggagggaggaagatggaggaagggactcaagttctcaagacaggaacagggatctcctcatgaaaaaaagaagagaggaaaatgctcaatcagcagaacctgagcagaatattgaggtcaacccagaagccagctcctcacccaccctcacccagactggcgccctcatcctggagaagacctgctagaccctaaggcaggtaggagagagggtggtccacagtcccccagctttagaaagtttgttcgctcccaatgtccatctacccctaggaatccccactagttaaacagaattgctagatccctgtggaaaatacctttccttgcccaccatcatccccagaaataataactattttagttgggtgtgagacatagagaataaaagggggcatggtgccagacttcatttcatacaaatagctttaaaggagaagaggggggaaggagtttaatttagtttctaaaatgtttagtaatttgattgtgatcatgtcagagcaactaattcattttataaaatatcatttcactatgctctataagtagaaattcaatttggttcaaccattattgagtgatataaataaagcactggacttaacaaagacagaaatacagaaatcagtagaacatggatcccaacctaaaacttactctcttgtcataaaggaaaggagataggagtttttgcataaataacaaggtatcaagacagaattaaattccaagctggctttgaatgctctattttgccttaaaaatttatttactagtctcagtaatacattagtaaaaatcatgtcacttaattaattgtgttagaatcaaagaaacatagagttgggcaatatacttcatcctacccatcccacccaaatcttactctactcatctcattctcattaattttgggaaatcatcagaagatgtgttcgttgagtaagagattaaaagaaataagctttttgacccctgccaacaccccatgcccagggtggtcaccctccaatacaataacatgccaggaagagtaagttgccctttctgatgccgtaatctgccatcatcttcccatcttccagtctctttccattgcaagtcacaatctgggtctcagggattatacccgtcttagtctcgatcattgctttcacttgtgccactgagctggaccttcgcacctggaggaggtgcctctttgcctcatcacctgactccacaagaaacaagggcagctcctcatcactgggcttcaccactttcagggtaaggtggatggtcttctctttgtcaatgccataagatgagaggcttctccgtggctttaagatcttggagcccagcaaaagaacctggtcctgcacaggaaccttggtcttagaccggacatgttctttgatttttttcacgctgtcatatgggttggcatcaaaggtcattaaatcccattcctcggaacggacatgcacctgggaagtgaaagccacaagacagttacctaggatgcctgcctcctttacacttctactccccaccacaatggctccccctcttccactatctatctggtcctctagctcctattcagtagccagtgtccctctctttcttggaacttcttttttggaattaccaagttacaacacaaataagataatttgtcccattcctttataatcacacctttttttcgatcttgagaatggaaaataaaatcctgagcccccaaccaactgaacggacgctcttttgctcagggggaccctagagaaactttaaaaacttagtcattgggccaagggtggtggcttacacctgtaatcccagaactttgggaagctgaggcaggctgatcaattgatgctgggagttcgagaccagcctggtgaacttggtgaaactctgtctctactaaaaatacaaaaattagccagtagtggtggcaagtccctgtaatcccagctactcaggaggctgaggcaggagaatcatttgaatccaggaggcagaggttgcagtgagtggagatggcactactgcactccagccagggcaacagagtgagactctgtctcaaaaatataaataaataaaacattcagtcatgatggaacaggaggttggatatgcctcattgtatcttctcccttttgcagtttagacacaactgaccagcaaagttagagattataagactgagagaatggattctttgtggcaataagatagcaaattataaacaagaccgagggctataacaggcaaaagttaagtcatgcatcccttacacttaaagaataaactatgttctgccacaaagttttttcttttttctctagcagctaaacaagcactggccttgacaggaacaatattaaaacaattacagctcaccctgtgttggggaacacaggctaactgaccccgtgttccacaagccataactacagttttaattggacaaaagactgatttcagtaattttctcctgataagagaccactgaccatggactggttctggctagtttacagaagctgtgcatttgaatgcctttgtgtccctgcttcaccttttcatgtataaggcctaactgtaatgcaattaaatgttaagtctccacttcagagtgaccatgggtggtatgtaacatgcaagcttattcaatatgcatgcattaggaccccctccatgaatattcattgcctctgctataacctattggatatgtatacttagcaaaccccttcagcataaattcctgtgtcacctttcctcctgcaaagtgcttgcttttggttttcaatcagaagcaaaacttcccagcctgtcagaatggctaccttgcagactataacctttcataagaaataaactccccttctaaatttatgaattgtgtgattttttttagttgacaatctttacatttcgtttttctgtgcatttcaatggatgtaaaaaacatacctttatccatctcaaaatgtaattagtgattttccaccttatttacctgcctctcctatccagataaagtttgtcaaatgtcaacaagtaaatacgaggcttcaaaagatgtacatcagactctaaaaacaactctcaaagagaatttccaaaatatgacagcctcatgaagatactcatagccataggataccctctgtcaatacttcagaaggaaatcctgggtaggacacataatcactgaactgttagtttcttttcaaatatcccactgctttataataataactcacatactaccgtgacactatgttcagtgtttcttgttaatttatatttcttgtgtttttatttctatctaatgagagacaggactagctggatttcctaggccgactaagaatccctaagcctagctggggaggtgactgcatccacctttaaacacggggcttgcaacttagctcacacctgaccaatcaggtagtaaagagagctcactaaaatgctaattaggcaaaaacaggaggtaaagatatagccaatcatctattgcctgagatcacagcgggagggacaatgatcgggat

SEQ ID NO: 132 - DNA, 6914 bp, Artificial Sequence
  subsequence
tatgtatacatgtgccatgctggtgcgctgcacccactaactcgtcatctagcattaggtatatctcccaatgctatccctcccccctccccccaccccaccacagtccccagagtgtgatattccccttcctgtgtccatgtgatctcattgttcagttcccacctatgagtgagaatatgcggtgtttggttttttgatcttgcgatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtaattctagttctagatccctgaggaatcgccacactgacttccacaatggttgaactggtttacagtcccaccaacagtgtaaaagtgttcctatttctccacatcctctccagcacctgttgtttcctgactttttaatgattgccattctaactggtgtgagatggtatctcatagtggttttgatttccatttctctgatggccagtgatgatgagcattttttcatgtgttttttggctgcataaatgtcttcttttgagaagtgtctgttcatgtccttcgcccactttttgatggggttgtttgtttttttattgtaaatttgtttgagttcattgtagattctggatattagccctttgtcagatgagtaggttgcgaaaattttctcccattttgtaggttgcctgttcactgtgatggtagtttcttttgctgtgcagaagctctttagtttaattagatcccatttgtcaattttgtcttttgttgccattgcttttggtgttttggacatgaagtccttgcccatgcctatgtcctgaatggtaatgcctaggttttcttctagggttttcatggttttaggtctaacgtttaaatctttaatccatcttgaattgatttttgtataaggtgtaaggaagggatccagtttcagctttctccatatggctagccagttttcccagcaccatttattaaatttcttttcttgttctcccgtgacttttgatatcactgcacacaactgttactgtaagatgaacattaaaaagtcacgtctggccagtacggggatcgaacccgcgaccttagcgttattagcaccacgctctaaccaactgagctaaccggccccgcttactaaaaccccccctaaacctcttgagaagtttaaaggcgaacgttatttcaatcaccaaagaaacttttctgaacttctcggaactggaaagaacctgcgatctttgctaacttgtttatgatcctcggcaaatttctccgaaccctgacatttcaattaaaatacaattctgaggaaggaaagttcaaatgtcacctgcttcactgtttcatgacaaggggtttcagtgaccacccttttaaacgcaacgccatcaccttccgccggtactttggcctgcccttctcactcctttctactttgcttccgtagcttctgagcaacccgtgaagaaattgacggaaaacggaaggaattaccgccagtttctttccacgggccgtcaaggccaatataagtccccacccaggctcctctgctcgtaacttccagcacgattcccactgtattagcccatgtttcccagaagtgagtatttgatccctaccccaccccatttttagaaaactcaggaaaacgctctcgtatttctatctgaaaaggacactttagagaaatacgttccattacagtctttgtgttcgcattgtattccttctcgatgaaacaggtataagtccattggtattttacgcacgacaagacaactttgcagcccgactcatgacctttagattaagaaacactctccgggagctctgctgatcgctgggtcttacgaggttgatgccttctgtcccaaagagaactatttccctgcgtgtttccgcctggctgcccacttctccagtagagaaacagctgttcctcgggattcatttttggaagttctttgggtcctgggtaatggggccgcatcctgcagcgtcgacaaggtggttgaatacgcaggaacacccacagtacccagggactaataaatagctcaatagatacgtttcgaataattaaataaaagaagtctcaaccaacccccttgctgtattacagatggtccagctctctgcctagactattgtgctaatgtcctgttccttctcccggcttttgaactagattctcacctctctccaatccatctttcgcagggctggcctgtctttccgcctcacttctgaaattctatccattgccgcctctgtgcaaaccgactctggttaatacttcagtcggtatggctcccctcttccgcactttgcactgcgagaaatgcccttctttgaggcagctggaggcctcctcctcctgagagcttaattgtggacacagccttgagtagaggcgaaaaggaaagaaggctgggagacaatcggggaaaaacacacacctttggtctggtttgaaagctggcgcccgaaaaggaacaagaaaaaaaacacgcttcggaagtgtctgagattgctgcggccataaagcagagcactaaccctggtactgatacgggaggggagcaagactccacccccgggcctgtgcccatgaacctaggtgaggaggggcacttcttcctccgcgcccaaatgttgcatttcccaagaccaccctggcccgccacgcccccatcctgtgcctataaaaacccccgagaccctagcgggcacagacacaagcggttggacatgaagaggaacacaccggcgggagaacacagaacacccaccctctctttagtcaccctggaggcctcggagaaggcggtttcagtgatccgtgaagagaccctgagtcttttacatcccggagaatgacaagagagtacctgggcgacgtaactttctgggttttccggagatgcggttttctacacgccaggggccaagcctgagaaataggtggacggcaaggacaggagctctctccgcgccacaaacgcccctgctcacacctgggttccttgattttcctgcctatataaccgagccacccatggccgggctccgagctctcattctgcggactgggacaaaggggttaactgtgatgtgctttgtataaccccaggattctgcacttgcccaagggcgaggtgcaaatccaaaatcacgcacgactgaacgccgagatcagctgagtcctgagtgcctcacgacacaaaatccgtatgatttttaaaagcgctgtatttgtgctgacaatgcatgtcagatcccatctgtctttagctcaatgtgtgacgttagacacgtcgttttgctcctcaactttcttatttggaaaaaaaaaaacgagtgttctagatttgcagagggccttttcagagctaagttccagtagctatactgtaagttgaactcgggcagtcctgttggtgaggaaaaaggacaaattgagaaatgaacaaacacagaagttgcaaaagccctgtgacttacagcacaggacaagtctacaaaccctgccatgccacactaagctcaagttgttttaacatcagtttactcagacccgatgatccaaggtaagaccaaagccaaacggatccagcaactgggcagatcttcatgttccagcagctaagttccacggaataaacctatgattcgcctagtttagaaattgtctagtccctcctcagaaggaccttactaactttccccctaaaagtgtcctatgaatagctccagtccccagaccctttaaattctggtctctgactcacccttgttttaggcagtactgggactccatagaggtacggctctttgctcagcaagtttaataaatccaaggtagtagaatcaatttgttttcttggtcgtcttgtttggagggagtgggtcttctcaatgttggttctgatccctgcctatggatatttatgaataaatagatatttgcactccatcatcatgtggagctacaagtgttaaatttaacgatctaattacctctaaaataaccactaaattcaacaatctgaaacttatctaattagttccacatcctggaatgaagggatttaataggtaataacaggtctcagtctctaatggagacataatcacacagacaccagatgaaagatgagtaaagtggaaagcaataggtagaacaataatttgctttatgctatgatttttggagcgaagcaagaaaattaatcaaggaaacaaagaagaaatccaagacttagtagtgttggatttgcgtttttgtcatatgacattacttgtattttacaggattcacttggtctccatctgacctcctcagagtagactgctctttcccaatcactcctgcaattaccctaggagatatgtgatcctaaagtaacggggagttttaaatttacttctaagtacacctgcatgatgccattagtcagggcagggtagctgaacaaactcagctctgaatcctttaagcagaggtaataattgctgacactatttaatcggattctgaacatgatgtgccacttttccttttgcgtctgaagtgacaccagccattttcacagtttcttccactaggaggtcctgggagaaaagtgataaaggtagattcaagaatggttagtttgataagagaaacataaaggcaagtatctttttcactcagttcaccttggttctaagagtgggtctatataaagaaatggaacatgcttctgctaattaatgttcaggcagaaaaagtcatgaattccactacaaacccatgtcccttcttccgaagacatgacaaattgatgagggtatgctttcttctgtcggtaaaacgaagtctgggatatagattgcaatattagcagtgccatagtgcagaatttctcataattaacacttttcacttgaatacataagaaaatagaattggaatgtaaagttaataaagtaatcatgttataaacaatatgaacataaaagcaaaaattacatggtctttggcatgcagtctttgatttggggaatgcattttttctattgttatcaggaagaaagaacagaaacagttcatattcacatggactagacaaaagtatgcatttatgttcttactccagaggtatatcaagtcccccaatttttcctaatctagtccaaagagaattgaactgtctgcaccaacagcaaaacattggtacactgtattgatagtatcatttaaatcttcgatgagcaagaagtgtcaagtaagttgcagattttgttaagccctgcatgctccagagggtgggagagaaacacagaaaccattcatcgacctggttcgtgaatgaaagtttttaggatctgtgaaattctgagacatttcctccaaagtaaagtctcattgttgtatctcgaagctcccgtcacttagaagggagcactgtgctcaccatgtttctctgagctgtgaaagcggcgcgttccacacttggagacccttctctgacacatttaccaacagatactgaattaaaatattaacaaattgaatccagcattatctgtaaagcataatgcaccatgagttaggggagattgcctcgggaataaaaaattagttaggcattgaaaagtcaattaaatgtaatataccacattaacaaaacaaaaaatacatgtaatcatctcactagatgcagaagaaatattggaccaaatttgccattcattcgtggtgtttaaaaaatatcccagtaagctaagaatagaaggaaaattcctcagatgaataatgggcgtttaagaaagtcctacaactagcaatagagttaatagtggaagaataaatatgtaccctttaatattggagaaaagtgcaaaatatctgtccttactacttctacattttactgaaagtcctagctagtgtcagaaagtaaggaacgcatgaatgaagaataaatggcatactcactttagaataagaaaaatatattcgtttgcaagcaacatgatcagatacacaaaaaatcctagtgtatccacaaaaataagcattataagtaaatgcagctatgtcacagattacagtatacaaaaattaattgtatttctatgtactagcatcaaaaaaatggaaaattaaattaccactttcaataggtttaaaacaaatgaaatatttagagataaacttgtcagtgtgcacaggacttgtacctaggaatctacaaaagatggctgagaaaaactcaggaaagcttgactaattggtgttgtttgacatatttacagattgaaaaaggtaatattattaagatatcatttttttccctctgagttgatctatagatttactacaatcccaacaaatatcccagcaggagatttttgttttgtttgttttgttttcttttctttttgtttgtttgtttttacagaaattgacaagcttattctgtaggacctagagtaacctaaataattaggaaaagaaaaacaaagttagaggacttacaccaggtgatgtcaagacttattataaagctaaggtaatcaaaataatgtggtattgtctcaaggatacttacccagattgacggaatagaacagatagtccagaaacagaccaacgtatgtggtcaactgatttatgacagaggtaaaatgtttttttaaacaaatgg

SEQ ID NO: 133 - DNA, 4834 bp, Artificial Sequence
  subsequence of HOXA7
aaaaacacatgcacccaattatggacaccaaaatatatacaattgtggaattaggtaaaaacacacacacagaaatacatacacagaaaaataagcacatactcatacaaatacacacataaaaatacattaaaaagatacatgacaccaatacatgggtacccaacacttggaccatcacaaggacagccaccccacttttgcttccccactgccccctgccctccagccatactcacctcccctttcccagtcccctctggataaggcagtccacatttttctttgtcaccacgcatctttattttcggttacataaaacacagctgggctgggaagtgtgccttccctgaaccccaggatggagctgagcagggtacaggacaacacaggagatgaagggcattgcggagggcattggacctccccacccactacagttaactcaagacaacataccatgctacaaagtcaccccattaacacatcctttccaagtcaagacactgccttacaaatgaactccaagactatagaaatgataaaaaaaaatcttgttcaaatatacagtatctgctattataggaaacatcagggcgtacatatttaacacagctgaacagtaagatacaggagccagaggaaaggacagcgaagctggaagcatctccacagtcctgctaagcagaagctaacccacagatctgcagccagctcaggaacattcccctccagaagtgggggttgatgggcctgagctgtgggtgccaagccagagaaggagggattgattctagggtgcaagcacttaggatgctttttggaataaatatattatttttcgatttaaatagatgccaataccctgatcctggacctcagcacattctcagggcagcctcagggaccccaaaagctgcgggctgtaagcagcaggggacttgcctgggagcagtcggcactaggtagcaggcaagccagccagcacaaaataggtagttttaggggagtaggtagtagtgagattcactttcttgcgggtctgggagggtggtgctgggtgtctgccagtgttgggatacatagggacttcctgggaatggaggccctctggggctggatacataggtagtttgggggtgcctcgagcagaggcctgtgctaggtagtattttggacgcgccagagcagggccggctggcctggggttgggggtgtcttttggggtcctcggaggcagagggaatccaaggcgacccagtctctgcggccgctcagtccacaaaagttgggagctggagtaggtgatgggggtgggtagagtgcaggttggggactgggttgcttttttgtttttgtttttgttttttacattttcttttatttttcccatttttgtaagtaaaaccagtgagtctcttaaagacgcttttccgactgtccggtgcagagagggccccggatcggcccctcattcctcctcgtcttcctcttcttcatcatcgtcctcctcgtcggccttgtccgcggcagcagtggcggcggcagagggcacggcgccctcgggagctgcggcggcagtcggaccttcgtccttatgctctttcttccacttcatgcggcggttctggaaccagatcttaatctggcgctcggtgaggcagagcgcgtgggcgatttcaatgcggcggcgccgcgtcaggtagcggttgaagtggaactccttctccagctccagcgtctggtagcgcgtgtaggtctggcggccccgcttcctgtcaggtcctgagaacagacatgcagacacatgaacacaaggacagacaagtagacagggcactcgttaggctgctgtcccagagcccgcaccttcctcctggcctagtccccagcgagcatccccctctgccccaggccccgaactgagctaggggaggagggggagtgttagggaaagaccccaactgcagtgccagacgcgcaggcagctctgtaatgagcaaaggcacagaatctcaactttacaaccgacctttccagccggctaagcttccacaatgtcctgcttcctctgacaaaggaaaactgtaaatatagagtgtgagcaagtgggaaacgctgcacttttgccattcaaagatgagcccggccattcccctgccttgctaggcaagtgggcgactcttcccagcagcctgagccctcatccccaggaccttcctagggcaccccgaccctctgtcctcattccctcgcccccatcttgaaatggaccctggcacagggtcgggtgagaggccctggagggcttggctctcctagcttttgagaaagaaatgtcaggcagcaaggaaaatgaggagagagagaagaagaaagggagggagggtgacagaggagggagaaagagagacagaatagcgaacaaacttaatgttaaaattccaagacaaatggagttaaataaatttacgaggatcgaacccattaattgggccataaaaagttttatgagcctcatttacatacaatgctatgggctccacgcaatggcgcctccgctccaattaaaaccagaaaggctgcgccgggagtcacggggctaccggctcgcaacagcctggctccgctcttccggccccgcgccccgcgctccgcgctccccagcgctgcgctccccgctcccggtcccgctccgccagcctggcccgcctagcgactgcgcctacctgaagaccgcatccaggggtagatgcggaaattggcctcagccgcgccatgcagcgcgccctcgtccgtcttgtcgcaggcgcctttggcgaggtcactgcagagcccggggatgttttggtcgtaggaggcgcagggcaggttgccgtaggcgtcggcgcccaggccgtagccggacgcaaaggggctctgataaagggggctgttgacattgtataagcccggaacggtcgaggcgaaggcgccggcgcccgccccgtagccgcttctctgtgagttgggagcaaaggagcaagaagtcggctcggcattttggaacagagaagcccccgccgtatatttgctaaaaagcgcgttcacataatacgaagaactcataattttgacctgtgatttgttgtccggcagctttcagtgtcggttttacgaggtagagtgatatatgataacattacacccccagatttacaccaaaccccattttcttttggacggagctcgccgcagcacgtgaccgcccacatgaccgcctccgccaatctcagcagtcctcacaggtggtctcgctccgcagggcccgcagccgcctagaatggaagggcaagaggctcaaatatgcggccaaagaatccgcccgcgcccggcgggcctggcgcgtcccgcggaaaaagacctggaggctccgcgggagcgcccagctggcggccaacctccgcactggggtctgcggacgccaggcggcccggccccacgcagcaccccccaccccgcccccccgccgactcctgctagtgagccctggaccaagcttgggatcctccccatccctctcctgtccgcctgcccagaccctggaagggtctctgtcccccgcaacagcctgccccgcggtggccttgtgggcaggactcagctatgagcagatcgactctgcccaagtcttctctcacccaggtccagtgggcgacaggccggacttagactcggatccagacggggaaggcgcagcatctcttgcagctgcagagagattgccaccgcaaactggagccatgtggttcgaataaagtcaacgtctcccagcttcctttccttaatcggaggcacactgtttatccgccctaaaggaagcagtgaaatatttatctattaatgagactcatttgccaacagatttattaacgtggggttcccctccctcctcccggacgctgtagtgctgcaggctctgtgccttcgctcctgggcacctggctggctccagcagtccgataaattgctaaagattcctttgtcctttccacaacttctggttcccctctggcgcatggggagccagggctgtttcccccagcttggaaaaatctcgggcctgcacccttccaggcactcccaatactggaaggtttctggggtaggccggggtgcctgggaacaatacatgctttagagcggatttggagagggggctctggcgtctagggactgcaacccactgtggacttccttttcttttgaagacaccgaaaacaaataggggaaaccaccccctaaaggccacccagctgtggaggcttcacctatccaagtaccagctcacatggagctgagcgcacagtggtctcacttcctcccatccaggcctccaattcctaccttccaggcctccgacccctgcaccatgttgcaccagctgaagcccctgccgcccaccgctcggtggaggatggtggggagaacccttcccctaaacgcctcataaactgcccggctgcgctggaagcccggccaccttttactgctcagctcgattacacaccataaacccgacctcacaatggaattgccccggaaattctcctgtaaattgtagagtttagttccttgtgtactgagcacttcccatcactttcagagggtccaggccgtccccgtttaccgcaccctctcagggggccccaggaatacaaaaggtggagggagggttcagatcgggacgccacggagccccagttcctgcgcagtgaaccaccggggcttgggtaggaaggcgggggctggtggggcaggtgggcgctgggctgtctcctcccgggccacggaa

SEQ ID NO: 134 - DNA, 6555 bp, Artificial Sequence
  subsequence of ZNF529
tctacctaagcaattggtctcctccgtaacaaatcaccgtgtgaaggcagactatttctatgtcatgagaatgcttaaacaaaaagagcacacaggagaagtgaacatactgtcaaggtgttatatgaaaattaactctaggaagggcatctgatgaggtactggaaagaaggagatctgtcttcataatgtcatcagtaactgggaagagaaggactagagaccaggtaaatagataagtcactatttggtgagcatttcaaatgaagttattccacatcctgaaaaacaaaaatgcatttcataaaacaaagaaaaaaatcaaaatgacctccagctttgctttaaaatgtgcaactacaatgcttttcttctctcagagctcttctcttggaatacagcaaaatgtggaaagaaagggaatcacaatatactcagcctttcttggagcccagacaggctaaataagaacacattcttcaccaccggcaagcttccataacccatgctgaatatgaccagatggaagtatgagaaagactggaatattgttgactcttcccttcataaagaaaagtcaggttcaccaaagcctgggactgaaatgtttctgtattcacaataatttcctttagacagctatgaaaatgctgtggatctgcaatattcaacttgcccccacagagcttccaaaagggatcttttggctaggcacggtggctcacgcctgtaatcccagcactttgggaagccgaggcgggtggatcacctgaggtcaggagttcgagaccagtctgaccaacatggtgaaaccccgtctctactaaaaatacaaaaatgagccgggcatggtggtgcatgcctgtaatcccagctactcgggaggctgaggcaggagaattgcttgaacccggaaggcggaggttgcaatgagccgagattgcgccactgcactccagcctgggcaacaagagcaaaactccatctcacacacacacacaaaatctttggagaaacgaatcctactcctggggcctcaatatcatatcttcccagaaccttgccttgggagcaattccagctctaacatctccatatcaaacacgaggctgtgcatatcagaacacagccttgtccatgtgaaacacaaggctgtgctttttcagttggaaacttcattctggatctcaaatatccaactttcttttcgatagccatttgactgcacactgtgcaacgtggttataaggtgaaatgactgaggcagtgaaagaaatcccagtataacaaactccaagaaaccacgtaacttaaaattggccccagaggttggcgaggctgcatgccactgtgtattatgcattatgaaagcgacaggggccatgctttagaacagaaggtctcagagtcttcacagacacagatcgagccatacggggaccttgtccacgtattcctgagccccagaactgcagaaatcaaactctacaaaagctgcaccaggccagttttgatcacagtctccagtgggtcacaacagcgctgtcacaggatcactgataacagcatgacacattcatagtcaccgctcagcgccgtcacacaaacggccccgcccccactcagtcccgcgggcccaatcggggacagtcagggctgtcccgggctgtgcacctgctgtgcacacacaccccctgctgtgcacaccccccgcccccccgggaccatcacaggcgcgcccactcacaccaccacacacggacgcagccacagaacggcacacacaaacacaatcacacagtctccctccccagcaggtgacttgcacacaggcctggggcttctgcctccacaaaccctctccgcgtcccatgaacccaggtcccctgccgtgagctcttctcgcatccagacccgggtcacctcaactcaccacatggaaaacgaacaccaggtcaacggaaaagggagccggggtcctggcagaagcgtagtgccagccgcaatagggcggccgtgggtgcaaacggaggggagcgccggcagctagcaccgcgcggcgactaacgggccgccccggagactcctgggagctcaggcccacgcgcgagtgcgcaggcgcagacgactcaccctgggccgggggtgaggcttggtgggtgccaggggtgctgaggaggccaaggagggcactgggagctccgttccgggagagctgtctaaggacgcggaaccgggtggggaggccagggaggtaaccaagagtccgagccgggcctggaaattctgaggaggaaaccgggaccgcggtatccagacgctccgatctgtacgagccgggcctcgggcgtggtccatgggcgtgaccttgagggtgacccggagcgagctccgcgaagggggattttgcccgttctgatcccgcggaagcgccatcgctggcggcgggtccccgcgcggagcgggcgctgtgtgttcgcgcgcgcgggcgcgcgctgaataggggtggcctcctgcgcccaacctgctgcattttaggtgcttgcctctctttgggatgttgtggccgattgtgtgtgaaaagtgaacggagtagtgttgctgcgcgaggtttcctctgcgtgtgtctatttttagattaggtgtgagtcgcatttgagactgcctttgagtgtggagaattgcaagtgatttgtaggtgtggttgtgactgcctggagtcgtgttagctgctgtctgaggttgtgaatgacaagtggggcctgtaggttggtggttctctgaaggatgtttgattgactgtgactctgcatgtgtttgtaacctactgttacttctggtggttttgtggccttagtgacctaaatatttgaatgattgaggaactctgttatgaccatggctagttggttatgtaagacttgattatctgaatagtagccattaagcctgcctccaaaactgtaactaagtagcaatttagcaaataaaatagagataacagtatcagaggggaagagatgattttcataattcccagattcatttcctggaaacaccatattaactaaaaccagtatgttgaaatgaatagcattccattggggacatgcaggaaattttttttttttttttttttttaccataactaccacttttgttgttgttttttgtttgttttaagacaaggtcttgctcttttgcccaggctggagtgtagtggcatgatctctgctcactgccacctctacctctgaagttcaagtgattctcctgccccagcttcctgagtagctgggactacaggcttgtgccaccacgctcggctagtttttgtatttttagtagagatggggtttcaccatgttgcccaggctggtcttgaactcctgacctcaagtgatcccctccccccgccccctcggcctgccaaagtactgggattacaggtgtgagccactgtgcctggcataactaccactttcaacacttgtatatgagtgaggtttcctactcccttaggcttgctaagataataaaaagtatttgatagatttttaataagtccagtctcactgtagtaattaaaatagttattttgcgggcctggtgcagtgactgatgcctgtaatcccagcattttgggaggccaaggtgggcagatcacctgagatcgagaattcgagaccagcctgaccaacatggagaaaccccatctctactaaaaatacaaaattagccgggcatggtggtgcatgcctgtaatcccagctactcaggaggctgaggcaggagaatcgcttgaacccgggaggtggaggttgcggtcagtcaagattgtaccattgcactccagcctgggcaacaagagtgaaactctgtctcaaaaaaaaaaaaaaaaaagattattctcaactacaacatagaccagataggcaaagatttaaaagttgtcaactgtcttcatcagggcttgggaaggtattctcatccactactatcagtgtaaattggcaaaaagtccaagaagaaccgtttggaaatacacagtaaacctgtggttagaggtagatggaaggcagagctgcagagggaaaaacacagtgttccctctcttcaagctaaaacaggttccccagaatcctgtatactttaccaagagactgggtttctggtgtttgtgagggagcctgatttgtctgttttcccactaactaccatcctggacatctgaagggtacacagggtggacaagtgctgaaatagtgaacattcatgcagatttaactgagttaattctgggagttctgagtcaagtatggtctcacatacgtatatcctccatctttgctttctcaggactgtttctttttccaaaagaggagctcaaaagagaaagttcatctagaaatctcaaagccatgtctcaggtgagatgatgtttcctctctttctgaaataactttttttcatggtgtatgaacatgagcttcactgtcctttaaccttccctgatattgggatttgcttccatgaaattagtcttcagtcagggccagattaggctgaggcaggcaagtggcatgaggtgaaatatttaaggggtaccctcagggctgtgccctcctaaattttgcattctaggtgcctcacctgcctcaccctgtatccagccctgttcacaatttctccattccaaccaaatcaacttaagccttccccaagtgtaccttctggttgtacatattcattgggtatatcattcatcatctcctttctcagtcttctcacaaacaaactgctggaatagaaaggtattgtcttgatttaaatgggaatatatggcttgaatgaatttaacttgtcctgatgctgatttgagacctaagctgtgagtagtcatggagcagacgtggaaaaaccttgatgatctccgctagcctaggttttgcaatggtacgttgggaaatagaagtggtagatcccagcattagtttctggaaagatgtcctgctttgtgaggtgtttaggatagcaatttgattgcaggcttgtaaaggaaacacgttttatgttcctcctctctgatcttttttcattcatgtctgctagccattcctacaaattgtgggctgccagtgaaaacctttccagtattccaatgctgttagagatttgttattttctttttagtttcttctaattttttatttgcgggtgcaggagaaggaaatccatatgccaggccacttttgaaatattctattgttgatcaaacagtactttgtcctcttttcatatctttcagaaaattcctctttagctcatccaccaacctttaaaatactggttttcctcagatgctaattggttctcttcttcttcacagtccgtttttctctacgcacaatcagattcactctgaaggctacacttagtgtgattctagtgactctcaaatacccctctccactctagacctctgtccagagtgtcagaaccgaatattcaacctccagtagctatcaccacctgtatgttccacagacaaagtacatttccaaagcttacctcattacttcctttggtccagcttacattttcttccgtttcccattttcactttctagtcccagcagccatgcatttgcctatgcagataaaatgcctgtatcttttccactctacagccaatttacgaccatctctgttgtaatggtggtcatgtaggagcagttgtcctctgtcagcccgctgacacttccttaatctaggagacatttctaaccatggttacttaattgtcatttccacctaacatgtgataagccttttccagtcttcaggagtattacctcaaaacttttatcacataacgtgtgtcaacttatgtttattgatgttctttacattccttaaagcaacagacccaaagtagggcatgcctcacagtagaatgtataagacggtctattggaaggaaataaaatactaacaatttacattcgagagcagtattcattaacaatgtagattttcataaatgcctattgatggtttataaatagctacaatgttgggaatgtgttcagtatatctttaagaccactgttctgtaggaaaaaaaaatctagtatcatgtcataatatccccattgcctatctccatataccttttccttcttagagctcaaagactgtttcctaacatactgtgccttttgctatttcctgatttactaactggtttttttctccccatgaatttttaaaagctgatgccctgtattcctacaaaacaaattcgttgtgagtacaaaacttttatgtcctcaatgaagcctcggagtacaaaatgaaatttttccttgttttgtgacactttaccttgtacgtatttctgtcattgcatctgtaaaacctactaagtgcatgtatttacatgtatgcccctctcttagcctataacttctcatgagcagggtcagggttttattcattttggtctaccaggcaaccagtcacattcaggacaaatagtgtgatctcagtatg

SEQ ID NO: 135 - DNA, 4378 bp, Artificial Sequence
  subsequence of PRLHR
ttgatctacacatcatgtgggtggtagcttattcttctgttctgtccttaagtaaacttctgccggtccttgatttctgggttgatcatgttccaagaatcactacagagcaggtgctgagcagtcagatgacagctgttgatctgttgcaagtgagcaattagagactgcaggagaatgccacgctacacagtttacattgagtattcagaaaaccaaatatttgagtgcccactttgtggcaaacactgaatgtgtacccacacatacacacacatacacaaacacacatacacagaggatgtctgtccttaggtacaattgaatgacctcttaagtgaattggaccttttcctctttatttatctatctcttcattgactaattcgtctcatcatttttaaatagtagttggggaggaaaggggagaaacaatagcaagacctttctaaagggagcttgtaggtgtttttgtactataaactcaagtggttttaaaacgaaacccttttggcatataaaggtcttactcaggaaatgctgaaatcctagatgtagagttttaatacatatttacctgaaattctaggaatgcaagggttcaaatggactaagcaatatcatggtactctcagctgggtcctttttctaaaggcaaactgaaaatctttaccaagcccaatgcattccttttaaatcccattcatattgttcatttcaccaacgagcaggaggaggggacgccaggaaggcaatcgtggttctgaaatacttttctgaacgtggagaaaaagttttgtttgtccttcaagggcataaagaagaaataagaaatcctcttccctctctttggcctcccccaagcaaagagcaagactctcaccaaagcccgcttaacattttacaaacacacaaggacaagacagacagccggacaagagggctgggctggaaatgttgccctatgttggcttagctagctctggtgctgagaataagcaccagattgacctcgagtggtgccctaggaggccagttgaagtggagctccttgaccaaggcctggctaagtggcatcagatgaccacgctgacggtcatattctggccatggggggctatcttgcggggccaagcgaccaacagtttgcgcagctcctcgcggaagctgtcgtgcagccaggcgtagatgaaggggttgtagcaggccgaactcatggcgagccagtggcagagcagctgcaccagcccaaaggcgtaagggtcgatggcgtgggggtcgaggtcccgcagcaggttgaagacgtgcagcggcagccagcagacggcgaacaccaccacgatcaccaccagcaagcagaaggtgcgccggcgccgagcgcggtcccagtcggcctggctctgggtcacgcagcccggcaccacgcggttgcggagcttcactgacacccggacgtaagacaggaggatgaccagcagagggagcaggtaggtgaccagcagcagcccccaggcgtagagctggcgctggcgctcctgggagccccagaactcctcgcagaggcgcacgtcgtgcggcttgagctccacgtgataggtgtgcacggcggcgggcagcgccagcaccgcggacagcgcccagatggccagcacagcgtaggcgctgaggcgcagcgagatgcgccgcctcagcgggtgcaccagcacgacgtagcggtccactgcgatggtggtgagcgtgaacaccgacacatagacggtgaccggctgcaggaagaagaccaggtggcacaggccgccgccgaacacccagccgcgtggctcgaaggcataggccagcgtgagcggcacgcaggcggtgcacatgagcacgtcggacaaggccaggttgccgatgaggaagttcgtcacgttgtgcagccggcgcacccgcgcgatcaccagcaccagcaggcagttgcccaccagccccacgaccaccacgacgctgtagagcagcacgatcagccccttcagctgatgcaccagctgcaggctctggaagggcgtgacggctggagcgtccgcgccagccaccgacccgttgcccgccgaggcctctgcgctctggttggcgggagttgtgaccgccggcggcagcccagaaaataagtcagaaaccctggggccccgagtggtcgatgaggccatggccacctgttcaaaggtaatcaaagtccgtcacttcccttgccatactccatccgacctcctacaccagccgcgcccctcccccatcgcctgagtcctcccttgggaataaccggccacagaacaacagcaaaagtagcaaaaagtgttgtcctctcacctctgtaaaataaacctaagcacggttagaacatacggtttatttaaacgcttccgcctctatttacggtcctcgcagtccacgcaggcagactcaggagcgcgatcctacctgggagtggggtttggagagcgggaaagcactcgcgggaagaaggggcagaatttctgctggccctcggtagtcctctgcccacgtcggtgatccggtctcgcttctccgcgggagctgaacggatccaaatgtttcccagtgggctagcagcttcttagagggactgggagcgcctcccctcagctccgccccccactccagctcccatgtatggaaaataggggtggagcgcgctctgccatgctcggtgcttagagaactagtgaagaggacagtgtggggaggatggaatccgaaacgggaggaggggccggcaaacctggccagagcagcgaagaggacgaatctctactgcaggagcttgcacggcccctggagggtcagggtccggaggagcgcacagtggaggagggcggaggagaggcgcgaagcgtcaggctatcgggaggagccggtgcagtcttaggagggaaggagcggggaaggaagtagcggtgggagattgcaatgcagctgggggcttgagagaggcacgactgacccgttcttaaaggctggatcctgcctagcggccaggttgcggagggcagagacgctagagggctcgggctgggatttaacagcagagcctggagctggtcgcggacctgaggaccctcaccctcccgtggccccgccacacgagccccctgcttggctgcagcgcgctcagcgggcagcgggtccggccaagccaaaggcaggagtcagcaccacggacagcttccgctggatctgccgcccctcgggcggatgatgtggggaggagcaggggaaaatgactagggaattcctgcccctctctggatccgggtgtttggggaaggaacctggtccggttcaaagacctgcctcgctgcctgctttctgggctggggctgagaagagtagggtggagtggaggcagctgagccgggggcctgggggtcggagactcatgacttttgctcctcttggtggtcaccgcactcagtgccaaggacggcgtcgtcttttacctcccggggtcagaaaatgcagctctcctgccagttattctacacaggcacacttcagtctggcccagcgactccccggctcaccctttctctcgctttctttctcggctaccgcgacctgtgccgtatctggctgcttctcccagcacagagtagttcctggactgagacttgacccctccctccttgactgaacacccacaggcctcttctcttcattttaggattttttaaaaatactggtaattgcagatttcagctagggtaacaaaagtaacaaacatttgagaagcttaggcgtttcataaaatcttcagtagtattaagtcctcattgaaatgcaaattagcctggcccacggggcaagagccaggggaggaggggcctttgtaagctccctccttagaagctatctcattccctcagcctcttgtttcctttttccagagcatggagcttcattgtctcctctctgataagtccttgtactttgtcctctggctgcagatattccagttagactggaaactaatatcaatttattccaccctccaaatttaaatgaatcactcatatagaaaaaagtcaatattggaacaaaagtaatcaatgttatgcacctctgtccatcctaagctcacggtccctgagttataatcaatttagctaaaaccagtgtgtgtgtatatatacatgtatatgttaggacaagatctcactctgtcacccaggctggagtgcagtggcacaatcatggctcactgcagcctcaacctcccaggccaaagccatcctcccacctctcagcctctcaagtagctggaactacgggtgcacaccaccatgcctgactaatttttgtttatttgtttcttttg

SEQ ID NO: 136 - DNA, 6673 bp, Artificial Sequence
  subsequence of ARL5C
tgtcaaaatgcagattctgacccagtgagtctgggcaggggtccaggcttttgcctttctttctttctttctttctttctttctttctttctttctttctttctctctttctttccttctctctctctctttctttctttcacagagtctcactctgtcgcccagtctggagtgtaggtacgatcatgtctcccaggttcaagcaattctcccgcctcggcctcctgagtagctgggattacaggagcacacccccacgccaggcttatttttgtatttttgtagaaacggggtttcgccatgttggctgggcgagtctcgaactcctgacctcaagtgatccatccaccttggactcccaaagtgctgggattacaggcatgagccaccgcatccggccacagttttgctttttttttttttttttccttgagatgtagtctctctctgtcgcccaggttggagtgcagtggcacaatctcagctcactgcaacctccgcctccctttgtcgcccaggctggagtacagtggcgcagtctcagctcactgcaacctctgcctcccgggttcaagtgattctccttcctcagcctccagagtaggtgggattacaggcatgtgccaccacgcccagctaatttttatatttttttagtagagatggggtttcgccatgttggccaggctggtctcgaactcctgtcctcaggtagatccacccacttcagcctctcaaagtgctgggattacaggcgtgagccaccttgctcagccctttgttgttgttgttgttgttgttgttgtatttttagtagagacgaggtttcaccatgttggccaggctgctcttgaactcctgatctcaggtgatccgcccgcctcggcctcccaaagtgctgcgattacaggcactttgagagcaacagcgcccggccatggttttgcattcctaataagctttcaggtgatgcctgtgtgactggtctgtagaccacactttgaggagcaagagtttagaaaaatgagacagacacagtgagggacatcagagaggtgctttgtttaaagtgacatagaaaatatacacatgcttttatatgtaaataatgcttctgaaaagttacgcaaagttacaataggaggaaaactaggagccagaggcaagaaggaaacttacttttcaccgtgtattcctccattgtttgaaattttcctttataattttttaaaatcattacatgtattactttttcatggaaaagcttctcctaagtaaagtactttggaggctgtaaaatgggagaggtgccatccagagagcgaatatcggggaaagctgcgatcggtatttactcagcaccacccatatgtgccaggcttgctagaccaggagctacagagcctcagtgagcctttgggtttgaagaaggggaagaaagttaactggcagaggggacaggggtgagaaaggagggacctgccgctagagggagcgaagagcagggaggcagtttgggacacttggcaggcctggagctgggtttggcagggcccccgggaagatggaggaaagcagtgggagctagggttgcctgtacggtgcaggatttgaatgccagaccgaggagccgcagtactaggaaggtccaagcagcgtgcccaatcgctcctccggactacagaggagcccgggaacccagggaagagctccagtgattggtccccctgcctgtcagtctggtgggaggcccagctctctaccctccccgcccgagagtcacttacaaccggtagagaatggtggtctttccttcattgtccagtcccacgatgatgaccgtgtgctctggaagcgtcggaggaagggcagcgtcagggccaaacccgaagaccaagggactgtgtgcccccaccagacctcccaggaaggagatagcgctccccgcaggctggggacctctgcccaccgtaccgcgtctgcttcccacccagctccgacacctgccccactccgggacggtggaaaagacagctgagcccccacctcccttcacattccagaaaagtgtctgaaaggcccggggcgcttcggggcttgccaagagacggtgtttagagaaagagcataacgcgaagtcacaatcgcaggaaactcgcagcagccccccatccccgccgctggctccgtttagcggggagaaaggagggtcgcccagctttgcgtcctggggcgcaccgaagcgccgggacccaagaggagcaggcagggacgggggcaggggaggccgcggggcagtcgaggagcgcccatatacgaccctcggagcccgaggtccccccgtgcgtccgaccacagatcagaggagtcccagaagagggcgagatcaaagcgctcgcttggatgccctgtgcgccccttaccctggttcccgaagatgctcattaacttggcgatcagctgtcccatggcacttcccgggccggacaggtgcaggagggctcagcggcctcaggagcggagtcggccctggtattcggagctccgctcccccgggagggtctggcagattttgcctacgaagttagggaagccccacacgtcaccaacctgacctgggaaccctctgggacgctggcccttcgtgggcactatggacaccccagtgactaagggctgcctgtcccgggcccaagaggtgcaaggaatatgggggttccaggctccagccctccccctcgccctgcgaaaactcctgagttctccgggtggactgctccactaccgcaaatcaggggagactcagcactgcgcgcggaaccggatcccagctctccctcctctgagtgccatgtctagggcactagactgaagcaaaaccccaggcgacccactttctgtacctaccagctgtgtagtacagacagggctcaccgatcctctctgagctgggagaatattccaaccctccacagatgtgcagttggttcactagcgtgggtcttgtgggcccactcaaattgcattcccctggggaagggcagggtctccccttgatgcttggcaacttctaggataaatcagtactcctacaattcacggacaaacctactgcccaggcacagcttcctgatccccaaaagtgggcgcctgaagacaaggatggagcatgaagagagatcctccggctggagaccctgcagagaggccccagatacctggcagaggagtacatgggccaacagagaaccaggaggccaggcctgaggagggaatggaaggtggagtttcctgctgttgcttgtgaatttctttctttgcttaagctggggggaaaaaaacaaaaaatcagataacaaaaggagaagtccaaaaggcaacgtgccctcaggctgcaggaatggttagcaggagatccagccaacacaccagggagccagctccttccacccgactgtactgccttcctgcagcctccactaaggtaacaaccctccattaaggtaacaactttctaactggtctccctgcatcttcctctcctttcccaaggcatcctctccatggcccctattaaccttcctaaagcacagctcagggtctgtaactcccccagctcatagatcttcaatggctccctattgctcactggattcagatcagtctccacttgacattcaagagcttccacgaacagcccccacctgccttcacaggtttatctcccaggctctgtatctactggttcctgcctatacatgcctggctctttctgatggtgcctttgcttctgcatttcctccttgagtgcctttcaccttctcagaaaggagaatgcctgagacccattagctgccctgccataaaagccacctgcttccccagccctgacttggttctccagccacatggggcttctccctacactgggcttatagacgggcaagtctgtttcctcccatcaactgtgagtaccagggtgggattcatatattgttaaccagcatatgacctcccccgccagcccaatcactccctgcctggtccagaaccctgcacacaggaggaatgtgttgctgtcagactgatgtagaatgagttaagctacagcctgagaaatggcacttggagtattaaaaaaggaatgtgaacacaagtttaagaggattgaggaaggctgggaagtttgcgggaagaagaaggaaaatagctgactaaatacagactttcatcccaccctgcttggtgcccagagttagaaggatagagttgactgccagagcccatctgggctgattgcatttcagccttaggagagaaggagccaagtctggggctggggggtggggcaccaagggtgctgagggaagcagcagtgtcttaagcattttaccatctgtttcctggggttagtgggatgcggggaggaacatcttagtgcatgctgccctccccacctcctctttgcagaattgaaagtaacactctgcaagctggaaacacattcagtaaacagacaaggagagggcatctctctgagcatactctggctctggaagctgcctgattaaaaaaaataataattaaaagagaaaagaagaaaaaaaaaagaaaagaaaatagaaaaacagacgagaagatctgcctccactttgaggcccaggacctggatcttaagaagcattcattcgttcattcattcatttgacaaatatgagccaggaaatacatgaagtagacaaaacagacctagtccaggtgtttatgtttttgttttgttttgttttgttttgtttttttgagacagaaccttgctctgtcacccaggctggagtccagtggcaggatcgcggctcactgcagcctcaacctcccaggctcaagtgatcctcccacctcagcttcctgagtagctgggagtagaggtgaaagccaccactcccagctattttttttttttttttttttttgtagagatgggcttcgccattggtctccaactcctggactcaaacaatctcccgcctcagcctcccaaagtgctgggattacaggtatgagccactgctcccattcccagtctgggtctttatggagcttgtggtctagagagagagacagatacacacacacacacccctctgagagacagtaggaaggcctgatttagaaagaaagcaaggcacccttctctgaagaaaatgagaggtgaacctagctggacttcctgggtcgagcggggacttggagaacttttctgtcttacaagaggattgtaaaacgcaccaatcagcgctctgtagctagcaagaggattgtaaaatgcaccaatcagtgctctgtaaaaacgcaccaatcagtgctctgtagctagcaagaggattgtaaaatgcaccaatcagtgctctggaaaatgcaccaatcagcactctgtaaaacgcaccaatcagcaggatcctaaaagtagccaatcacagggaggattgaaaaaaagggcactctggcagggtgcagtggctcgggcctgtaatcccagcactttgggaggccgaggggggcagatcacgaggtcaggagttcgagaccagcctgaccaacatggtgaaaccccatctctactaaaaatgcaaaaattagctgggcgtggtagtgcccacctgtaatcccagctactcaggaggctgaggcaggagaattgcttgaacccgggaggcagaggttgcagtgagccgagatcgcgccattgcactccagcctgggcaacacagtgggactccatctcaaaaaaaaaaaaaaaaaaaaaaaaaagaaagaaaagaaaagaaaggaaaaaaaaagaaaaagaaaaaagcgcactccgataggacagaaacagaacatgggcggggccaataagggaataaaagctggccacctcccagccagtagcagcaatgtgctcgggtcccgttccacgctgtggaagctttagttctattgcattcttttgctctttacaataaatcttgctgttgcttaatctttgtgtccgtgccatctttaagagctgtaacactcaccgcgaaggtccgcggctccattcttgaagtcagcgagaccacgaaccccaccagcaggaaccaactccggacacaaaaagattattggagctaagacaaaatttttctctaagatactgaactgggacagccccttgtccctggaccatggtggcctcagcctagttctctttagtgtagaacagtggaaaccttaagccacatgtaggacctgggcctctcccacatcacctcattctgggacagaacccactgcaaaaacaggagcagctagtcagggaaggaaacaaagggtcccaggtagcagaagtccccagtccctcagttcctgcctggtctcagctgacttcatggttgtcgaaatcctcacacatggacaaccactaacttgctgatggtgtagccagagttaattgagagcttctgattaccaagaaacaagaatctgcccccatctctcgctatctccaagccagctagaaatgcctttcctgcaagttccaggcaaatgcaatttcttttctctcttttttttttttttttttagacagagtctccctccatcgcccaggctgtagtgtagtggctccatctcagctcactgcaacctcccc

SEQ ID NO: 137 - DNA, 1006 bp, Artificial Sequence
  subsequence of PAX9
cggagttcggtcgattcgatgttggggtttaaagtttttttagttatacgtttgggtcgagtcgtttttagagttttcggtttttgttttttttgggcgggtgtatataaggattcggttagcgtcgagtgtattggttttttttatttagtgtagtttttttttttatattttcggttcggtttttagttttttattgagtaaatatttatttagagagcgagtcgtttttcggaggaggtttcgacgcggttagggaaaggttttttggtttttttttggtttgagttagttgtaggttttattggtgttttggttttggttttaaatttttttttttagtcgaaattttttcgatttttttgagattttttgagtttagagtgagtagtgtaattaggcggtagattttagttgatttttgtttttgttttaggttttgtatttttatatttatcgggattgattagattttagtgttttttttttttttttttttattttttttttttgttttagattcgtcgtttttcggtcggttttagcgggtcgttgattatttcgttttagtattcggttcgcgtttcgggaaggttttagtattatttttttttgttgggatttcgttgattttgtttgtagtttcggttttttttgtttttagagcggggttttcggtttgtcgcggatatttagtggtttaaaatatttaaaattgattttttttgcgtttttgagaatgttaagagtatttagtatttgtaatcgtcggtatgagtggggtttgcggggagtcgttagagttgcggataggttttttgtttattgagttgttatttttggtacgtttaggggattcgtagtagttgaggttatatggttatagttaagtttagtaggtggaaagggagagatggtattgaatttatgtgagaattgggaagatgtgtggtagttgaaggagttggtaggaataagagggagttgtatgttatttttatataat

SEQ ID NO: 138 - DNA, 1001 bp, Artificial Sequence
  subsequence of HOXD10
tggtttttttgagagtcgaagtttcgtgtagtttttcggggcgtataggaattcggagttaggtaggaggatcgagcgggaggataacgtagggattaatttatttatggggattcgggttcggttagttgtttggtttttcggttttcgcggttttttttgttttcgtttttttagagatattttggtcgttttttagttttaagggattttttttttggatttttggggtgggatgttttagggttcgcggaattcgatttattttttcgttgggttgttcgggattaaattaattagtttgcgtttttattcgtttgttttggattcggtttttttaagcgcgttttgtttggttgtgtgtggggggaatgtttttgtgcgttggcgttagggtattttggttttttttttcgttcgtgcgtgtttatttggaggtttttagagttgagatttttttttcgtcggttgggtttaggggtcgaagcgggggacgcgagtggggcgggttggtcgagcgagttttggagaggcggataggagggcggcggagagcgttgggtcggttgtttttagcgcgtattatcgcgggcgcgtagtagatgtcgttgttgttcgtgtttattcggtcggtcggttaggttttggagtacgtgattcgagaggaggttgcggtttaaggttatttttaaattttattggtttggttgttatgtggtcggtagaggtatttataattatacggggaatgtttttttagagatgttagtttataaaggatataattttttttttttaaatttttttttaaaatgtttttttttaatagtttttttgttgttaatatttttttagtagattttttgattagtgtttgtaggagtgatagtttttattttagtagcgttagtatgtatatgttattatttagcgtagatatggggatttatggaatgtaaatttgtggattgttttcgtttttggttaaaagagaagtgaat

SEQ ID NO: 139 - DNA, 468 bp, Artificial Sequence
  subsequence of TBX20
gtggtttttgttttttattagtgcgtttttagtttggggtttttattttttttagagggattttggaaaatgggacggttgtcgaagagaaggcgaaggttagggcgcggagttttacggtaattacgcgacgcgtagtgggagagtgcgaattttatggtagcgttcggattaagcggatatagatttttttggttttataaaggcggttgtgacgaggattttttacggttttgttggagggcgttttgttagttttgtttagtttgaataatttaaatttttcgaattcgtagggtaattcgtttcgtttggagtcgtgtaaatttaggttgatatatatatttaagttgtatatgtaaatatttgtttttttttttagttagtcggtttggttattagaagttttttcgtgggttttaaaattaaaataataataataatttttaaaattttaattttttattg

SEQ ID NO: 140 - DNA, 1003 bp, Artificial Sequence
  subsequence of CACNA1B
tcgagggtatttaggggcggtgcggtcggtagagtgggtttcggggacggttcgggttggcgttgagcggtcgtcgaggatttataggttcgttcgtttttgggttcggtttagtttcggggtgggaggcgcggggcgagggaggcgtagtcggaggggtcggggcggggcgggggcggtcgcggggcggggtcggcgcggtagggtgggcggggtcggcgcggattggtcgggtttcgttacgtgatcggttttttatagggcgtggcgcggggtcgcggagtcgggtgaggcggcggcggttgcggcggtggggtcgggcgaggttcgttgcggtttcggcggtttcgtggttgtttcgttttgagcgtttggcgcgtttcgcgttttttttgtcggggtcgttgggtcggggatgtacgcggggttcgggagttatggttcgtttcggggacgagttgggcggtcgttatgggggtttcggcggcggagagcgggttcggggcggcggggtcggcggggcggggggttcgggtttcggggggttgtagttcggttagcgggttttttataagtaatcgatcgcgtagcgcgcgcggattatggcgttgtataattttatttcggttaagtagaattgttttatcgttaatcgttcgtttttcgtttttagcgaggataacgtcgttcgtaaatacgcgaagcgtattatcgagtggttatattttgtcgggcggggtcgggcggggtcgggcggggatcggggtgggggtcggggtcggggttattttttcggtggtcgggagggcgcgatttggggaggcgtcgggagacgggcgaggggggtacggagcgttttagggtttttcggagcggcgggtgcgggagtattttcgtgagtgcggcgggcgcggggcggagtttttttgtttcggattcgttttttcgtacgtcgggttttggggattttggtcggtgtatagtgcgcggttagtttggaggg

SEQ ID NO: 141 - DNA, 1058 bp, Artificial Sequence
  subsequence of ZIC1
aagtaatagggtaggagaataagtcgtaaacgtgaaaattagaaattaggagttaggtggaagtaattcgaaatcgtgtgtagttttgggtaatacgagtttttagatttgatgttaaaaatcgattttttttattattaatttttttaaatataaaaataaaatgtagatttaattgtattagggattataatagagttaagatatttataattttattgtagatgtaagaatttattgacgtttcgggtttttaagtttgtaaagtgttaattttgggttgttggttttttgttttattttttttttatttgtttagtttttgaaaaacggtcgcggtatttttttttaattagattttttttattttattttggtattaggggagaagttttttaagtgcgagtttgagggttgtgatcggcgtttcgttaatagtagcgatcgtaagaagtatatgtacgtgtatacgagcgataagttttatttttgtaagatgtgcgataagttttatacgtattttagttcgttgcgtaaatatatgaaggtaatcgtcgtattttcgtcgttttttttgaggtaggagtttttttggttttcggtttggggtcgggcggcgagtggtagataggcggtggcgggagttagaggaagcgggaaggtaaaggttttatttagtggaattgggtagagaaggtagggcgggatttggaaaaggggatgggagtgtttaaggtcgttttaaatttttggtttaataataaaagttaaaagaggcgagtatgagtaaaggtcgttattgaatttggtttagagttagggtttttgtagggggttgacgttgcggaaattgttgtttttgtagttgttagtgagacgtaagaattggatttttgcgtttaggtttgagttttggtttatttaaatatggataagtaaagtttagggaaagaaagtgtagtttttggttttatatttagtttttttttttttagatttattttttttttgtgagttttggggattttgatttttttaggagtacggtttttgtaggt

SEQ ID NO: 142 - DNA, 851 bp, Artificial Sequence
  subsequence of SIM1
tttttgttttatttatttttagtttttataagcggtagaggtttcgggggcgtttattttttaattgtttttatgtttatcgtttaatttagcgatatcgtcggttttttcgttttttatcggtagagaggagtagagtattttcggtttttttttttggattatgaattggaggaggggtgcgtggtagaaagaggaggatgtaagttttgggacgagtgaggtgattttagaattttggtttttcgtagttgttacgaagttagtttattattatttttaattggaagtcgaaaattgtttttcgtaatttttttatagtttaagggtttagttttaattttatttcggggtcgtttttttttcgtttttttttaatgttatttataagttttttcgttttttattggtttggcgcggttaggggcgaggcgtttattggttaatagggttgagtgatacgagtcggtttcgagtcgtttttcgcgcgtttaggtttcgggttttgaattttattattcgcgggatcgttggatttttaatgagtttagggtgtcgggtcggcgttgttcggttgtcgtcgttgttcgcggttttttgggttttttcgacgtcgtgcgcgattcgcgtttttcgagttttttggggtcgggtggttgagcgtttagtttagcgcggcggtttagtagaatcgggaacgttcggaggtagcggaggtcgggtttgcgggttaggtttatcggcggaggaggtagggttcgtttaaaggtttatttcgttggtagtcgttggtcgacggcgtgcgcgaggtcggtttggggagttattttttttttttcgtttttttgaaa

SEQ ID NO: 143 - DNA, 1068 bp, Artificial Sequence
  subsequence of DLX1
ttcgtaggcggtagttacggtcggattgagtttttggtaggttaaggtttggatttcggttttttttttattataatttttttttggttttttgagagttgttttcgtgggttgacggcggcgggaggttcggttttcgagtttttggtttttatgggggaagggaggaaggagggatgtttttgtttttttggtagggagttgttagttggcgtagggttgggaggttttatttttgttgttttgcggtttttttttttttttttgtgatttaggtaggtttagtggttttagtttgagttacgttgttgttcgtttttgtaggttaagatttggttttaaaataagcgatttaagtttaagaagttgatgaagtagggtggggcggttttggagggtagtgcgttggttaacggtcgggttttgtttgttggttttttattcgtgtcgttcggttggaattttaattttttattcgggaagggtttaggaggaaacgcgggtttttatatttttagttatatatcgtggtattttttagcgtattaagaagttatgtagtaattttaatttatgtgaggttgttcgttcgttttttttttgttttttcggtttaggtttttttcgtttttaggtttatttatttcgttcggaaaagaaggatttagagggaagaaggaatagtggaggcgggacgttttttattttttcggagtttcgcgaggttcggtttagtaatttttcggtattcgcgttttagtttgaattttggtttgggtcgagtagtggtagtagagagtggtttcggagggaagttattgttatttgagatagtttaagtagtaagataaattcgttttattcgattcgtcgatttttagttttgtgggattattaggaaaaaataaaataaaaataaaatgtagaaaaagtaaaagtttttttttgttttgttagttttttgtttttttttgttttgtttgtgcgttggtaaagtttaggtttttattcgttcgttgtttttattttgcggttttagtaaaaagttataaggtttgagcggttcgg

SEQ ID NO: 144 - DNA, 1008 bp, Artificial Sequence
  subsequence of LMX1A
ttaggtcgcgagaagagggcggtatttatgagacgtcgattagtttcgagattaaaaatttagttttgggtatttttaattatttgttatttagacgtttacgaataagttcgttcgtttttcgcggttttggggaattcggtgtttagtgcgtgaggtttggggcggtttgttggatttatttgggtagggacgatttataagtattgacggatagatagtgatggggtttaatttagtgtatgaagaggggcgttagtttttttatcgcggattaggttttagagagcggggttttagagtttagcgttaagcggaaagagagtgcgtttaggacgtacggtttgaatatttatttagtagcgaggagaaggaggtcgaggtgtcgatcgcgttttggaagtttttttttatttttaggtcgtttagtatgttcgggtcgggtcgggaggatttgtagaggagaagaaacgatgcgtttgacgttcgtgttcgttgggattcggcgttagtagttatcgtatttttgggaaagaattgagggagtgtttagggcgattagaattagttaggaggatagggtttagttaagagaatgtagggtgggaggagagattagttatagcgaattgttttggttgatttgattttatttgaagttaatacgttatgtatttaggttttcgttttttaattgcgttttttttttttttgttttttttatttttatttatattttttgttttaggttttttatttcgaattcgatttcgttttaatttatacgaaggtgggttttcgggacgtttttgtaggaacgtagttattggggtatattgggtatataaagagtgggtattttttttcgagcgatcgggtttaggtacgcgggacgatggggtttgtaatttcgcgttttagtcgtttcgttgcggtttttatttgttttaggtcgagaaggggtattgtaagggattcggagggcgtttcgttcgtttttggattttt

SEQ ID NO: 145 - DNA, 1007 bp, Artificial Sequence
  subsequence of DLX6AS1
tcgtggcgcgttattttggtatgttgtgtgcgggttatattatttttagttttttattttcgatattggtaggaaagtttgatagttaggtttgaagtttagattttatttcgtaagcgtagagatattatattaagtgggatttttcgttcgttttattttttagtttttgattgtttttgagtttagattgggtcgtttagtaggttttagtaggatttttattatgtattataattattattttagtgtagttgttttattggaatagagtgtaaggattttagttattaattattaggggaaggttgagagttttgaagttataattcggataattttagttttttcggtttttttagtaattaaataaatgagaaaaaaaaatttttttgtaagtagaggtagttgtattttggatatagtgtttaattgtagggatttttcgaggttttgttacgatatattttaattggggtgtagttagagttgtaggaagttggcgaggaaagcggtgtatttgtgagtataaagcgaaaaatatagataagtatagggtgagggtgtagggggtgaaacgtttttacggtgttaataataagttgtagggtataaagtaataaaattgttacgtttatttgttggtattgaggatattataaatagtttcggcgcgtagttcgcgtgtatcgagagcgtattagggttgggagtgggaaggtggggtgggaaattttttttttttttgatatttttttttgttttttattttttatattttttttttatttagtttatttttttttttttatgaagtttttttgttgttgttttaatttattgtcgatttgtaaacgtgtagatcgtgttattggttcgcgtgttaaagtgtattttttgttaaaaggtggggaatagggtacgttatttaaaaatgaatttagagttattgaatcggttttttagatttaagaatttaagcgtaacgggttgggaaaaggaggggg

SEQ ID NO: 146 - DNA, 973 bp, Artificial Sequence
  subsequence of MIR196A1
ttatattcgtatttatcgtaaaaacgtagttgtgtaggtcgagggcggcggcggatttatttgggcggcggcggtgtcggtagtgcgcggtcgtcggaaggtgcggagttagcggtcgcggtttagtttaggttttttttcgttttcgtttagaagcgagggttcgagtggttacgtgttcggtagggaagttcgaggtagcgagggagtttatttggtcgttcgttttttttttcgggtataaattttggggatcggaaatataataaaattttatcgtgttgatgaattttaaacggtttatatttttttttgttttgaaaagaaagacgttcgggggcggattaaaaggaaggggaaataaatttacgagcggtagaagtattcgtttcggtttttttttttgttcgtatattttgttcgcgtggagagaagtagttttagttttcgggatttggttttcgtcgaatcggtcgttaggcgcgtttttttttcggaggagtcggcgttgatttagttcgcgttcggtagagattggaagtcgtcgtttatacggaaaatttatagagtttcgcgtagattttatgatcggttcggttcgtcgggtttgggatgaggttttttgtttttcgttagagtagaattgggttttttggcgtttttttgcgaaagtttaggtttttttaattacgttcggtggtttttattttttgtattttttcgttgaaagcgggtttttttttttttttttttttttgatagagttttgttgtgttatttaggttggagtgtagtggcgcgatttcggtttattgtaattttcgtttttcggtttaagcgatttttttgttttagtttttcgagtagttggaattataggcgttcgttattacgttcggttaatttttgtatttttagtagagacggggttttataatattggttaagttgattttgaatttttgatttcgtg

SEQ ID NO: 147 - DNA, 1050 bp, Artificial Sequence
  subsequence of FEZF2
ttttaaaaggaggtaattggcgttcgcgtttttttcgggaaagtgcgagcggtttgcgaatagggaaagagggaagagacgggagggggagggggaggaatcgtgatggttttgtcggtggaaggaaagggggggggggatttaaatttaaatttaggtagagtttcgtgcgtgatttataattttggaggtttgtaggtttgggggacgcggggagggcggagggtttcggacgcggcgtttttaaagggtttttgggtattagtttagtagtcgaagtattttttagtttttcgtagtcgagttgggtatcgtgttaataaattgttatttatttacggaatcggcgttagtttcgaatacgcgtaaatgataattattttattaggatgtggcgtatggacgcgggcgtcgcgtttggaggggggaatttgttaatggggaaatattaatttatgtaaaagtaatgagttcgttttggttggcgtcggaggtcgggagaaagtttttattgattaaggacgatttcgaagcgttacggagagtaaggtcgttcgtcggtatttcgggggtttcggcgtcgcgtaggtgcgtttttagttttatggttagttttgggtttgaggttgggggagattaaatcgagagttggaagattttcggagattttaatagattagaattgttgatttatgatcgggtttttgaggtaaagtcgagttttgtttttggtttagggttgcgtgcggggaggattcggagggtagtttttttagttgatgggtatttattgtttgtataattgagaaggttttatttttatttgtttaaggagtagattgaaatagtagaatttttttttggattttattaaaacgtttttttttttaaggttatttagtcgaattaatttttcgaagtgatttatagagttgaattttgttaaggtagtttttttatttttagttattaaagtatcgcgtggaatcgggttttgtgtttttttaaaataaagagaaggattattagagaggagagggagggggtttg

SEQ ID NO: 148 - DNA, 974 bp, Artificial Sequence
  subsequence of STAG3
cggatagggggcggggttcgaggcggggtttcgttaataatggttcggagtcggttttaggggcgggggtaaggaagaaattaagttaatgtagcgcggaagttcggtaggggggcgggattcggttggtatttaggaggttggtgaaataggggcgtggtttttttcgggggcgtggcgattcggtttaaaaggcggggtttgggtggaatttagtttatggtaattgagaagttagattaaaggggttattagtagtttggtttaaaattcgtttaatggagtaggagattgagggtagcgggggtgggatttgggtgggggcggggttaaagcgggtcgttcgcgtgtttaagtttacgtgagagggtaggacgtttgagagtttgaggttatacgaggttgtggagcggcgtgatttaaacgtggcgcgtattagttcgtatatttttaaatttcgcgatagttattggttttgggcgagtcgttgggattgcgtttgcgtatagcgtgttttgtacgttttcgcgtttttttggaatttttcgttttcggaagggtagcggcgggcgtttgtgtggaaggtggggtggttagagatattcgagggatttgaggtggggattttttttgaattttgttcggttagaagttatttttttaggttttgataggtttttttttgatttaagaataggttttttcgcgattttttcggttttgatattaggagttttttcgggtcgtgttttaatggttttcgttatagagttggagaattttttttttaggtttttatagattgagagtttatttttttaagcgtttggaaggttttttattttggagtcgtttattttaggggtagtcgtagatatttttattttttggaaaaataggaaacgcgtatttgttttttttttttttgcgtttttagttttatttttatggcgtttttatttttaaatttttaa

SEQ ID NO: 149 - DNA, 981 bp, Artificial Sequence
  subsequence of PTPRN2
taataatattgaaggattcgagggtggttttttcgtggttttgaggttaacgttgtagcgtatgtagtaaatatttgttaaggatttattgtgtgttcggtatattaaggtaggtaataaaggatttagtgtatgttcggtacgtagaggtaggtaataaaggatttattgtaggttcggtacgtagaggtaggtaataaacgattagttttagaagtagatgtgtttgttgtaaattatttttttacggtttagtagaggtataataagtagcgaattttttttagggtttttcgtgaatacggtattttacgttgttatagcgacgttattttagttttgagtcgtaataaagcggtagtgtttgtgttttatttttggttattttgttgagaattatgtcgattaggacgtaggtgtcgtttcggtttgtatcgttattgttaatagaaggagagaaatgaaagtgttgtcgtttatacggaagttcgatttataataaagttagtgatagtttattttttttttgtttacgggggtcggcgagaggaaaggatggcggatgtgagagggggaggaaggacgcgggttgggagaatttaagtgaggggagaagggaggtttttgggggtttcgagtttttgagtagtttgtagtttgtgggatgagttgttgtgtttagatgtgatttttttttgtttgtttttttttttttggttaaagatgtggtaaattttgttagaataaaatgaattaagcgtttgattgtgttagagttgatggtatttgtattgaaaatatttgtaaggtgttttttttagtgttgtcgggtaatttttcgagcggagaggggagggcgttgggcggttgggtttcgttggtttatgtacgtagggatattttaggtatatttttttaggttgtttgttattttttttaaacgtaggtttttcgttttagaaggagaaattgatataatag

SEQ ID NO: 150 - DNA, 1004 bp, Artificial Sequence
  subsequence of BARHL1
ggggtatcggatttgaattcgttttttaatttttttttcgaatatatttattagacgtagatgtaaatgtataaatatagaggtatttatatatttaatttaaatagttatatatcgatatatatattaatataatttgtagttatatacgtacgtacgtttagttttatagttattcgtacgtttttagtagaggtaggaatggtgttttttataaataagttttagaatatatcgttttattaataataaatattgattattgtttcgggtattaataattaaggtttgttatagtaattatataattatgatgatgatgaataatttagggagggaaatagggttggtgtggttgagtttattttagtgggattttggttggttcgtttagttttatttgtaggtgagtttagggtttaggtttgatttttttaaattttaggtcgttggttggttaaggtttttggattttggggttagatagtgcgtagggttcgtagtgcgagttttggaggatggtaggtgtagtttgttttagtcggtagggcgtgcgagtcgggagcgggtttttttcggttgttattttaggtttgttcgtttttgttagtttgtagttcggttagtttgtagaggaattaatcgtatttttttttttgaagagtatttttttatttttttaggtttttttagggtgattttatttttataaagaggtacgcgtagggaggagtattttttatttagttttaagggttcgttttgcgattttttgggtagggtaaggcgtagttagagtcgggttttggggttagatagtagggtttaaaatttaggtttttttttgaaaatcggtttagattttagtttttttttttgtaaagtggggtaagagttacggaaggtcggtggaagataaaaggagaggatgtacgtaaggcgtgcgatggtgtttggtattcggttatttattaataaatgagtacgattttttttggtttttt

SEQ ID NO: 151 - DNA, 1064 bp, Artificial Sequence
  subsequence of CCDC140
tgtttatgggtttataaattagttgttatttgttataaatatgtataaatggaggttataaggatgtgggggtgttggggatttaaagtttcggaattaggaggcgatttattttttttttttttcggttttttttttttttgtcgagcgttttgagatttatatgtatttagatgattagttcggttttttttatcggttagtggttagaggtttttggatattcggagaggggtgggagggtatttttaggttttggatagttgggttatatttaaagatttaatgaatttaaattttttgtggtttgtgttttcgaagttcgatttgggatggttaggatagtaaagttggttgaggttgtttggtttagggcgttcgtacgtcgttaaatttaattcggtttagagtcgtgatttcgtagaatagggcgtaataggttttaggaatgtcgggtgattttagttattttttttttgttttcgagttttgggttttttttttatttttttttgaagttttttcggagtttaagttattttatatttaaatttcgtagttggatgggagtttaggttatttttttattcgtagtcggtgggttaaggagttttggtgttaaatttggtcgcggtataggcgttaattggtaaagggatgatttagcggtggggtttggaaggcgagggttcgcgttttgtatttttgaattttggtttgggaggtagtgatttttatttgggttaataaattatttagaaagtattagtgggattttttataacggtgttgtgaacgagtgtagggtttatttacgttttgtgtacgttcgaaatgaaacgggagttttgaagacgatatttggataatttgataaaaaaaaaaaaaaggtgggcgggggagatagtttttattgttttttttgcgtttttgtttttagttttagagatattataggtgaaaataatagcgggttggttgtttaggtttgagtggagcgtgtttttgttagtttgaaagttttttttcgtagttcgtttggatgcgtgcgtttatagtttagt

SEQ ID NO: 152 - DNA, 1780 bp, Artificial Sequence
  subsequence
cgtttcgttggggtttttttcgttttgtaaagggagcggggagttgggggagttttgtatattagtttttttggagttgtagacgaggagtttcgcgttttcgggagtttttcggcggttttttttttaacgcgcgattgtgtagttttttttcgtcgtttttattcgcggttcgttttcgttttttttttttagtttcgttagtcggagcgcgtatatcgggatttcggtacgtgggcgcgcgggaggtggcggtcgttgttgtcgttgttgtcgttgtcggagttcgaatttaagttttagttggagtcggagtcggagtcggcgagatcgttttttatgttgcgggagttgcggtcgtttttcgtttagtttttggttttaggcgagtttggcgagtcggcgaagtatcggcggggaggaggattagaataggaggaggggtacggcggattgaagcgagttgggttgtgagtaagggatatttatagtttggagaaatagtttcgtttttttgcgcgttgtttgttttagtcgttattgggggttttaagtagcgcgatgttgtttcgttttttttaggcggcggtcggcggaggtttttcgtagtcgtttggtcggcgtcggtttttatttcgttggtaagttttttgtttatttcggagggcgtattcggacgttcgagtcggagcgagcgcgaagttcgaagttcgtttttagagtcgttaatttttttgtgagttttttttttcgtcgtagtttgcgttagatttgggagcgatgcgtttttattcgggcggcgcgttttttgttaattcgttatttatgggatcgattgtttttagttgttcggtttggagtgcgtagttttcgtcgcgtggtttaggagaggagcgagatattaaggcgtttcgcggatttcgcgcgtattagtcggtttggtggttttgcgggatagcgtttatatttaggggtgagtaggtcgttaatggaggttttttttatttttagttagcggtttgtatggacggtaaagagacgggagaaagttgggtttttttgcgtttaatttggtattggtagaggaatgtaatattgttttttttaattcgattgtcgcgggggtttttttagttttttttatttttaagttaaggagggagttttttttcgttttttaaggagaaaaggggttttttagagtagatcgttgatttttttattaacgtgttatttggggttttttagtcgttcgcgcgttaagttagtttttagtataagcgtttagagattcgtagtcgtttgggcgcgttaagagatcgtgggtagatttagtttttagggtagtttaagcgcgttttattaaagaaaaaagattgttggtatattaaagaagtgtgtgtgtgagagagaaagagatagagaaagagatagagagatatgggtgggggtattaggaaaggtagttcgagtaggaggtgttagtgaaatttattttttgttgacggatttagttttcggaatgttcgattgttttgattcgcggtgcggtgcgggttttaggggcgtataatatagggtgtttagatagagtttaataggtggtttgcgcgggtattttgtggttaggcgcgggggaggggaacggtttttttttcgagtttcgggttgtttttgttgtttttaggggtgtatttgaatatggataaggaaatgcgttttttgtttttaggttttatttggggtgagtgtgtgaggtatttaaatgttttttgtcgga

SEQ ID NO: 153 - DNA, 1038 bp, Artificial Sequence
  subsequence of GATA2
gtcggtatataggaggggtaggtggggatggggtggtgtgtagtaggttgggtgtttatagtagttaggtttgggcgtaggggattgttattttttatttttatgtttttcgttagtgatatttggtatttgacgtcgtttttgtttttttttcgggttgtattatttttcgcggaagatgaggttggagacgtagttttcgtggtgttagggttaggagatatttttttgggtggcgtgggtgggaagtcgaaaaggtgggagttagagtgggttgttgtaggggtgagggaggttattgagttttcgttgtttttttcgtttttatttttagtttttgggtatatagagagtgggtttttagggtttttagtagttgaggggtgtagtggcgttttggagaaggggtttacggtttaggggttgtggtggtgggtcgtagcggtagagagggttgttttgttttcgtttagttagggtaaattcgggttgtgtaataagtgtgggcggtatatttggttttcggttaggcgggttgcgggtaaagagagagaggattagggtgggtagaaagattagggtaggtagagttagggacgtttttgatagatattgagattacgatttttagaattagtagttattttttttttaaagaaagttagaaatataatattttatcggtaataattaggaatgttagtttaagttgaaggataagtggtatagaaggaattttatcggatagattttatagggaatttttataggttagttggaagtgggtagaaattttgtgggttttagattttttttaatcggtcgttgtttttatttttttcgttttaattttttagtagttcgatttttgcggattttatattcgggaagtaagtagacgggttttttttttttttttcgtttggcgcgcggcgtttgggtttttattattacgggtttagtgtttatcgtgcgcggggttgtaggagacgcgcgttcgcgcgtgagcggggttggtatagtaggggttgttttgcg

SEQ ID NO: 154 - DNA, 1100 bp, Artificial Sequence
  subsequence of ADCYAP1
gaggtaaaaagggaacgcgagttagggagtttgattcgttttgaatgaaaagaaagagaaattaaattaaattttttagttatttaaaatttttaggtttttagggaggttttgttataattttttttaagtatgattgtttttgggggaggggaaaggggtggttgtatttattgaaaatttaaatcgaaataataaatggttaaatgtggatatttatggatttaaatagttttgtttacgttagagaaattgagagtatagggtttgtgtgaagtttatttcggtagaaggtaatattttaataaagttcgtgggaaaatagattatattttcgttatgaataagttatgtagtgaaaaatattgtttatagtttgtcgatttatattattattacgttttttaattcggcgtgaggagggagaggagtgtttatatttgattaggaattgtaggatcgatgtaaattttagggtagtagttagattggtatatgtagggtttttcggttattttttttgtatgtcgcgggtgagaggaatagcgaggataatttagcgtaaatatacgaagggtcggattttaagggggtagcgttgggagaaaggttaggtttgaagcgcgcgtcgtttgttcggattttatttcgggtttttttcgtagggtttggtgttaggagattttgcgtggggaggggggtatcgaggggttgtcgtttcggttttttttacggttgtttttaggtagaggcgggcgacgcggtgggtagtgcgagtttcgggtttttttcgaaggttttcgcgtggggtggggttcgtttgtttttcgcggcgattgaatttgtgtttttcgtttcgttatttttttttcgatttttttgtttgtagtgggagtttcggcggcggcgcgggggacgacgcggagtcgttttttaagcgttattcggacgggatttttacggatagttatagtcgttatcggaaataaatggttgttaagaaatatttggcggtcgttttagggaagaggtataaataaagggttaaaaataaaggacgtcgaatagtttatttgtagcgatgggttattagttatttt

SEQ ID NO: 155 - DNA, 963 bp, Artificial Sequence
  subsequence of TBX5
gtagagaataattattgtattagggttcgtcgaggtacgaggtattttattttgttttgtaattacgacgattattatttttagtttgattaatgggtataatttttaagtagcgtagtggtggatgttcgtaaatttttgcgtatcgttggaaatttattaggttgagttgtaaaacgtatcgcgtagacgtttttggtggcgtcgagagaagagttaggtttgtttagtatagagtcggagagcgtcgggtttttcggaagggtaagtttttcgttaaggggtttcgagggagttggacgtttgaatttggatttgtttttagtttcggggttcgattttgggttttgcgcgtttttaatttttagggtttttcgaagtatggtttggttttaggttcggttttgtaaggattggaacggtagtaaaatgtgtagggaggtagtcggtcggtagagttgcggcgggagttaaggttaggttcgcggggagagcgggtagtttttagcgtcggttataagtttttaggttagttgggtcgtagatttttttgtttttagagagtataattcgcgtttttttttttagttaggttgtagtggttgtttcgagtttcgttttcgttttttaagttgttaataacgatatgtttttaaattcgaggttcgtgtttgtttttagatgttaagaacgtaattcgaaattttttttttaaattttaggtcgacgagatgagtttttatttgatttttgagtcgaggtgggtcggaaatcgaggtttaggtttcgtcggggttgtaaggaaaaggggaaatttcgagcgtagcgttttttttttgtggttttttttttcggtatttcggattgcgggttttgtagttatttggatcggtatttaaaggattttgtaagtttagttttatagattggtttttttagacgtttcgaagttcgtattacgaa

SEQ ID NO: 156 - DNA, 837 bp, Artificial Sequence
  subsequence
ttagtttttatttatatttttgttacgacgggtaattgggtatgtgtaggggaaaaatagtaacgttaaaacgtaattgtgtaaatgaggatagagagtgcgaaaggagggagaggcgaggagttgttttatttttagggaggttttggggagattgattagttttaaggatagatcgttgggaagggaaaaacggtttatatcgaattggatgtcggatggaaatttttttgcgttattagattgcgtttagtatagtagatggtacgagtacgtgcggcgtttagtttaggttttcggaggtagttgagttggaaatttcgacggaaagtatttataagtttttattttgcgttggtttattcgcgtgtacgtttattttttacgcgcgtttttggtttagaagcgtatagatgtttattgtttagagtcggtatcgttggggagatcgagcgatttgcgcggcgtatagtgcggcgttggtagggttttgggttttcggtcgggggttcgagcggttaagggatgggggtgggggcggggagagtggggggagggcgaaagatcgtcgagaggaggggggagtgggtggattaatgatgaaaaagttttttttattttagttttttaattaaatgtatggaaagaatcgaggcgagtatatttggttttaatttatagttttttgatggtattaaatgttttttttttagattagggttggaagttttgggttaattatggtcgtttggagtttagaaattatttatatatattcgtatttttttttttttttagtcgagttttttgattataggacgaaaaaaaaaaaaagtt

SEQ ID NO: 157 - DNA, 1012 bp, Artificial Sequence
  subsequence of NECAB1
tttgtgattttgggaaattttatatttgaatattatttaataatttttgttttatagtaaacgtttcgaagtttattttaggtagagtttatagtatcgtttttgttagtagtattgttataggtaagttttagaattttttggggtgagatcggtgtaattattttattgatgaggaaaagtgaggtttataggttaagtttttcgagcgtaagataatatcgaatggtttaatttttatattaaattagttgtttttcgattgttggtattcgtgaattttgttttcgtttttggggaaggtcgggtagataggttattgggattaaaagtagagtaggtagaatttttttgagtttttttttatttatggtatttatttaggtgaggttgggggcgtttttattttgcgttttggttgtttaggataaacgttttgttattcgttttttttttttgttcggacgtttttttttatttttgtatttggggtcgtcgtggggttggtaggggtttttaggttatttttagtcgtggtcgatggtcgtgtcgggggtagagttcgttttttttcgcgcgttgttacgtgcggggatgatatggatttagacgtcgttaattacgggaaggaggaggcgagagttggagcgcgtgggagcgtagaggtcggaggagggagggggggatatcgagcgcggagagcgcggagagcgcggagggaggcgcgcgcgggagcgaatattttttcggatttagagttcggcggcggcgaagtagtagttgcggtcgcgtttttgttagagtcggtgcgttcgtttagtttcgtttcgtttgaggtcgttagggttttcgaggatggaagatttttaggagatatcgtcgttttttaataatttttcggaggagtttagttttgttttgtatttgtttaagggtatgtcgatttttttcgacgtaagtatagatggtgggagttgggcggttgttttttagtatttttttttttttcgaa

SEQ ID NO: 158 - DNA, 1017 bp, Artificial Sequence
  subsequence of ADRB1
ttcgagtcgttgttttagtagtggatagcgggtatgggtttgttgatggcgtttatcgtgttgtttatcgtggcgggtaatgtgttggtgatcgtggttatcgttaagacgtcgcggttgtagacgtttattaatttttttattatgtttttggttagcgtcgatttggttatggggttgttggtggtgtcgttcggggttattatcgtggtgtggggtcgttgggagtacggtttttttttttgcgagttgtggattttagtggacgtgttgtgcgtgacggttagtatcgagattttgtgtgttattgttttggatcgttatttcgttattatttcgttttttcgttattagagtttgttgacgcgcgcgcgggcgcggggtttcgtgtgtatcgtgtgggttatttcggttttggtgttttttttgtttatttttatgtattggtggcgggcggagagcgacgaggcgcgtcgttgttataacgattttaagtgttgcgatttcgttattaatcgggtttacgttatcgtttcgttcgtagtttttttttacgtgtttttgtgtattatggttttcgtgtatttgcgggtgtttcgcgaggtttagaagtaggtgaagaagatcgatagttgcgagcgtcgttttttcggcggtttagcgcggtcgttttcgttttcgttttcgttcgttttcgcgttcgcgtcgtcgttcggattttcgcgtttcgtcgtcgtcgtcgttatcgtttcgttggttaacgggcgtgcgggtaagcggcggttttcgcgtttcgtggttttgcgcgagtagaaggcgtttaagacgttgggtattattatgggcgtttttacgttttgttggttgtttttttttttggttaacgtggtgaaggttttttatcgcgagttggtgttcgatcgttttttcgttttttttaattggttgggttacgttaattcggtttttaattttattatttattgtcgtagtttcgattttcgtaaggttttttag

SEQ ID NO: 159 - DNA, 1030 bp, Artificial Sequence
  subsequence of OTX2
ggcggttttcggtttaggtcgattcgtaagcgaatcgagttttcggcgcgcgggtttaaggaggcgtttggttttttattattgttgttttaatttatttatttagttatatttgtatttttttgtttcggattttaaaaaggtttttgggatttatttaaacgattttaattaaatttgggggttaaaacgtaaagatcgcgggagaagtttagaacggcgttgatataaaaataaaattataaataaaattttttaaaatattttagattatattcgtagcgtttcgacgacgttttgtagaagcgacgatttttaggagtacgttttttgttttttttttattatcgtttttatttttagattatatttttttttttgtaacgatcgttattatattttaaaatttgtaagagaaaaaatatattcgtttatagaattttacgttcgtagaggggtatcggtcggtttattggaggtattggggtgatggtcgatagatacggggatacgtagatatggatatagataagatagacggatatttattttatagttttatgggaggttagaaaaagttaaatattttttaaattttaaatagttagttattttaaaagaaattaagagtattgagttagaaattttgtagtttttatttttttatgaaaagttaaatataattaaaaaaaattttttttaaagaacgaaaatgaggaaggcggttagagttttaaatacgcgtgggtttttttgtaatatatataaagtagatgtggcgagtgagggttgttttagtaatttttatttttaattatagtggaggttgtgttggagttgaggtcggtttcgtttttagggagatttgttgagaaagcgtttttttggtaattttttgacgtaaattttttaattaatggtagggagagaatgattgatatatttaagttagagggaaaataataaaaatatataatagggggaggaaaataggtcgttgttgtacggggggacgagtaggtaataaatt

SEQ ID NO: 160 - DNA, 1076 bp, Artificial Sequence
  subsequence of NKX2-6
aagtgaatttttgagatttagggttttattttcgttaggggtgtgtgaagtatattgggtgttaggagaggatgggaatgggattcgagaggtttttttttggatttttcgagagaaaatggagagagtttcgaatttaggagataggaggcgtattttttttatgtatttatggattacgttttcgtttttatatttttttcgtaggaggtaagatttgagcgtttatttacgtggtttttttattcgtttcgcgtttatttttaagattgttttatagggatttttaggaggtttcgaattatttagttttttgttatcgtcgtcgttattagcgttgtgaatttttgattttcgcggttttgcgtttatttttaggtattgaaagtttttcgggttttttcgtattcgcggatgtggcgaagtcgcggggtagtttcgttcgcgttttagtcgtaggatgtttttgatcgagaagggggtggaggtgacggggtttagtagtatttcgaaggcggatggggcggggtcgaggaggttcgggtgaggagcggtattttgaatttttcgttttgtcgttgtaggtttcgtagatagatttaagttttgggatagacgtttagcgttttagatagcgtttttttttgggttatgttggtaggttcgggtttagggtcgggtgacgagatcgtagttttttattggttttcgtagaaattacgtgttagttttgtattttttttttttagcgtttttttttcgcgttcgtcgaattttttttagtttttagtcgtttttttgggaggtttggttcgtattgacggtttttaaagtaaagaaaacgtggagagatagtgttgcgcgcggtttggattttcgttaatacggtattagcgggagttttcgaggttaatttagggtttacggggatttttttagtggatatgcggggattggaggtggagtttttgttgtgtaattcgattcggtttatttagtttggttttagtttaattttgtatttttatggcggaggaaagttaagatagagatagttgggattcgcggaatttgag

SEQ ID NO: 161 - DNA, 927 bp, Artificial Sequence
  subsequence of SIM1
ttgatagagtttttttttcggaatgaatttaatatttttgataaaatatatattatagattcgatattattatcgtttttattttgataggaagaggaggaagataagcgtgtgttttgtttttttaaaatttttttttattttaataaataaacgttatattttaatagaaatgtattttttttgtagtttttttcgggaatttttatttttttaattaattgaggtgttttgtaggtaatgggggagggtggcggaggttttgttttatgatttgattacgatttggtgattgaggaggggaaagtttttttaggtcgaggttattgcggtttttgaatagtttgttttaattaattgtgtattttttgagagagtgtaggagagtttggtttagggggtaattttgaaaattttaaattttatgtattttgtcgggtttgtatattttcgttaaatgtgtattgtgtaagtttttttcgggttgaggcgtcgagtggttttcgtgtttagttttcgggcgttttcgtagtcgagttttttagagaaaaaaagagagggaaattggtgtaattagttcgaattaagtttttagagtagttattggtaggaggtttttttacgtattaaggcgtattcgattttatttttggtattgttgttcgttttgtttgcggtttttttcggttaaggagtttgtgtggttgatttgagggtatcggcgagcgcgtcgggttaggttttgttttacgtagtagtttggattttatagtgtgtgtttgtattcggtagtgttttgattttttttgtttgttgatttcgggttagagatatcgcggtcgtatttagggaatttattaatgttattgtaaatgtggggtggaggtcgaaggttatatcgttgtttgttttttttttttttttttt

SEQ ID NO: 162 - DNA, 927 bp, Artificial Sequence
  subsequence of DRD4
ataggtagatgtaggtttagttttttggttgtcgtgggatatatatatatatatattgttatagttattgtttattatatatatttagtgtagatgttggtatatttttagaaggaggtttatagttcgtaggggagatttgggttggataaaatttaggggaggggagggtgtgtggggattaggtttttgttgagaattttggggggaagtttgagggggaattgggggatggagtttatattttatattaggtttggttttcgagtgggtcggttttggtgttagtttttttgcggttagagaaaagtagtttagggttgagttggagacgcggtgttttcgattgtgggggagggggattcgaggtttttttttgatggatatagtgaatttaggcggttggggtagagattagtagtacgggatacgcgtgatttgtgtttttttcgagtcgtagacgttatagtgacgacgtttaagtttttaatttttttaaatcggcgggaaggattagaggggttgtttgtttttttgtttttggagagagttattttacgtggagttttacgttgggtattgggtacggtttttatatttttaattataatttatttttaagataattatgatgtggatattattattatttttgttttatagatgaagaaatagaaatgaattaacgtgttcgagttttttatagagaatttagttaatagagtagttagtttcgtttttaggtttagcggggattttgtttattttaggttggggaggggttttttttgagttcgatgatattaggttcgtttttagggaattttttaggtcgtttttatgtttgtaataaaagggagattaaggttagaagtcgtgatttacgtttgtaattttaggattttgggaggtaaaggtaggaggata

SEQ ID NO: 163 - DNA, 810 bp, Artificial Sequence
  subsequence of TBX15
gaaaaggtttggacggtcgaaataaatttttttttaattttttttttatcgattaataaaaataattttttagtattaaatttaaatatcgtaacgggttataaaaatacggagaatttataaaattttatttttgtaggttattcgttaatcgtattattattagtttcgggagtatggaaattgaattgttattgtttaaagagaaaatgtaagcgatagttgttttttttttgagttggatagttttgtggttgagatttttaagtttttgagttttagatcgtttttattttagtgtaggtcgttgtttttttttcgggtttgttagcgtgtgaagcggggtttattttttagttttggttgtttagtaggagtattttttagtatggttttagaagttcgggttggaaagtttttgaaatcgttaaattaagttgcgagcgttgtatgtgcgcgcgcgtatatttaagattttttcgataatagttgttttttcgggttgtttttgtatagttgaaattttggtatttaatataaaacgttaaagttggtttttttttttttagggttttgttaaaatatagatatgatattttttttgttttttatatgtttcgtgtatataatatatatgggttattgtaggatttgagatataaattaatcgtatttatattttaaatatatagagatgtgggttttgaagagtgatttaaggagtaaggggtttaatatttatttttgttttcgtaaacgttaaaggagtagaaatataattatgttttattatcgaaaagttggtgggag

SEQ ID NO: 164 - DNA, 1080 bp, Artificial Sequence
  subsequence of TRH
ttttttttttttttttttttgttttattttcgttcgtttgcgttttatattggttttgcggtcgtgtttttttcgtgtgtgtttgttttgtgttttttcgtcgttttcgtgtggatttcggtttttgtttcgttttttagttttttcgtgcgtttgtgtttaatcgtgcgttttttttaatttttttgtttttttttttgttttttatacgcgtgttttcgttaatagatttttagaggtggtgtagaaacgatttcgcgtcggcgttttattttgcggttagtgttttcgcgtttcggtttcggtttttatcgttttcgttttagattttcggaggagtaggcgggcggggtttcgcggggtcggttgtcgttagcgttttttttcggcggtcgcgattttttttcgttgattttattcgagtcgtcgtttggcgtagatataagcggcggtttatttgaagagggttcggtaggcgttcggggtttttagcgttgtagatttttgatttgtcgattgcggatttcgagttttcggatttcggatttattttgtggagtttatttttggtaggtaatcgttttaattttttttttttcgtagacggtgttcgggagtattggaaagggagttttttcggttcgtttttattttcggttttttgagagttttttggagaatagggtcggtgtataggcgggcgttgtgttcgtattttcgggtttttttcgagcgtaggtcggggattcgacgggaggtacggcgcgggtatatcggaggttttcgttgttgggtatttaggtcgttagagttcgtagcgcggggtgggagtttcgcggtattttttgtttgggtgtcgtcggatagagttgggtatttagggcgtcggtttgagaatttgggagttcgaattttaattttaacgttagaaatagaattttagcgttgaagttaggattttagcgttagaattagatttttggtttagttatgaaagtttagtattagaaatagaattttagttttgtaaatagattttgagttttagaatttgaattttagcgatagaaatgggatttt

SEQ ID NO: 165 - DNA, 1050 bp, Artificial Sequence
  subsequence of FBP1
gtaagaggattgtttgagtttaggagtttgaggttgtagtgagttagtgagaaatttgatttgggtttttaattgtagaaagagtgaaggaggataaagaagaaatggggaaggaaggtatttgagttagtatttttttgtttgaggttagggcgggtatgacgggtaggttaggtgtttatatttggtcggttgagaggggtacgaggtaggttgtttagggtagagaagaggaatgtaagatggaattaggtgtatatttgtgagacggatttttaggtgggtttttttaaggttaagggaggagatgaggatttgagtgaggttgagttagtttttggagagaggggttattagatagagagcgagagaggcgtttagatattagacggatagatagaggttgacggtaggaggtttagagggtttaacgttagttcgtcgggtaggttttttaggtagatagataggacggggtttatcgtttaaggttcgatttataggtgcgcgatgttcgttttgcgtatcgtcgaagagatggttttgattgttgtgtagagcgagttgagtagttgggttaattcgttcgtgtcgcgggtttttttgtttttttttatgacgaagcgggttagggtgttgacgttcgtgtcgaagggcgtttggttagttatgtttgaatcgggtagagcgcggggttgtaggtgtaagcggtaggtgcggggttgtaggtgcgggcggtaagagagggtagtaggtattggtcgtaggtgcggagttgtaggtgcgggcggtaggtgcgggtcgcgggatttggcgggaggattgatagatcgattgtcgtttcgagtgttcgtagcgttcgtggtgtaattgggttaggttaggcgtcggcgggtgaatcgcggaaatttttagacgcgggtcggtttttcgttttcgggaatatttttgcgtttcgtttatttcgcggattagatcgcggtttcgtttgtttggatttttagataaggtcggggattttttttgcggttttttaattttcgtttttat

SEQ ID NO: 166 - DNA, 968 bp, Artificial Sequence
  subsequence
tatttcgtggtgtttttttttttcgttttatagttaagtttagaggaatagggtatcgtttcgcgcggtttcggagttatattcggatttgggaatttcgtgtcgggtttagaggattttattgggcgggttggggtttatttttaataaatttagttgtttatatatatttttattattatttttacgcgcgcgcgcgtatatatatatatatatatatagagggatttgttaattattgagatagcgatttagaaaggtgtaaatttatttggtgtttatttaattgtaaagtttttagatttatataaaatattagcgagttttggaggtttgagacgtttcgcgttcgatttacggcgcgaatattagcgtttcggtcgggtcgtaggggtcggtgtttagcgggtcggggtttggcgagttaggtagcgggtcggttgagtttttgcgtttcgcggtcgcgtggtttattcgtttatttttttattattggttgggatttgtttttttttaaagtgtgatgttcggtgttttattttaggttacgcggagttttttttcggcgtgtcgggttttgcgtttttgtttagtggtttatttcggttcgttgcgttcgttttcgttttaagtgtttaggacggagcgaggggttgtgggtcggcgtagggacgttggttgtttatttttaggagtcgatagaaggtgttttcggcgttacgcgtttaattttggggttgatttaaataattaataagtttagagaaattttacgaggggaatttttttttttagtattaataaagtttagtaattgggtttgttggttatttaagttcgagttcgatttttttagttttttttttttttttttttttttatttagaggtaacgtttttaaaaatcggtaattgcgtgtgcgtttggggtgtgtaagtgtgagtgtagatgtgtgaaagttatttgtt

SEQ ID NO: 167 - DNA, 1080 bp, Artificial Sequence
  subsequence of BCAN
tagtttttttttacgttcgcgtagttttcgtcgtagttttagttgtagttgtaggattgagtcgtgtattcggaggagattttcggaggaggcgataaatttcgtagtgtcgcgatttaattttagttttgggtaggtgagtgttttcgtagtttcgtcgttcgtcgtggggtcggggatagggagaagggagtgtttgtttggtttgcgtttttcgtttgttagtttttgtttcgaggttttggggtatttaattcgtcgatttttgatatcgtagcggggtaggttgttggatagtttcgagcgtttgtagttgttgttgttatttttgatttatatgtttttagttttgttagtggtagtttttttgttgttcgtagtttgattagtaatttttcgggttttcgtattttttttgttgcgcgtttttgttttagcgcggtcgtcgagattttcgattttgttttaggtagggcggtagcgtttcggattagtttttgttttcgtgtttttacgtataggtagttttaggagtagcggttagtaatttttttggggatattttattgtagttcggtaggatagtcgattagagtcgttttagggggtggtcggagtgtttatttcggttggaattttaattcggttttttgttttgtgattttggggtagttagttgatttttttgaatttcgggttttttatcggtgtaatgagtaaaatataaataattttttttttatagttgaggttatttataggaagtatttaggagaattggtttaggtagatattagtaattattagttggaattcggttattattattgtatttttttttagtattgttaggggtttgggaatttttttttttaggtatttttatattttgttaggtatttttttatttattggaatagttttgtttatatggttttaataggattaagagattggcgttaagtttaaggggtattattattatttgtattatttagattgtagatgatgatagtaagattagggaatagtgttatttttagatttatttatagtattattagtgtttatgttgttatg

SEQ ID NO: 168 - DNA, 1016 bp, Artificial Sequence
  subsequence of TP73
ttttaggttagggtttaggtttcgtataggttaggagtgattttggtgggttttttttttttttcgttttttgtttattttggttgggggtgtaggggttagggtgtggtgtggttagatttttaggtttagggcgattttttttgttttttttgttttattgttttttgtttttaatgcgtcggtttttcgtaggatttgggggttttgaagattttcgagtagtatcgtatgattatttggcggggtttgtaggatttgaagtagggttacgattatagtatcgcgtagtagttgtttcgttttagtaacgcggttattatttttatcggcggtttaggggaattgtagcgttagcgggttatggaggtcgtgtattttcgcgtgcgttatattattattatttttaatcgcggcggtttaggcggcggttttgacgagtgggcggatttcggtttcgatttgttcgattgtaaggttcgtaagtagtttattaaggaggagtttacggaggtcgagatttattgagggtttcgtttggttgtagtttgcgttatcgtttagagatttaagttgtttttttttttttttttgtgtgtttaaaattgttttaggaggtaggattttcgggttgtgttcggggaaaggtaaggttcggtttatttttaggtattttataggttttaggaaaggtttagttatcgaagtcgtttgtggatagtttgagttatttgtagaattttttggagttgttttagtgttgggtttgtggggcgggggttggtttatttttagttttgttattgtttcggcgtgttttatggtaggcgtgggtggggatcgtagcgtcggtttcgatttttaggttttattttagagattgttattttttaattaggcgaggtttttttaaaggaaaggattttttttgttgatggattgttaaaaagtattttgcgatattttttggttttggatagtagtgagtagttaagtgattgtgtttgaaatattag

SEQ ID NO: 169 - DNA, 1058 bp, Artificial Sequence
  subsequence of MIIP
gtttttcgagtagttggattataggtacgtgtttggtttattttttgtagagacggggttttgttatgttgtttaggttgggtatttttttttttttttttttttttgagatggagttttgcgttttgttttgttatttaggttggagtgtagtggcgcgatttcggtttattgtaatttttgtttttttagtagttgggtttaagcgatttttttgttttagtttttttagtagttgggattataggcgtcggttatttcgttcggttaatttttgtatttttaggtttattggcgtgagtaatcgcggtcggttttaatattatatttttttgtttattgtgtagtttttattttttggcgtaaagcgggtattcgttaaaaatttgaatgaaagtataaagtaggtcgggcgtagtggtttatgtttgtaattttagtattttgggaggtcggggcgggcggattacgaggttaggagttcgagattagtttggttaatatagtgaaattttgtttttattaaaaatataaaaaaaaaaaaaaaaaaaaaaaaaaaaaaaaaaaaagtcgggtatggtggcgttcgtttgtagttttagttattcgggaggttgaggtaggagaattaatcgtttgaattcgggaggtagagattgtggtgagtcgagatcgcgttattgtattttagtttgggtaatagagtgagatttcgtttttaaaaaaaaaaaaaaaaagaaaagtatgaaacgatgagtataatgagtagttagtcgtttgttttatttatttcgtcgtttcggattgtcgtttgcgtcgagtttaggtcgtttagtttgattgaaattgtcgttgttgaatttagcgtatttagtttcgtagttgattaatcggagttcgttttttcggggttcgttttattttaggaatcgattttcgtatcggtttcgaaaattgattaatcggttgtgtagtttcgtttttagtttcgtttttacgttgaaattgatatttcggatcgtcgagtaacgtttattagacgtttcggttttgttttt

SEQ ID NO: 170 - DNA, 1166 bp, Artificial Sequence
  subsequenc of HOXC4
ttcggtttgggttcgaggtttcgaggcggttttataagtgtgaatttttgtttataaacggcgacgtatacgtaaacgtttatatatttaggtagatatattcggaggcgtgggggaatatattttagacgaggttcgagagaatttgggggttagatttattttaatttaagttaagatattttttttatttttatttttttttagaggcgcgattgggattttgcgttaagttttagtgggggaggggatcgttttacgattttttgtttaaattttgattcgtcgtttgtgggggggcggggatgggggaagaaatttgtttttattaaatttttaaaaatatagtttgtatttagattaaatcgtttgggttagagttgacggtgaattttattttttttttttttataattttttttttttttttttttgttttcgttaaatttttttttcgtagttattataaaggtttttgaggtattttttcgcgggaagaaatggtatttttttttttttttttttatttttttatttttagtaggatttgtgtgtcggtagaggcgtttgacgaggggttaatttcgttttttttgtttacgatcgagaaaagcgtttggttttttttaaatgggtttagttcgtcgtttgtttaggttgtcgcgcgttttttcgtcgtttaggtacggtttagtggttgatgtttcgcggatttagtttgcggtaggtcggaattcggagtttgtgggatttgaaaagggtaaagggagtgcggtgggatataagaggggttcgagaggttcgaaaaagggagttttttttttggagtaatgtgggaatcgtcgatcggtgaggtttttatttttttttttcgtttgtattttgttcgttttcgtttttgtggatggtcgttgatgggggggaatataagtgttttttgttcgttttcgaaagaaaattttttttagagaaggttatttaggttagtggagatttatttagaatattagaagggtttttttttattcgaggggcgggtttgggcgaggttgaggtattttttttaattttagaacgattttattgagtgtatttatagcgagatttggaggtgttagagttcgaggttggtcgtttaatagtattattttgaggtgtttttatat

SEQ ID NO: 171 - DNA, 1334 bp, Artificial Sequence
  subsequence of HOXA7
tcggtttcgcgtttcgcgtttcgcgttttttagcgttgcgtttttcgttttcggtttcgtttcgttagtttggttcgtttagcgattgcgtttatttgaagatcgtatttaggggtagatgcggaaattggttttagtcgcgttatgtagcgcgttttcgttcgttttgtcgtaggcgtttttggcgaggttattgtagagttcggggatgttttggtcgtaggaggcgtagggtaggttgtcgtaggcgtcggcgtttaggtcgtagtcggacgtaaaggggttttgataaagggggttgttgatattgtataagttcggaacggtcgaggcgaaggcgtcggcgttcgtttcgtagtcgtttttttgtgagttgggagtaaaggagtaagaagtcggttcggtattttggaatagagaagttttcgtcgtatatttgttaaaaagcgcgtttatataatacgaagaatttataattttgatttgtgatttgttgttcggtagtttttagtgtcggttttacgaggtagagtgatatatgataatattatatttttagatttatattaaattttatttttttttggacggagttcgtcgtagtacgtgatcgtttatatgatcgttttcgttaattttagtagtttttataggtggtttcgtttcgtagggttcgtagtcgtttagaatggaagggtaagaggtttaaatatgcggttaaagaattcgttcgcgttcggcgggtttggcgcgtttcgcggaaaaagatttggaggtttcgcgggagcgtttagttggcggttaattttcgtattggggtttgcggacgttaggcggttcggttttacgtagtattttttatttcgttttttcgtcgatttttgttagtgagttttggattaagtttgggattttttttattttttttttgttcgtttgtttagattttggaagggtttttgtttttcgtaatagtttgtttcgcggtggttttgtgggtaggatttagttatgagtagatcgattttgtttaagtttttttttatttaggtttagtgggcgataggtcggatttagattcggatttagacggggaaggcgtagtattttttgtagttgtagagagattgttatcgtaaattggagttatgtggttcgaataaagttaacgttttttagttttttttttttaatcggaggtatattgtttattcgttttaaaggaagtagtgaaatatttatttattaatgagatttatttgttaatagatttattaacgtggggttttttttttttttttcggacgttgtagtgttgtaggttttgtgttttt

SEQ ID NO: 172 - DNA, 1181 bp, Artificial Sequence
  subsequence of BST1
aaagagggtatttgttaaagttgtcgttttttggagttagaaaagtattttttttgatgatgattgatttttttttattttttttttaattttgcggtttttttggggtttttttttcgtggatagcggttaagattagtttgggttatagtgtattggttttgttttgggtgatgttttaagtttatttttttttaattttatttatttattaattttttttttagatagagttttattttgtcgtttagggttggagtgtagtggtgtaattattgttcgttgtagtttcgattttttggggttaagtttttttatattttatttcgtaggtagttgagattatagatgcgtattattaggattagttaagttttgtatttttttgtagagaggaggttttttatttgtatgttatttaggtttgtttcgaatttttatatttaagcgattcgttcgtttaggttttttaaagtgttgggattataggtatgagtcgtcgcgtttcgttttacgtttagttttgaaattgtttggaacgggaaacggtaaatagcgagatattcgagcgagagtttcgttttgtattagtttgcggaatcgttttggtagaaggagagaaggggagtggaggaagtacgggattggagggattaaagtttttcgatggcggtttaggggtgcgcggtatcgcggttgttttagttgttgttgtagtttttgtttttattgttgttgttggcggcgggcggggcgcgcgcgcggtggcgcggggagggtattagcgtatatttgcgggatatttttttgggtcgttgcgtcgagtatcgcgtattgttgagtttcgagtagcggtgaggtagtcggtcgggtggaaggggagtcggaaagaggtaacggtggggagggtttggggaggggaaaattggcgttaaagttcggggtgagggggtaatgagagaggttttgaggggagaggtgagtgtggagatagcgatggttttgaacgatgggggcgagtgaggagggggttgtgaagtgtgtttgtaggtgaggggtgcgttgtgcggggtttagagatgagtgtgtgtgtgtgcgcgtatatatatattgtagaggtgaggggtgtgtgcgtgtgttttagaggtgaggggggtgtgtgtgtgcgcgcgttttagagg

SEQ ID NO: 173 - DNA, 1039 bp, Artificial Sequence
  subsequence of C1orf114
aggtattattaacgaaatgaatattattgtttttttatggaagttataatttcgggagtgatttttttttttagggataaaattattattttttacgtttaagaattttgagaggggaaagaatatgataaaaatttagtttaagttatttttagacgagtattttttatatatagtgaagaaaggtgttattagatttttttattagtgttagattcggtggatttcggattttagaggataaggcgttattagaaagtttttttttttattatttatttttttttttagttatattagacgtttatatttttattttttattttattattaatattttcgtatcgttttttttttttttttttttatattttttttgtttgtgtaaaatgcggcggttgggattatagagatggtcgggggagtttttttttttttttttttttaagttttttatattgaggtaaaggagatttcggtatttgttttcgcggtagttaggattatattgttatggtaatagcgtgtttttgcgttttttattcgggtttttttaattacgagagcgataaaaatttgcgtaggcgtatgtaatatttttagattatcgatttttttcggtatttatttcggtgtttgcgaagggttagtgaagttgaggtttttaaggggacgattaaggaatttacgtattattaacgttatagagattcgcgaagtgtcgttgtgttggttgtgaaattttttttttgtagttgtttttcgttagatttagaattttttaggataatttttaaaattatatttttatatgttatgtgttttatagttaacgtagaaaagtagtttttagtttttattagtagataagtaaattgttaatgagttattatatttttgatttttagttttttttttttttaattaaaattaaaattttatttaagagagttgttaaagaaaataggatttttgttttagttttgaggtatgttaaaatggttaattagaatttttttaaattttgtttatttttatg

SEQ ID NO: 174 - DNA, 1028 bp, Artificial Sequence
  subsequence of FOXG1
ttgttttcgtcgtcgttatcgttattatcggtcgtcgttttggacggggttaaagcggacgggttgggcggtaagggcgagtcgggcggcgggtcgggggagttggcgttcgtcgggtcggacgagaaggagaagggcgtcggcgtcgggggggaggagaagaagggggcgggcgagggcggtaaggacggggaggggggtaaggagggcgagaagaagaacggtaagtacgagaagtcgtcgtttagttataacgcgtttattatgatggttattcggtagagtttcgagaagcggtttacgtttaacggtatttacgagtttattatgaagaattttttttattatcgcgagaataagtagggttggtagaattttattcgttataatttgttttttaataagtgtttcgtgaaggtgtcgcgttattacgacgattcgggtaagggtaattattggatgttggattcgtcgagcgacgacgtgtttatcggcggtattacgggtaagttgcggcgtcgttttattatttcgcgggttaagttggtttttaagcgcggtgcgcgttttatttttatcggttttatttttatggatcgcgtcggttttttttattggtttatgtcgttttttttgtttttgtattattttcgcgttagtagtattttgagttataacggtattacgtcggtttattttagttattttatgttttatagtttcgtgttgatttagaattcgttgggtaataattattttttttttatcgttaacggtttgagcgtggatcggttggttaacggggagatttcgtacgttacgtattattttacggtcgtcgcgttagtcgtttcggtgttttgcggtttgtcggtgttttgttttgggatttatttttttaatttttgtttcgttaatttgttcgcgggttagattagttatttttttttttacgtttcgtattcgttaatgatttcgtagagtagtacgtttatgagcgttagggtcgcgtttttttttacgtcgtr

SEQ ID NO: 175 - DNA, 1017 bp, Artificial Sequence
  subsequence of ATP4A
acgagggaacggggcggggttgaggagaggggcggggtcgagagttcggtgcgggttttgagagttgcggggaagggtgaaggtggaagatggaggttttgtgtggaggggtttttggtagaaggtaagcgttaaggcggggttaggtgtagatttggagttttggacgtagggaatgaatagaattagggtgcggagttgggttgggggcggatttggagagcgaggtgttttttatggatagtttcgtcgaggagaagttgtgggcggggttgggtggtgggcggggttttgtttttggtggacggggttataggcggagcgggagatggggtggggtttggttgcggagagaaggggtaaggagcgaagtttttcgtggttcgttgatgtgggtgtggtttggggcggggttcgaggtgggcgggtttaggtcgtgggcggggtcggttgcgtatttggaatttgttggtggagttgaagggtatttcgtagatttttgggaagcggtttcggtagtttatggcgttgtttagcgttagtttcgagaatttgagtagcgtcgttttcgatgcgtttttaattacgatgcgttgggagcggggatcggtgttaggggcgaagtcggttatattagtttttcgggattttttggaggttttttggtttttattttgggtataggtattgtattttggtcggatttgaaggcggcgcggttgtatagggtgagtattcggtatagcgttcgttacgttttcgaggattggttaaacgtttgttttgtagattaggcgtttaggttgggtttcgtacggcggttttttcggattagaatcgagttttttttttttaggtttatatcgcgggttttttttttagatttgggtggaattgggttttatttaattttgagggatttagttttcggatgatttttttttttagattcggtagcgagttttttttgagatttggagtcgagtcgtttcgttttcgtataaagttttttttatt

SEQ ID NO: 176 - DNA, 1161 bp, Artificial Sequence
  subsequence of NOTCH1
tttataaatatagaagaatttttttattttacgtaggaaaattttggtttttagaatttgtttgtttttttagaatagataaaagtttttatttggggttagataaaatagtatatataaataaaaaggtagtgtttttgtgtaaaataaaagtatataaataaatattaaaaaaaattaaaattttcgtttttattttgtataaaaatatgtgttttaaaaaggtttttttggtcggttttggtatttatagagcgtatatagacgttcgaaggtttgggaaaggaagtcggggtttcgtggggcgcgtcgtttatttgaaggttttcggaatgcgggcgatttgggattgtatgttggtgggagggttggagacgttttcggattagtcggagacgttggaatgcggggacgagttggattattggttaggggatttaggggacggggtgaggaaggggtgtttaggtatttgtagttggtggttgggggtgttgtttataggcgaggagtagttgtgttgcgaggggggcgttaggaattgggttgcggttacgggtgggattagcgaggatggtagcgacgtgggtagggcggggtttttttggggtagaatagtgtgtatcgttaggttgttggggtttagtggttgtacgtttgtttggttcggttttttatttaggaagtttcggtttaggtggtcgttggttgttgagtttacgttaaggtgcggttgtggtggtggtggtggcggttgtaggttttgttgttgttggatgtttgttggttgtaggttttgttgttgtatttgtaagttttgtggttgtatttgttgggtttgtattaggtgaggttgggtggttagtcgggtgttgggtaggttttggtagtttattatttgggatagggcgttggtagtaaggttattgtgtagcgggtttattatgttatgttgtagggagggggtttgtgtgtttagggggtttggtgttatattttttcgtagagggttgtattggttcggtattatgtcgttttgtagtcgggatagttattcgtattgattatttaaattggtggattcgtttatagtgaaatttagggttttttcgttgttggagtttaggacggtgttggtgttagaggttataggtaggtggg

SEQ ID NO: 177 - DNA, 981 bp, Artificial Sequence
  subsequence
ggtttcgtcgaatgcgtttttttggcggtgatggttttggatcgcgcggtcgtagtgtgtcgttcgttgcgttatgcggggttcgttttttcgcgtttatgtcgtacgttggttagcgttttttggttaagcggttttattaattcggttgcgtaaatcgcgtttttggttgagcggtcgttgtgcgcgtttcgtttgttggattattttatttgtgagttgtcggcgttgtttaagttggtttgcggaggcgacggagatattatcgagaattagatgttcgtcgttcgcgtggttattttgttgttgtcgtttgtcgttattttggttttttacggtgtcgtggttcgagttgtttgttgtatgcggtttagcggaggtcggaggagggcggtgggtacgtgtgggttttatttgatagtcgtttgtttgttttacggttcggttatttatatttatttgtagttcgcgtagcgttataattaggtacggggtaagttcgtatcgtttttttatatcgtggttatatttgtttttaattcgtttatttatatttttaggaataagaaagtgaagggggtagcgaggaggttgttgcggagtttggggagaggttaggttgggtagtgagtagttggggaggggagaaagtattaagttagaatttaaggatggaaatattttttagtgagttagtttagattttaggttgtttatttttgtatgataatttgtaagatttgttttaaggagtttaatgggggatatgtttttttttcgtgaggaaatgtttagtttttgagggaaaatttttaaattttttatatatttaggtttagggaaggaaaatttattttttataattttacgcgtagggaaaatgatggacgtgatgttcgtttttagttttttttttatttgatggaagattatggaagattttttggtttttgtaattagaagttttaagttgataagaaa

SEQ ID NO: 178 - DNA, 864 bp, Artificial Sequence
  subsequence
aatgtttttttttgaggtagttggaggttttttttttttgagagtttaattgtggatatagttttgagtagaggcgaaaaggaaagaaggttgggagataatcggggaaaaatatatatttttggtttggtttgaaagttggcgttcgaaaaggaataagaaaaaaaatacgtttcggaagtgtttgagattgttgcggttataaagtagagtattaattttggtattgatacgggaggggagtaagattttattttcgggtttgtgtttatgaatttaggtgaggaggggtattttttttttcgcgtttaaatgttgtattttttaagattattttggttcgttacgtttttattttgtgtttataaaaattttcgagattttagcgggtatagatataagcggttggatatgaagaggaatatatcggcgggagaatatagaatatttattttttttttagttattttggaggtttcggagaaggcggttttagtgattcgtgaagagattttgagttttttatatttcggagaatgataagagagtatttgggcgacgtaattttttgggtttttcggagatgcggttttttatacgttaggggttaagtttgagaaataggtggacggtaaggataggagtttttttcgcgttataaacgtttttgtttatatttgggttttttgatttttttgtttatataatcgagttatttatggtcgggtttcgagtttttattttgcggattgggataaaggggttaattgtgatgtgttttgtataattttaggattttgtatttgtttaagggcgaggtgtaaatttaaaattacgtacgattgaacgtcgagattagttgag

SEQ ID NO: 179 - DNA, 1004 bp, Artificial Sequence
  subsequence of HOXA7
tatttaggggtagatgcggaaattggttttagtcgcgttatgtagcgcgttttcgttcgttttgtcgtaggcgtttttggcgaggttattgtagagttcggggatgttttggtcgtaggaggcgtagggtaggttgtcgtaggcgtcggcgtttaggtcgtagtcggacgtaaaggggttttgataaagggggttgttgatattgtataagttcggaacggtcgaggcgaaggcgtcggcgttcgtttcgtagtcgtttttttgtgagttgggagtaaaggagtaagaagtcggttcggtattttggaatagagaagttttcgtcgtatatttgttaaaaagcgcgtttatataatacgaagaatttataattttgatttgtgatttgttgttcggtagtttttagtgtcggttttacgaggtagagtgatatatgataatattatatttttagatttatattaaattttatttttttttggacggagttcgtcgtagtacgtgatcgtttatatgatcgttttcgttaattttagtagtttttataggtggtttcgtttcgtagggttcgtagtcgtttagaatggaagggtaagaggtttaaatatgcggttaaagaattcgttcgcgttcggcgggtttggcgcgtttcgcggaaaaagatttggaggtttcgcgggagcgtttagttggcggttaattttcgtattggggtttgcggacgttaggcggttcggttttacgtagtattttttatttcgttttttcgtcgatttttgttagtgagttttggattaagtttgggattttttttattttttttttgttcgtttgtttagattttggaagggtttttgtttttcgtaatagtttgtttcgcggtggttttgtgggtaggatttagttatgagtagatcgattttgtttaagtttttttttatttaggtttagtgggcgataggtcggatttagattcggatttagacggggaaggcgtagtattt

SEQ ID NO: 180 - DNA, 963 bp, Artificial Sequence
  subsequence of ZNF529
tatttatattattatatacggacgtagttatagaacggtatatataaatataattatatagtttttttttttagtaggtgatttgtatataggtttggggtttttgtttttataaatttttttcgcgttttatgaatttaggttttttgtcgtgagtttttttcgtatttagattcgggttattttaatttattatatggaaaacgaatattaggttaacggaaaagggagtcggggttttggtagaagcgtagtgttagtcgtaatagggcggtcgtgggtgtaaacggaggggagcgtcggtagttagtatcgcgcggcgattaacgggtcgtttcggagatttttgggagtttaggtttacgcgcgagtgcgtaggcgtagacgatttattttgggtcgggggtgaggtttggtgggtgttaggggtgttgaggaggttaaggagggtattgggagtttcgtttcgggagagttgtttaaggacgcggaatcgggtggggaggttagggaggtaattaagagttcgagtcgggtttggaaattttgaggaggaaatcgggatcgcggtatttagacgtttcgatttgtacgagtcgggtttcgggcgtggtttatgggcgtgattttgagggtgattcggagcgagtttcgcgaagggggattttgttcgttttgatttcgcggaagcgttatcgttggcggcgggttttcgcgcggagcgggcgttgtgtgttcgcgcgcgcgggcgcgcgttgaataggggtggttttttgcgtttaatttgttgtattttaggtgtttgtttttttttgggatgttgtggtcgattgtgtgtgaaaagtgaacggagtagtgttgttgcgcgaggttttttttgcgtgtgtttatttttagattaggtgtgagtcgtatttgagattgtttttgagtgtggagaattgtaagtgatttgtaggtgtg

SEQ ID NO: 181 - DNA, 956 bp, Artificial Sequence
  subsequence of PRLHR
tttcgcgggagttgaacggatttaaatgttttttagtgggttagtagttttttagagggattgggagcgtttttttttagtttcgttttttattttagtttttatgtatggaaaataggggtggagcgcgttttgttatgttcggtgtttagagaattagtgaagaggatagtgtggggaggatggaattcgaaacgggaggaggggtcggtaaatttggttagagtagcgaagaggacgaatttttattgtaggagtttgtacggtttttggagggttagggttcggaggagcgtatagtggaggagggcggaggagaggcgcgaagcgttaggttatcgggaggagtcggtgtagttttaggagggaaggagcggggaaggaagtagcggtgggagattgtaatgtagttgggggtttgagagaggtacgattgattcgtttttaaaggttggattttgtttagcggttaggttgcggagggtagagacgttagagggttcgggttgggatttaatagtagagtttggagttggtcgcggatttgaggatttttattttttcgtggtttcgttatacgagttttttgtttggttgtagcgcgtttagcgggtagcgggttcggttaagttaaaggtaggagttagtattacggatagttttcgttggatttgtcgtttttcgggcggatgatgtggggaggagtaggggaaaatgattagggaatttttgtttttttttggattcgggtgtttggggaaggaatttggttcggtttaaagatttgtttcgttgtttgttttttgggttggggttgagaagagtagggtggagtggaggtagttgagtcgggggtttgggggtcggagatttatgatttttgttttttttggtggttatcgtatttagtgttaaggacggcgtcgttttttatttttcggggttagaaaatgtag

SEQ ID NO: 182 - DNA, 1037 bp, Artificial Sequence
  subsequence of ARL5C
gagggagcgaagagtagggaggtagtttgggatatttggtaggtttggagttgggtttggtagggttttcgggaagatggaggaaagtagtgggagttagggttgtttgtacggtgtaggatttgaatgttagatcgaggagtcgtagtattaggaaggtttaagtagcgtgtttaatcgttttttcggattatagaggagttcgggaatttagggaagagttttagtgattggtttttttgtttgttagtttggtgggaggtttagttttttatttttttcgttcgagagttatttataatcggtagagaatggtggttttttttttattgtttagttttacgatgatgatcgtgtgttttggaagcgtcggaggaagggtagcgttagggttaaattcgaagattaagggattgtgtgtttttattagattttttaggaaggagatagcgtttttcgtaggttggggatttttgtttatcgtatcgcgtttgttttttatttagtttcgatatttgttttatttcgggacggtggaaaagatagttgagtttttatttttttttatattttagaaaagtgtttgaaaggttcggggcgtttcggggtttgttaagagacggtgtttagagaaagagtataacgcgaagttataatcgtaggaaattcgtagtagttttttattttcgtcgttggtttcgtttagcggggagaaaggagggtcgtttagttttgcgttttggggcgtatcgaagcgtcgggatttaagaggagtaggtagggacgggggtaggggaggtcgcggggtagtcgaggagcgtttatatacgattttcggagttcgaggttttttcgtgcgttcgattatagattagaggagttttagaagagggcgagattaaagcgttcgtttggatgttttgtgcgttttttattttggttttcgaagatgtttattaatttggcgattagttgttttatggtatttttcgggtcggataggtgtaggagggtttagcggttttaggagcggagtt

SEQ ID NO: 183 - DNA, 1006 bp, Artificial Sequence
  subsequence of PAX9
tggagtttggttgatttgatgttggggtttaaagtttttttagttatatgtttgggttgagttgtttttagagtttttggtttttgttttttttgggtgggtgtatataaggatttggttagtgttgagtgtattggttttttttatttagtgtagtttttttttttatatttttggtttggtttttagttttttattgagtaaatatttatttagagagtgagttgttttttggaggaggttttgatgtggttagggaaaggttttttggtttttttttggtttgagttagttgtaggttttattggtgttttggttttggttttaaatttttttttttagttgaaatttttttgatttttttgagattttttgagtttagagtgagtagtgtaattaggtggtagattttagttgatttttgtttttgttttaggttttgtatttttatatttattgggattgattagattttagtgttttttttttttttttttttattttttttttttgttttagatttgttgttttttggttggttttagtgggttgttgattattttgttttagtatttggtttgtgttttgggaaggttttagtattatttttttttgttgggattttgttgattttgtttgtagttttggttttttttgtttttagagtggggtttttggtttgttgtggatatttagtggtttaaaatatttaaaattgattttttttgtgtttttgagaatgttaagagtatttagtatttgtaattgttggtatgagtggggtttgtggggagttgttagagttgtggataggttttttgtttattgagttgttatttttggtatgtttaggggatttgtagtagttgaggttatatggttatagttaagtttagtaggtggaaagggagagatggtattgaatttatgtgagaattgggaagatgtgtggtagttgaaggagttggtaggaataagagggagttgtatgttatttttatataat

SEQ ID NO: 184 - DNA, 1001 bp, Artificial Sequence
  subsequence of HOXD10
tggtttttttgagagttgaagttttgtgtagttttttggggtgtataggaatttggagttaggtaggaggattgagtgggaggataatgtagggattaatttatttatggggatttgggtttggttagttgtttggttttttggtttttgtggttttttttgtttttgtttttttagagatattttggttgttttttagttttaagggattttttttttggatttttggggtgggatgttttagggtttgtggaatttgatttatttttttgttgggttgtttgggattaaattaattagtttgtgtttttatttgtttgttttggatttggtttttttaagtgtgttttgtttggttgtgtgtggggggaatgtttttgtgtgttggtgttagggtattttggtttttttttttgtttgtgtgtgtttatttggaggtttttagagttgagattttttttttgttggttgggtttaggggttgaagtgggggatgtgagtggggtgggttggttgagtgagttttggagaggtggataggagggtggtggagagtgttgggttggttgtttttagtgtgtattattgtgggtgtgtagtagatgttgttgttgtttgtgtttatttggttggttggttaggttttggagtatgtgatttgagaggaggttgtggtttaaggttatttttaaattttattggtttggttgttatgtggttggtagaggtatttataattatatggggaatgtttttttagagatgttagtttataaaggatataattttttttttttaaatttttttttaaaatgtttttttttaatagtttttttgttgttaatatttttttagtagattttttgattagtgtttgtaggagtgatagtttttattttagtagtgttagtatgtatatgttattatttagtgtagatatggggatttatggaatgtaaatttgtggattgtttttgtttttggttaaaagagaagtgaat

SEQ ID NO: 185 - DNA, 468 bp, Artificial Sequence
  subsequence of TBX20
gtggtttttgttttttattagtgtgtttttagtttggggtttttattttttttagagggattttggaaaatgggatggttgttgaagagaaggtgaaggttagggtgtggagttttatggtaattatgtgatgtgtagtgggagagtgtgaattttatggtagtgtttggattaagtggatatagatttttttggttttataaaggtggttgtgatgaggattttttatggttttgttggagggtgttttgttagttttgtttagtttgaataatttaaattttttgaatttgtagggtaatttgttttgtttggagttgtgtaaatttaggttgatatatatatttaagttgtatatgtaaatatttgtttttttttttagttagttggtttggttattagaagtttttttgtgggttttaaaattaaaataataataataatttttaaaattttaattttttattg

SEQ ID NO: 186 - DNA, 1003 bp, Artificial Sequence
  subsequence of CACNA1B
ttgagggtatttaggggtggtgtggttggtagagtgggttttggggatggtttgggttggtgttgagtggttgttgaggatttataggtttgtttgtttttgggtttggtttagttttggggtgggaggtgtggggtgagggaggtgtagttggaggggttggggtggggtgggggtggttgtggggtggggttggtgtggtagggtgggtggggttggtgtggattggttgggttttgttatgtgattggttttttatagggtgtggtgtggggttgtggagttgggtgaggtggtggtggttgtggtggtggggttgggtgaggtttgttgtggttttggtggttttgtggttgttttgttttgagtgtttggtgtgttttgtgttttttttgttggggttgttgggttggggatgtatgtggggtttgggagttatggtttgttttggggatgagttgggtggttgttatgggggttttggtggtggagagtgggtttggggtggtggggttggtggggtggggggtttgggttttggggggttgtagtttggttagtgggttttttataagtaattgattgtgtagtgtgtgtggattatggtgttgtataattttattttggttaagtagaattgttttattgttaattgtttgttttttgtttttagtgaggataatgttgtttgtaaatatgtgaagtgtattattgagtggttatattttgttgggtggggttgggtggggttgggtggggattggggtgggggttggggttggggttatttttttggtggttgggagggtgtgatttggggaggtgttgggagatgggtgaggggggtatggagtgttttagggttttttggagtggtgggtgtgggagtatttttgtgagtgtggtgggtgtggggtggagtttttttgttttggatttgtttttttgtatgttgggttttggggattttggttggtgtatagtgtgtggttagtttggaggg

SEQ ID NO: 187 - DNA, 1058 bp, Artificial Sequence
  subsequence of ZIC1
aagtaatagggtaggagaataagttgtaaatgtgaaaattagaaattaggagttaggtggaagtaatttgaaattgtgtgtagttttgggtaatatgagtttttagatttgatgttaaaaattgattttttttattattaatttttttaaatataaaaataaaatgtagatttaattgtattagggattataatagagttaagatatttataattttattgtagatgtaagaatttattgatgttttgggtttttaagtttgtaaagtgttaattttgggttgttggttttttgttttattttttttttatttgtttagtttttgaaaaatggttgtggtatttttttttaattagattttttttattttattttggtattaggggagaagttttttaagtgtgagtttgagggttgtgattggtgttttgttaatagtagtgattgtaagaagtatatgtatgtgtatatgagtgataagttttatttttgtaagatgtgtgataagttttatatgtattttagtttgttgtgtaaatatatgaaggtaattgttgtatttttgttgttttttttgaggtaggagtttttttggtttttggtttggggttgggtggtgagtggtagataggtggtggtgggagttagaggaagtgggaaggtaaaggttttatttagtggaattgggtagagaaggtagggtgggatttggaaaaggggatgggagtgtttaaggttgttttaaatttttggtttaataataaaagttaaaagaggtgagtatgagtaaaggttgttattgaatttggtttagagttagggtttttgtagggggttgatgttgtggaaattgttgtttttgtagttgttagtgagatgtaagaattggatttttgtgtttaggtttgagttttggtttatttaaatatggataagtaaagtttagggaaagaaagtgtagtttttggttttatatttagtttttttttttttagatttattttttttttgtgagttttggggattttgatttttttaggagtatggtttttgtaggt

SEQ ID NO: 188 - DNA, 851 bp, Artificial Sequence
  subsequence of SIM1
tttttgttttatttatttttagtttttataagtggtagaggttttgggggtgtttattttttaattgtttttatgtttattgtttaatttagtgatattgttggtttttttgttttttattggtagagaggagtagagtatttttggtttttttttttggattatgaattggaggaggggtgtgtggtagaaagaggaggatgtaagttttgggatgagtgaggtgattttagaattttggttttttgtagttgttatgaagttagtttattattatttttaattggaagttgaaaattgttttttgtaatttttttatagtttaagggtttagttttaattttattttggggttgttttttttttgtttttttttaatgttatttataagtttttttgttttttattggtttggtgtggttaggggtgaggtgtttattggttaatagggttgagtgatatgagttggttttgagttgttttttgtgtgtttaggttttgggttttgaattttattatttgtgggattgttggatttttaatgagtttagggtgttgggttggtgttgtttggttgttgttgttgtttgtggttttttgggtttttttgatgttgtgtgtgatttgtgttttttgagttttttggggttgggtggttgagtgtttagtttagtgtggtggtttagtagaattgggaatgtttggaggtagtggaggttgggtttgtgggttaggtttattggtggaggaggtagggtttgtttaaaggtttattttgttggtagttgttggttgatggtgtgtgtgaggttggtttggggagttatttttttttttttgtttttttgaaa

SEQ ID NO: 189 - DNA, 1068 bp, Artificial Sequence
  subsequence of DLX1
tttgtaggtggtagttatggttggattgagtttttggtaggttaaggtttggattttggttttttttttattataatttttttttggttttttgagagttgtttttgtgggttgatggtggtgggaggtttggtttttgagtttttggtttttatgggggaagggaggaaggagggatgtttttgtttttttggtagggagttgttagttggtgtagggttgggaggttttatttttgttgttttgtggtttttttttttttttttgtgatttaggtaggtttagtggttttagtttgagttatgttgttgtttgtttttgtaggttaagatttggttttaaaataagtgatttaagtttaagaagttgatgaagtagggtggggtggttttggagggtagtgtgttggttaatggttgggttttgtttgttggttttttatttgtgttgtttggttggaattttaattttttatttgggaagggtttaggaggaaatgtgggtttttatatttttagttatatattgtggtattttttagtgtattaagaagttatgtagtaattttaatttatgtgaggttgtttgtttgttttttttttgtttttttggtttaggttttttttgtttttaggtttatttattttgtttggaaaagaaggatttagagggaagaaggaatagtggaggtgggatgttttttatttttttggagttttgtgaggtttggtttagtaattttttggtatttgtgttttagtttgaattttggtttgggttgagtagtggtagtagagagtggttttggagggaagttattgttatttgagatagtttaagtagtaagataaatttgttttatttgatttgttgatttttagttttgtgggattattaggaaaaaataaaataaaaataaaatgtagaaaaagtaaaagtttttttttgttttgttagttttttgtttttttttgttttgtttgtgtgttggtaaagtttaggtttttatttgtttgttgtttttattttgtggttttagtaaaaagttataaggtttgagtggtttgg

SEQ ID NO: 190 - DNA, 1008 bp, Artificial Sequence
  subsequence of LMX1A
ttaggttgtgagaagagggtggtatttatgagatgttgattagttttgagattaaaaatttagttttgggtatttttaattatttgttatttagatgtttatgaataagtttgtttgttttttgtggttttggggaatttggtgtttagtgtgtgaggtttggggtggtttgttggatttatttgggtagggatgatttataagtattgatggatagatagtgatggggtttaatttagtgtatgaagaggggtgttagtttttttattgtggattaggttttagagagtggggttttagagtttagtgttaagtggaaagagagtgtgtttaggatgtatggtttgaatatttatttagtagtgaggagaaggaggttgaggtgttgattgtgttttggaagtttttttttatttttaggttgtttagtatgtttgggttgggttgggaggatttgtagaggagaagaaatgatgtgtttgatgtttgtgtttgttgggatttggtgttagtagttattgtatttttgggaaagaattgagggagtgtttagggtgattagaattagttaggaggatagggtttagttaagagaatgtagggtgggaggagagattagttatagtgaattgttttggttgatttgattttatttgaagttaatatgttatgtatttaggtttttgttttttaattgtgttttttttttttttgttttttttatttttatttatattttttgttttaggttttttattttgaatttgattttgttttaatttatatgaaggtgggtttttgggatgtttttgtaggaatgtagttattggggtatattgggtatataaagagtgggtatttttttttgagtgattgggtttaggtatgtgggatgatggggtttgtaattttgtgttttagttgttttgttgtggtttttatttgttttaggttgagaaggggtattgtaagggatttggagggtgttttgtttgtttttggattttt

SEQ ID NO: 191 - DNA, 1007 bp, Artificial Sequence
  subsequence of DLX6AS1
ttgtggtgtgttattttggtatgttgtgtgtgggttatattatttttagttttttatttttgatattggtaggaaagtttgatagttaggtttgaagtttagattttattttgtaagtgtagagatattatattaagtgggattttttgtttgttttattttttagtttttgattgtttttgagtttagattgggttgtttagtaggttttagtaggatttttattatgtattataattattattttagtgtagttgttttattggaatagagtgtaaggattttagttattaattattaggggaaggttgagagttttgaagttataatttggataattttagtttttttggtttttttagtaattaaataaatgagaaaaaaaaatttttttgtaagtagaggtagttgtattttggatatagtgtttaattgtagggattttttgaggttttgttatgatatattttaattggggtgtagttagagttgtaggaagttggtgaggaaagtggtgtatttgtgagtataaagtgaaaaatatagataagtatagggtgagggtgtagggggtgaaatgtttttatggtgttaataataagttgtagggtataaagtaataaaattgttatgtttatttgttggtattgaggatattataaatagttttggtgtgtagtttgtgtgtattgagagtgtattagggttgggagtgggaaggtggggtgggaaattttttttttttttgatatttttttttgttttttattttttatattttttttttatttagtttatttttttttttttatgaagtttttttgttgttgttttaatttattgttgatttgtaaatgtgtagattgtgttattggtttgtgtgttaaagtgtattttttgttaaaaggtggggaatagggtatgttatttaaaaatgaatttagagttattgaattggttttttagatttaagaatttaagtgtaatgggttgggaaaaggaggggg

SEQ ID NO: 192 - DNA, 973 bp, Artificial Sequence
  subsequence of MIR196A1
ttatatttgtatttattgtaaaaatgtagttgtgtaggttgagggtggtggtggatttatttgggtggtggtggtgttggtagtgtgtggttgttggaaggtgtggagttagtggttgtggtttagtttaggtttttttttgtttttgtttagaagtgagggtttgagtggttatgtgtttggtagggaagtttgaggtagtgagggagtttatttggttgtttgtttttttttttgggtataaattttggggattggaaatataataaaattttattgtgttgatgaattttaaatggtttatatttttttttgttttgaaaagaaagatgtttgggggtggattaaaaggaaggggaaataaatttatgagtggtagaagtatttgttttggtttttttttttgtttgtatattttgtttgtgtggagagaagtagttttagtttttgggatttggtttttgttgaattggttgttaggtgtgttttttttttggaggagttggtgttgatttagtttgtgtttggtagagattggaagttgttgtttatatggaaaatttatagagttttgtgtagattttatgattggtttggtttgttgggtttgggatgaggttttttgttttttgttagagtagaattgggttttttggtgtttttttgtgaaagtttaggtttttttaattatgtttggtggtttttattttttgtatttttttgttgaaagtgggtttttttttttttttttttttttgatagagttttgttgtgttatttaggttggagtgtagtggtgtgattttggtttattgtaatttttgttttttggtttaagtgatttttttgttttagttttttgagtagttggaattataggtgtttgttattatgtttggttaatttttgtatttttagtagagatggggttttataatattggttaagttgattttgaatttttgattttgtg

SEQ ID NO: 193 - DNA, 1050 bp, Artificial Sequence
  subsequence of FEZF2
ttttaaaaggaggtaattggtgtttgtgttttttttgggaaagtgtgagtggtttgtgaatagggaaagagggaagagatgggagggggagggggaggaattgtgatggttttgttggtggaaggaaagggggggggggatttaaatttaaatttaggtagagttttgtgtgtgatttataattttggaggtttgtaggtttgggggatgtggggagggtggagggttttggatgtggtgtttttaaagggtttttgggtattagtttagtagttgaagtattttttagttttttgtagttgagttgggtattgtgttaataaattgttatttatttatggaattggtgttagttttgaatatgtgtaaatgataattattttattaggatgtggtgtatggatgtgggtgttgtgtttggaggggggaatttgttaatggggaaatattaatttatgtaaaagtaatgagtttgttttggttggtgttggaggttgggagaaagtttttattgattaaggatgattttgaagtgttatggagagtaaggttgtttgttggtattttgggggttttggtgttgtgtaggtgtgtttttagttttatggttagttttgggtttgaggttgggggagattaaattgagagttggaagatttttggagattttaatagattagaattgttgatttatgattgggtttttgaggtaaagttgagttttgtttttggtttagggttgtgtgtggggaggatttggagggtagtttttttagttgatgggtatttattgtttgtataattgagaaggttttatttttatttgtttaaggagtagattgaaatagtagaatttttttttggattttattaaaatgtttttttttttaaggttatttagttgaattaattttttgaagtgatttatagagttgaattttgttaaggtagtttttttatttttagttattaaagtattgtgtggaattgggttttgtgtttttttaaaataaagagaaggattattagagaggagagggagggggtttg

SEQ ID NO: 194 - DNA, 974 bp, Artificial Sequence
  subsequence of STAG3
tggatagggggtggggtttgaggtggggttttgttaataatggtttggagttggttttaggggtgggggtaaggaagaaattaagttaatgtagtgtggaagtttggtaggggggtgggatttggttggtatttaggaggttggtgaaataggggtgtggttttttttgggggtgtggtgatttggtttaaaaggtggggtttgggtggaatttagtttatggtaattgagaagttagattaaaggggttattagtagtttggtttaaaatttgtttaatggagtaggagattgagggtagtgggggtgggatttgggtgggggtggggttaaagtgggttgtttgtgtgtttaagtttatgtgagagggtaggatgtttgagagtttgaggttatatgaggttgtggagtggtgtgatttaaatgtggtgtgtattagtttgtatatttttaaattttgtgatagttattggttttgggtgagttgttgggattgtgtttgtgtatagtgtgttttgtatgtttttgtgtttttttggaattttttgtttttggaagggtagtggtgggtgtttgtgtggaaggtggggtggttagagatatttgagggatttgaggtggggattttttttgaattttgtttggttagaagttatttttttaggttttgataggtttttttttgatttaagaataggtttttttgtgatttttttggttttgatattaggagtttttttgggttgtgttttaatggtttttgttatagagttggagaattttttttttaggtttttatagattgagagtttatttttttaagtgtttggaaggttttttattttggagttgtttattttaggggtagttgtagatatttttattttttggaaaaataggaaatgtgtatttgttttttttttttttgtgtttttagttttatttttatggtgtttttatttttaaatttttaa

SEQ ID NO: 195 - DNA, 981 bp, Artificial Sequence
  subsequence of PTPRN2
taataatattgaaggatttgagggtggtttttttgtggttttgaggttaatgttgtagtgtatgtagtaaatatttgttaaggatttattgtgtgtttggtatattaaggtaggtaataaaggatttagtgtatgtttggtatgtagaggtaggtaataaaggatttattgtaggtttggtatgtagaggtaggtaataaatgattagttttagaagtagatgtgtttgttgtaaattatttttttatggtttagtagaggtataataagtagtgaattttttttagggttttttgtgaatatggtattttatgttgttatagtgatgttattttagttttgagttgtaataaagtggtagtgtttgtgttttatttttggttattttgttgagaattatgttgattaggatgtaggtgttgttttggtttgtattgttattgttaatagaaggagagaaatgaaagtgttgttgtttatatggaagtttgatttataataaagttagtgatagtttattttttttttgtttatgggggttggtgagaggaaaggatggtggatgtgagagggggaggaaggatgtgggttgggagaatttaagtgaggggagaagggaggtttttgggggttttgagtttttgagtagtttgtagtttgtgggatgagttgttgtgtttagatgtgatttttttttgtttgtttttttttttttggttaaagatgtggtaaattttgttagaataaaatgaattaagtgtttgattgtgttagagttgatggtatttgtattgaaaatatttgtaaggtgttttttttagtgttgttgggtaattttttgagtggagaggggagggtgttgggtggttgggttttgttggtttatgtatgtagggatattttaggtatatttttttaggttgtttgttattttttttaaatgtaggttttttgttttagaaggagaaattgatataatag

SEQ ID NO: 196 - DNA, 1004 bp, Artificial Sequence
  subsequence of BARHL1
ggggtattggatttgaatttgttttttaattttttttttgaatatatttattagatgtagatgtaaatgtataaatatagaggtatttatatatttaatttaaatagttatatattgatatatatattaatataatttgtagttatatatgtatgtatgtttagttttatagttatttgtatgtttttagtagaggtaggaatggtgttttttataaataagttttagaatatattgttttattaataataaatattgattattgttttgggtattaataattaaggtttgttatagtaattatataattatgatgatgatgaataatttagggagggaaatagggttggtgtggttgagtttattttagtgggattttggttggtttgtttagttttatttgtaggtgagtttagggtttaggtttgatttttttaaattttaggttgttggttggttaaggtttttggattttggggttagatagtgtgtagggtttgtagtgtgagttttggaggatggtaggtgtagtttgttttagttggtagggtgtgtgagttgggagtgggttttttttggttgttattttaggtttgtttgtttttgttagtttgtagtttggttagtttgtagaggaattaattgtatttttttttttgaagagtatttttttatttttttaggtttttttagggtgattttatttttataaagaggtatgtgtagggaggagtattttttatttagttttaagggtttgttttgtgattttttgggtagggtaaggtgtagttagagttgggttttggggttagatagtagggtttaaaatttaggtttttttttgaaaattggtttagattttagtttttttttttgtaaagtggggtaagagttatggaaggttggtggaagataaaaggagaggatgtatgtaaggtgtgtgatggtgtttggtatttggttatttattaataaatgagtatgattttttttggtttttt

SEQ ID NO: 197 - DNA, 1064 bp, Artificial Sequence
  subsequence of CCDC140
tgtttatgggtttataaattagttgttatttgttataaatatgtataaatggaggttataaggatgtgggggtgttggggatttaaagttttggaattaggaggtgatttatttttttttttttttggttttttttttttttgttgagtgttttgagatttatatgtatttagatgattagtttggttttttttattggttagtggttagaggtttttggatatttggagaggggtgggagggtatttttaggttttggatagttgggttatatttaaagatttaatgaatttaaattttttgtggtttgtgtttttgaagtttgatttgggatggttaggatagtaaagttggttgaggttgtttggtttagggtgtttgtatgttgttaaatttaatttggtttagagttgtgattttgtagaatagggtgtaataggttttaggaatgttgggtgattttagttattttttttttgtttttgagttttgggttttttttttatttttttttgaagtttttttggagtttaagttattttatatttaaattttgtagttggatgggagtttaggttatttttttatttgtagttggtgggttaaggagttttggtgttaaatttggttgtggtataggtgttaattggtaaagggatgatttagtggtggggtttggaaggtgagggtttgtgttttgtatttttgaattttggtttgggaggtagtgatttttatttgggttaataaattatttagaaagtattagtgggattttttataatggtgttgtgaatgagtgtagggtttatttatgttttgtgtatgtttgaaatgaaatgggagttttgaagatgatatttggataatttgataaaaaaaaaaaaaaggtgggtgggggagatagtttttattgttttttttgtgtttttgtttttagttttagagatattataggtgaaaataatagtgggttggttgtttaggtttgagtggagtgtgtttttgttagtttgaaagtttttttttgtagtttgtttggatgtgtgtgtttatagtttagt

SEQ ID NO: 198 - DNA, 1780 bp, Artificial Sequence
  subsequence
tgttttgttggggttttttttgttttgtaaagggagtggggagttgggggagttttgtatattagtttttttggagttgtagatgaggagttttgtgtttttgggagttttttggtggttttttttttaatgtgtgattgtgtagtttttttttgttgtttttatttgtggtttgtttttgttttttttttttagttttgttagttggagtgtgtatattgggattttggtatgtgggtgtgtgggaggtggtggttgttgttgttgttgttgttgttgttggagtttgaatttaagttttagttggagttggagttggagttggtgagattgttttttatgttgtgggagttgtggttgttttttgtttagtttttggttttaggtgagtttggtgagttggtgaagtattggtggggaggaggattagaataggaggaggggtatggtggattgaagtgagttgggttgtgagtaagggatatttatagtttggagaaatagttttgtttttttgtgtgttgtttgttttagttgttattgggggttttaagtagtgtgatgttgttttgttttttttaggtggtggttggtggaggttttttgtagttgtttggttggtgttggtttttattttgttggtaagttttttgtttattttggagggtgtatttggatgtttgagttggagtgagtgtgaagtttgaagtttgtttttagagttgttaatttttttgtgagtttttttttttgttgtagtttgtgttagatttgggagtgatgtgtttttatttgggtggtgtgttttttgttaatttgttatttatgggattgattgtttttagttgtttggtttggagtgtgtagtttttgttgtgtggtttaggagaggagtgagatattaaggtgttttgtggattttgtgtgtattagttggtttggtggttttgtgggatagtgtttatatttaggggtgagtaggttgttaatggaggttttttttatttttagttagtggtttgtatggatggtaaagagatgggagaaagttgggtttttttgtgtttaatttggtattggtagaggaatgtaatattgttttttttaatttgattgttgtgggggtttttttagttttttttatttttaagttaaggagggagtttttttttgttttttaaggagaaaaggggttttttagagtagattgttgatttttttattaatgtgttatttggggttttttagttgtttgtgtgttaagttagtttttagtataagtgtttagagatttgtagttgtttgggtgtgttaagagattgtgggtagatttagtttttagggtagtttaagtgtgttttattaaagaaaaaagattgttggtatattaaagaagtgtgtgtgtgagagagaaagagatagagaaagagatagagagatatgggtgggggtattaggaaaggtagtttgagtaggaggtgttagtgaaatttattttttgttgatggatttagtttttggaatgtttgattgttttgatttgtggtgtggtgtgggttttaggggtgtataatatagggtgtttagatagagtttaataggtggtttgtgtgggtattttgtggttaggtgtgggggaggggaatggttttttttttgagttttgggttgtttttgttgtttttaggggtgtatttgaatatggataaggaaatgtgttttttgtttttaggttttatttggggtgagtgtgtgaggtatttaaatgttttttgttgga

SEQ ID NO: 199 - DNA, 1038 bp, Artificial Sequence
  subsequence of GATA2
gttggtatataggaggggtaggtggggatggggtggtgtgtagtaggttgggtgtttatagtagttaggtttgggtgtaggggattgttattttttatttttatgttttttgttagtgatatttggtatttgatgttgtttttgttttttttttgggttgtattattttttgtggaagatgaggttggagatgtagtttttgtggtgttagggttaggagatatttttttgggtggtgtgggtgggaagttgaaaaggtgggagttagagtgggttgttgtaggggtgagggaggttattgagtttttgttgttttttttgtttttatttttagtttttgggtatatagagagtgggtttttagggtttttagtagttgaggggtgtagtggtgttttggagaaggggtttatggtttaggggttgtggtggtgggttgtagtggtagagagggttgttttgtttttgtttagttagggtaaatttgggttgtgtaataagtgtgggtggtatatttggtttttggttaggtgggttgtgggtaaagagagagaggattagggtgggtagaaagattagggtaggtagagttagggatgtttttgatagatattgagattatgatttttagaattagtagttattttttttttaaagaaagttagaaatataatattttattggtaataattaggaatgttagtttaagttgaaggataagtggtatagaaggaattttattggatagattttatagggaatttttataggttagttggaagtgggtagaaattttgtgggttttagattttttttaattggttgttgtttttattttttttgttttaattttttagtagtttgatttttgtggattttatatttgggaagtaagtagatgggtttttttttttttttttgtttggtgtgtggtgtttgggtttttattattatgggtttagtgtttattgtgtgtggggttgtaggagatgtgtgtttgtgtgtgagtggggttggtatagtaggggttgttttgtg

SEQ ID NO: 200 - DNA, 1100 bp, Artificial Sequence
  subsequence of ADCYAP1
gaggtaaaaagggaatgtgagttagggagtttgatttgttttgaatgaaaagaaagagaaattaaattaaattttttagttatttaaaatttttaggtttttagggaggttttgttataattttttttaagtatgattgtttttgggggaggggaaaggggtggttgtatttattgaaaatttaaattgaaataataaatggttaaatgtggatatttatggatttaaatagttttgtttatgttagagaaattgagagtatagggtttgtgtgaagtttattttggtagaaggtaatattttaataaagtttgtgggaaaatagattatatttttgttatgaataagttatgtagtgaaaaatattgtttatagtttgttgatttatattattattatgttttttaatttggtgtgaggagggagaggagtgtttatatttgattaggaattgtaggattgatgtaaattttagggtagtagttagattggtatatgtagggttttttggttattttttttgtatgttgtgggtgagaggaatagtgaggataatttagtgtaaatatatgaagggttggattttaagggggtagtgttgggagaaaggttaggtttgaagtgtgtgttgtttgtttggattttattttgggttttttttgtagggtttggtgttaggagattttgtgtggggaggggggtattgaggggttgttgttttggttttttttatggttgtttttaggtagaggtgggtgatgtggtgggtagtgtgagttttgggttttttttgaaggtttttgtgtggggtggggtttgtttgttttttgtggtgattgaatttgtgttttttgttttgttattttttttttgatttttttgtttgtagtgggagttttggtggtggtgtgggggatgatgtggagttgttttttaagtgttatttggatgggatttttatggatagttatagttgttattggaaataaatggttgttaagaaatatttggtggttgttttagggaagaggtataaataaagggttaaaaataaaggatgttgaatagtttatttgtagtgatgggttattagttatttt

SEQ ID NO: 201 - DNA, 963 bp, Artificial Sequence
  subsequence of TBX5
gtagagaataattattgtattagggtttgttgaggtatgaggtattttattttgttttgtaattatgatgattattatttttagtttgattaatgggtataatttttaagtagtgtagtggtggatgtttgtaaatttttgtgtattgttggaaatttattaggttgagttgtaaaatgtattgtgtagatgtttttggtggtgttgagagaagagttaggtttgtttagtatagagttggagagtgttgggttttttggaagggtaagttttttgttaaggggttttgagggagttggatgtttgaatttggatttgtttttagttttggggtttgattttgggttttgtgtgtttttaatttttagggttttttgaagtatggtttggttttaggtttggttttgtaaggattggaatggtagtaaaatgtgtagggaggtagttggttggtagagttgtggtgggagttaaggttaggtttgtggggagagtgggtagtttttagtgttggttataagtttttaggttagttgggttgtagatttttttgtttttagagagtataatttgtgtttttttttttagttaggttgtagtggttgttttgagttttgtttttgttttttaagttgttaataatgatatgtttttaaatttgaggtttgtgtttgtttttagatgttaagaatgtaatttgaaattttttttttaaattttaggttgatgagatgagtttttatttgatttttgagttgaggtgggttggaaattgaggtttaggttttgttggggttgtaaggaaaaggggaaattttgagtgtagtgttttttttttgtggtttttttttttggtattttggattgtgggttttgtagttatttggattggtatttaaaggattttgtaagtttagttttatagattggtttttttagatgttttgaagtttgtattatgaa

SEQ ID NO: 202 - DNA, 837 bp, Artificial Sequence
  subsequence
ttagtttttatttatatttttgttatgatgggtaattgggtatgtgtaggggaaaaatagtaatgttaaaatgtaattgtgtaaatgaggatagagagtgtgaaaggagggagaggtgaggagttgttttatttttagggaggttttggggagattgattagttttaaggatagattgttgggaagggaaaaatggtttatattgaattggatgttggatggaaatttttttgtgttattagattgtgtttagtatagtagatggtatgagtatgtgtggtgtttagtttaggtttttggaggtagttgagttggaaattttgatggaaagtatttataagtttttattttgtgttggtttatttgtgtgtatgtttattttttatgtgtgtttttggtttagaagtgtatagatgtttattgtttagagttggtattgttggggagattgagtgatttgtgtggtgtatagtgtggtgttggtagggttttgggtttttggttgggggtttgagtggttaagggatgggggtgggggtggggagagtggggggagggtgaaagattgttgagaggaggggggagtgggtggattaatgatgaaaaagttttttttattttagttttttaattaaatgtatggaaagaattgaggtgagtatatttggttttaatttatagttttttgatggtattaaatgttttttttttagattagggttggaagttttgggttaattatggttgtttggagtttagaaattatttatatatatttgtatttttttttttttttagttgagttttttgattataggatgaaaaaaaaaaaaagtt

SEQ ID NO: 203 - DNA, 1012 bp, Artificial Sequence
  subsequence of NECAB1
tttgtgattttgggaaattttatatttgaatattatttaataatttttgttttatagtaaatgttttgaagtttattttaggtagagtttatagtattgtttttgttagtagtattgttataggtaagttttagaattttttggggtgagattggtgtaattattttattgatgaggaaaagtgaggtttataggttaagttttttgagtgtaagataatattgaatggtttaatttttatattaaattagttgttttttgattgttggtatttgtgaattttgtttttgtttttggggaaggttgggtagataggttattgggattaaaagtagagtaggtagaatttttttgagtttttttttatttatggtatttatttaggtgaggttgggggtgtttttattttgtgttttggttgtttaggataaatgttttgttatttgttttttttttttgtttggatgtttttttttatttttgtatttggggttgttgtggggttggtaggggtttttaggttatttttagttgtggttgatggttgtgttgggggtagagtttgtttttttttgtgtgttgttatgtgtggggatgatatggatttagatgttgttaattatgggaaggaggaggtgagagttggagtgtgtgggagtgtagaggttggaggagggagggggggatattgagtgtggagagtgtggagagtgtggagggaggtgtgtgtgggagtgaatatttttttggatttagagtttggtggtggtgaagtagtagttgtggttgtgtttttgttagagttggtgtgtttgtttagttttgttttgtttgaggttgttagggtttttgaggatggaagatttttaggagatattgttgttttttaataattttttggaggagtttagttttgttttgtatttgtttaagggtatgttgattttttttgatgtaagtatagatggtgggagttgggtggttgttttttagtattttttttttttttgaa

SEQ ID NO: 204 - DNA, 1017 bp, Artificial Sequence
  subsequence of ADRB1
tttgagttgttgttttagtagtggatagtgggtatgggtttgttgatggtgtttattgtgttgtttattgtggtgggtaatgtgttggtgattgtggttattgttaagatgttgtggttgtagatgtttattaatttttttattatgtttttggttagtgttgatttggttatggggttgttggtggtgttgtttggggttattattgtggtgtggggttgttgggagtatggtttttttttttgtgagttgtggattttagtggatgtgttgtgtgtgatggttagtattgagattttgtgtgttattgttttggattgttattttgttattattttgtttttttgttattagagtttgttgatgtgtgtgtgggtgtggggttttgtgtgtattgtgtgggttattttggttttggtgttttttttgtttatttttatgtattggtggtgggtggagagtgatgaggtgtgttgttgttataatgattttaagtgttgtgattttgttattaattgggtttatgttattgttttgtttgtagtttttttttatgtgtttttgtgtattatggtttttgtgtatttgtgggtgttttgtgaggtttagaagtaggtgaagaagattgatagttgtgagtgttgtttttttggtggtttagtgtggttgtttttgtttttgtttttgtttgtttttgtgtttgtgttgttgtttggatttttgtgttttgttgttgttgttgttattgttttgttggttaatgggtgtgtgggtaagtggtggtttttgtgttttgtggttttgtgtgagtagaaggtgtttaagatgttgggtattattatgggtgtttttatgttttgttggttgtttttttttttggttaatgtggtgaaggttttttattgtgagttggtgtttgattgtttttttgttttttttaattggttgggttatgttaatttggtttttaattttattatttattgttgtagttttgatttttgtaaggttttttag

SEQ ID NO: 205 - DNA, 1030 bp, Artificial Sequence
  subsequence of OTX2
ggtggtttttggtttaggttgatttgtaagtgaattgagtttttggtgtgtgggtttaaggaggtgtttggttttttattattgttgttttaatttatttatttagttatatttgtatttttttgttttggattttaaaaaggtttttgggatttatttaaatgattttaattaaatttgggggttaaaatgtaaagattgtgggagaagtttagaatggtgttgatataaaaataaaattataaataaaattttttaaaatattttagattatatttgtagtgttttgatgatgttttgtagaagtgatgatttttaggagtatgttttttgttttttttttattattgtttttatttttagattatatttttttttttgtaatgattgttattatattttaaaatttgtaagagaaaaaatatatttgtttatagaattttatgtttgtagaggggtattggttggtttattggaggtattggggtgatggttgatagatatggggatatgtagatatggatatagataagatagatggatatttattttatagttttatgggaggttagaaaaagttaaatattttttaaattttaaatagttagttattttaaaagaaattaagagtattgagttagaaattttgtagtttttatttttttatgaaaagttaaatataattaaaaaaaattttttttaaagaatgaaaatgaggaaggtggttagagttttaaatatgtgtgggtttttttgtaatatatataaagtagatgtggtgagtgagggttgttttagtaatttttatttttaattatagtggaggttgtgttggagttgaggttggttttgtttttagggagatttgttgagaaagtgtttttttggtaattttttgatgtaaattttttaattaatggtagggagagaatgattgatatatttaagttagagggaaaataataaaaatatataatagggggaggaaaataggttgttgttgtatggggggatgagtaggtaataaatt

SEQ ID NO: 206 - DNA, 1076 bp, Artificial Sequence
  subsequence of NKX2-6
aagtgaatttttgagatttagggttttatttttgttaggggtgtgtgaagtatattgggtgttaggagaggatgggaatgggatttgagaggtttttttttggattttttgagagaaaatggagagagttttgaatttaggagataggaggtgtattttttttatgtatttatggattatgtttttgtttttatattttttttgtaggaggtaagatttgagtgtttatttatgtggtttttttatttgttttgtgtttatttttaagattgttttatagggatttttaggaggttttgaattatttagttttttgttattgttgttgttattagtgttgtgaatttttgatttttgtggttttgtgtttatttttaggtattgaaagttttttgggtttttttgtatttgtggatgtggtgaagttgtggggtagttttgtttgtgttttagttgtaggatgtttttgattgagaagggggtggaggtgatggggtttagtagtattttgaaggtggatggggtggggttgaggaggtttgggtgaggagtggtattttgaattttttgttttgttgttgtaggttttgtagatagatttaagttttgggatagatgtttagtgttttagatagtgtttttttttgggttatgttggtaggtttgggtttagggttgggtgatgagattgtagttttttattggtttttgtagaaattatgtgttagttttgtattttttttttttagtgttttttttttgtgtttgttgaattttttttagtttttagttgtttttttgggaggtttggtttgtattgatggtttttaaagtaaagaaaatgtggagagatagtgttgtgtgtggtttggatttttgttaatatggtattagtgggagtttttgaggttaatttagggtttatggggatttttttagtggatatgtggggattggaggtggagtttttgttgtgtaatttgatttggtttatttagtttggttttagtttaattttgtatttttatggtggaggaaagttaagatagagatagttgggatttgtggaatttgag

SEQ ID NO: 207 - DNA, 927 bp, Artificial Sequence
  subsequence of SIM1
ttgatagagttttttttttggaatgaatttaatatttttgataaaatatatattatagatttgatattattattgtttttattttgataggaagaggaggaagataagtgtgtgttttgtttttttaaaatttttttttattttaataaataaatgttatattttaatagaaatgtattttttttgtagttttttttgggaatttttatttttttaattaattgaggtgttttgtaggtaatgggggagggtggtggaggttttgttttatgatttgattatgatttggtgattgaggaggggaaagtttttttaggttgaggttattgtggtttttgaatagtttgttttaattaattgtgtattttttgagagagtgtaggagagtttggtttagggggtaattttgaaaattttaaattttatgtattttgttgggtttgtatatttttgttaaatgtgtattgtgtaagttttttttgggttgaggtgttgagtggtttttgtgtttagtttttgggtgtttttgtagttgagttttttagagaaaaaaagagagggaaattggtgtaattagtttgaattaagtttttagagtagttattggtaggaggtttttttatgtattaaggtgtatttgattttatttttggtattgttgtttgttttgtttgtggttttttttggttaaggagtttgtgtggttgatttgagggtattggtgagtgtgttgggttaggttttgttttatgtagtagtttggattttatagtgtgtgtttgtatttggtagtgttttgattttttttgtttgttgattttgggttagagatattgtggttgtatttagggaatttattaatgttattgtaaatgtggggtggaggttgaaggttatattgttgtttgttttttttttttttttttt

SEQ ID NO: 208 - DNA, 927 bp, Artificial Sequence
  subsequence of DRD4
ataggtagatgtaggtttagttttttggttgttgtgggatatatatatatatatattgttatagttattgtttattatatatatttagtgtagatgttggtatatttttagaaggaggtttatagtttgtaggggagatttgggttggataaaatttaggggaggggagggtgtgtggggattaggtttttgttgagaattttggggggaagtttgagggggaattgggggatggagtttatattttatattaggtttggtttttgagtgggttggttttggtgttagtttttttgtggttagagaaaagtagtttagggttgagttggagatgtggtgtttttgattgtgggggagggggatttgaggtttttttttgatggatatagtgaatttaggtggttggggtagagattagtagtatgggatatgtgtgatttgtgttttttttgagttgtagatgttatagtgatgatgtttaagtttttaatttttttaaattggtgggaaggattagaggggttgtttgtttttttgtttttggagagagttattttatgtggagttttatgttgggtattgggtatggtttttatatttttaattataatttatttttaagataattatgatgtggatattattattatttttgttttatagatgaagaaatagaaatgaattaatgtgtttgagttttttatagagaatttagttaatagagtagttagttttgtttttaggtttagtggggattttgtttattttaggttggggaggggttttttttgagtttgatgatattaggtttgtttttagggaattttttaggttgtttttatgtttgtaataaaagggagattaaggttagaagttgtgatttatgtttgtaattttaggattttgggaggtaaaggtaggaggata

SEQ ID NO: 209 - DNA, 810 bp, Artificial Sequence
  subsequence of TBX15
gaaaaggtttggatggttgaaataaatttttttttaattttttttttattgattaataaaaataattttttagtattaaatttaaatattgtaatgggttataaaaatatggagaatttataaaattttatttttgtaggttatttgttaattgtattattattagttttgggagtatggaaattgaattgttattgtttaaagagaaaatgtaagtgatagttgttttttttttgagttggatagttttgtggttgagatttttaagtttttgagttttagattgtttttattttagtgtaggttgttgttttttttttgggtttgttagtgtgtgaagtggggtttattttttagttttggttgtttagtaggagtattttttagtatggttttagaagtttgggttggaaagtttttgaaattgttaaattaagttgtgagtgttgtatgtgtgtgtgtgtatatttaagatttttttgataatagttgtttttttgggttgtttttgtatagttgaaattttggtatttaatataaaatgttaaagttggtttttttttttttagggttttgttaaaatatagatatgatattttttttgttttttatatgttttgtgtatataatatatatgggttattgtaggatttgagatataaattaattgtatttatattttaaatatatagagatgtgggttttgaagagtgatttaaggagtaaggggtttaatatttatttttgtttttgtaaatgttaaaggagtagaaatataattatgttttattattgaaaagttggtgggag

SEQ ID NO: 210 - DNA, 1080 bp, Artificial Sequence
  subsequence of TRH
ttttttttttttttttttttgttttatttttgtttgtttgtgttttatattggttttgtggttgtgttttttttgtgtgtgtttgttttgtgtttttttgttgtttttgtgtggattttggtttttgttttgttttttagtttttttgtgtgtttgtgtttaattgtgtgttttttttaatttttttgtttttttttttgttttttatatgtgtgtttttgttaatagatttttagaggtggtgtagaaatgattttgtgttggtgttttattttgtggttagtgtttttgtgttttggttttggtttttattgtttttgttttagatttttggaggagtaggtgggtggggttttgtggggttggttgttgttagtgtttttttttggtggttgtgatttttttttgttgattttatttgagttgttgtttggtgtagatataagtggtggtttatttgaagagggtttggtaggtgtttggggtttttagtgttgtagatttttgatttgttgattgtggattttgagtttttggattttggatttattttgtggagtttatttttggtaggtaattgttttaatttttttttttttgtagatggtgtttgggagtattggaaagggagtttttttggtttgtttttatttttggttttttgagagttttttggagaatagggttggtgtataggtgggtgttgtgtttgtatttttgggttttttttgagtgtaggttggggatttgatgggaggtatggtgtgggtatattggaggtttttgttgttgggtatttaggttgttagagtttgtagtgtggggtgggagttttgtggtattttttgtttgggtgttgttggatagagttgggtatttagggtgttggtttgagaatttgggagtttgaattttaattttaatgttagaaatagaattttagtgttgaagttaggattttagtgttagaattagatttttggtttagttatgaaagtttagtattagaaatagaattttagttttgtaaatagattttgagttttagaatttgaattttagtgatagaaatgggatttt

SEQ ID NO: 211 - DNA, 1050 bp, Artificial Sequence
  subsequence of FBP1
gtaagaggattgtttgagtttaggagtttgaggttgtagtgagttagtgagaaatttgatttgggtttttaattgtagaaagagtgaaggaggataaagaagaaatggggaaggaaggtatttgagttagtatttttttgtttgaggttagggtgggtatgatgggtaggttaggtgtttatatttggttggttgagaggggtatgaggtaggttgtttagggtagagaagaggaatgtaagatggaattaggtgtatatttgtgagatggatttttaggtgggtttttttaaggttaagggaggagatgaggatttgagtgaggttgagttagtttttggagagaggggttattagatagagagtgagagaggtgtttagatattagatggatagatagaggttgatggtaggaggtttagagggtttaatgttagtttgttgggtaggttttttaggtagatagataggatggggtttattgtttaaggtttgatttataggtgtgtgatgtttgttttgtgtattgttgaagagatggttttgattgttgtgtagagtgagttgagtagttgggttaatttgtttgtgttgtgggtttttttgtttttttttatgatgaagtgggttagggtgttgatgtttgtgttgaagggtgtttggttagttatgtttgaattgggtagagtgtggggttgtaggtgtaagtggtaggtgtggggttgtaggtgtgggtggtaagagagggtagtaggtattggttgtaggtgtggagttgtaggtgtgggtggtaggtgtgggttgtgggatttggtgggaggattgatagattgattgttgttttgagtgtttgtagtgtttgtggtgtaattgggttaggttaggtgttggtgggtgaattgtggaaatttttagatgtgggttggttttttgtttttgggaatatttttgtgttttgtttattttgtggattagattgtggttttgtttgtttggatttttagataaggttggggattttttttgtggttttttaatttttgtttttat

SEQ ID NO: 212 - DNA, 968 bp, Artificial Sequence
  subsequence
tattttgtggtgttttttttttttgttttatagttaagtttagaggaatagggtattgttttgtgtggttttggagttatatttggatttgggaattttgtgttgggtttagaggattttattgggtgggttggggtttatttttaataaatttagttgtttatatatatttttattattatttttatgtgtgtgtgtgtatatatatatatatatatatagagggatttgttaattattgagatagtgatttagaaaggtgtaaatttatttggtgtttatttaattgtaaagtttttagatttatataaaatattagtgagttttggaggtttgagatgttttgtgtttgatttatggtgtgaatattagtgttttggttgggttgtaggggttggtgtttagtgggttggggtttggtgagttaggtagtgggttggttgagtttttgtgttttgtggttgtgtggtttatttgtttatttttttattattggttgggatttgtttttttttaaagtgtgatgtttggtgttttattttaggttatgtggagtttttttttggtgtgttgggttttgtgtttttgtttagtggtttattttggtttgttgtgtttgtttttgttttaagtgtttaggatggagtgaggggttgtgggttggtgtagggatgttggttgtttatttttaggagttgatagaaggtgtttttggtgttatgtgtttaattttggggttgatttaaataattaataagtttagagaaattttatgaggggaatttttttttttagtattaataaagtttagtaattgggtttgttggttatttaagtttgagtttgatttttttagttttttttttttttttttttttttatttagaggtaatgtttttaaaaattggtaattgtgtgtgtgtttggggtgtgtaagtgtgagtgtagatgtgtgaaagttatttgtt

SEQ ID NO: 213 - DNA, 1080 bp, Artificial Sequence
  subsequence of BCAN
tagtttttttttatgtttgtgtagtttttgttgtagttttagttgtagttgtaggattgagttgtgtatttggaggagatttttggaggaggtgataaattttgtagtgttgtgatttaattttagttttgggtaggtgagtgtttttgtagttttgttgtttgttgtggggttggggatagggagaagggagtgtttgtttggtttgtgttttttgtttgttagtttttgttttgaggttttggggtatttaatttgttgatttttgatattgtagtggggtaggttgttggatagttttgagtgtttgtagttgttgttgttatttttgatttatatgtttttagttttgttagtggtagtttttttgttgtttgtagtttgattagtaattttttgggtttttgtattttttttgttgtgtgtttttgttttagtgtggttgttgagatttttgattttgttttaggtagggtggtagtgttttggattagtttttgtttttgtgtttttatgtataggtagttttaggagtagtggttagtaatttttttggggatattttattgtagtttggtaggatagttgattagagttgttttagggggtggttggagtgtttattttggttggaattttaatttggttttttgttttgtgattttggggtagttagttgatttttttgaattttgggttttttattggtgtaatgagtaaaatataaataattttttttttatagttgaggttatttataggaagtatttaggagaattggtttaggtagatattagtaattattagttggaatttggttattattattgtatttttttttagtattgttaggggtttgggaatttttttttttaggtatttttatattttgttaggtatttttttatttattggaatagttttgtttatatggttttaataggattaagagattggtgttaagtttaaggggtattattattatttgtattatttagattgtagatgatgatagtaagattagggaatagtgttatttttagatttatttatagtattattagtgtttatgttgttatg

SEQ ID NO: 214 - DNA, 1017 bp, Artificial Sequence
  subsequence of TP73
tttttaggttagggtttaggttttgtataggttaggagtgattttggtgggtttttttttttttttgttttttgtttattttggttgggggtgtaggggttagggtgtggtgtggttagatttttaggtttagggtgattttttttgttttttttgttttattgttttttgtttttaatgtgttggttttttgtaggatttgggggttttgaagatttttgagtagtattgtatgattatttggtggggtttgtaggatttgaagtagggttatgattatagtattgtgtagtagttgttttgttttagtaatgtggttattatttttattggtggtttaggggaattgtagtgttagtgggttatggaggttgtgtatttttgtgtgtgttatattattattatttttaattgtggtggtttaggtggtggttttgatgagtgggtggattttggttttgatttgtttgattgtaaggtttgtaagtagtttattaaggaggagtttatggaggttgagatttattgagggttttgtttggttgtagtttgtgttattgtttagagatttaagttgtttttttttttttttttgtgtgtttaaaattgttttaggaggtaggatttttgggttgtgtttggggaaaggtaaggtttggtttatttttaggtattttataggttttaggaaaggtttagttattgaagttgtttgtggatagtttgagttatttgtagaattttttggagttgttttagtgttgggtttgtggggtgggggttggtttatttttagttttgttattgttttggtgtgttttatggtaggtgtgggtggggattgtagtgttggttttgatttttaggttttattttagagattgttattttttaattaggtgaggtttttttaaaggaaaggattttttttgttgatggattgttaaaaagtattttgtgatattttttggttttggatagtagtgagtagttaagtgattgtgtttgaaatattag

SEQ ID NO: 215 - DNA, 1058 bp, Artificial Sequence
  subsequence of MIIP
gttttttgagtagttggattataggtatgtgtttggtttattttttgtagagatggggttttgttatgttgtttaggttgggtatttttttttttttttttttttttgagatggagttttgtgttttgttttgttatttaggttggagtgtagtggtgtgattttggtttattgtaatttttgtttttttagtagttgggtttaagtgatttttttgttttagtttttttagtagttgggattataggtgttggttattttgtttggttaatttttgtatttttaggtttattggtgtgagtaattgtggttggttttaatattatatttttttgtttattgtgtagtttttattttttggtgtaaagtgggtatttgttaaaaatttgaatgaaagtataaagtaggttgggtgtagtggtttatgtttgtaattttagtattttgggaggttggggtgggtggattatgaggttaggagtttgagattagtttggttaatatagtgaaattttgtttttattaaaaatataaaaaaaaaaaaaaaaaaaaaaaaaaaaaaaaaaaaagttgggtatggtggtgtttgtttgtagttttagttatttgggaggttgaggtaggagaattaattgtttgaatttgggaggtagagattgtggtgagttgagattgtgttattgtattttagtttgggtaatagagtgagattttgtttttaaaaaaaaaaaaaaaaagaaaagtatgaaatgatgagtataatgagtagttagttgtttgttttatttattttgttgttttggattgttgtttgtgttgagtttaggttgtttagtttgattgaaattgttgttgttgaatttagtgtatttagttttgtagttgattaattggagtttgtttttttggggtttgttttattttaggaattgatttttgtattggttttgaaaattgattaattggttgtgtagttttgtttttagttttgtttttatgttgaaattgatattttggattgttgagtaatgtttattagatgttttggttttgttttt

SEQ ID NO: 216 - DNA, 1166 bp, Artificial Sequence
  subsequence of HOXC4
tttggtttgggtttgaggttttgaggtggttttataagtgtgaatttttgtttataaatggtgatgtatatgtaaatgtttatatatttaggtagatatatttggaggtgtgggggaatatattttagatgaggtttgagagaatttgggggttagatttattttaatttaagttaagatattttttttatttttatttttttttagaggtgtgattgggattttgtgttaagttttagtgggggaggggattgttttatgattttttgtttaaattttgatttgttgtttgtgggggggtggggatgggggaagaaatttgtttttattaaatttttaaaaatatagtttgtatttagattaaattgtttgggttagagttgatggtgaattttattttttttttttttataattttttttttttttttttttgtttttgttaaattttttttttgtagttattataaaggtttttgaggtatttttttgtgggaagaaatggtatttttttttttttttttttatttttttatttttagtaggatttgtgtgttggtagaggtgtttgatgaggggttaattttgttttttttgtttatgattgagaaaagtgtttggttttttttaaatgggtttagtttgttgtttgtttaggttgttgtgtgtttttttgttgtttaggtatggtttagtggttgatgttttgtggatttagtttgtggtaggttggaatttggagtttgtgggatttgaaaagggtaaagggagtgtggtgggatataagaggggtttgagaggtttgaaaaagggagttttttttttggagtaatgtgggaattgttgattggtgaggtttttattttttttttttgtttgtattttgtttgtttttgtttttgtggatggttgttgatgggggggaatataagtgttttttgtttgtttttgaaagaaaattttttttagagaaggttatttaggttagtggagatttatttagaatattagaagggtttttttttatttgaggggtgggtttgggtgaggttgaggtattttttttaattttagaatgattttattgagtgtatttatagtgagatttggaggtgttagagtttgaggttggttgtttaatagtattattttgaggtgtttttatat

SEQ ID NO: 217 - DNA, 1334 bp, Artificial Sequence
  subsequence of HOXA7
ttggttttgtgttttgtgttttgtgttttttagtgttgtgttttttgtttttggttttgttttgttagtttggtttgtttagtgattgtgtttatttgaagattgtatttaggggtagatgtggaaattggttttagttgtgttatgtagtgtgtttttgtttgttttgttgtaggtgtttttggtgaggttattgtagagtttggggatgttttggttgtaggaggtgtagggtaggttgttgtaggtgttggtgtttaggttgtagttggatgtaaaggggttttgataaagggggttgttgatattgtataagtttggaatggttgaggtgaaggtgttggtgtttgttttgtagttgtttttttgtgagttgggagtaaaggagtaagaagttggtttggtattttggaatagagaagtttttgttgtatatttgttaaaaagtgtgtttatataatatgaagaatttataattttgatttgtgatttgttgtttggtagtttttagtgttggttttatgaggtagagtgatatatgataatattatatttttagatttatattaaattttatttttttttggatggagtttgttgtagtatgtgattgtttatatgattgtttttgttaattttagtagtttttataggtggttttgttttgtagggtttgtagttgtttagaatggaagggtaagaggtttaaatatgtggttaaagaatttgtttgtgtttggtgggtttggtgtgttttgtggaaaaagatttggaggttttgtgggagtgtttagttggtggttaatttttgtattggggtttgtggatgttaggtggtttggttttatgtagtattttttattttgtttttttgttgatttttgttagtgagttttggattaagtttgggattttttttattttttttttgtttgtttgtttagattttggaagggtttttgttttttgtaatagtttgttttgtggtggttttgtgggtaggatttagttatgagtagattgattttgtttaagtttttttttatttaggtttagtgggtgataggttggatttagatttggatttagatggggaaggtgtagtattttttgtagttgtagagagattgttattgtaaattggagttatgtggtttgaataaagttaatgttttttagttttttttttttaattggaggtatattgtttatttgttttaaaggaagtagtgaaatatttatttattaatgagatttatttgttaatagatttattaatgtggggtttttttttttttttttggatgttgtagtgttgtaggttttgtgttttt

SEQ ID NO: 218 - DNA, 1181 bp, Artificial Sequence
  subsequence of BST1
aaagagggtatttgttaaagttgttgttttttggagttagaaaagtattttttttgatgatgattgatttttttttattttttttttaattttgtggtttttttggggttttttttttgtggatagtggttaagattagtttgggttatagtgtattggttttgttttgggtgatgttttaagtttatttttttttaattttatttatttattaattttttttttagatagagttttattttgttgtttagggttggagtgtagtggtgtaattattgtttgttgtagttttgattttttggggttaagtttttttatattttattttgtaggtagttgagattatagatgtgtattattaggattagttaagttttgtatttttttgtagagaggaggttttttatttgtatgttatttaggtttgttttgaatttttatatttaagtgatttgtttgtttaggttttttaaagtgttgggattataggtatgagttgttgtgttttgttttatgtttagttttgaaattgtttggaatgggaaatggtaaatagtgagatatttgagtgagagttttgttttgtattagtttgtggaattgttttggtagaaggagagaaggggagtggaggaagtatgggattggagggattaaagttttttgatggtggtttaggggtgtgtggtattgtggttgttttagttgttgttgtagtttttgtttttattgttgttgttggtggtgggtggggtgtgtgtgtggtggtgtggggagggtattagtgtatatttgtgggatatttttttgggttgttgtgttgagtattgtgtattgttgagttttgagtagtggtgaggtagttggttgggtggaaggggagttggaaagaggtaatggtggggagggtttggggaggggaaaattggtgttaaagtttggggtgagggggtaatgagagaggttttgaggggagaggtgagtgtggagatagtgatggttttgaatgatgggggtgagtgaggagggggttgtgaagtgtgtttgtaggtgaggggtgtgttgtgtggggtttagagatgagtgtgtgtgtgtgtgtgtatatatatattgtagaggtgaggggtgtgtgtgtgtgttttagaggtgaggggggtgtgtgtgtgtgtgtgttttagagg

SEQ ID NO: 219 - DNA, 1039 bp, Artificial Sequence
  subsequence of C1orf114
aggtattattaatgaaatgaatattattgtttttttatggaagttataattttgggagtgatttttttttttagggataaaattattattttttatgtttaagaattttgagaggggaaagaatatgataaaaatttagtttaagttatttttagatgagtattttttatatatagtgaagaaaggtgttattagatttttttattagtgttagatttggtggattttggattttagaggataaggtgttattagaaagtttttttttttattatttatttttttttttagttatattagatgtttatatttttattttttattttattattaatatttttgtattgttttttttttttttttttttatattttttttgtttgtgtaaaatgtggtggttgggattatagagatggttgggggagtttttttttttttttttttttaagttttttatattgaggtaaaggagattttggtatttgtttttgtggtagttaggattatattgttatggtaatagtgtgtttttgtgttttttatttgggtttttttaattatgagagtgataaaaatttgtgtaggtgtatgtaatatttttagattattgattttttttggtatttattttggtgtttgtgaagggttagtgaagttgaggtttttaaggggatgattaaggaatttatgtattattaatgttatagagatttgtgaagtgttgttgtgttggttgtgaaattttttttttgtagttgttttttgttagatttagaattttttaggataatttttaaaattatatttttatatgttatgtgttttatagttaatgtagaaaagtagtttttagtttttattagtagataagtaaattgttaatgagttattatatttttgatttttagttttttttttttttaattaaaattaaaattttatttaagagagttgttaaagaaaataggatttttgttttagttttgaggtatgttaaaatggttaattagaatttttttaaattttgtttatttttatg

SEQ ID NO: 220 - DNA, 1027 bp, Artificial Sequence
  subsequence of FOXG1
ttgtttttgttgttgttattgttattattggttgttgttttggatggggttaaagtggatgggttgggtggtaagggtgagttgggtggtgggttgggggagttggtgtttgttgggttggatgagaaggagaagggtgttggtgttgggggggaggagaagaagggggtgggtgagggtggtaaggatggggaggggggtaaggagggtgagaagaagaatggtaagtatgagaagttgttgtttagttataatgtgtttattatgatggttatttggtagagttttgagaagtggtttatgtttaatggtatttatgagtttattatgaagaattttttttattattgtgagaataagtagggttggtagaattttatttgttataatttgttttttaataagtgttttgtgaaggtgttgtgttattatgatgatttgggtaagggtaattattggatgttggatttgttgagtgatgatgtgtttattggtggtattatgggtaagttgtggtgttgttttattattttgtgggttaagttggtttttaagtgtggtgtgtgttttatttttattggttttatttttatggattgtgttggttttttttattggtttatgttgttttttttgtttttgtattatttttgtgttagtagtattttgagttataatggtattatgttggtttattttagttattttatgttttatagttttgtgttgatttagaatttgttgggtaataattattttttttttattgttaatggtttgagtgtggattggttggttaatggggagattttgtatgttatgtattattttatggttgttgtgttagttgttttggtgttttgtggtttgttggtgttttgttttgggatttatttttttaatttttgttttgttaatttgtttgtgggttagattagttatttttttttttatgttttgtatttgttaatgattttgtagagtagtatgtttatgagtgttagggttgtgtttttttttatgttgt

SEQ ID NO: 221 - DNA, 1017 bp, Artificial Sequence
  subsequence of ATP4A
atgagggaatggggtggggttgaggagaggggtggggttgagagtttggtgtgggttttgagagttgtggggaagggtgaaggtggaagatggaggttttgtgtggaggggtttttggtagaaggtaagtgttaaggtggggttaggtgtagatttggagttttggatgtagggaatgaatagaattagggtgtggagttgggttgggggtggatttggagagtgaggtgttttttatggatagttttgttgaggagaagttgtgggtggggttgggtggtgggtggggttttgtttttggtggatggggttataggtggagtgggagatggggtggggtttggttgtggagagaaggggtaaggagtgaagttttttgtggtttgttgatgtgggtgtggtttggggtggggtttgaggtgggtgggtttaggttgtgggtggggttggttgtgtatttggaatttgttggtggagttgaagggtattttgtagatttttgggaagtggttttggtagtttatggtgttgtttagtgttagttttgagaatttgagtagtgttgtttttgatgtgtttttaattatgatgtgttgggagtggggattggtgttaggggtgaagttggttatattagttttttgggattttttggaggttttttggtttttattttgggtataggtattgtattttggttggatttgaaggtggtgtggttgtatagggtgagtatttggtatagtgtttgttatgtttttgaggattggttaaatgtttgttttgtagattaggtgtttaggttgggttttgtatggtggtttttttggattagaattgagttttttttttttaggtttatattgtgggttttttttttagatttgggtggaattgggttttatttaattttgagggatttagtttttggatgatttttttttttagatttggtagtgagttttttttgagatttggagttgagttgttttgtttttgtataaagttttttttatt

SEQ ID NO: 222 - DNA, 1161 bp, Artificial Sequence
  subsequence of NOTCH1
tttataaatatagaagaatttttttattttatgtaggaaaattttggtttttagaatttgtttgtttttttagaatagataaaagtttttatttggggttagataaaatagtatatataaataaaaaggtagtgtttttgtgtaaaataaaagtatataaataaatattaaaaaaaattaaaatttttgtttttattttgtataaaaatatgtgttttaaaaaggtttttttggttggttttggtatttatagagtgtatatagatgtttgaaggtttgggaaaggaagttggggttttgtggggtgtgttgtttatttgaaggtttttggaatgtgggtgatttgggattgtatgttggtgggagggttggagatgtttttggattagttggagatgttggaatgtggggatgagttggattattggttaggggatttaggggatggggtgaggaaggggtgtttaggtatttgtagttggtggttgggggtgttgtttataggtgaggagtagttgtgttgtgaggggggtgttaggaattgggttgtggttatgggtgggattagtgaggatggtagtgatgtgggtagggtggggtttttttggggtagaatagtgtgtattgttaggttgttggggtttagtggttgtatgtttgtttggtttggttttttatttaggaagttttggtttaggtggttgttggttgttgagtttatgttaaggtgtggttgtggtggtggtggtggtggttgtaggttttgttgttgttggatgtttgttggttgtaggttttgttgttgtatttgtaagttttgtggttgtatttgttgggtttgtattaggtgaggttgggtggttagttgggtgttgggtaggttttggtagtttattatttgggatagggtgttggtagtaaggttattgtgtagtgggtttattatgttatgttgtagggagggggtttgtgtgtttagggggtttggtgttatatttttttgtagagggttgtattggtttggtattatgttgttttgtagttgggatagttatttgtattgattatttaaattggtggatttgtttatagtgaaatttagggtttttttgttgttggagtttaggatggtgttggtgttagaggttataggtaggtggg

SEQ ID NO: 223 - DNA, 981 bp, Artificial Sequence
  subsequence
ggttttgttgaatgtgtttttttggtggtgatggttttggattgtgtggttgtagtgtgttgtttgttgtgttatgtggggtttgtttttttgtgtttatgttgtatgttggttagtgttttttggttaagtggttttattaatttggttgtgtaaattgtgtttttggttgagtggttgttgtgtgtgttttgtttgttggattattttatttgtgagttgttggtgttgtttaagttggtttgtggaggtgatggagatattattgagaattagatgtttgttgtttgtgtggttattttgttgttgttgtttgttgttattttggttttttatggtgttgtggtttgagttgtttgttgtatgtggtttagtggaggttggaggagggtggtgggtatgtgtgggttttatttgatagttgtttgtttgttttatggtttggttatttatatttatttgtagtttgtgtagtgttataattaggtatggggtaagtttgtattgtttttttatattgtggttatatttgtttttaatttgtttatttatatttttaggaataagaaagtgaagggggtagtgaggaggttgttgtggagtttggggagaggttaggttgggtagtgagtagttggggaggggagaaagtattaagttagaatttaaggatggaaatattttttagtgagttagtttagattttaggttgtttatttttgtatgataatttgtaagatttgttttaaggagtttaatgggggatatgttttttttttgtgaggaaatgtttagtttttgagggaaaatttttaaattttttatatatttaggtttagggaaggaaaatttattttttataattttatgtgtagggaaaatgatggatgtgatgtttgtttttagttttttttttatttgatggaagattatggaagattttttggtttttgtaattagaagttttaagttgataagaaa

SEQ ID NO: 224 - DNA, 864 bp, Artificial Sequence
  subsequence
aatgtttttttttgaggtagttggaggttttttttttttgagagtttaattgtggatatagttttgagtagaggtgaaaaggaaagaaggttgggagataattggggaaaaatatatatttttggtttggtttgaaagttggtgtttgaaaaggaataagaaaaaaaatatgttttggaagtgtttgagattgttgtggttataaagtagagtattaattttggtattgatatgggaggggagtaagattttatttttgggtttgtgtttatgaatttaggtgaggaggggtatttttttttttgtgtttaaatgttgtattttttaagattattttggtttgttatgtttttattttgtgtttataaaaatttttgagattttagtgggtatagatataagtggttggatatgaagaggaatatattggtgggagaatatagaatatttattttttttttagttattttggaggttttggagaaggtggttttagtgatttgtgaagagattttgagttttttatattttggagaatgataagagagtatttgggtgatgtaattttttgggttttttggagatgtggttttttatatgttaggggttaagtttgagaaataggtggatggtaaggataggagttttttttgtgttataaatgtttttgtttatatttgggttttttgatttttttgtttatataattgagttatttatggttgggttttgagtttttattttgtggattgggataaaggggttaattgtgatgtgttttgtataattttaggattttgtatttgtttaagggtgaggtgtaaatttaaaattatgtatgattgaatgttgagattagttgag

SEQ ID NO: 225 - DNA, 1004 bp, Artificial Sequence
  subsequence of HOXA7
tatttaggggtagatgtggaaattggttttagttgtgttatgtagtgtgtttttgtttgttttgttgtaggtgtttttggtgaggttattgtagagtttggggatgttttggttgtaggaggtgtagggtaggttgttgtaggtgttggtgtttaggttgtagttggatgtaaaggggttttgataaagggggttgttgatattgtataagtttggaatggttgaggtgaaggtgttggtgtttgttttgtagttgtttttttgtgagttgggagtaaaggagtaagaagttggtttggtattttggaatagagaagtttttgttgtatatttgttaaaaagtgtgtttatataatatgaagaatttataattttgatttgtgatttgttgtttggtagtttttagtgttggttttatgaggtagagtgatatatgataatattatatttttagatttatattaaattttatttttttttggatggagtttgttgtagtatgtgattgtttatatgattgtttttgttaattttagtagtttttataggtggttttgttttgtagggtttgtagttgtttagaatggaagggtaagaggtttaaatatgtggttaaagaatttgtttgtgtttggtgggtttggtgtgttttgtggaaaaagatttggaggttttgtgggagtgtttagttggtggttaatttttgtattggggtttgtggatgttaggtggtttggttttatgtagtattttttattttgtttttttgttgatttttgttagtgagttttggattaagtttgggattttttttattttttttttgtttgtttgtttagattttggaagggtttttgttttttgtaatagtttgttttgtggtggttttgtgggtaggatttagttatgagtagattgattttgtttaagtttttttttatttaggtttagtgggtgataggttggatttagatttggatttagatggggaaggtgtagtattt

SEQ ID NO: 226 - DNA, 963 bp, Artificial Sequence
  subsequence of ZNF529
tatttatattattatatatggatgtagttatagaatggtatatataaatataattatatagtttttttttttagtaggtgatttgtatataggtttggggtttttgtttttataaattttttttgtgttttatgaatttaggttttttgttgtgagttttttttgtatttagatttgggttattttaatttattatatggaaaatgaatattaggttaatggaaaagggagttggggttttggtagaagtgtagtgttagttgtaatagggtggttgtgggtgtaaatggaggggagtgttggtagttagtattgtgtggtgattaatgggttgttttggagatttttgggagtttaggtttatgtgtgagtgtgtaggtgtagatgatttattttgggttgggggtgaggtttggtgggtgttaggggtgttgaggaggttaaggagggtattgggagttttgttttgggagagttgtttaaggatgtggaattgggtggggaggttagggaggtaattaagagtttgagttgggtttggaaattttgaggaggaaattgggattgtggtatttagatgttttgatttgtatgagttgggttttgggtgtggtttatgggtgtgattttgagggtgatttggagtgagttttgtgaagggggattttgtttgttttgattttgtggaagtgttattgttggtggtgggtttttgtgtggagtgggtgttgtgtgtttgtgtgtgtgggtgtgtgttgaataggggtggttttttgtgtttaatttgttgtattttaggtgtttgtttttttttgggatgttgtggttgattgtgtgtgaaaagtgaatggagtagtgttgttgtgtgaggttttttttgtgtgtgtttatttttagattaggtgtgagttgtatttgagattgtttttgagtgtggagaattgtaagtgatttgtaggtgtg

SEQ ID NO: 227 - DNA, 956 bp, Artificial Sequence
  subsequence of PRLHR
ttttgtgggagttgaatggatttaaatgttttttagtgggttagtagttttttagagggattgggagtgtttttttttagttttgttttttattttagtttttatgtatggaaaataggggtggagtgtgttttgttatgtttggtgtttagagaattagtgaagaggatagtgtggggaggatggaatttgaaatgggaggaggggttggtaaatttggttagagtagtgaagaggatgaatttttattgtaggagtttgtatggtttttggagggttagggtttggaggagtgtatagtggaggagggtggaggagaggtgtgaagtgttaggttattgggaggagttggtgtagttttaggagggaaggagtggggaaggaagtagtggtgggagattgtaatgtagttgggggtttgagagaggtatgattgatttgtttttaaaggttggattttgtttagtggttaggttgtggagggtagagatgttagagggtttgggttgggatttaatagtagagtttggagttggttgtggatttgaggatttttatttttttgtggttttgttatatgagttttttgtttggttgtagtgtgtttagtgggtagtgggtttggttaagttaaaggtaggagttagtattatggatagtttttgttggatttgttgttttttgggtggatgatgtggggaggagtaggggaaaatgattagggaatttttgtttttttttggatttgggtgtttggggaaggaatttggtttggtttaaagatttgttttgttgtttgttttttgggttggggttgagaagagtagggtggagtggaggtagttgagttgggggtttgggggttggagatttatgatttttgttttttttggtggttattgtatttagtgttaaggatggtgttgttttttattttttggggttagaaaatgtag

SEQ ID NO: 228 - DNA, 1037 bp, Artificial Sequence
  subsequence of ARL5C
gagggagtgaagagtagggaggtagtttgggatatttggtaggtttggagttgggtttggtagggtttttgggaagatggaggaaagtagtgggagttagggttgtttgtatggtgtaggatttgaatgttagattgaggagttgtagtattaggaaggtttaagtagtgtgtttaattgtttttttggattatagaggagtttgggaatttagggaagagttttagtgattggtttttttgtttgttagtttggtgggaggtttagttttttattttttttgtttgagagttatttataattggtagagaatggtggttttttttttattgtttagttttatgatgatgattgtgtgttttggaagtgttggaggaagggtagtgttagggttaaatttgaagattaagggattgtgtgtttttattagattttttaggaaggagatagtgttttttgtaggttggggatttttgtttattgtattgtgtttgttttttatttagttttgatatttgttttattttgggatggtggaaaagatagttgagtttttatttttttttatattttagaaaagtgtttgaaaggtttggggtgttttggggtttgttaagagatggtgtttagagaaagagtataatgtgaagttataattgtaggaaatttgtagtagttttttatttttgttgttggttttgtttagtggggagaaaggagggttgtttagttttgtgttttggggtgtattgaagtgttgggatttaagaggagtaggtagggatgggggtaggggaggttgtggggtagttgaggagtgtttatatatgatttttggagtttgaggtttttttgtgtgtttgattatagattagaggagttttagaagagggtgagattaaagtgtttgtttggatgttttgtgtgttttttattttggtttttgaagatgtttattaatttggtgattagttgttttatggtattttttgggttggataggtgtaggagggtttagtggttttaggagtggagtt

SEQ ID NO: 229 - DNA, 20 bp, Artificial Sequence
  forward primer of PAX9
cagcgggtcgctgactacct

SEQ ID NO: 230 - DNA, 20 bp, Artificial Sequence
  reverse primer of PAX9
cgaagctgcagacagggtca

SEQ ID NO: 231 - DNA, 19 bp, Artificial Sequence
  forward primer of HOXD10
cctggagaggcggacagga

SEQ ID NO: 232 - DNA, 20 bp, Artificial Sequence
  reverse primer of HOXD10
gcacggacaacagcgacatc

SEQ ID NO: 233 - DNA, 20 bp, Artificial Sequence
  forward primer of TBX20
gcgacgcgtagtgggagagt

SEQ ID NO: 234 - DNA, 20 bp, Artificial Sequence
  reverse primer of TBX20
ccgtgggaagtcctcgtcac

SEQ ID NO: 235 - DNA, 19 bp, Artificial Sequence
  forward primer of CACNA1B
ggccagcgggtcctctaca

SEQ ID NO: 236 - DNA, 19 bp, Artificial Sequence
  reverse primer of CACNA1B
cgagcggttgacggtgaag

SEQ ID NO: 237 - DNA, 20 bp, Artificial Sequence
  forward primer of ZIC1
tctcttggctctcggcttgg

SEQ ID NO: 238 - DNA, 20 bp, Artificial Sequence
  reverse primer of ZIC1
aacctttgccttcccgcttc

SEQ ID NO: 239 - DNA, 20 bp, Artificial Sequence
  forward primer of SIM1
tggccaatagggctgagtga

SEQ ID NO: 240 - DNA, 19 bp, Artificial Sequence
  reverse primer of SIM1
cggtcccgcgggtagtaag

SEQ ID NO: 241 - DNA, 19 bp, Artificial Sequence
  forward primer of DLX1
gaggaaacgcgggctccta

SEQ ID NO: 242 - DNA, 19 bp, Artificial Sequence
  reverse primer of DLX1
ggcgggcaacctcacataa

SEQ ID NO: 243 - DNA, 19 bp, Artificial Sequence
  forward primer of LMX1A
gcttccctatcgcggacca

SEQ ID NO: 244 - DNA, 19 bp, Artificial Sequence
  reverse primer of LMX1A
ctcctcgctgctgggtgag

SEQ ID NO: 245 - DNA, 23 bp, Artificial Sequence
  forward primer of DLX6AS1
gccacgtctacctgctggtattg

SEQ ID NO: 246 - DNA, 19 bp, Artificial Sequence
  reverse primer of DLX6AS1
ccaccccaccttcccactc

SEQ ID NO: 247 - DNA, 20 bp, Artificial Sequence
  forward primer of MIR196A1
ccgcctacacggaaaatcca

SEQ ID NO: 248 - DNA, 20 bp, Artificial Sequence
  reverse primer of MIR196A1
cgagaagcagggagcctcat

SEQ ID NO: 249 - DNA, 20 bp, Artificial Sequence
  forward primer of FEZF2
aagcgtcacggagagcaagg

SEQ ID NO: 250 - DNA, 20 bp, Artificial Sequence
  reverse primer of FEZF2
gtctcccccagcctcagacc

SEQ ID NO: 251 - DNA, 19 bp, Artificial Sequence
  forward primer of STAG3
agggcaggacgcctgagag

SEQ ID NO: 252 - DNA, 20 bp, Artificial Sequence
  reverse primer of STAG3
tcgcgaggtttggaagtgtg

SEQ ID NO: 253 - DNA, 20 bp, Artificial Sequence
  forward primer of PTPRN2
gaagcccgacccacaacaaa

SEQ ID NO: 254 - DNA, 20 bp, Artificial Sequence
  reverse primer of PTPRN2
cctctcacatccgccatcct

SEQ ID NO: 255 - DNA, 19 bp, Artificial Sequence
  forward primer of BARHL1
tggaggatggcaggtgcag

SEQ ID NO: 256 - DNA, 19 bp, Artificial Sequence
  reverse primer of BARHL1
gaagcgagcaggcctgaaa

SEQ ID NO: 257 - DNA, 20 bp, Artificial Sequence
  forward primer of CCDC140
gagtcttgggcctccccttt

SEQ ID NO: 258 - DNA, 19 bp, Artificial Sequence
  reverse primer of CCDC140
gtggcctggactcccatcc

SEQ ID NO: 259 - DNA, 19 bp, Artificial Sequence
  forward primer
gaagcgagctgggctgtga

SEQ ID NO: 260 - DNA, 19 bp, Artificial Sequence
  reverse primer
cccagtagcggctggagca

SEQ ID NO: 261 - DNA, 18 bp, Artificial Sequence
  forward primer of GATA2
cgtccagccagggcaaac

SEQ ID NO: 262 - DNA, 20 bp, Artificial Sequence
  reverse primer of GATA2
ttctgcccaccctgatcctc

SEQ ID NO: 263 - DNA, 23 bp, Artificial Sequence
  forward primer of ADCYAP1
gggctctccggttactttctctg

SEQ ID NO: 264 - DNA, 19 bp, Artificial Sequence
  reverse primer of ADCYAP1
agcgctgcccccttgagat

SEQ ID NO: 265 - DNA, 20 bp, Artificial Sequence
  forward primer of TBX5
aatgtgcagggaggcagtcg

SEQ ID NO: 266 - DNA, 19 bp, Artificial Sequence
  reverse primer of TBX5
agctggcctgggagcttgt

SEQ ID NO: 267 - DNA, 20 bp, Artificial Sequence
  forward primer
acagcagatggcacgagcac

SEQ ID NO: 268 - DNA, 20 bp, Artificial Sequence
  reverse primer
tgggagcttgtgggtgcttt

SEQ ID NO: 269 - DNA, 22 bp, Artificial Sequence
  forward primer of NECAB1
aacaatccttgccccatagcaa

SEQ ID NO: 270 - DNA, 20 bp, Artificial Sequence
  reverse primer of NECAB1
ggcagtgctgctgacaaagg

SEQ ID NO: 271 - DNA, 19 bp, Artificial Sequence
  forward primer of ADRB1
cgtcaccaaccgggcctac

SEQ ID NO: 272 - DNA, 19 bp, Artificial Sequence
  reverse primer of ADRB1
cggaacacccgcaggtaca

SEQ ID NO: 273 - DNA, 21 bp, Artificial Sequence
  forward primer of OTX2
ccgcctacagaactccacgtt

SEQ ID NO: 274 - DNA, 22 bp, Artificial Sequence
  reverse primer of OTX2
tctacgtgtccccgtgtctgtc

SEQ ID NO: 275 - DNA, 20 bp, Artificial Sequence
  forward primer of NKX2-6
agaagggggtggaggtgacg

SEQ ID NO: 276 - DNA, 20 bp, Artificial Sequence
  reverse primer of NKX2-6
aagttcagggtgccgctcct

SEQ ID NO: 277 - DNA, 22 bp, Artificial Sequence
  forward primer of SIM1
accccaaatcccatgtatcctg

SEQ ID NO: 278 - DNA, 20 bp, Artificial Sequence
  reverse primer of SIM1
ggctgaacacgaaggccact

SEQ ID NO: 279 - DNA, 19 bp, Artificial Sequence
  forward primer of DRD4
gaccagcagcacgggacac

SEQ ID NO: 280 - DNA, 19 bp, Artificial Sequence
  reverse primer of DRD4
aatccttcccgccgatttg

SEQ ID NO: 281 - DNA, 19 bp, Artificial Sequence
  forward primer of TBX15
tggctgttcagcaggagca

SEQ ID NO: 282 - DNA, 21 bp, Artificial Sequence
  reverse primer of TBX15
acaacgctcgcaacttggttt

SEQ ID NO: 283 - DNA, 20 bp, Artificial Sequence
  forward primer of TRH
actcctgacctgccgactgc

SEQ ID NO: 284 - DNA, 20 bp, Artificial Sequence
  reverse primer of TRH
cggttacctgccaggagtgg

SEQ ID NO: 285 - DNA, 20 bp, Artificial Sequence
  forward primer of FBP1
accgccgaagagatggcttt

SEQ ID NO: 286 - DNA, 20 bp, Artificial Sequence
  reverse primer of FBP1
tcatggaggagggcaggaag

SEQ ID NO: 287 - DNA, 19 bp, Artificial Sequence
  forward primer
gtgtgatgcccggtgtcct

SEQ ID NO: 288 - DNA, 19 bp, Artificial Sequence
  reverse primer
gagccgaggtgagccactg

SEQ ID NO: 289 - DNA, 20 bp, Artificial Sequence
  forward primer of BCAN
gcgttccggatcagtccttg

SEQ ID NO: 290 - DNA, 19 bp, Artificial Sequence
  reverse primer of BCAN
agcggctctgatcggctgt

SEQ ID NO: 291 - DNA, 19 bp, Artificial Sequence
  forward primer of TP73
cgccacaccatcaccatcc

SEQ ID NO: 292 - DNA, 19 bp, Artificial Sequence
  reverse primer of TP73
tccttgatgggctgcttgc

SEQ ID NO: 293 - DNA, 23 bp, Artificial Sequence
  forward primer of MIIP
cagcctccctagtagctgggatt

SEQ ID NO: 294 - DNA, 20 bp, Artificial Sequence
  reverse primer of MIIP
cggttgctcacgccagtaaa

SEQ ID NO: 295 - DNA, 20 bp, Artificial Sequence
  forward primer of HOXC4
agaaaagcgcttggctctcc

SEQ ID NO: 296 - DNA, 18 bp, Artificial Sequence
  reverse primer of HOXC4
cactgggccgtgcctgag

SEQ ID NO: 297 - DNA, 20 bp, Artificial Sequence
  forward primer of HOXA7
cctccgccaatctcagcagt

SEQ ID NO: 298 - DNA, 20 bp, Artificial Sequence
  reverse primer of HOXA7
ttgagcctcttgcccttcca

SEQ ID NO: 299 - DNA, 20 bp, Artificial Sequence
  forward primer of BST1
gaacgggaaacggcaaacag

SEQ ID NO: 300 - DNA, 20 bp, Artificial Sequence
  reverse primer of BST1
gtcccgtgcttcctccactc

SEQ ID NO: 301 - DNA, 20 bp, Artificial Sequence
  forward primer of C1orf114
gtgcctctgcgtcctccatc

SEQ ID NO: 302 - DNA, 20 bp, Artificial Sequence
  reverse primer of C1orf114
ggggtcggtggtctgaggat

SEQ ID NO: 303 - DNA, 19 bp, Artificial Sequence
  forward primer of FOXG1
acaagcagggctggcagaa

SEQ ID NO: 304 - DNA, 18 bp, Artificial Sequence
  reverse primer of FOXG1
tgcccgggtcgtcgtagt

SEQ ID NO: 305 - DNA, 20 bp, Artificial Sequence
  forward primer of ATP4A
gcgcacctggaacttgttgg

SEQ ID NO: 306 - DNA, 19 bp, Artificial Sequence
  reverse primer of ATP4A
cggcgctgctcaagttctc

SEQ ID NO: 307 - DNA, 18 bp, Artificial Sequence
  forward primer of NOTCH1
ggaactgggctgcggtca

SEQ ID NO: 308 - DNA, 22 bp, Artificial Sequence
  reverse primer of NOTCH1
gcacactattctgccccaggag

SEQ ID NO: 309 - DNA, 19 bp, Artificial Sequence
  forward primer
acggctcggccatctacac

SEQ ID NO: 310 - DNA, 19 bp, Artificial Sequence
  reverse primer
agcgggttgagagcaggtg

SEQ ID NO: 311 - DNA, 19 bp, Artificial Sequence
  forward primer
agcgggcacagacacaagc

SEQ ID NO: 312 - DNA, 19 bp, Artificial Sequence
  reverse primer
cgaggcctccagggtgact

SEQ ID NO: 313 - DNA, 20 bp, Artificial Sequence
  forward primer of HOXA7
cctccgccaatctcagcagt

SEQ ID NO: 314 - DNA, 20 bp, Artificial Sequence
  reverse primer of HOXA7
ttgagcctcttgcccttcca

SEQ ID NO: 315 - DNA, 20 bp, Artificial Sequence
  forward primer of ZNF529
gtgctgaggaggccaaggag

SEQ ID NO: 316 - DNA, 20 bp, Artificial Sequence
  reverse primer of ZNF529
ccggctcggactcttggtta

SEQ ID NO: 317 - DNA, 18 bp, Artificial Sequence
  forward primer of PRLHR
acacgagccccctgcttg

SEQ ID NO: 318 - DNA, 19 bp, Artificial Sequence
  reverse primer of PRLHR
agcggaagctgtccgtggt

SEQ ID NO: 319 - DNA, 20 bp, Artificial Sequence
  forward primer of ARL5C
cgggacggtggaaaagacag

SEQ ID NO: 320 - DNA, 19 bp, Artificial Sequence
  reverse primer of ARL5C
ctcttggcaagccccgaag

SEQ ID NO: 321 - DNA, 900 bp, Artificial Sequence
  subsequence of SHOX2
gtaatgttaattagcttcatcttaaagaaaaaccaaacaccagagcaaataatgactcctttgcggagaaggagttgtaagggttggggagaggtaattagtgtcataaaagcctcactacaaagaagtttagaatttctctgttctgctgtgcagcagagtgaaacacagtagcagaataattgtgcaccattatcatgataatctaatttgcttctttagaaaaagccactgtttgcagttaggatagtaggaaaacatctagaaaataactaaaatgtaaataaaagtatgaaacaacaataaagttcacttcttaagagaaaaaaagaagagcactcagcgaaaggagaatgaagtatatataaagcaagtaactcttaggtactttgttctcctggatgttacctgcttaaacttgacaacttttacaggcagtatgacagattttttaaaattcaaataaagcttgcttttgtattccactttcaataataaagcatattgattgactcacaaattaaagtttttaaaaccccattaaagtaaaaatactgcttcagagctttcattggtatgtttttgaaagcaagtgtcatagtcaatgtaaatcttcaaaaatttctctgctatttagtacaacagagggtggattttgtgccagggtggcatcctttgtgtaattcacatggcctgaggcttcttgcctttttttcccctagaagagttggaattaagatactaataatcataactgtaataattatcatcatgtttttaaggggttgctgagcctctggagatctcagtgcacagagcgaagcaggagaggcttaaaaaggaaggctctgctgctgatgctgtgatgaccaaggcagcctattattacttttcctcttc

(19) United States
(12) Patent Application Publication
(10) Pub. No.: US 2017/0283887 A1
(43) Pub. Date: Oct. 5, 2017

(54) DIAGNOSIS OF LUNG CANCER

(71) Applicant: AIT Austrian Institute of Technology GmbH, Wien (AT)

(72) Inventors: Matthias Wielscher, Wien (AT); Andreas Weinhäusel, Neckenmarkt (AT); Klemens Vierlinger, Wien (AT); Christa Noehammer, Wien (AT); Rolf Ziesche, Neusiedl am See (AT); Andrea Gsur, Wien (AT)

(21) Appl. No.: 15/529,338

(22) PCT Filed: Nov. 24, 2015
(86) PCT No.: PCT/EP2015/077478

§ 371 Date: May 24, 2017

(30) Foreign Application Priority Data
Nov. 25, 2014 (EP) 14194732.5

(51) Int. Cl. C12Q 1/68

(52) U.S. Cl. CPC C12Q 1/6886; C12Q 2600/154; C12Q 2600/118

(57) ABSTRACT

The present invention relates to specifically methylated nucleic acids, methods of diagnosing lung cancer or a predisposition therefor, and a corresponding kit.

The present invention relates to specifically methylated nucleic acids, methods of diagnosing lung cancer or a predisposition therefor, and a corresponding kit.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Cell-free DNA methylation analysis from blood is useful as minimally invasive diagnostic tool for early detection or disease monitoring. It reduces the need for biopsies or sophisticated imaging methods, which, in case of the lung, can introduce sampling artefacts due to the large area and the complex branching structure of the lung (Alberg et al., Chest 143, e1S-29S (2013)). On the other hand, biopsies were and are established and useful means for early detection or disease monitoring.

Lung diseases are very common. Various studies report a lifetime risk for lung cancer of 6-8% (Fauci, Harrison's principles of internal medicine, McGraw-Hill Medical New York (2008)), which is leading the number of cancer associated deaths worldwide (Alberg (2013), loc. cit.). Evidence derived from minimal invasive tests may aid in early diagnosis and reduce death rates.

A major prerequisite for early diagnosis or monitoring of lung diseases are suitable molecular markers. In this regard, DNA methylation is highly tissue specific (Esteller, N Engl J Med 358, 1148-1159 (2008)). Furthermore, DNA methylation changes can be detected early in cancer development (Brock et al., N Engl J Med 358, 1118-1128 (2008)), which makes it more suitable for early detection than mutation analysis (Brock (2008), loc. cit., Shinozaki et al. Clinical cancer research, 13, 2068-2074 (2007)). For example in cancer, high frequencies of specific methylation alterations were observed already in the early onset of cancer development (Brock (2008), loc. cit.). This frequency is lower in mutation analysis of cancer-derived cell free DNA (cfDNA) (Brock (2008), loc. cit., Shinozaki (2007), loc. cit.).

Commonly used methods for DNA methylation analysis rely on bisulfite conversion of DNA, which allows detection of single CpGs.

Methylation of certain genes in a lung cancer context has been described in the art. Rauch et al. (Cancer Research 66, 7939-7947 (2006)) describe the application of a methylated-CpG island recovery assay (MIRA) to lung cancer cells. Especially homeodomain containing genes were discovered. Kalari et al. (Oncogene 32, 3559-3568 (2013)) characterize the DNA methylation landscape of small cell lung cancer. A further example is EP 2 253 714 which describes the methylation of the PCDHGA12 gene in lung cancer cells.

US2010/0273151 describes a large number of genes which are differentially methylated when comparing medulloblastoma samples with normal cerebellum. Similarly, US2011/0318738 describes a host of genes which are differentially methylated in FAP adenoma tissue. The medical indication which is lung cancer is not mentioned in these documents.

The present inventors discovered new markers as well as previously unknown disease-relevant methylation positions in markers which, as such, were previously described. Moreover, not only differentially methylated genes and positions are provided, but also methods of diagnosing and disease monitoring.

In view of the prior art, the technical problem underlying the present invention can be seen in the provision of alternative or improved means and methods for diagnosing lung cancer.

The technical problem has been solved by the subject-matter of the claims enclosed herewith. Accordingly, in a first aspect, the present invention relates to a nucleic acid comprising or consisting of

(a) the sequence
(1) from position 7 to position 101 of SEQ ID NO: 4, wherein the cytidine in at least one CpG dinucleotide comprised in said sequence is methylated;
(2) from position 11 to position 71 of SEQ ID NO: 2, wherein the cytidine in at least one CpG dinucleotide comprised in said sequence is methylated;
(3) from position 37 to position 81 of SEQ ID NO: 3, wherein the cytidine in at least one CpG dinucleotide comprised in said sequence is methylated;
(4) from position 4 to position 84 of SEQ ID NO: 1, wherein the cytidine in at least one CpG dinucleotide comprised in said sequence is methylated;
(5) from position 28 to position 69 of SEQ ID NO: 5, wherein the cytidine in at least one CpG dinucleotide comprised in said sequence is methylated;
(6) from position 39 to position 85 of SEQ ID NO: 6, wherein the cytidine in at least one CpG dinucleotide comprised in said sequence is methylated;
(7) from position 10 to position 53 of SEQ ID NO: 7, wherein the cytidine in at least one CpG dinucleotide comprised in said sequence is methylated;
(8) from position 13 to position 71 of SEQ ID NO: 8, wherein the cytidine in at least one CpG dinucleotide comprised in said sequence is methylated;
(9) from position 5 to position 71 of SEQ ID NO: 9, wherein the cytidine in at least one CpG dinucleotide comprised in said sequence is methylated;
(10) from position 2 to position 60 of SEQ ID NO: 10, wherein the cytidine in at least one CpG dinucleotide comprised in said sequence is methylated;
(11) from position 22 to position 63 of SEQ ID NO: 11, wherein the cytidine in at least one CpG dinucleotide comprised in said sequence is methylated;
(12) from position 45 to position 67 of SEQ ID NO: 12, wherein the cytidine in at least one CpG dinucleotide comprised in said sequence is methylated;
(13) from position 58 to position 78 of SEQ ID NO: 13, wherein the cytidine in at least one CpG dinucleotide comprised in said sequence is methylated;
(14) from position 31 to position 66 of SEQ ID NO: 14, wherein the cytidine in at least one CpG dinucleotide comprised in said sequence is methylated;
(15) from position 41 to position 72 of SEQ ID NO: 15, wherein the cytidine in at least one CpG dinucleotide comprised in said sequence is methylated;
(16) from position 53 to position 82 of SEQ ID NO: 16, wherein the cytidine in at least one CpG dinucleotide comprised in said sequence is methylated;
(17) from position 20 to position 74 of SEQ ID NO: 17, wherein the cytidine in at least one CpG dinucleotide comprised in said sequence is methylated;
(18) from position 9 to position 98 of SEQ ID NO: 18, wherein the cytidine in at least one CpG dinucleotide comprised in said sequence is methylated;
(19) from position 23 to position 85 of SEQ ID NO: 19, wherein the cytidine in at least one CpG dinucleotide comprised in said sequence is methylated;
(20) from position 20 to position 28 of SEQ ID NO: 20, wherein the cytidine in at least one CpG dinucleotide comprised in said sequence is methylated;
(21) from position 24 to position 61 of SEQ ID NO: 21, wherein the cytidine in at least one CpG dinucleotide comprised in said sequence is methylated;
(22) from position 11 to position 84 of SEQ ID NO: 22, wherein the cytidine in at least one CpG dinucleotide comprised in said sequence is methylated;
(23) from position 2 to position 86 of SEQ ID NO: 23, wherein the cytidine in at least one CpG dinucleotide comprised in said sequence is methylated;
(24) from position 43 to position 80 of SEQ ID NO: 24, wherein the cytidine in at least one CpG dinucleotide comprised in said sequence is methylated;
(25) from position 24 to position 70 of SEQ ID NO: 25, wherein the cytidine in at least one CpG dinucleotide comprised in said sequence is methylated;
(26) from position 44 to position 94 of SEQ ID NO: 26, wherein the cytidine in at least one CpG dinucleotide comprised in said sequence is methylated;
(27) from position 44 to position 67 of SEQ ID NO: 27, wherein the cytidine in at least one CpG dinucleotide comprised in said sequence is methylated;
(28) from position 20 to position 43 of SEQ ID NO: 28, wherein the cytidine in at least one CpG dinucleotide comprised in said sequence is methylated;
(29) from position 3 to position 71 of SEQ ID NO: 29, wherein the cytidine in at least one CpG dinucleotide comprised in said sequence is methylated;
(30) from position 11 to position 64 of SEQ ID NO: 30, wherein the cytidine in at least one CpG dinucleotide comprised in said sequence is methylated;
(31) from position 7 to position 118 of SEQ ID NO: 31, wherein the cytidine in at least one CpG dinucleotide comprised in said sequence is methylated;
(32) from position 25 to position 95 of SEQ ID NO: 32, wherein the cytidine in at least one CpG dinucleotide comprised in said sequence is methylated;
(33) from position 29 to position 46 of SEQ ID NO: 33, wherein the cytidine in at least one CpG dinucleotide comprised in said sequence is methylated;
(34) from position 8 to position 72 of SEQ ID NO: 34, wherein the cytidine in at least one CpG dinucleotide comprised in said sequence is methylated;
(35) from position 5 to position 57 of SEQ ID NO: 35, wherein the cytidine in at least one CpG dinucleotide comprised in said sequence is methylated;
(36) from position 43 to position 67 of SEQ ID NO: 36, wherein the cytidine in at least one CpG dinucleotide comprised in said sequence is methylated;
(37) from position 21 to position 63 of SEQ ID NO: 37, wherein the cytidine in at least one CpG dinucleotide comprised in said sequence is methylated;
(38) from position 27 to position 86 of SEQ ID NO: 38, wherein the cytidine in at least one CpG dinucleotide comprised in said sequence is methylated;
(39) from position 2 to position 110 of SEQ ID NO: 39, wherein the cytidine in at least one CpG dinucleotide comprised in said sequence is methylated;
(40) from position 13 to position 59 of SEQ ID NO: 40, wherein the cytidine in at least one CpG dinucleotide comprised in said sequence is methylated;
(41) from position 32 to position 107 of SEQ ID NO: 41, wherein the cytidine in at least one CpG dinucleotide comprised in said sequence is methylated;
(42) from position 3 to position 48 of SEQ ID NO: 42, wherein the cytidine in at least one CpG dinucleotide comprised in said sequence is methylated;
(43) from position 5 to position 57 of SEQ ID NO: 43, wherein the cytidine in at least one CpG dinucleotide comprised in said sequence is methylated;
(44) from position 40 to position 106 of SEQ ID NO: 44, wherein the cytidine in at least one CpG dinucleotide comprised in said sequence is methylated;
(45) from position 26 to position 90 of SEQ ID NO: 45, wherein the cytidine in at least one CpG dinucleotide comprised in said sequence is methylated; or
(46) from position 68 to position 74 of SEQ ID NO: 46, wherein the cytidine in at least one CpG dinucleotide comprised in said sequence is methylated;
or
   (b) a sequence having at least 80% sequence identity to a sequence of (a).

The term “nucleic acid” has its art-establish meaning. It relates to a polycondensate of nucleotides. The nucleic acid is preferably DNA. Accordingly, the group of nucleotide building blocks comprises or consists of adenine, guanine, cytosine and thymidine, commonly abbreviated as A, G, C and T.

While not being preferred, it is envisaged that one, more or all nucleotides in any nucleic acid (including probes and primers) of the invention may comprise one or more art-established modifications. Art-established modifications include modifications of the 2′-position of the sugar moiety which is desoxyribose in case of DNA. An example of a 2′-modification is 2′-O-methyl. Another art-established modification of the sugar phosphate backbone is the replacement of oxygen with sulfur in a phosphate moiety. Correspondingly modified phosphates are known, for example, as phosphorothioate.

Further included are nucleic acid mimicking molecules known in the art such as synthetic or semisynthetic derivatives of DNA or RNA and mixed polymers, to be used as sense and/or antisense strands. They may contain additional non-natural or derivatized nucleotide bases, as will be readily appreciated by those skilled in the art. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), locked nucleic acid (LNA) (see, for example, Braasch and Corey, Chemistry & Biology 8, 1-7 (2001)) and peptide nucleic acid (PNA).

LNA is an RNA derivative in which the ribose ring is constrained by a methylene linkage between the 2′-oxygen and the 4′-carbon.

For the purposes of the present invention, a peptide nucleic acid (PNA) is a polyamide type of DNA analog. The monomeric units for the corresponding derivatives of adenine, guanine, thymine and cytosine are available commercially (for example from Perceptive Biosystems). PNA is a synthetic DNA-mimic with an amide backbone in place of the sugar-phosphate backbone of DNA or RNA. As a consequence, certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. Furthermore, they are stable under acidic conditions and resistant to proteases (Demidov et al. (1994), Biochem. Pharmacol., 48, 1310-1313). Their electrostatically neutral backbone increases the binding strength to complementary DNA as compared to the stability of the corresponding DNA-DNA duplex (Wittung et al. (1994), Nature 368, 561-563; Ray and Norden (2000), Faseb J., 14, 1041-1060). In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (Tₘ) by 8°−20° C., vs. 4°−16° C. for the DNA/DNA 15-mer duplex. Thereby discrimination between perfect matches and mismatches is improved. For its uncharged nature, PNA also permits the hybridisation of DNA samples at low salt or no-salt conditions, since no inter-strand repulsion as between two negatively charged DNA strands needs to be counteracted. As a consequence, the target DNA has fewer secondary structures under hybridisation conditions and is more accessible to probe molecules.

PNA chimera according to the present invention are molecules comprising one or more PNA portions. The remainder of the chimeric molecule may comprise one or more DNA portions (PNA-DNA chimera).

A further modification in accordance with the invention is the conversion of cytosine to uracil. When an accordingly modified nucleic acid is amplified by conventional techniques, uracil is converted into thymidine. The conversion of C into U can be performed experimentally by treatment with bisulfite. Alternatively, it can be performed in silico by corresponding software programs. Either method is well-established in the art. To the extent cytosine is methylated, it is not amenable to conversion. Accordingly, correspondingly modified sequences of the present invention are those where all Cs are converted into Ts, including those in CpG dinucleotides which in principle are amenable to methylation, but are not methylated under the circumstances given. Also provided are modified sequences where one, more or all of the Cs within CpG dinucleotides are methylated and accordingly not amenable to conversion from C to T. In the latter sequences, only those Cs are converted into Ts which are not methylated in the parent sequence. Preferred sequences in this regard are subject of a preferred embodiment detailed further below. Not only C/T converted sequences, but also C/U converted sequence are embraced by the invention.

Methylation of DNA typically occurs in regions where CpG dinucleotides are overrepresented as compared to their average occurrence within a given genome. For the purposes of the present invention, we refer to such regions where CpG dinucleotides occur more frequently as “CpG clusters”. We note that there exists also the art-established term “CpG island”. Generally, the term “CpG island” designates a stretch of up to 500 base pairs in length where more than 60% of the base sequence is present in the form of CpG dinucleotides. There may be flanking regions on one or both sides of a given CpG island where CpG dinucleotides are still overrepresented as compared to the genome average. Such flanking sequences are generally referred to as “CpG shores”. The term “CpG cluster” as used herein differs from these art-established definitions in that there is no minimum requirement as regards the frequency of CpG dinucleotide motifs. A CpG cluster in accordance with the present invention is a sequence of typically 100 nucleotides or less which contains at least two CpG dinucleotide motifs which are amenable to methylation. While CpG islands according to the art-established definition have a length of up to 500 base pairs the CpG clusters disclosed above are of length below 100 base pairs, we note that regions with an elevated frequency of CpG dinucleotides may be have lengths of up to 1000 or even 3000 bases. The abbreviation “CpG” designates a dinucleotide motif occurring within a given DNA strand. Typically, methylated bases are cytosines comprised in such dinucleotide motifs. To give an example, the subsequence from position 4 to position 84 of SEQ ID NO: 1 comprises five CpG dinucleotide motifs. The subsequence of SEQ ID NO: 1 from position 4 to position 84 accordingly defines a CpG cluster, in this specific case a previously unknown CpG cluster in the genomic DNA of the PAX9 gene. Generally speaking, the first aspect defines in each of items (1) to (46) a disease-associated CpG cluster.

The enzymatically catalyzed process of methylation of cytosine yields 5-methyl cytosine. Methylated forms of DNA may be further derivatized within a cell to give rise to cytosines which bear a hydroxymethyl group in the 5 position. “Methylated cytosine” in accordance with the present invention is either 5-methyl cytosine or 5-hydroxymethyl cytosine, 5-methyl cytosine being preferred. The enzymes catalyzing methylation of DNA are known as DNA methyl transferases. DNA modification by the process of methylation is a form of epigenetic gene regulation. DNA methylation as well as hydroxymethylation is frequently associated with development and/or disease.

If a position is methylated in accordance with the present invention, it is generally or preferably methylated to a high degree, for example more than 60%, more than 70%, more than 80%, more than 90%, more than 95%, more than 98%, more than 99% or complete; i.e. 100% methylation. As such, the methylation status of a particular position can generally be viewed as a Boolean variable: a given position is either unmethylated or methylated. The term “unmethylated” in accordance with the present invention refers to low degrees of methylation, preferably less than 20%, less than 10%, less than 5%, less than 2% or less than 1%. Also included within the term “unmethylated” is the complete absence of methylation or the absence of detectable methylation. Furthermore, it is generally observed that all cytosines of the CpG motifs of a given CpG cluster are either methylated or not. Accordingly, it is preferred that within a given CpG cluster, the degree of methylation does not vary among the individual CpG motifs by more than 40%, more than 30%, more than 20%, more than 10%, more than 5%, more than 2%, or more than 1%. In other words, preferably either are all positions within a given CpG cluster methylated or they are not.

The preferred percentages given above relate in particular to those cases where a sample is directly drawn from a tumor, for example by means of a biopsy. Having said that, we note that in accordance with the preferred embodiment detailed further below, the methods in accordance with the present invention are performed by using samples of cell-free DNA. While in patients suffering from lung cancer, (i) the amount of cell-free DNA is generally increased, and (ii) cell-free DNA originating from the cancer is present among the cell-free DNA comprised in serum, it will also be generally true that a significant amount of cell-free DNA which does not originate from the cancer is present in the sample. The cell-free DNA obtained from a lung cancer patient typically contains a mixture of methylated and non-methylated DNA. Accordingly, while the nucleic acid defined in accordance with the first aspect of the invention, when originating from a lung cancer, will generally be methylated, the corresponding sequences originating from other parts of the body of the patient will generally not be methylated.

Under such circumstances, the methods of diagnosing and disease monitoring in accordance with the present invention can nevertheless be practiced. A preferred measure of the methylation status is the fold change (F.C.). The fold change is the ratio of the amount of methylated DNA in a sample from a patient suffering from lung cancer over the amount of methylated DNA from an individual which does not suffer from lung cancer for a given gene, fragment thereof, CpG cluster or CpG dinucleotide, respectively. In addition, logarithms of the fold change may be given. The fold change data in the tables enclosed herewith are logarithms. The term “amount” includes molar concentration, mass per unit volume (w/v %), mass, number of molecules, and relative amount with respect to (a) reference gene(s). The terms “patient” and “individual” refer to single persons as well as averages of a cohort and population averages. A preferred sample is plasma. A preferred individual who does not suffer from lung cancer is a healthy and/or non-cancer-control individual. Preferred forms of lung cancer are given further below. Consistent with the use of fold changes for the purpose of diagnosis and disease monitoring, the respective CpG dinucleotide motifs and CpG clusters are also referred to as being differentially methylated. In case of the markers in accordance with the present invention, the diseased state, i.e. lung cancer, is characterized by a higher degree of methylation as compared to the healthy state, and, more generally any non-cancer state. Owing to the background of cell-free DNA originating from healthy tissue, though, the above mentioned high percentages of methylation, although typically observed in biopsies, will not be observed in cell-free DNA samples.

Having said that, the use of a control (in case of the fold change as defined above a sample from an individual which does not suffer from lung cancer) is not indispensable when analyzing any type of sample such as cell-free DNA. To explain further, the method of the present invention may be calibrated. Calibration may be for a given type of sample, a given workflow of sample processing, and/or a given gene, CpG cluster or CpG dinucleotide. Calibration results in a threshold value for a given measure of the methylation status. The threshold distinguishes diseased from non-diseased samples as well as methylated from non-methylated samples. For the sake of completeness we note that also in the scenario described above (background of non-methylated markers in cell-free DNA samples) a given CpG dinucleotide, CpG cluster or gene may be classified as being methylated, for example either based on fold change or the above mentioned calibration. In such context, the status “methylated” refers to a status where it is the contribution to the total amount of cell-free DNA which contribution originates from the tumor, metastasis and/or circulating tumor cells, and accounts for a component of methylated DNA. This component is detected and provides for a classification as being methylated based on fold change or comparison with the above mentioned threshold value.

The fold change is a generally applicable measure and is not confined to the analysis of cell-free DNA.

If not indicated otherwise, the term “methylation” as used in the following refers to the state of being methylated, rather than to the chemical reaction.

Nucleic acids in accordance with the first aspect may either consist of the specifically indicated subsequence of a given entry of the sequence listing (e.g. from position 4 to position 84 of SEQ ID NO: 1), or may comprise said subsequence. In the latter case, one or two flanking sequences may be present upstream, downstream or upstream and downstream to the specifically indicated subsequence. Flanking sequences preferably are in the range of 100, 200, 300, 400 or 500 base pairs. To the extent the first aspect refers to nucleic acids comprising the specifically defined sequences, it is preferred that the nucleic acid as a whole is a part of a gene or corresponds to an entire gene, but generally does not extend beyond the length of an entire gene. This will become apparent in view of preferred embodiments described further below. In particular, a nucleic acid in accordance with the first aspect does not extend to entire chromosomes.

Preferred levels of sequence identity are at least 85%, at least 90%, at least 95%, at least 98% or at least 99%.

Two nucleotide sequences can be aligned electronically using suitable computer programs known in the art. Such programs comprise BLAST (Altschul et al. (1990), J. Mol. Biol. 215, 403-410), variants thereof such as WU-BLAST (Altschul & Gish (1996), Methods Enzymol. 266, 460-480), FASTA (Pearson & Lipman (1988), Proc. Natl. Acad. Sci. USA 85, 2444-2448) or implementations of the Smith-Waterman algorithm (SSEARCH, Smith & Waterman (1981), J. Mol. Biol. 147, 195-197). These programs, in addition to providing a pairwise sequence alignment, also report the sequence identity level (usually in percent identity) and the probability for the occurrence of the alignment by chance (P-value). Programs such as CLUSTALW (Higgins et al. (1994), Nucleic Acids Res. 22, 4673-4680) can be used to align more than two sequences. Preferably, default settings of the respective computer programs as published are used. A preferred alignment program is BLAST (loc. cit.).

To the extent the first aspect relates to nucleic acids the sequence of which is not identical to any of the entries of the sequence listing, at least 50% of the CpG dinucleotide motifs in accordance with the invention are present in said sequence. Preferably, at least 70%, at least 80%, at least 90% or all of the CpG dinucleotide motifs which are present in any one of SEQ ID NOs: 1 to 46 are also present in a sequence in accordance with item (b) and exhibiting 80% or more sequence identity to any one of the sequences of SEQ ID NOs: 1 to 46.

Each of SEQ ID NOs: 1 to 46 is a subsequence of a given gene which subsequence comprises a CpG cluster. The genes, designated by art-established gene symbols, and their correspondence to the sequences of SEQ ID NOs: 1 to 46 are given in Table 1 below. Table 1 furthermore includes the performance measures fold change (F.C.), area under the curve (AUC), sensitivity and specificity of prediction. P-values are determined using a t-test corrected for multiple testing according to Benjamini Hochberg; F.C. indicates the log difference between case and control. AUC is area under curve derived from Receiver operator characteristics curve analysis; Sens indicates Sensitivity derived from ROC-curve analysis and Spec indicates Specificity.

TABLE 1

| Gene symbol | SEQ ID NO: | P. value | F.C. | AUC | Sens | Spec |
|---|---|---|---|---|---|---|
| PAX9 | 4 | 7.05E−13 | 14.58 | 0.88 | 0.95 | 0.80 |
| HOXD10 | 2 | 1.30E−10 | 12.68 | 0.87 | 0.94 | 0.79 |
| TBX20 | 3 | 2.33E−03 | 1.5 | 0.86 | 0.93 | 0.78 |
| CACNA1B | 1 | 1.97E−06 | 15.98 | 0.81 | 0.90 | 0.71 |
| ZIC1 | 5 | 2.05E−07 | 10.42 | 0.77 | 0.87 | 0.68 |
| SIM1 | 6 | 4.99E−08 | 9.65 | 0.76 | 0.86 | 0.67 |
| DLX1 | 7 | 7.04E−06 | 12.52 | 0.73 | 0.83 | 0.64 |
| LMX1A | 8 | 3.54E−07 | 12.36 | 0.75 | 0.85 | 0.66 |
| DLX6AS1 | 9 | 1.55E−07 | 11.57 | 0.83 | 0.92 | 0.74 |
| MIR196A1 | 10 | 3.54E−07 | 16 | 0.81 | 0.90 | 0.72 |
| FEZF2 | 11 | 1.90E−08 | 15.68 | 0.80 | 0.89 | 0.71 |
| STAG3 | 12 | 7.68E−05 | 1.77 | 0.75 | 0.85 | 0.65 |
| PTPRN2 | 13 | 6.03E−03 | 1.16 | 0.83 | 0.92 | 0.74 |
| BARHL1 | 14 | 8.38E−06 | 12.86 | 0.75 | 0.85 | 0.65 |
| CCDC140 | 15 | 8.33E−09 | 6.4 | 0.84 | 0.93 | 0.76 |
| CpG138 | 16 | 1.24E−07 | 13.52 | 0.80 | 0.89 | 0.70 |
| GATA2 | 17 | 1.55E−07 | 11.27 | 0.77 | 0.86 | 0.67 |
| ADCYAP1 | 18 | 9.88E−07 | 2.09 | 0.80 | 0.89 | 0.71 |
| TBX5 | 19 | 2.41E−06 | 9.17 | 0.72 | 0.82 | 0.63 |
| CpG38 | 20 | 1.30E−05 | 14.8 | 0.74 | 0.84 | 0.63 |
| NECAB1 | 21 | 1.57E−05 | 8.82 | 0.74 | 0.84 | 0.64 |
| ADRB1 | 22 | 1.99E−05 | 13.63 | 0.72 | 0.83 | 0.62 |
| OTX2 | 23 | 2.52E−05 | 1.44 | 0.80 | 0.89 | 0.70 |
| NKX2-6 | 24 | 5.06E−05 | 2.32 | 0.77 | 0.87 | 0.67 |
| SIM1 | 25 | 1.11E−04 | 7.28 | 0.73 | 0.83 | 0.63 |
| DRD4 | 26 | 1.11E−04 | 11.74 | 0.70 | 0.81 | 0.58 |
| TBX15 | 27 | 1.42E−04 | 11.84 | 0.69 | 0.80 | 0.59 |
| TRH | 28 | 1.48E−04 | 1.42 | 0.72 | 0.82 | 0.61 |
| FBP1 | 29 | 1.71E−04 | 14.52 | 0.75 | 0.85 | 0.64 |
| CpG615 | 30 | 1.71E−04 | 1.1 | 0.80 | 0.89 | 0.71 |
| BCAN | 31 | 7.87E−04 | 1.34 | 0.84 | 0.92 | 0.76 |
| TP73 | 32 | 8.39E−04 | 14.16 | 0.70 | 0.81 | 0.59 |
| MIIP | 33 | 1.09E−03 | 0.41 | 0.60 | 0.72 | 0.49 |
| HOXC4 | 34 | 1.33E−03 | 8.04 | 0.68 | 0.79 | 0.58 |
| HOXA7 | 35 | 1.44E−03 | 2.76 | 0.81 | 0.90 | 0.71 |
| BST1 | 36 | 1.54E−03 | 2.11 | 0.74 | 0.85 | 0.64 |
| C1orf114 | 37 | 1.75E−03 | 14.65 | 0.67 | 0.78 | 0.55 |
| FOXG1 | 38 | 2.96E−03 | 13.52 | 0.64 | 0.75 | 0.52 |
| ATP4A | 39 | 5.40E−03 | 0.71 | 0.71 | 0.82 | 0.60 |
| NOTCH1 | 40 | 1.10E−02 | 3.84 | 0.69 | 0.81 | 0.58 |
| CpG80 | 41 | 1.21E−02 | 1.15 | 0.73 | 0.84 | 0.63 |
| N/A | 42 | 2.10E−02 | 1.61 | 0.77 | 0.88 | 0.67 |
| HOXA7 | 43 | 2.26E−02 | 2.25 | 0.78 | 0.88 | 0.67 |
| ZNF529 | 44 | 3.31E−02 | 0.05 | 0.55 | 0.67 | 0.43 |
| PRLHR | 45 | 4.96E−02 | 0 | 0.59 | 0.70 | 0.48 |
| ARL5C | 46 | 1.06E−01 | 0.15 | 0.58 | 0.67 | 0.49 |

To the extent reference is made to CpG numbers in this table, these are the numbers according to the UCSC browser (J Mol Biol.; 196(2):261-82 (1987)).

Table 2 provides structural and functional information about certain particularly preferred genes.

TABLE 2

| Gene symbol | Description |
|---|---|
| PAX9 | This gene is a member of the paired box (PAX) family of transcription |
| | factors. Members of this gene family typically contain a paired box domain, |
| | an octapeptide, and a paired-type homeodomain. These genes play critical |
| | roles during fetal development and cancer growth. The specific function of |
| | the paired box 9 gene is unknown but it may involve development of |
| | stratified squamous epithelia as well as various organs and skeletal |
| | elements. |
| HOXD10 | This gene is a member of the Abd-B homeobox family and encodes a |
| | protein with a homeobox DNA-binding domain. It is included in a cluster of |
| | homeobox D genes located on chromosome 2. The encoded nuclear |
| | protein functions as a sequence-specific transcription factor that is |
| | expressed in the developing limb buds and is involved in differentiation and |
| | limb development. Mutations in this gene have been associated with Wilm's |
| | tumor and congenital vertical talus (also known as “rocker-bottom foot” |
| | deformity or congenital convex pes valgus) and/or a foot deformity |
| | resembling that seen in Charcot-Marie-Tooth disease. |
| TBX20 | This gene encodes a T-box family member. The T-box family members |
| | share a common DNA binding domain, termed the T-box, and they are |
| | transcription factors involved in the regulation of developmental processes. |
| | This gene is essential for haart development. Mutations in this gene are |
| | associated with diverse cardiac pathologies, including defects in septation, |
| | valvulogenesis and cardiomyopathy. Alternatively spliced transcript variants |
| | encoding different isoforms have been found for this gene. |
| CACNA1B | The protein encoded by this gene is the pore-forming subunit of an N-type |
| | voltage-dependent calcium channel, which controls neurotransmitter |
| | release from neurons. The encoded protein forms a complex with alpha-2, |
| | beta, and delta subunits to form the high-voltage activated channel. This |
| | channel is sensitive to omega-conotoxin-GVIA and omega-agatoxin-IIIA but |
| | insensitive to dihydropyridines. Two transcript variants encoding different |
| | isoforms have been found for this gene. |
| ZIC1 | This gene encodes a member of the ZIC family of C2H2-type zinc finger |
| | proteins. Members of this family are important during development. |
| | Aberrant expression of this gene is seen in medulloblastoma, a childhood |
| | brain tumor. This gene is closely linked to the gene encoding zinc finger |
| | protein of the cerebellum 4, a related family member on chromosome 3. |
| | This gene encodes a transcription factor that can bind and transactivate the |
| | apolipoprotein E gene. |
| SIM1 | SIM1 and SIM2 genes are Drosophila single-minded (sim) gene homologs. |
| | SIM1 transcript was detected only in fetal kidney out of various adult and |
| | fetal tissues tested. Since the sim gene plays an important role in |
| | Drosophila development and has peak levels of expression during the |
| | period of neurogenesis, it was proposed that the human SIM gene is a |
| | candidate for involvement in certain dysmorphic features (particularly the |
| | facial and skull characteristics), abnormalities of brain development, and/or |
| | mental retardation of Down syndrome. |
| DLX1 | This gene encodes a member of a homeobox transcription factor gene |
| | family similar to the Drosophila distal-less gene. The encoded protein is |
| | localized to the nucleus where it may function as a transcriptional regulator |
| | of signals from multiple TGF-(17) superfamily members. The encoded |
| | protein may play a role in the control of craniofacial patterning and the |
| | differentiation and survival of inhibitory neurons in the forebrain. This gene |
| | is located in a tail-to-tail configuration with another member of the family on |
| | the long arm of chromosome 2. Alternatively spliced transcript variants |
| | encoding different isoforms have been described. |
| LMX1A | This gene encodes a homeodomain and LIM-domain containing protein. |
| | The encoded protein is a transcription factor that acts as a positive regulator |
| | of insulin gene transcription. This gene also plays a role in the development |
| | of dopamine producing neurons during embryogenesis. Mutations in this |
| | gene are associated with an increased risk of developing Parkinson's |
| | disease. Alternate splicing results in multiple transcript variants. |
| DLX6AS1 | DLX6-AS1 DLX6 antisense RNA 1. |
| MIR196A1 | microRNAs (miRNAs) are short (20-24 nt) non-coding RNAs that are |
| | involved in post-transcriptional regulation of gene expression in multicellular |
| | organisms by affecting both the stability and translation of mRNAs. miRNAs |
| | are transcribed by RNA polymerase II as part of capped and polyadenylated |
| | primary transcripts (pri-miRNAs) that can be either protein-coding or non- |
| | coding. The primary transcript is cleaved by the Drosha ribonuclease III |
| | enzyme to produce an approximately 70-nt stem-loop precursor miRNA |
| | (pre-miRNA), which is further cleaved by the cytoplasmic Dicer ribonuclease |
| | to generate the mature miRNA and antisense miRNA star (miRNA*) |
| | products. The mature miRNA is incorporated into a RNA-induced silencing |
| | complex (RISC), which recognizes target mRNAs through imperfect base |
| | pairing with the miRNA and most commonly results in translational inhibition |
| | or destabilization of the target mRNA. The RefSeq represents the predicted |
| | microRNA stem-loop. |
| STAG3 | The protein encoded by this gene is expressed in the nucleus and is a |
| | subunit of the cohesion complex which regulates the cohesion of sister |
| | chromatids during cell division. A mutation in this gene is associated with |
| | premature ovarian failure. Alternate splicing results in multiple transcript |
| | variants encoding distinct isoforms. This gene has multiple pseudogenes. |
| PTPRN2 | The protein encoded by this gene is a member of the protein tyrosine |
| | phosphatase (PTP) family. PTPs are known to be signaling molecules that |
| | regulate a variety of cellular processes including cell growth, differentiation, |
| | mitotic cycle, and oncogenic transformation. This PTP possesses an |
| | extracellular region, a single transmembrane region, and a single |
| | intracellular catalytic domain, and thus represents a receptor-type PTP. The |
| | catalytic domain of this PTP is most closely related to PTPRN/IA-2beta. |
| | PTP and PTPRN are both found to be major autoantigens associated with |
| | insulin-dependent diabetes mellitus. Three alternatively splice transcript |
| | variants of this gene, which encode distinct proteins, have been reported. |

The subject-matter of the first aspect relates to novel cancer-associated CpG clusters. The diseased state is characterized by methylation for all genes in accordance with the present invention. By determining the methylation status of one or more of said cancer-associated CpG clusters or specific cytidine(s) within one or more CpG clusters, lung cancer or a predisposition therefor may be diagnosed. This is the subject of the second aspect of the present invention and is detailed further below. Nucleic acids in accordance with the present invention may be used as such, uses including the mentioned diagnostic applications. Alternatively, they may serve as starting point for amplification reactions, said amplification reactions being further detailed below, as well as for amplification independent methods of determining the methylation status, such amplification independent methods also being further detailed below. Certain methods of the present invention provide for amplification being specifically applied to methylated templates.

In a preferred embodiment, at least one, preferably all cytidines at positions

(1) 7, 32, 37, 39, 41, 43, 53, 71 and 100 in SEQ ID NO: 4 is/are methylated;
(2) 11, 23, 26, 33, 41, 54, 56, 66 and 70 in SEQ ID NO: 2 is/are methylated;
(3) 37, 41, 50 and 80 in SEQ ID NO: 3 is/are methylated;
(4) 4, 38, 40, 45 and 83 in SEQ ID NO: 1 is/are methylated;
(5) 28, 46, 52 and 68 in SEQ ID NO: 5 is/are methylated;
(6) 39, 46, 48, 50, 61, 82 and 84 in SEQ ID NO: 6 is/are methylated;
(7) 10 and 52 in SEQ ID NO: 7 is/are methylated;
(8) 13, 32, 50, 57 and 70 in SEQ ID NO: 8 is/are methylated;
(9) 5, 46, 48, 55 and 70 in SEQ ID NO: 9 is/are methylated;
(10) 2, 29, 31, 56 and 59 in SEQ ID NO: 10 is/are methylated;
(11) 22, 29, 49, 52, 54 and 62 in SEQ ID NO: 11 is/are methylated;
(12) 45, 59, 64 and 66 in SEQ ID NO: 12 is/are methylated;
(13) 58 and 77 in SEQ ID NO: 13 is/are methylated;
(14) 31, 48, 54 and 65 in SEQ ID NO: 14 is/are methylated;
(15) 41 and 71 in SEQ ID NO: 15 is/are methylated;
(16) 53, 63, 65 and 81 in SEQ ID NO: 16 is/are methylated;
(17) 20, 42, 58, 66 and 73 in SEQ ID NO: 17 is/are methylated;
(18) 9, 31, 61 and 97 in SEQ ID NO: 18 is/are methylated;
(19) 23, 34, 37, 56, 58, 67, 81 and 84 in SEQ ID NO: 19 is/are methylated;
(20) 20, 24 and 27 in SEQ ID NO: 20 is/are methylated;
(21) 24 and 60 in SEQ ID NO: 21 is/are methylated;
(22) 11, 49 and 83 in SEQ ID NO: 22 is/are methylated;
(23) 2, 18 and 85 in SEQ ID NO: 23 is/are methylated;
(24) 43, 52, 68 and 79 in SEQ ID NO: 24 is/are methylated;
(25) 24 and 69 in SEQ ID NO: 25 is/are methylated;
(26) 44, 50, 64 and 93 in SEQ ID NO: 26 is/are methylated;
(27) 44 and 66 in SEQ ID NO: 27 is/are methylated;
(28) 20, 35 and 42 in SEQ ID NO: 28 is/are methylated;
(29) 3, 68 and 70 in SEQ ID NO: 29 is/are methylated;
(30) 11, 33, 46 and 63 in SEQ ID NO: 30 is/are methylated;
(31) 7, 58, 95 and 117 in SEQ ID NO: 31 is/are methylated;
(32) 25, 27, 30, 39, 42, 59 and 94 in SEQ ID NO: 32 is/are methylated;
(33) 29, 32 and 45 in SEQ ID NO: 33 is/are methylated;
(34) 8, 37, 40, 56, 58, 60, 68 and 71 in SEQ ID NO: 34 is/are methylated;
(35) 5, 41, 50 and 56 in SEQ ID NO: 35 is/are methylated;
(36) 43, 60 and 66 in SEQ ID NO: 36 is/are methylated;
(37) 21, 56 and 62 in SEQ ID NO: 37 is/are methylated;
(38) 27, 67, 69 and 85 in SEQ ID NO: 38 is/are methylated;
(39) 2, 56, 62 and 109 in SEQ ID NO: 39 is/are methylated;
(40) 13, 47 and 58 in SEQ ID NO: 40 is/are methylated;
(41) 32, 34, 39 and 106 in SEQ ID NO: 41 is/are methylated;
(42) 3, 19, 44 and 47 in SEQ ID NO: 42 is/are methylated;
(43) 5, 41, 50 and 56 in SEQ ID NO: 43 is/are methylated;
(44) 40, 61, 67 and 105 in SEQ ID NO: 44 is/are methylated;
(45) 26, 28, 35, 42, 48 and 89 in SEQ ID NO: 45 is/are methylated; or
(46) 68 and 73 in SEQ ID NO: 46 is/are methylated.

This preferred embodiment explicitly lists the cytidines comprised in CpG motifs, said CpG motifs comprised in a CpG cluster in accordance with the present invention. To give an example, in case of SEQ ID NO: 1, it is particularly preferred that all cytidines specifically indicated, namely those at positions 4, 38, 40, 45 and 83 are methylated. In a less preferred embodiment, only one, only two, only three, only four, only five, only six, only seven, or only eight cytidines are methylated. It is understood that the latter statement, when referring to a given integer number, is only applicable to those CpG clusters which contain a number of CpG motifs which number of CpG motifs is larger than the specific integer number under consideration in the latter statement. To provide an example, in case of SEQ ID NO: 1, a specific embodiment relates to four out of the five CpG dinucleotides being methylated. As regards SEQ ID NO: 2, owing to the presence of nine CpG dinucleotide motifs, an embodiment is possible which is not possible in the case of SEQ ID NO: 1, namely that eight out of nine CpG dinucleotides are methylated.

In general, and as noted above, methylation of the various CpG dinucleotides comprised in a given CpG cluster of the invention is correlated. Accordingly, the cytidines in all indicated CpG dinucleotide motifs will generally be fully methylated or methylated to very high degree in a sample obtained from a patient having lung cancer or from an individual having a predisposition to develop lung cancer. Said sample may be a lung cell from the tumor. Alternatively, it may be taken from a metastasis of a patient suffering from lung cancer. Also, it may be a circulating tumor cell from a patient suffering from lung cancer.

Furthermore, the above applies mutatis mutandis to any of the measures of methylation status disclosed herein such as fold change. To explain further, any given measure of methylation determined for the CpG dinucleotide motifs within a given CpG cluster in accordance with the present invention in general will be correlated. Preferably, any given measure of methylation determined for CpG dinucleotides within a given CpG cluster do not vary by more than 20%.

In a second aspect, the present invention provides a method of diagnosing lung cancer or a predisposition therefor, said method comprising determining the methylation status of cytidine in
(1) at least one CpG dinucleotide in a nucleic acid comprising or consisting of the sequence from position 7 to position 101 of SEQ ID NO: 4;
(2) at least one CpG dinucleotide in a nucleic acid comprising or consisting of the sequence from position 11 to position 71 of SEQ ID NO: 2;
(3) at least one CpG dinucleotide in a nucleic acid comprising or consisting of the sequence from position 37 to position 81 of SEQ ID NO: 3;
(4) at least one CpG dinucleotide in a nucleic acid comprising or consisting of the sequence from position 4 to position 84 of SEQ ID NO: 1;
(5) at least one CpG dinucleotide in a nucleic acid comprising or consisting of the sequence from position 28 to position 69 of SEQ ID NO: 5;
(6) at least one CpG dinucleotide in a nucleic acid comprising or consisting of the sequence from position 39 to position 85 of SEQ ID NO: 6;
(7) at least one CpG dinucleotide in a nucleic acid comprising or consisting of the sequence from position 10 to position 53 of SEQ ID NO: 7;
(8) at least one CpG dinucleotide in a nucleic acid comprising or consisting of the sequence from position 13 to position 71 of SEQ ID NO: 8;
(9) at least one CpG dinucleotide in a nucleic acid comprising or consisting of the sequence from position 5 to position 71 of SEQ ID NO: 9;
(10) at least one CpG dinucleotide in a nucleic acid comprising or consisting of the sequence from position 2 to position 60 of SEQ ID NO: 10;
(11) at least one CpG dinucleotide in a nucleic acid comprising or consisting of the sequence from position 22 to position 63 of SEQ ID NO: 11;
(12) at least one CpG dinucleotide in a nucleic acid comprising or consisting of the sequence from position 45 to position 67 of SEQ ID NO: 12;
(13) at least one CpG dinucleotide in a nucleic acid comprising or consisting of the sequence from position 58 to position 78 of SEQ ID NO: 13;
(14) at least one CpG dinucleotide in a nucleic acid comprising or consisting of the sequence from position 31 to position 66 of SEQ ID NO: 14;
(15) at least one CpG dinucleotide in a nucleic acid comprising or consisting of the sequence from position 41 to position 72 of SEQ ID NO: 15;
(16) at least one CpG dinucleotide in a nucleic acid comprising or consisting of the sequence from position 53 to position 82 of SEQ ID NO: 16;
(17) at least one CpG dinucleotide in a nucleic acid comprising or consisting of the sequence from position 20 to position 74 of SEQ ID NO: 17;
(18) at least one CpG dinucleotide in a nucleic acid comprising or consisting of the sequence from position 9 to position 98 of SEQ ID NO: 18;
(19) at least one CpG dinucleotide in a nucleic acid comprising or consisting of the sequence from position 23 to position 85 of SEQ ID NO: 19;
(20) at least one CpG dinucleotide in a nucleic acid comprising or consisting of the sequence from position 20 to position 28 of SEQ ID NO: 20;
(21) at least one CpG dinucleotide in a nucleic acid comprising or consisting of the sequence from position 24 to position 61 of SEQ ID NO: 21;
(22) at least one CpG dinucleotide in a nucleic acid comprising or consisting of the sequence from position 11 to position 84 of SEQ ID NO: 22;
(23) at least one CpG dinucleotide in a nucleic acid comprising or consisting of the sequence from position 2 to position 86 of SEQ ID NO: 23;
(24) at least one CpG dinucleotide in a nucleic acid comprising or consisting of the sequence from position 43 to position 80 of SEQ ID NO: 24;
(25) at least one CpG dinucleotide in a nucleic acid comprising or consisting of the sequence from position 24 to position 70 of SEQ ID NO: 25;
(26) at least one CpG dinucleotide in a nucleic acid comprising or consisting of the sequence from position 44 to position 94 of SEQ ID NO: 26;
(27) at least one CpG dinucleotide in a nucleic acid comprising or consisting of the sequence from position 44 to position 67 of SEQ ID NO: 27;
(28) at least one CpG dinucleotide in a nucleic acid comprising or consisting of the sequence from position 20 to position 43 of SEQ ID NO: 28;
(29) at least one CpG dinucleotide in a nucleic acid comprising or consisting of the sequence from position 3 to position 71 of SEQ ID NO: 29;
(30) at least one CpG dinucleotide in a nucleic acid comprising or consisting of the sequence from position 11 to position 64 of SEQ ID NO: 30;
(31) at least one CpG dinucleotide in a nucleic acid comprising or consisting of the sequence from position 7 to position 118 of SEQ ID NO: 31;
(32) at least one CpG dinucleotide in a nucleic acid comprising or consisting of the sequence from position 25 to position 95 of SEQ ID NO: 32;
(33) at least one CpG dinucleotide in a nucleic acid comprising or consisting of the sequence from position 29 to position 46 of SEQ ID NO: 33;
(34) at least one CpG dinucleotide in a nucleic acid comprising or consisting of the sequence from position 8 to position 72 of SEQ ID NO: 34;
(35) at least one CpG dinucleotide in a nucleic acid comprising or consisting of the sequence from position 5 to position 57 of SEQ ID NO: 35;
(36) at least one CpG dinucleotide in a nucleic acid comprising or consisting of the sequence from position 43 to position 67 of SEQ ID NO: 36;
(37) at least one CpG dinucleotide in a nucleic acid comprising or consisting of the sequence from position 21 to position 63 of SEQ ID NO: 37;
(38) at least one CpG dinucleotide in a nucleic acid comprising or consisting of the sequence from position 27 to position 86 of SEQ ID NO: 38;
(39) at least one CpG dinucleotide in a nucleic acid comprising or consisting of the sequence from position 2 to position 110 of SEQ ID NO: 39;
(40) at least one CpG dinucleotide in a nucleic acid comprising or consisting of the sequence from position 13 to position 59 of SEQ ID NO: 40;
(41) at least one CpG dinucleotide in a nucleic acid comprising or consisting of the sequence from position 32 to position 107 of SEQ ID NO: 41;
(42) at least one CpG dinucleotide in a nucleic acid comprising or consisting of the sequence from position 3 to position 48 of SEQ ID NO: 42;
(43) at least one CpG dinucleotide in a nucleic acid comprising or consisting of the sequence from position 5 to position 57 of SEQ ID NO: 43;
(44) at least one CpG dinucleotide in a nucleic acid comprising or consisting of the sequence from position 40 to position 106 of SEQ ID NO: 44;
(45) at least one CpG dinucleotide in a nucleic acid comprising or consisting of the sequence from position 26 to position 90 of SEQ ID NO: 45; and/or
(46) at least one CpG dinucleotide in a nucleic acid comprising or consisting of the sequence from position 68 to position 74 of SEQ ID NO: 46;
   wherein methylation of the cytidine(s) or increased methylation of the cytidine(s) as compared to a non-cancer subject is indicative of lung cancer or a predisposition therefor, and wherein any diagnostic method practiced on the human or animal body is excluded.

In a related aspect (third aspect), the present invention provides a method of monitoring treatment of lung cancer or of a predisposition therefor, said method comprising determining the methylation status of cytidine, cytidine being defined as in accordance with the second aspect, wherein no methylation of cytidine(s) or decreased methylation of the cytidine(s) as compared to a patient suffering from lung cancer or an individual having a predisposition therefor, respectively, is indicative of an amelioration of lung cancer or a decreased predisposition therefor, respectively, and wherein any diagnostic method practiced on the human or animal body is excluded.

Accordingly, while hypermethylation is indicative of the diseased state, a decrease of methylation or absence of methylation can be used as indicators of successful therapeutic treatment.

Determining a state as being methylated will in generally be sufficient for diagnostic purposes. In other words, a reference state for comparison is generally not compulsory. Yet, the second aspect of the present invention also presents the alternative of increased methylation as compared to a non-cancer subject. The non-cancer subject may be a single individual. Alternatively, the term “non-cancer subject” is a population average. The non-cancer state is characterized by low, preferably less than 40%, less than 30%, less than 20%, less than 10%, less than 5%, less than 2%, or less than 1% methylation. This applies to all genes or CpG clusters of the present invention, respectively. The term “non-cancer subject” embraces, but is not confined to healthy individuals. We note, though, that healthy individuals are preferred. Generally speaking, non-cancer subjects may also include diseased persons, provided that they do not suffer from lung cancer.

Since, as noted above, diagnosis may be performed with a sample drawn from a subject or patient, it is understood that diagnostic methods practiced on the human or animal body are excluded. Furthermore, we note that the actual process of drawing the sample does not fall under the terms of the present claims. As such, the methods of the present invention also do not extend to invasive or surgical methods.

The present disclosure also provides a method of diagnosing lung cancer or a predisposition therefor, said method comprising (a) taking a sample from an individual, and (b) determining the methylation status of cytidine as defined in accordance with the second aspect.

The individual or subject may be a human or an animal, preferably a mammal. The term “individual” includes patients such as patients suffering from lung diseases, preferably lung cancer, as well as healthy or apparently healthy individuals. The terms “individual” and “subject” are used equivalently herein.

The term “lung cancer” has its art-established meaning and relates to any malignant disease affecting a tissue of the lung. Further information in this respect may be taken from the background section herein above and references cited therein. It is understood that in patients suffering from lung cancer, diseased cells include diseased cells in the lung, but are not confined thereto. Rather, diseased cells may also embrace cells from metastasis as well as circulating tumor cells. Any of these cells may be comprised in the sample to be analyzed using the methods of the present invention.

As detailed further below, the term “lung cancer” includes inter alia early stages. Not only early stages can be diagnosed, but furthermore a predisposition for developing lung cancer. In other words, also in the absence of clinical symptoms or where clinical symptoms are difficult to detect, the method of the present invention permits diagnosis.

The method is preferably effected with cell-free DNA. Cell-free DNA may be obtained from plasma. Cell-free DNA, as a matter of fact, is a constituent of plasma. Typical amounts as found in healthy and diseased persons are reported in the examples enclosed herewith.

At least in this preferred embodiment relating to a plasma sample, more invasive methods such as taking a biopsy are actually dispensable. Yet, the present invention may be worked using biopsies such as lung biopsies as well. Also, the method of the invention may be used for cell-free DNA testing, followed by confirmatory biopsy testing, wherein in at least one out of cell-free DNA testing and biopsy testing or in either case the respective samples (cell-free DNA; cells comprised in the biopsy) are analyzed with the methods in accordance with the present invention. Alternatively, the biopsy may be analyzed according to histological parameters.

To the extent the method of the second aspect provides for determining the methylation status of more than one CpG dinucleotide in a given CpG cluster, and/or a methylation status of more than one CpG cluster, it is understood that a diagnosis of lung cancer or a predisposition therefor is provided in those instances, where at least 70%, at least 80%, or at least 90%, preferably all CpG dinucleotides the methylation status of which is to be determined are in fact methylated. Determining the methylation status of more than one CpG dinucleotides is a means of increasing accuracy of the method of diagnosing in accordance with the second aspect. The above defined fold change is a preferred measure of the methylation status.

In a preferred embodiment of the second and third aspect, the cytidines the methylation status of which is to be determined, are those defined in accordance with the preferred embodiments of the first aspect herein above.

In a preferred embodiment of both first and second aspect, said nucleic acid comprises or consists of any one of SEQ ID NOs: 1 to 46, 47 to 92 or 93 to 136.

This preferred embodiment provides sequences of increasing length and comprising the respective CpG cluster located in the subsequences of SEQ ID NOs: 1 to 46 as defined in accordance with the first aspect.

In a related aspect (fourth aspect), the present invention provides a nucleic acid comprising or consisting of (a) the sequence of any one of SEQ ID NOs: 137 to 182 or 183 to 228; or (b) a sequence heaving at least 80% sequence identity to a sequence of (a).

With regard to this embodiment, the following applies. As noted above, Cs are amenable to conversion to T, to the extent they are not methylated. The sequences of SEQ ID NOs. 137 to 182 are derivatives of sequences 1 to 46 wherein all Cs are converted into Ts with the exception of those Cs which occur within CpG dinucleotide motifs specified in accordance with the present invention; see column “C/T converted (1)” in Table 3. In the sequences of SEQ ID NOs: 183 to 228, all Cs have been converted into Ts in their entirety; see column “C/T converted (2)” in Table 3. The latter correspond to the complete absence of methylation in which case all Cs are amenable to conversion, for example by means of bisulfite. In terms of length, the sequences of SEQ ID NOs: 137 to 182 as well as 183 to 228 correspond to SEQ ID NOs: 47 to 92.

As noted above, also sequences where the results of conversion of a C is not a T, but instead a U are subject of the present invention.

We furthermore note that SEQ ID NOs: 1 to 46 define shorter subsequences within SEQ ID NOs: 47 to 92. While C/T as well as C/U converted sequences as disclosed above correspond in length to SEQ ID NOs: 47 to 92, it is understood that also subsequences of the C/T and C/U converted sequences which subsequences correspond to SEQ ID NOs: 1 to 46 are subject of the present invention. “Correspondence” in this case means identical length and sequence identity with exception of the converted positions. Furthermore, the first aspect of the present invention defines specific subsequences within SEQ ID NOs: 1 to 46. C/T and

C/U converted sequences corresponding to these specific subsequences of SEQ ID NOs: 1 to 46 are also embraced by the present invention.

Table 3 below explains the correspondence between these sequences.

TABLE 3

| | | Medium | | C/T | C/T |
|---|---|---|---|---|---|
| Gene | PCR | length | Long | converted | converted |
| symbol | product | construct | construct | (1) | (2) |
| PAX9 | 4 | 47 | 93 | 137 | 183 |
| HOXD10 | 2 | 48 | 94 | 138 | 184 |
| TBX20 | 3 | 49 | 95 | 139 | 185 |
| CACNA1B | 1 | 50 | 96 | 140 | 186 |
| ZIC1 | 5 | 51 | 97 | 141 | 187 |
| SIM1 | 6 | 52 | — | 142 | 188 |
| DLX1 | 7 | 53 | 98 | 143 | 189 |
| LMX1A | 8 | 54 | — | 144 | 190 |
| DLX6AS1 | 9 | 55 | 99 | 145 | 191 |
| MIR196A1 | 10 | 56 | 100 | 146 | 192 |
| FEZF2 | 11 | 57 | 101 | 147 | 193 |
| STAG3 | 12 | 58 | 102 | 148 | 194 |
| PTPRN2 | 13 | 59 | 103 | 149 | 195 |
| BARHL1 | 14 | 60 | 104 | 150 | 196 |
| CCDC140 | 15 | 61 | 105 | 151 | 197 |
| N/A | 16 | 62 | 106 | 152 | 198 |
| GATA2 | 17 | 63 | 107 | 153 | 199 |
| ADCYAP1 | 18 | 64 | 108 | 154 | 200 |
| TBX5 | 19 | 65 | 109 | 155 | 201 |
| N/A | 20 | 66 | 110 | 156 | 202 |
| NECAB1 | 21 | 67 | 111 | 157 | 203 |
| ADRB1 | 22 | 68 | 112 | 158 | 204 |
| OTX2 | 23 | 69 | 113 | 159 | 205 |
| NKX2-6 | 24 | 70 | 114 | 160 | 206 |
| SIM1 | 25 | 71 | 115 | 161 | 207 |
| DRD4 | 26 | 72 | 116 | 162 | 208 |
| TBX15 | 27 | 73 | 117 | 163 | 209 |
| TRH | 28 | 74 | 118 | 164 | 210 |
| FBP1 | 29 | 75 | 119 | 165 | 211 |
| N/A | 30 | 76 | 120 | 166 | 212 |
| BCAN | 31 | 77 | 121 | 167 | 213 |
| TP73 | 32 | 78 | 122 | 168 | 214 |
| MIIP | 33 | 79 | 123 | 169 | 215 |
| HOXC4 | 34 | 80 | 124 | 170 | 216 |
| HOXA7 | 35 | 81 | 125 | 171 | 217 |
| BST1 | 36 | 82 | 126 | 172 | 218 |
| C1orf114 | 37 | 83 | 127 | 173 | 219 |
| FOXG1 | 38 | 84 | 128 | 174 | 220 |
| ATP4A | 39 | 85 | 129 | 175 | 221 |
| NOTCH1 | 40 | 86 | 130 | 176 | 222 |
| N/A | 41 | 87 | 131 | 177 | 223 |
| N/A | 42 | 88 | 132 | 178 | 224 |
| HOXA7 | 43 | 89 | 133 | 179 | 225 |
| ZNF529 | 44 | 90 | 134 | 180 | 226 |
| PRLHR | 45 | 91 | 135 | 181 | 227 |
| ARL5C | 46 | 92 | 136 | 182 | 228 |

Each of the nucleic acid sequences in accordance with the present invention may be fused to a heterologous sequence.

In another preferred embodiment of the method in accordance with the second aspect, the methylation status of cytidines according to

(1) items (4) and (9);
(2) items (4) and (2);
(3) items (4) and (6);
(4) items (2) and (9);
(5) items (1) and (4);
(6) items (2) and (5);
(7) items (4) and (13);
(8) items (2) and (10);
(9) items (2) and (6);
(10) items (10) and (11);
(11) items (2) and (1);
(12) items (9) and (11);
(13) items (9) and (10);
(14) items (4) and (5);
(15) items (5) and (6);
(16) items (10) and (14);
(17) items (1) and (11);
(18) items (9) and (13);
(19) items (7) and (11);
(20) items (4), (2) and (9);
(21) items (4), (2) and (6);
(22) items (4), (9) and (10);
(23) items (9), (10) and (11);
(24) items (4), (9) and (11);
(25) items (2), (6) and (10);
(26) items (4), (5) and (9);
(27) items (2), (9) and (10);
(28) items (4), (2) and (10);
(29) items (1), (4) and (9);
(30) items (4), (2) and (12);
(31) items (1), (2) and (4);
(32) items (2), (10) and (14);
(33) items (2), (10) and (11);
(34) items (2), (5) and (10);
(35) items (4), (2) and (5);
(36) items (2), (5) and (9);
(37) items (2), (5) and (6);
(38) items (2), (6) and (11);
(39) items (4), (9) and (12); or
(40) items (4), (2) and (7)
as defined in accordance with the second aspect is to be determined. These preferred combinations of markers are also given in the table below together with their prediction performance. The performance measure in this case is the difference of AUC values: AUC (combination)−AUC (random). The term “random” designates a combination of two or three markers, respectively, which has been chosen at random from the markers of the invention and tested on the same serum data set.

TABLE 4

| MARKER 1 | MARKER 2 | MARKER 3 | AUC_diff |
|---|---|---|---|
| PAX9 | DLX6AS1 | | 0.1446281 |
| PAX9 | HOXD10 | | 0.13553719 |
| PAX9 | SIM1 | | 0.12093664 |
| HOXD10 | DLX6AS1 | | 0.11818182 |
| PAX9 | CACNA1B | | 0.1261708 |
| HOXD10 | ZIC1 | | 0.11763085 |
| PAX9 | PTPRN2 | | 0.10220386 |
| HOXD10 | MIR196A1 | | 0.11184573 |
| HOXD10 | SIM1 | | 0.11404959 |
| MIR196A1 | FEZF2 | | 0.11652893 |
| HOXD10 | CACNA1B | | 0.1077135 |
| DLX6AS1 | FEZF2 | | 0.10137741 |
| DLX6AS1 | MIR196A1 | | 0.10082645 |
| PAX9 | ZIC1 | | 0.09614325 |
| ZIC1 | SIM1 | | 0.07217631 |
| MIR196A1 | BARHL1 | | 0.07713499 |
| CACNA1B. | FEZF2 | | 0.07988981 |
| DLX6AS1 | PTPRN2 | | 0.08126722 |
| DLX1 | FEZF2 | | 0.07272727 |
| PAX9 | HOXD10 | DLX6AS1 | 0.1677686 |
| PAX9 | HOXD10 | SIM1 | 0.15619835 |
| PAX9 | DLX6AS1 | MIR196A1 | 0.15812672 |
| DLX6AS1 | MIR196A1 | FEZF2 | 0.16804408 |
| PAX9 | DLX6AS1 | FEZF2 | 0.16143251 |
| HOXD10 | SIM1. | MIR196A1 | 0.15922865 |
| PAX9 | ZIC1 | DLX6AS1 | 0.15179063 |
| HOXD10 | DLX6AS1 | MIR196A1 | 0.16101928 |
| PAX9 | HOXD10 | MIR196A1 | 0.14517906 |
| PAX9 | CACNA1B. | DLX6AS1 | 0.15881543 |
| PAX9 | HOXD10 | STAG3 | 0.15757576 |
| PAX9 | HOXD10 | CACNA1B | 0.14545455 |
| HOXD10 | MIR196A1 | BARHL1 | 0.15495868 |
| HOXD10 | MIR196A1 | FEZF2 | 0.14958678 |
| HOXD10 | ZIC1 | MIR196A1 | 0.1476584 |
| PAX9 | HOXD10 | ZIC1 | 0.1399449 |
| HOXD10 | ZIC1 | DLX6AS1 | 0.13663912 |
| HOXD10 | ZIC1 | SIM1 | 0.13719008 |
| HOXD10 | SIM1. | FEZF2 | 0.13471074 |
| PAX9 | DLX6AS1 | STAG3 | 0.13829201 |
| PAX9 | HOXD10 | DLX1 | 0.13939394 |

As noted above, the determination of the methylation status of more than one cytidine, is in general a means of increasing accuracy of diagnosis. The subject-matter of the above preferred embodiments are specific combinations of CpG clusters which were found to provide particularly accurate diagnosis.

In a further preferred embodiment of the method of the invention (a) the methylation status of no further nucleotides is to be determined; or (b) said method furthermore comprises determining the methylation status of cytidine in at least on CpG dinucleotide in a nucleic acid comprising or consisting of the sequence of SEQ ID NO: 321, wherein methylation of cytidines or increased methylation of cytidines as compared to a non-cancer subject is indicative of lung cancer, cytidines being those defined herein above and cytidine(s) in the sequence of SEQ ID NO: 321.

More generally speaking, while the use of the sequence of SEQ ID NO: 321 is preferred, it is understood that not only nucleic acids comprising or consisting of the sequence of SEQ ID NO: 321 may be used, but instead any nucleic acid comprising or consisting of the sequence of Shox2 or a subsequence thereof, said subsequence having a minimal length of 50 bases.

This embodiment provides two alternatives. In the first alternative (a) it is established that the cytidines identified in the preceding embodiments constitute a closed list. In the alternative (b) the determination of cytidine methylation status may be extended to one or more further genes, a particularly preferred further gene being Shox2. To the extent the methylation status of one or more cytidines in the Shox2 sequence is to be determined, it is understood that diagnosis is based on the methylation status of all cytidines analyzed, namely those in accordance with the preceding embodiments together with those in the Shox2 sequence.

In a further preferred embodiment, said determining is effected in a sample taken from a subject, said sample preferably being a bodily fluid, more preferably plasma, serum or sputum; a biopsy taken from said cancer; a biopsy taken from a metastasis; or a circulating tumor cell.

In a particularly preferred embodiment, prior to said determining, (a) cell-free DNA comprised in said sample is enriched or isolated; (b) cell-free DNA is subjected to digestion with one or more methylation-sensitive restriction enzymes; and/or (c) cell-free DNA is pre-amplified.

Methods for enriching or isolating DNA from a sample are well-known in the art. A preferred method is described in the examples enclosed herewith.

Digestion in accordance with item (b) is preferably effected by bringing enriched or isolated cell-free DNA into contact with one or more of the mentioned enzymes. Alternatively, a sample, as it is obtained from a subject, may be brought into contact with said enzymes. Preferred enzymes are disclosed further below. Digestion with methylation-sensitive restriction enzymes is a particularly advantageous approach for analyzing the methylation status of DNA. In a specific example, the high multiplexing properties of MSRE digested DNA allow to perform 96 qPCR assays in parallel on cfDNA derived from only 400 μL patient serum. Methylation sensitive restriction enzymes are characterized in that they are not able to cleave at the normal cleavage site in those cases where the cytidines comprised in the cleavage site are methylated.

Item (c) provides for a pre-amplification step. Pre-amplification is typically applied after enzymatic digestion. It is a means of enriching and/or increasing the amount of methylated DNA, in particular prior to the subsequent actual step of determining, preferably by amplification, which yields the desired readout.

In a further preferred embodiment, said determining is performed by one or more of the following procedures: (a) a support-based methylation assay; (b) amplification, preferably PCR, more preferably conventional or real-time PCR, of DNA digested in accordance with the invention and optionally pre-amplified in accordance with the invention; (c) a methylated CpG island recovery assay; (d) a bisulfite assay or bisulfite sequencing; (e) a proximity hybridization assay; and (f) nanopore sequencing. Bisulfite-based methods are reviewed in Egger et al., Expert Rev. Mol. Diagn. 12, 75-92 (2012).

This preferred embodiment includes the above-mentioned option of amplification for determining the methylation status, together with other alternatives. The other alternatives are known in the art. As an example, we refer to Rauch et al. (loc. cit.), where a methylated CpG island recovery assay is described. Further art-established methods for determining the methylation status include MSRE (or HELP), MSP, qMSP, MethyLight, HeavyMethyl, methyl-BEAMing, COBRA, MBD, MEDIP, MLPA, Maldi TOF and McBC (i.e. CHARM).

In a particularly preferred embodiment, (a) cell-free DNA comprised in said sample is isolated; (b) the isolated DNA obtained in (a) is subjected to digestion with one or more methylation-sensitive restriction enzymes; (c) the digested DNA obtained in (b) is pre-amplified; (d) the pre-amplified DNA obtained in (c) is amplified.

In an alternative preferred embodiment, said methylation status is determined without any amplification. This can be done, for example, by nanopore sequencing. This is a means of directly detecting methylated as well as hydroxymethylated cytidine which does not require a hybridization assay either. Other amplification independent methods of determining the methylation status rely on detecting methylated and/or hydroxymethylated cytidines with antibodies or aptamers specific for methylated and hydroxymethylated cytidines, respectively. Other proteins specifically binding such modified cytidines are methyl-binding domain proteins. Upon binding of any of said binders (antibodies, aptamers, methyl-binding domain proteins), proximity hybridization assays may be used in order to determine which positions are methylated.

Enriching or isolating of DNA prior to any of the above amplification independent methods is preferred, but not required. Similarly, the above disclosed amplification independent methods may be used in combination with digestion by methylation-sensitive restriction enzymes, but may also be used without prior digestion.

This embodiment defines a particularly preferred sequence of steps leading from a sample, preferably a plasma sample, to amplified DNA, wherein the amplified DNA, owing to the use of restriction enzymes in accordance with step (b) of this particularly preferred embodiment, consists of or is enriched with regard to methylated DNA.

In principle, any methylation sensitive restriction enzyme may be used. Preferably, said methylation-sensitive restriction enzymes are selected from HpaII, HinP1I, AciI and HpyCH4IV.

So far, the methylation status of cytidines has been described herein as a disease-relevant parameter useful for diagnosis of lung cancer. In addition thereto, the amount of cell-free DNA comprised in the sample, preferably a serum sample, may be used as a further predictive parameter.

Accordingly, in a preferred embodiment, furthermore the amount of cell-free DNA in said sample is determined.

In a particularly preferred embodiment, an elevated amount of cell-free DNA as compared to a healthy subject is indicative of disease.

To the extent both methylation status of cytidines and the amount of cell-free DNA are used in the method of diagnosis of the second aspect of the present invention, provided below are specific combinations of parameters. In a preferred embodiment (a) the methylation status of cytidine(s) according to (1) item (1) as defined in the second aspect of the invention; (2) item (2) as defined in the second aspect of the invention; (3) items (1) and (2) as defined in the second aspect of the invention; (4) items (1) and (9) as defined in the second aspect of the invention; (5) items (2) and (5) as defined in the second aspect of the invention; or (6) items (1) and (4) as defined in the second aspect of the invention; and (b) the amount of cell-free DNA in said sample are determined.

In a preferred embodiment of the method of the second aspect, said lung cancer is selected from adenocarcinoma, squamous cell carcinoma, small cell lung carcinoma, large cell lung carcinoma, early stage lung cancer (stage I or II) and late stage lung cancer (stage III or IV).

In a fifth aspect, the present invention provides a kit comprising or consisting of one or more of the following sets of primers:
(1) nucleic acids comprising or consisting of the sequences of SEQ ID NO: 229 and 230 or sequences having at least 80% sequence identity thereto;
(2) nucleic acids comprising or consisting of the sequences of SEQ ID NO: 231 and 232 or sequences having at least 80% sequence identity thereto;
(3) nucleic acids comprising or consisting of the sequences of SEQ ID NO: 233 and 234 or sequences having at least 80% sequence identity thereto;
(4) nucleic acids comprising or consisting of the sequences of SEQ ID NO: 235 and 236 or sequences having at least 80% sequence identity thereto;
(5) nucleic acids comprising or consisting of the sequences of SEQ ID NO: 237 and 238 or sequences having at least 80% sequence identity thereto;
(6) nucleic acids comprising or consisting of the sequences of SEQ ID NO: 239 and 240 or sequences having at least 80% sequence identity thereto;
(7) nucleic acids comprising or consisting of the sequences of SEQ ID NO: 241 and 242 or sequences having at least 80% sequence identity thereto;
(8) nucleic acids comprising or consisting of the sequences of SEQ ID NO: 243 and 244 or sequences having at least 80% sequence identity thereto;
(9) nucleic acids comprising or consisting of the sequences of SEQ ID NO: 245 and 246 or sequences having at least 80% sequence identity thereto;
(10) nucleic acids comprising or consisting of the sequences of SEQ ID NO: 247 and 248 or sequences having at least 80% sequence identity thereto;
(11) nucleic acids comprising or consisting of the sequences of SEQ ID NO: 249 and 250 or sequences having at least 80% sequence identity thereto;
(12) nucleic acids comprising or consisting of the sequences of SEQ ID NO: 251 and 252 or sequences having at least 80% sequence identity thereto;
(13) nucleic acids comprising or consisting of the sequences of SEQ ID NO: 253 and 254 or sequences having at least 80% sequence identity thereto;
(14) nucleic acids comprising or consisting of the sequences of SEQ ID NO: 255 and 256 or sequences having at least 80% sequence identity thereto;
(15) nucleic acids comprising or consisting of the sequences of SEQ ID NO: 257 and 258 or sequences having at least 80% sequence identity thereto;
(16) nucleic acids comprising or consisting of the sequences of SEQ ID NO: 259 and 260 or sequences having at least 80% sequence identity thereto;
(17) nucleic acids comprising or consisting of the sequences of SEQ ID NO: 261 and 262 or sequences having at least 80% sequence identity thereto;
(18) nucleic acids comprising or consisting of the sequences of SEQ ID NO: 263 and 264 or sequences having at least 80% sequence identity thereto;
(19) nucleic acids comprising or consisting of the sequences of SEQ ID NO: 265 and 266 or sequences having at least 80% sequence identity thereto;
(20) nucleic acids comprising or consisting of the sequences of SEQ ID NO: 267 and 268 or sequences having at least 80% sequence identity thereto;
(21) nucleic acids comprising or consisting of the sequences of SEQ ID NO: 269 and 270 or sequences having at least 80% sequence identity thereto;
(22) nucleic acids comprising or consisting of the sequences of SEQ ID NO: 271 and 272 or sequences having at least 80% sequence identity thereto;
(23) nucleic acids comprising or consisting of the sequences of SEQ ID NO: 273 and 274 or sequences having at least 80% sequence identity thereto;
(24) nucleic acids comprising or consisting of the sequences of SEQ ID NO: 275 and 276 or sequences having at least 80% sequence identity thereto;
(25) nucleic acids comprising or consisting of the sequences of SEQ ID NO: 277 and 278 or sequences having at least 80% sequence identity thereto;
(26) nucleic acids comprising or consisting of the sequences of SEQ ID NO: 279 and 280 or sequences having at least 80% sequence identity thereto;
(27) nucleic acids comprising or consisting of the sequences of SEQ ID NO: 281 and 282 or sequences having at least 80% sequence identity thereto;
(28) nucleic acids comprising or consisting of the sequences of SEQ ID NO: 283 and 284 or sequences having at least 80% sequence identity thereto;
(29) nucleic acids comprising or consisting of the sequences of SEQ ID NO: 285 and 286 or sequences having at least 80% sequence identity thereto;
(30) nucleic acids comprising or consisting of the sequences of SEQ ID NO: 287 and 288 or sequences having at least 80% sequence identity thereto;
(31) nucleic acids comprising or consisting of the sequences of SEQ ID NO: 289 and 290 or sequences having at least 80% sequence identity thereto;
(32) nucleic acids comprising or consisting of the sequences of SEQ ID NO: 291 and 292 or sequences having at least 80% sequence identity thereto;
(33) nucleic acids comprising or consisting of the sequences of SEQ ID NO: 293 and 294 or sequences having at least 80% sequence identity thereto;
(34) nucleic acids comprising or consisting of the sequences of SEQ ID NO: 295 and 296 or sequences having at least 80% sequence identity thereto;
(35) nucleic acids comprising or consisting of the sequences of SEQ ID NO: 297 and 298 or sequences having at least 80% sequence identity thereto;
(36) nucleic acids comprising or consisting of the sequences of SEQ ID NO: 299 and 300 or sequences having at least 80% sequence identity thereto;
(37) nucleic acids comprising or consisting of the sequences of SEQ ID NO: 301 and 302 or sequences having at least 80% sequence identity thereto;
(38) nucleic acids comprising or consisting of the sequences of SEQ ID NO: 303 and 304 or sequences having at least 80% sequence identity thereto;
(39) nucleic acids comprising or consisting of the sequences of SEQ ID NO: 305 and 306 or sequences having at least 80% sequence identity thereto;
(40) nucleic acids comprising or consisting of the sequences of SEQ ID NO: 307 and 308 or sequences having at least 80% sequence identity thereto;
(41) nucleic acids comprising or consisting of the sequences of SEQ ID NO: 309 and 310 or sequences having at least 80% sequence identity thereto;
(42) nucleic acids comprising or consisting of the sequences of SEQ ID NO: 311 and 312 or sequences having at least 80% sequence identity thereto;
(43) nucleic acids comprising or consisting of the sequences of SEQ ID NO: 313 and 314 or sequences having at least 80% sequence identity thereto;
(44) nucleic acids comprising or consisting of the sequences of SEQ ID NO: 315 and 316 or sequences having at least 80% sequence identity thereto;
(45) nucleic acids comprising or consisting of the sequences of SEQ ID NO: 317 and 318 or sequences having at least 80% sequence identity thereto;
(46) nucleic acids comprising or consisting of the sequences of SEQ ID NO: 319 and 320 or sequences having at least 80% sequence identity thereto.

The two sequences specified in each item of the third aspect are forward and reverse primer useful for amplifying the sequence of a given CpG cluster. The order of items corresponds to the order of items in the first aspect.

Preferred levels of sequence identity are given herein above as well as means and methods for determining sequence identity.

In a preferred embodiment of the kit of the invention, the kit furthermore comprises one or more of the following: (a) a manual with instructions for performing the method of the second aspect; (b) one or more buffered solutions or reagents for the preparation thereof; (c) one or more reaction vessels.

Furthermore comprised within the kit of the present invention, for example as part of the manual, may be information about threshold values as defined further above. Such threshold values are useful to distinguish between diseased and non-diseased states and render the use of control samples dispensable.

If not expressly indicated otherwise, preferred embodiments of one particular aspect of the present invention give rise to corresponding preferred embodiments of all other aspects.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all attached claims.

The figures show:

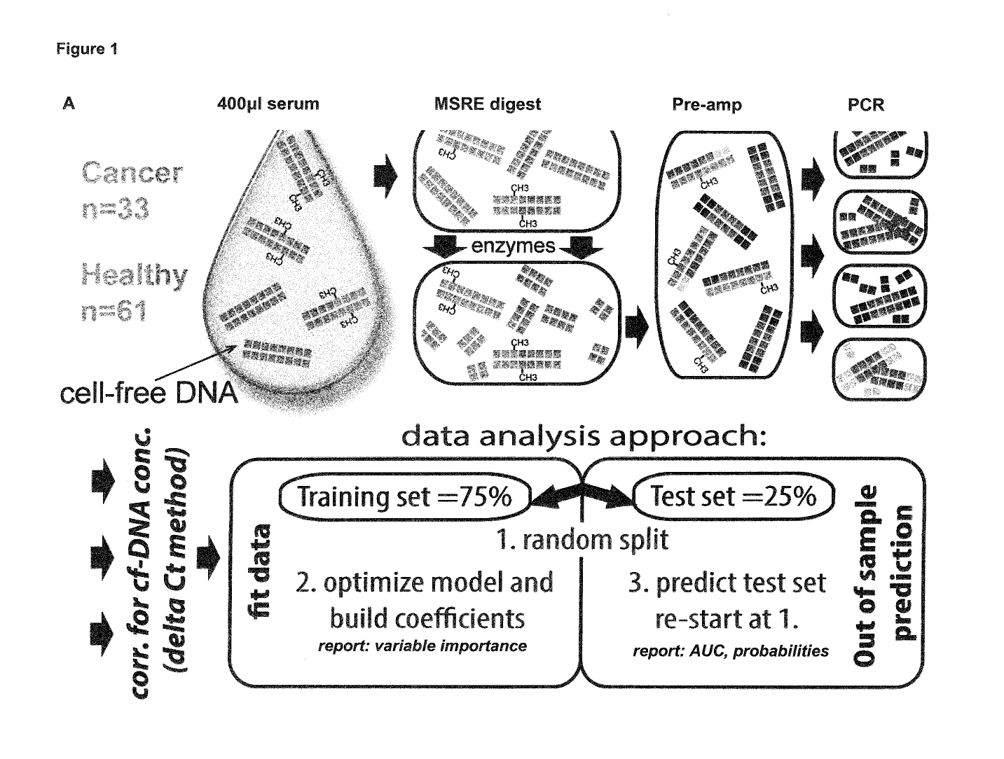

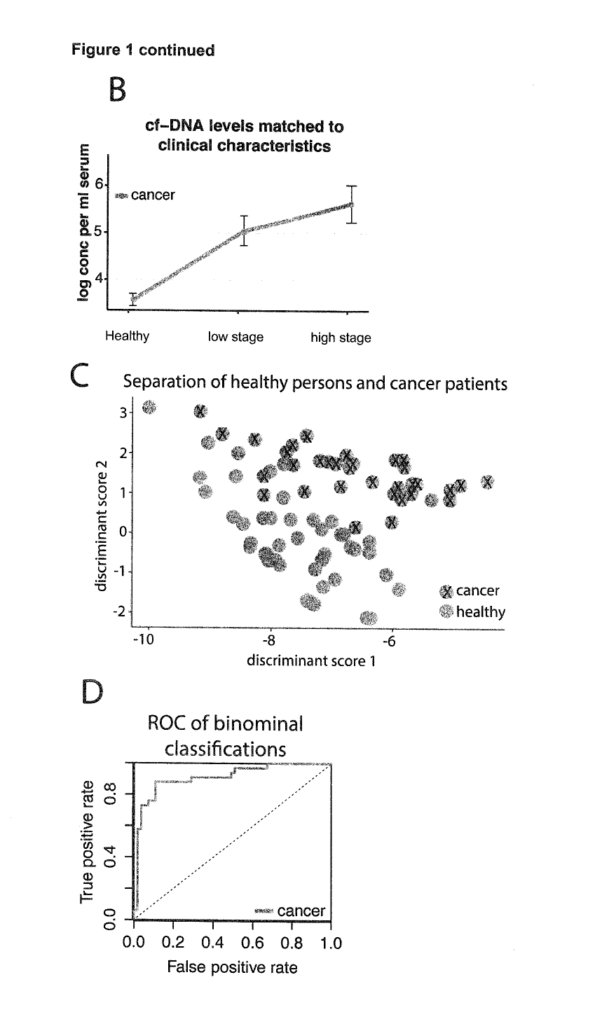

FIG. 1: Overview of cfDNA analysis.

(A) cfDNA processing workflow. Colored strings represent cfDNA with attached CH₃ indicating methylated DNA. Color represents DNA origin: purple from healthy tissue red from neoplastic tissue. Methyl-sensitive restriction enzyme (MSRE) digest is the key step of the workflow: the unmethylated DNA gets digested and only methylated DNA serves as a target in the preamplification step (Pre-amp) of the PCR. Additional to already existing color-code newly introduced colors indicate PCR-primers for methylation target loci and grey color indicates amplified DNA. One multiplexed preamplification is performed per sample, which subsequently is split into single reactions for qPCR detection (PCR). The lower panel shows our prediction and resampling approach, which is described in more detail in the supplemental material. (B) Isolated cfDNA per ml serum/plasma. Values are log 2 transformed. Error bars indicate the standard deviation of the mean. Low stage cancer represents patients diagnosed with cancer of stage I or II, high stage indicates stage III or IV. (C) Fisher discriminant analysis was performed using the top 30 markers. Subsequently the data were projected to the 2 most informative projection directions (discriminant scores). The plot shows the separation of patient samples based on the transformed data, which may be interpreted similarly to a Principal Component Analysis. (D) ROC-curve analysis shows the quality of separation of each analyzed disease versus healthy controls.

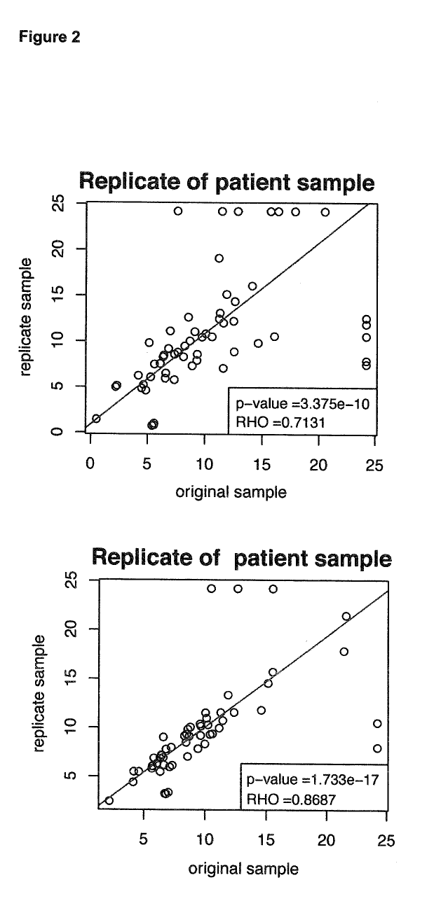

FIG. 2: Delta Ct normalized values of two representative samples (out of 16). Replicates were introduced at the level of cf-DNA extraction from serum; subsequently samples were manipulated in two independent experimental fractions. P-value reflects Spearman P-value of correlation. Red line indicates linear regression line of data points. Double negative values (i.e. no PCR signal in both samples) were removed before analysis, to leave spearman's rho and P value unbiased.

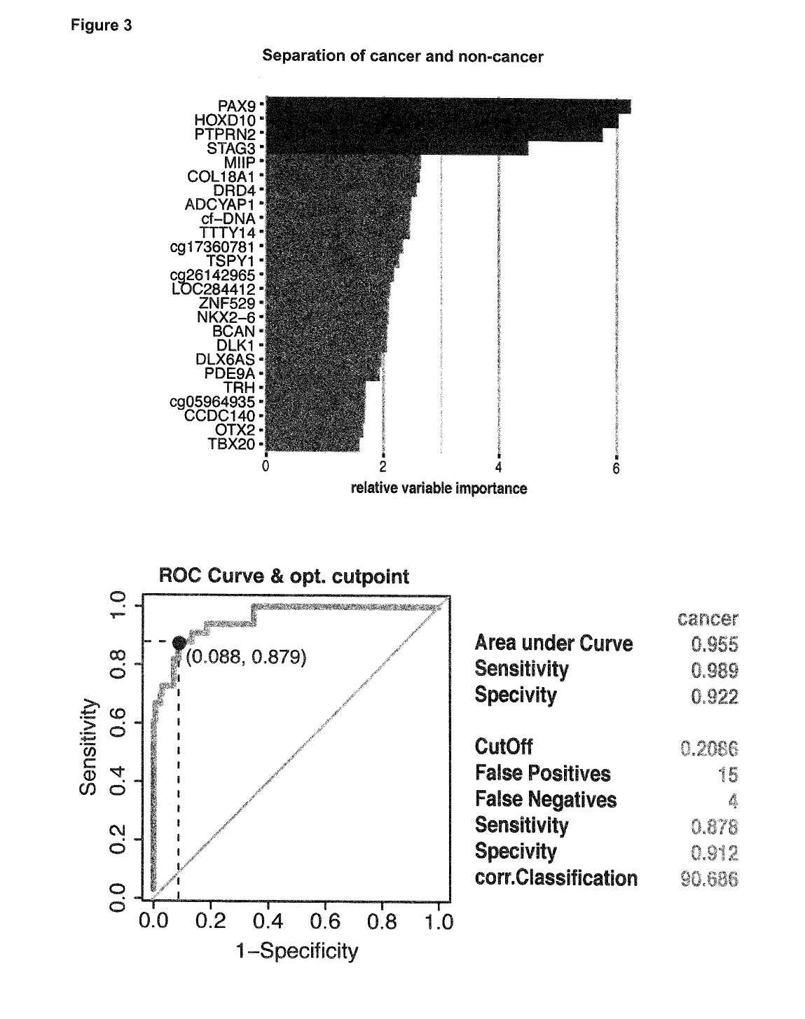

FIG. 3: Differential diagnosis approach, where each disease is separated from all patient samples. Bar plots indicate the relative variable importance for each model. Model distinguishes between cancer and residual samples. The relative variable importance reflects the contribution of each variable to the prediction of the sample membership. ROC curve indicates the quality of separation including the chosen cut off value, which would be used for the diagnosis of future patient samples. Values at cut points reflect achieved specificity and sensitivity for the chosen cut off value. The rightmost panels summarizes the ROC curve analysis starting with the values derived from conventional ROC curve analysis. The section below, starting with “CutOff”, lists the values gained by the application of the specific cut off values.

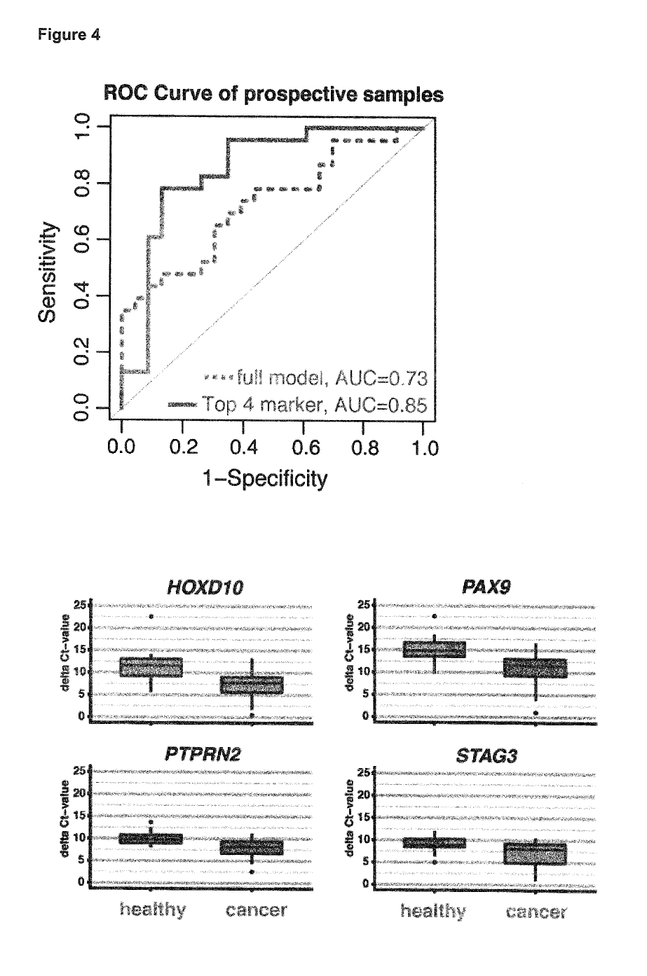

FIG. 4: Proof of Principle: Prospective sample prediction. ROC curve analysis of 46 serum samples, which were classified in a prospective analysis setting. Prediction is based on coefficients derived from the model presented FIG. 2. The dashed line represents the separation of cancer and non-cancer patients, using all 64 variables. The solid line represents the prediction based on the top 4 markers. Boxplots show the normalized Ct values of these top 4 markers.

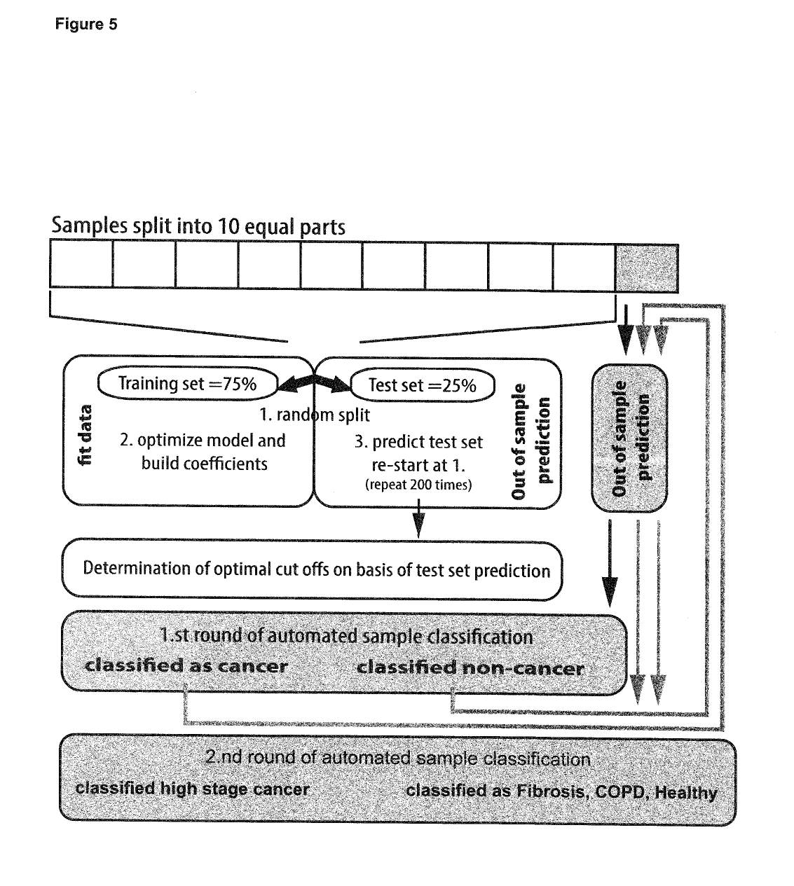

FIG. 5: Scheme to simulate prospective sample data.

The examples illustrate the invention.

### EXAMPLE 1

Materials and Methods

Patients

TABLE 5
Clinical Characteristics of patient serum/plasma samples.

| | |
|---|---|
| Original set | PoP set |
| Patients | Patients |

| Healthy | N = 61 | (29.9%) | N = 23 | (50%) |
|---|---|---|---|---|
| healthy | n = 27 | (13.2%) | n = 23 | (50%) |
| COPD at risk | n = 34 | (16.6%) | — | |
| Lung Cancer | N = 33 | (16.1%) | N = 23 | (50%) |
| low stage (I-II) | n = 9 | (4.4%) | n = 8 | (17.4%) |
| High stage (III-IV) | n = 15 | (7.3%) | n = 12 | (26%) |
| Lung Cancer | N = 33 | (16.1%) | N = 23 | (50%) |
| AdenoCa | n = 11 | (5.4%) | n = 9 | (19.5%) |
| SqCC | n = 8 | (3.9%) | n = 4 | (8.6%) |
| SCLC | n = 7 | (3.4%) | n = 6 | (13%) |
| LCLC | n = 7 | (3.4%) | n = 4 | (8.7%) |

| | | | |
|---|---|---|---|
| COPD | N = 42 | (20.5%) | — |
| GOLD 1-2 | n = 31 | (15.1%) | — |
| GOLD 3-4 | n = 11 | (5.4%) | — |
| Fibrosis | N = 68 | (33.3%) | — |
| UIP | n = 27 | (13.2%) | — |
| NSIP | n = 11 | (5.4%) | — |
| HP | n = 22 | (10.7%) | — |

In sum, 250 serum samples were analyzed. 204 within the original set and 46 within the PoP (proof of principle) set. Information on the characteristic histopathologic pattern of IPF was not available for 8 fibrosis patients and cancer staging information was not available for 7 sera of the original set and 3 sera of the PoP set. AdenoCa, lung adenocarcinoma; SqCC: squamous cell carcinoma, SCLC: small cell lung cancer, LCLC: large cell lung cancer.

Cf-DNA Isolation

Cell free DNA was isolated using Roche High pure template preparation kit (Roche) with a modified isolation protocol published previously (Wielscher, et al., BMC Clin Pathol 11, 11 (2011)). DNA was eluted in 55 μl. After DNA concentration measurement with Pico Green (Invitrogen) of 1 μl sample, the residual volume was reduced to approximately 10 μl using Microcon-10 Centrifugal Filters Ultracel YM-10 (Millipore) according to manufacturer's instruction.

MSRE Digestion (Methylation Sensitive Step

To be able to distinguish between methylated and unmethylated DNA the isolated cell free DNA was digested with four methylsensitive restriction enzymes namely HpaII (ThermoFisher), Hin6I (ThermoFisher), AciI (NewEnglandBiolabs) and HpyCHIV4 (NewEnglandBiolabs). The reaction was carried out in a total volume of 12 μl consisting of 8 μl sample material, 1.2 μl reaction buffer Tango (ThermoFisher), 0.4 μl of each restriction enzyme (HpaII, Hin6I, AciI, HpyCHIV4 each 10 U/μl) and 1.2 μl water. Reaction was incubated at 37° C. for 16 h and followed by heat inactivation.

Pre-Amplification (Multiplexed Amplification of 96 Targets Per Sample)

10 μl of MSRE digestion reaction was subjected to multiplexed pre-amplification step. Preamplification reaction was performed in a volume of 20 μl [..] 25 nM of each pair of the primer pairs were added per reaction. 19 cycles of preamplification were performed. Subsequent to amplification samples were diluted 1:5 with water to avoid primer contamination in the qPCR reaction.

qPCR Read Out (in nl Scale on Fluidigm Biomark)

96.96 Dynamic Array™ IFC chips for Gene Expression (Fluidigm) were primed at an ICF controller HX (Fluidigm). The sample mix was 3 μl 2×TaqMan GeneExpression MasterMix (ABI), 0.3 μl 20×DNA Binding Dye Sample Loading Reagent (Fluidigm), 0.3 μl 20× EvaGreen Binding Dye (Biotium), 0.9 μl of water and 1.5 μl of the preamplified sample. The assay mix was 3 μl 2× Assay Loading Reagent (Fluidigm), 0.3 μl water and 2.7 μl primermix (20 μM per primer pair). 6 μl sample mix and 6 μl assay mix were loaded to Fluidigm Priming station. The qPCR was performed by BioMark™ HD Reader (Fluidigm). The thermal mixing and UNG activation was 50° C. for 120 sec, at 70° C. for 1800 sec and 25° C. for 600 sec; followed by 120 sec at 50° C. for 120. The PCR was 40 cycles of 95° C. for 15 sec and 60 sec at 68° C. followed by a temperature gradient from 65° C. to 95° C. for melting curve analysis.

Data Analysis

Ct Value Preprocessing and Data Normalization

Prior to delta Ct normalization and supervised data analysis assays that produced less than 50 datapoints (out of 204 samples) were excluded. A total of 29 assays were excluded before subsequent analysis, which lead to 63 predictor variables for presented analysis. PCR fragments (i.e. Ct-values) exhibiting a Tm variance above +/−1.5 were excluded from further data analysis. On the sample side no sample had to be excluded as all 204 showed at least three positive internal controls.

Data were corrected for different cf-DNA concentrations by delta Ct normalization. For delta Ct normalization the geometric mean of the Ct values produced by assays for the SNRPN, H19, JUB and IRF4 locus were subtracted from the Ct values produced by the 63 predictor variables (Supplemental table 7). Missing values due to the lack of PCR signal of qPCR assay for several samples were replaced by 40 (i.e. number of PCR cycles) minus the geometric mean of internal controls of all analyzed samples (i.e. 24.233). After delta Ct normalization the log 2 transformed cell free DNA concentration values were added to the dataset.

Data Analysis Approach

To avoid over fitting of the data exclusively probabilities derived from an out of sample prediction are reported in the present study. This was achieved as follows. The 192 samples (outlying observation excluded from training set) were split randomly into 2 parts, a training set consisting of approximately 75% of all patients' samples and test set consisting of 25% of the 192 samples. The training set was used to build a fitted model of PCR data yielding coefficients for the 64 predictor variables (incl. log 2 transformed cf-DNA concentration). Subsequently the test set was predicted on basis of the 64 coefficients computed from the training set (FIG. 1A). This simply represents the scheme of an out of sample prediction, because none of the test set samples was used to fit the data. The procedure was repeated 200 times, to assure that every sample was is represented in the test set prediction. On average each sample was present in the test set 26 times. Thus gained approximately 26 probabilities of each sample for their diagnostic group membership were averaged before subsequent analysis. On basis of the test set prediction the correct classification rates and ROC-curve analysis was performed. On basis of training sets the variable importance was determined.

Lasso Penalized Logistic Regression

Binominal classification comparing each disease group to normal controls (FIG. 1) was performed on basis of linear models using lasso penalized logistic regression taken from the R package glmnet. The optimal lambda value was determined by a 5 fold cross validation within training set. This determination process was optimized towards the maximum AUC accessible for the separation of case and control. If the number of cases was not equal to the number of controls weights to compensate the imbalance were assigned.

$${weights}_{cases} = \frac{{N}_{all samples} \times \frac{L}{2}}{{N}_{cases}} \& {weights}_{contr .} = \frac{{N}_{all samples} \times \frac{L}{2}}{{N}_{contr .}}$$

Thus specified lambda values and resulting coefficients were used to perform the out of samples prediction.

Visualization of Patient Group Separation

The Fisher discriminant scores in FIG. 1C were produced as follows. To reduce noise for graphical display of data exclusively the top 30 variables distinguishing between diseased persons and controls were used for computation of projection directions. A fisher discriminant analysis was performed, where projection directions of the maximum separation between the sample groups based on the top 30 variables were identified and plotted. The plot shows the transformed data, which may be interpreted similar to a Principal Component analysis.

Generalized Boosted Regression Models

Differential multinomial classifications were performed using boosted trees method. Here the idea of gradient boosting, where a number of weak classifiers are combined to a powerful committee, which is achieved by specific weighting of the samples, is applied to classification trees (Friedmann, Annals of Statistics, 1189-1232 (2001)). In the present study the gbm function build trees with at least 6 observations per terminal node of each tee. The optimal iteration was determined by 5 fold cross validation, in sum 4000 trees were computed (i.e. iterations) with a learning rate of 0.001. Best iteration of the learning process was used to predict the test set samples.

Determination of Optimal Cutpoints

The optimal cut off values for diagnostic purposes were determined on basis of predicted averaged group membership probability derived from test set predictions. This was achieved using the R package OptimalCutpoints. As criteria to appoint the optimal cut off value Youden's Index was used for separation of cancer samples.

Software

All computations and statistical analyses were performed using R 3.0.2 (http://www.r-project.org) (Team, R foundation of Statistical Computing (2005)) and Bioconductor 2.13 (http://www.bioconductor.org) (Gentleman, Genome biology, 5, R80 (2004)).

### EXAMPLE 2

Results

All patients 204 patients from the original set and 46 patients from the PoP set are given in Table 1. Herein presented are the results derived from normal controls (i.e. healthy persons and COPD Gold stage 0) and cancer patients. Markers and prediction models specific for fibrosis or COPD are not discussed in the following section.

cfDNA Amounts in Patient Sera

No difference in total cfDNA concentrations between serum and plasma was measured. In healthy controls 12.5 ng/ml were isolated on average. The most pronounced cfDNA increase was observed in cancer patients (FIG. 1B). In early stage (i.e. stage I and stage II) cancer patients the cfDNA amount increased 2.8 fold to 35.6 ng/ml, in late stage cancer patients (stage III, stage IV) an average of 47.4 ng/ml was observed.

cfDNA Reveals Disease Specific Methylation Signature

To overcome the challenging task of cfDNA methylation analysis in patient serum, a MSRE based approach was chosen (FIG. 1A). To introduce a difference between methylated and unmethylated cfDNA, serum isolates were digested with a mix of four different methyl sensitive restriction enzymes, which fragmented the unmethylated DNA and leave the methylated DNA fraction intact. Digested DNA was then preamplified in a reaction with 96 primer pairs. Finally qPCR read out was performed in 6 nl single reactions (FIG. 1A). In total, 204 serum/plasma samples were randomized before DNA isolation step and processed in 3 independent experimental fractions. A mean Spearman's rank correlation coefficient of 0.69 (+/−0.158) was observed analyzing 16 replicate samples distributed to all three experimental fractions, indicating the high reproducibility of the applied method.

On the basis of differential methylation observed in patient serum, a penalized logistic regression was performed. Therefore, the qPCR Ct values of 63 candidate loci and the cfDNA amounts were used to create a model based on the training set (75% of samples) and prediction was performed on the remaining 25% of the samples, this process was repeated 200 times and the resulting probabilities of sample class membership were averaged (FIG. 1A). To estimate the diagnostic value of tested methylation marks, each disease group was compared to healthy (FIG. 1D). An area under curve of 0.91 for cancer patients versus healthy controls was measured. Quality of separation of diseased patients from normal controls are also illustrated as the top 2 projection directions derived from the 30 most frequent modeled variables that underwent a Fisher discriminant analysis (FIG. 1C).

Cancer Classification

As depicted in FIG. 3 regression tree based gradient boosting, embedded in the same resampling strategy as described in the previous section, outperformed comparable methods like L1 regression, support vector machines or random forests. This process yielded a stable model and an area under curve of 0.95, which was achieved when separating all non-cancer patients from cancer patients (Table 1).

To assess the quality and the stability of the methylation markers, an independent sample set was analyzed (PoP-set, table 1). The samples were analyzed based on the models given in FIG. 2 embedded into the herein presented automated prediction procedure, which results in a prospective analysis setting where the novel samples (PoP set) were used solely to get predicted and had no influence on the model building process. The ROC curve analysis (FIG. 4) shows that the smaller model consisting of the top 4 markers (FIG. 2B) outperforms the full model yielding an AUC of 0.85 with a sensitivity of 0.97 and specificity of 0.73 (FIG. 4). Although only cancer and normal sera were available for the PoP-set, the whole analysis pipeline was tested and a separation of the PoP samples into all possible groups was performed. Solely one cancer case was not classified as cancer, 13 healthy controls were correct classified, whereas two healthy cases were rated as COPD patients and 8 healthy controls as cancer.

1. A nucleic acid comprising
(a) the sequence
(1) from position 7 to position 101 of SEQ ID NO: 4, wherein the cytidine in at least one CpG dinucleotide comprised in said sequence is methylated;
(2) from position 11 to position 71 of SEQ ID NO: 2, wherein the cytidine in at least one CpG dinucleotide comprised in said sequence is methylated;
(3) from position 37 to position 81 of SEQ ID NO: 3, wherein the cytidine in at least one CpG dinucleotide comprised in said sequence is methylated;
(4) from position 4 to position 84 of SEQ ID NO: 1, wherein the cytidine in at least one CpG dinucleotide comprised in said sequence is methylated;
(5) from position 28 to position 69 of SEQ ID NO: 5, wherein the cytidine in at least one CpG dinucleotide comprised in said sequence is methylated;
(6) from position 39 to position 85 of SEQ ID NO: 6, wherein the cytidine in at least one CpG dinucleotide comprised in said sequence is methylated;
(7) from position 10 to position 53 of SEQ ID NO: 7, wherein the cytidine in at least one CpG dinucleotide comprised in said sequence is methylated;
(8) from position 13 to position 71 of SEQ ID NO: 8, wherein the cytidine in at least one CpG dinucleotide comprised in said sequence is methylated;
(9) from position 5 to position 71 of SEQ ID NO: 9, wherein the cytidine in at least one CpG dinucleotide comprised in said sequence is methylated;
(10) from position 2 to position 60 of SEQ ID NO: 10, wherein the cytidine in at least one CpG dinucleotide comprised in said sequence is methylated;
(11) from position 22 to position 63 of SEQ ID NO: 11, wherein the cytidine in at least one CpG dinucleotide comprised in said sequence is methylated;
(12) from position 45 to position 67 of SEQ ID NO: 12, wherein the cytidine in at least one CpG dinucleotide comprised in said sequence is methylated;
(13) from position 58 to position 78 of SEQ ID NO: 13, wherein the cytidine in at least one CpG dinucleotide comprised in said sequence is methylated;
(14) from position 31 to position 66 of SEQ ID NO: 14, wherein the cytidine in at least one CpG dinucleotide comprised in said sequence is methylated;
(15) from position 41 to position 72 of SEQ ID NO: 15, wherein the cytidine in at least one CpG dinucleotide comprised in said sequence is methylated;
(16) from position 53 to position 82 of SEQ ID NO: 16, wherein the cytidine in at least one CpG dinucleotide comprised in said sequence is methylated;
(17) from position 20 to position 74 of SEQ ID NO: 17, wherein the cytidine in at least one CpG dinucleotide comprised in said sequence is methylated;
(18) from position 9 to position 98 of SEQ ID NO: 18, wherein the cytidine in at least one CpG dinucleotide comprised in said sequence is methylated;
(19) from position 23 to position 85 of SEQ ID NO: 19, wherein the cytidine in at least one CpG dinucleotide comprised in said sequence is methylated;
(20) from position 20 to position 28 of SEQ ID NO: 20, wherein the cytidine in at least one CpG dinucleotide comprised in said sequence is methylated;
(21) from position 24 to position 61 of SEQ ID NO: 21, wherein the cytidine in at least one CpG dinucleotide comprised in said sequence is methylated;
(22) from position 11 to position 84 of SEQ ID NO: 22, wherein the cytidine in at least one CpG dinucleotide comprised in said sequence is methylated;
(23) from position 2 to position 86 of SEQ ID NO: 23, wherein the cytidine in at least one CpG dinucleotide comprised in said sequence is methylated;
(24) from position 43 to position 80 of SEQ ID NO: 24, wherein the cytidine in at least one CpG dinucleotide comprised in said sequence is methylated;
(25) from position 24 to position 70 of SEQ ID NO: 25, wherein the cytidine in at least one CpG dinucleotide comprised in said sequence is methylated;
(26) from position 44 to position 94 of SEQ ID NO: 26, wherein the cytidine in at least one CpG dinucleotide comprised in said sequence is methylated;
(27) from position 44 to position 67 of SEQ ID NO: 27, wherein the cytidine in at least one CpG dinucleotide comprised in said sequence is methylated;
(28) from position 20 to position 43 of SEQ ID NO: 28, wherein the cytidine in at least one CpG dinucleotide comprised in said sequence is methylated;
(29) from position 3 to position 71 of SEQ ID NO: 29, wherein the cytidine in at least one CpG dinucleotide comprised in said sequence is methylated;
(30) from position 11 to position 64 of SEQ ID NO: 30, wherein the cytidine in at least one CpG dinucleotide comprised in said sequence is methylated;
(31) from position 7 to position 118 of SEQ ID NO: 31, wherein the cytidine in at least one CpG dinucleotide comprised in said sequence is methylated;
(32) from position 25 to position 95 of SEQ ID NO: 32, wherein the cytidine in at least one CpG dinucleotide comprised in said sequence is methylated;
(33) from position 29 to position 46 of SEQ ID NO: 33, wherein the cytidine in at least one CpG dinucleotide comprised in said sequence is methylated;
(34) from position 8 to position 72 of SEQ ID NO: 34, wherein the cytidine in at least one CpG dinucleotide comprised in said sequence is methylated;
(35) from position 5 to position 57 of SEQ ID NO: 35, wherein the cytidine in at least one CpG dinucleotide comprised in said sequence is methylated;
(36) from position 43 to position 67 of SEQ ID NO: 36, wherein the cytidine in at least one CpG dinucleotide comprised in said sequence is methylated;
(37) from position 21 to position 63 of SEQ ID NO: 37, wherein the cytidine in at least one CpG dinucleotide comprised in said sequence is methylated;
(38) from position 27 to position 86 of SEQ ID NO: 38, wherein the cytidine in at least one CpG dinucleotide comprised in said sequence is methylated;
(39) from position 2 to position 110 of SEQ ID NO: 39, wherein the cytidine in at least one CpG dinucleotide comprised in said sequence is methylated;
(40) from position 13 to position 59 of SEQ ID NO: 40, wherein the cytidine in at least one CpG dinucleotide comprised in said sequence is methylated;
(41) from position 32 to position 107 of SEQ ID NO: 41, wherein the cytidine in at least one CpG dinucleotide comprised in said sequence is methylated;
(42) from position 3 to position 48 of SEQ ID NO: 42, wherein the cytidine in at least one CpG dinucleotide comprised in said sequence is methylated;
(43) from position 5 to position 57 of SEQ ID NO: 43, wherein the cytidine in at least one CpG dinucleotide comprised in said sequence is methylated;
(44) from position 40 to position 106 of SEQ ID NO: 44, wherein the cytidine in at least one CpG dinucleotide comprised in said sequence is methylated;
(45) from position 26 to position 90 of SEQ ID NO: 45, wherein the cytidine in at least one CpG dinucleotide comprised in said sequence is methylated; or
(46) from position 68 to position 74 of SEQ ID NO: 46, wherein the cytidine in at least one CpG dinucleotide comprised in said sequence is methylated;
or
(b) a sequence heaving at least 80% sequence identity to a sequence of (a).

2. The nucleic acid of claim 1, wherein at least one of the cytidines at positions
(1) 7, 32, 37, 39, 41, 43, 53, 71 and 100 in SEQ ID NO: 4 is/are methylated;
(2) 11, 23, 26, 33, 41, 54, 56, 66 and 70 in SEQ ID NO: 2 is/are methylated;
(3) 37, 41, 50 and 80 in SEQ ID NO: 3 is/are methylated;
(4) 4, 38, 40, 45 and 83 in SEQ ID NO: 1 is/are methylated;
(5) 28, 46, 52 and 68 in SEQ ID NO: 5 is/are methylated;
(6) 39, 46, 48, 50, 61, 82 and 84 in SEQ ID NO: 6 is/are methylated;
(7) 10 and 52 in SEQ ID NO: 7 is/are methylated;
(8) 13, 32, 50, 57 and 70 in SEQ ID NO: 8 is/are methylated;
(9) 5, 46, 48, 55 and 70 in SEQ ID NO: 9 is/are methylated;
(10) 2, 29, 31, 56 and 59 in SEQ ID NO: 10 is/are methylated;
(11) 22, 29, 49, 52, 54 and 62 in SEQ ID NO: 11 is/are methylated;
(12) 45, 59, 64 and 66 in SEQ ID NO: 12 is/are methylated;
(13) 58 and 77 in SEQ ID NO: 13 is/are methylated;
(14) 31, 48, 54 and 65 in SEQ ID NO: 14 is/are methylated;
(15) 41 and 71 in SEQ ID NO: 15 is/are methylated;
(16) 53, 63, 65 and 81 in SEQ ID NO: 16 is/are methylated;
(17) 20, 42, 58, 66 and 73 in SEQ ID NO: 17 is/are methylated;
(18) 9, 31, 61 and 97 in SEQ ID NO: 18 is/are methylated;
(19) 23, 34, 37, 56, 58, 67, 81 and 84 in SEQ ID NO: 19 is/are methylated;
(20) 20, 24 and 27 in SEQ ID NO: 20 is/are methylated;
(21) 24 and 60 in SEQ ID NO: 21 is/are methylated;
(22) 11, 49 and 83 in SEQ ID NO: 22 is/are methylated;
(23) 2, 18 and 85 in SEQ ID NO: 23 is/are methylated;
(24) 43, 52, 68 and 79 in SEQ ID NO: 24 is/are methylated;
(25) 24 and 69 in SEQ ID NO: 25 is/are methylated;
(26) 44, 50, 64 and 93 in SEQ ID NO: 26 is/are methylated;
(27) 44 and 66 in SEQ ID NO: 27 is/are methylated;
(28) 20, 35 and 42 in SEQ ID NO: 28 is/are methylated;
(29) 3, 68 and 70 in SEQ ID NO: 29 is/are methylated;
(30) 11, 33, 46 and 63 in SEQ ID NO: 30 is/are methylated;
(31) 7, 58, 95 and 117 in SEQ ID NO: 31 is/are methylated;
(32) 25, 27, 30, 39, 42, 59 and 94 in SEQ ID NO: 32 is/are methylated;
(33) 29, 32 and 45 in SEQ ID NO: 33 is/are methylated;
(34) 8, 37, 40, 56, 58, 60, 68 and 71 in SEQ ID NO: 34 is/are methylated;
(35) 5, 41, 50 and 56 in SEQ ID NO: 35 is/are methylated;
(36) 43, 60 and 66 in SEQ ID NO: 36 is/are methylated;
(37) 21, 56 and 62 in SEQ ID NO: 37 is/are methylated;
(38) 27, 67, 69 and 85 in SEQ ID NO: 38 is/are methylated;
(39) 2, 56, 62 and 109 in SEQ ID NO: 39 is/are methylated;
(40) 13, 47 and 58 in SEQ ID NO: 40 is/are methylated;
(41) 32, 34, 39 and 106 in SEQ ID NO: 41 is/are methylated;
(42) 3, 19, 44 and 47 in SEQ ID NO: 42 is/are methylated;
(43) 5, 41, 50 and 56 in SEQ ID NO: 43 is/are methylated;
(44) 40, 61, 67 and 105 in SEQ ID NO: 44 is/are methylated;
(45) 26, 28, 35, 42, 48 and 89 in SEQ ID NO: 45 is/are methylated; or
(46) 68 and 73 in SEQ ID NO: 46 is/are methylated.

3. A method of diagnosing lung cancer or a predisposition therefor, said method comprising determining the methylation status of cytidine in
(1) at least one CpG dinucleotide in a nucleic acid comprising or consisting of the sequence from position 7 to position 101 of SEQ ID NO: 4;
(2) at least one CpG dinucleotide in a nucleic acid comprising or consisting of the sequence from position 11 to position 71 of SEQ ID NO: 2;
(3) at least one CpG dinucleotide in a nucleic acid comprising or consisting of the sequence from position 37 to position 81 of SEQ ID NO: 3;
(4) at least one CpG dinucleotide in a nucleic acid comprising or consisting of the sequence from position 4 to position 84 of SEQ ID NO: 1;
(5) at least one CpG dinucleotide in a nucleic acid comprising or consisting of the sequence from position 28 to position 69 of SEQ ID NO: 5;
(6) at least one CpG dinucleotide in a nucleic acid comprising or consisting of the sequence from position 39 to position 85 of SEQ ID NO: 6;
(7) at least one CpG dinucleotide in a nucleic acid comprising or consisting of the sequence from position 10 to position 53 of SEQ ID NO: 7;
(8) at least one CpG dinucleotide in a nucleic acid comprising or consisting of the sequence from position 13 to position 71 of SEQ ID NO: 8;
(9) at least one CpG dinucleotide in a nucleic acid comprising or consisting of the sequence from position 5 to position 71 of SEQ ID NO: 9;
(10) at least one CpG dinucleotide in a nucleic acid comprising or consisting of the sequence from position 2 to position 60 of SEQ ID NO: 10;
(11) at least one CpG dinucleotide in a nucleic acid comprising or consisting of the sequence from position 22 to position 63 of SEQ ID NO: 11;
(12) at least one CpG dinucleotide in a nucleic acid comprising or consisting of the sequence from position 45 to position 67 of SEQ ID NO: 12;
(13) at least one CpG dinucleotide in a nucleic acid comprising or consisting of the sequence from position 58 to position 78 of SEQ ID NO: 13;
(14) at least one CpG dinucleotide in a nucleic acid comprising or consisting of the sequence from position 31 to position 66 of SEQ ID NO: 14;
(15) at least one CpG dinucleotide in a nucleic acid comprising or consisting of the sequence from position 41 to position 72 of SEQ ID NO: 15;
(16) at least one CpG dinucleotide in a nucleic acid comprising or consisting of the sequence from position 53 to position 82 of SEQ ID NO: 16;
(17) at least one CpG dinucleotide in a nucleic acid comprising or consisting of the sequence from position 20 to position 74 of SEQ ID NO: 17;
(18) at least one CpG dinucleotide in a nucleic acid comprising or consisting of the sequence from position 9 to position 98 of SEQ ID NO: 18;
(19) at least one CpG dinucleotide in a nucleic acid comprising or consisting of the sequence from position 23 to position 85 of SEQ ID NO: 19;
(20) at least one CpG dinucleotide in a nucleic acid comprising or consisting of the sequence from position 20 to position 28 of SEQ ID NO: 20;
(21) at least one CpG dinucleotide in a nucleic acid comprising or consisting of the sequence from position 24 to position 61 of SEQ ID NO: 21;
(22) at least one CpG dinucleotide in a nucleic acid comprising or consisting of the sequence from position 11 to position 84 of SEQ ID NO: 22;
(23) at least one CpG dinucleotide in a nucleic acid comprising or consisting of the sequence from position 2 to position 86 of SEQ ID NO: 23;
(24) at least one CpG dinucleotide in a nucleic acid comprising or consisting of the sequence from position 43 to position 80 of SEQ ID NO: 24;
(25) at least one CpG dinucleotide in a nucleic acid comprising or consisting of the sequence from position 24 to position 70 of SEQ ID NO: 25;
(26) at least one CpG dinucleotide in a nucleic acid comprising or consisting of the sequence from position 44 to position 94 of SEQ ID NO: 26;
(27) at least one CpG dinucleotide in a nucleic acid comprising or consisting of the sequence from position 44 to position 67 of SEQ ID NO: 27;
(28) at least one CpG dinucleotide in a nucleic acid comprising or consisting of the sequence from position 20 to position 43 of SEQ ID NO: 28;
(29) at least one CpG dinucleotide in a nucleic acid comprising or consisting of the sequence from position 3 to position 71 of SEQ ID NO: 29;
(30) at least one CpG dinucleotide in a nucleic acid comprising or consisting of the sequence from position 11 to position 64 of SEQ ID NO: 30;
(31) at least one CpG dinucleotide in a nucleic acid comprising or consisting of the sequence from position 7 to position 118 of SEQ ID NO: 31;
(32) at least one CpG dinucleotide in a nucleic acid comprising or consisting of the sequence from position 25 to position 95 of SEQ ID NO: 32;
(33) at least one CpG dinucleotide in a nucleic acid comprising or consisting of the sequence from position 29 to position 46 of SEQ ID NO: 33;
(34) at least one CpG dinucleotide in a nucleic acid comprising or consisting of the sequence from position 8 to position 72 of SEQ ID NO: 34;
(35) at least one CpG dinucleotide in a nucleic acid comprising or consisting of the sequence from position 5 to position 57 of SEQ ID NO: 35;
(36) at least one CpG dinucleotide in a nucleic acid comprising or consisting of the sequence from position 43 to position 67 of SEQ ID NO: 36;
(37) at least one CpG dinucleotide in a nucleic acid comprising or consisting of the sequence from position 21 to position 63 of SEQ ID NO: 37;
(38) at least one CpG dinucleotide in a nucleic acid comprising or consisting of the sequence from position 27 to position 86 of SEQ ID NO: 38;
(39) at least one CpG dinucleotide in a nucleic acid comprising or consisting of the sequence from position 2 to position 110 of SEQ ID NO: 39;
(40) at least one CpG dinucleotide in a nucleic acid comprising or consisting of the sequence from position 13 to position 59 of SEQ ID NO: 40;
(41) at least one CpG dinucleotide in a nucleic acid comprising or consisting of the sequence from position 32 to position 107 of SEQ ID NO: 41;
(42) at least one CpG dinucleotide in a nucleic acid comprising or consisting of the sequence from position 3 to position 48 of SEQ ID NO: 42;
(43) at least one CpG dinucleotide in a nucleic acid comprising or consisting of the sequence from position 5 to position 57 of SEQ ID NO: 43;
(44) at least one CpG dinucleotide in a nucleic acid comprising or consisting of the sequence from position 40 to position 106 of SEQ ID NO: 44;
(45) at least one CpG dinucleotide in a nucleic acid comprising or consisting of the sequence from position 26 to position 90 of SEQ ID NO: 45; and/or
(46) at least one CpG dinucleotide in a nucleic acid comprising or consisting of the sequence from position 68 to position 74 of SEQ ID NO: 46;
wherein methylation of the cytidine(s) or increased methylation of the cytidine(s) as compared to a non-cancer subject is indicative of lung cancer or a predisposition therefor, and wherein any diagnostic method practiced on the human or animal body is excluded.

4. A method of monitoring treatment of lung cancer or of a predisposition therefor, said method comprising determining the methylation status of cytidine as in claim 3, wherein no methylation of cytidine(s) or decreased methylation of the cytidine(s) as compared to a patient suffering from lung cancer or an individual having a predisposition therefor, respectively, is indicative of an amelioration of lung cancer or a decreased predisposition therefor, respectively, and wherein any diagnostic method practiced on the human or animal body is excluded.

5. The method of claim 3, wherein the cytidine(s) the methylation status of which is to be determined are wherein at least one, preferably all cytidines at positions
(1) 7, 32, 37, 39, 41, 43, 53, 71 and 100 in SEQ ID NO: 4 is/are methylated;
(2) 11, 23, 26, 33, 41, 54, 56, 66 and 70 in SEQ ID NO: 2 is/are methylated;
(3) 37, 41, 50 and 80 in SEQ ID NO: 3 is/are methylated;
(4) 4, 38, 40, 45 and 83 in SEQ ID NO: 1 is/are methylated;
(5) 28, 46, 52 and 68 in SEQ ID NO: 5 is/are methylated;
(6) 39, 46, 48, 50, 61, 82 and 84 in SEQ ID NO: 6 is/are methylated;
(7) 10 and 52 in SEQ ID NO: 7 is/are methylated;
(8) 13, 32, 50, 57 and 70 in SEQ ID NO: 8 is/are methylated;
(9) 5, 46, 48, 55 and 70 in SEQ ID NO: 9 is/are methylated;
(10) 2, 29, 31, 56 and 59 in SEQ ID NO: 10 is/are methylated;
(11) 22, 29, 49, 52, 54 and 62 in SEQ ID NO: 11 is/are methylated;
(12) 45, 59, 64 and 66 in SEQ ID NO: 12 is/are methylated;
(13) 58 and 77 in SEQ ID NO: 13 is/are methylated;
(14) 31, 48, 54 and 65 in SEQ ID NO: 14 is/are methylated;
(15) 41 and 71 in SEQ ID NO: 15 is/are methylated;
(16) 53, 63, 65 and 81 in SEQ ID NO: 16 is/are methylated;
(17) 20, 42, 58, 66 and 73 in SEQ ID NO: 17 is/are methylated;
(18) 9, 31, 61 and 97 in SEQ ID NO: 18 is/are methylated;
(19) 23, 34, 37, 56, 58, 67, 81 and 84 in SEQ ID NO: 19 is/are methylated;
(20) 20, 24 and 27 in SEQ ID NO: 20 is/are methylated;
(21) 24 and 60 in SEQ ID NO: 21 is/are methylated;
(22) 11, 49 and 83 in SEQ ID NO: 22 is/are methylated;
(23) 2, 18 and 85 in SEQ ID NO: 23 is/are methylated;
(24) 43, 52, 68 and 79 in SEQ ID NO: 24 is/are methylated;
(25) 24 and 69 in SEQ ID NO: 25 is/are methylated;
(26) 44, 50, 64 and 93 in SEQ ID NO: 26 is/are methylated;
(27) 44 and 66 in SEQ ID NO: 27 is/are methylated;
(28) 20, 35 and 42 in SEQ ID NO: 28 is/are methylated;
(29) 3, 68 and 70 in SEQ ID NO: 29 is/are methylated;
(30) 11, 33, 46 and 63 in SEQ ID NO: 30 is/are methylated;
(31) 7, 58, 95 and 117 in SEQ ID NO: 31 is/are methylated;
(32) 25, 27, 30, 39, 42, 59 and 94 in SEQ ID NO: 32 is/are methylated;
(33) 29, 32 and 45 in SEQ ID NO: 33 is/are methylated;
(34) 8, 37, 40, 56, 58, 60, 68 and 71 in SEQ ID NO: 34 is/are methylated;
(35) 5, 41, 50 and 56 in SEQ ID NO: 35 is/are methylated;
(36) 43, 60 and 66 in SEQ ID NO: 36 is/are methylated;
(37) 21, 56 and 62 in SEQ ID NO: 37 is/are methylated;
(38) 27, 67, 69 and 85 in SEQ ID NO: 38 is/are methylated;
(39) 2, 56, 62 and 109 in SEQ ID NO: 39 is/are methylated;
(40) 13, 47 and 58 in SEQ ID NO: 40 is/are methylated;
(41) 32, 34, 39 and 106 in SEQ ID NO: 41 is/are methylated;
(42) 3, 19, 44 and 47 in SEQ ID NO: 42 is/are methylated;
(43) 5, 41, 50 and 56 in SEQ ID NO: 43 is/are methylated;
(44) 40, 61, 67 and 105 in SEQ ID NO: 44 is/are methylated;
(45) 26, 28, 35, 42, 48 and 89 in SEQ ID NO: 45 is/are methylated; or
(46) 68 and 73 in SEQ ID NO: 46 is/are methylated.

6. The nucleic acid of claim 1, wherein said nucleic acid comprises any one of SEQ ID NOs: 1 to 136.

7. A nucleic acid comprising or consisting of
(a) the sequence of any one of SEQ ID NOs: 137 to 228; or
(b) a sequence heaving at least 80% sequence identity to a sequence of (a).

8. The method of claim 3, wherein the methylation status of cytidines according to
(47) (4) and (9);
(48) (4) and (2);
(49) (4) and (6);
(50) (2) and (9);
(51) (1) and (4);
(52) (2) and (5);
(53) (4) and (13);
(54) (2) and (10);
(55) (2) and (6);
(56) (10) and (11);
(57) (2) and (1);
(58) (9) and (11);
(59) (9) and (10);
(60) (4) and (5);
(61) (5) and (6);
(62) (10) and (14);
(63) (1) and (11);
(64) (9) and (13);
(65) (7) and (11);
(66) (2) and (9);
(67) (4), (2) and (6);
(68) (4), (9) and (10);
(69) (9), (10) and (11);
(70) (4), (9) and (11);
(71) (2), (6) and (10);
(72) (4), (5) and (9);
(73) (2), (9) and (10);
(74) (4), (2) and (10);
(75) (1), (4) and (9);
(76) (4), (2) and (12);
(77) (1), (2) and (4);
(78) (2), (10) and (14);
(79) (2), (10) and (11);
(80) (2), (5) and (10);
(81) (4), (2) and (5);
(82) (2), (5) and (9);
(83) (2), (5) and (6);
(84) (2), (6) and (11);
(85) (4), (9) and (12); or
(86) (4), (2) and (7)
is determined.

9. The method of claim 3, wherein
(a) the methylation status of no further nucleotides is to be determined; or
(b) said method furthermore comprises determining the methylation status of cytidine in at least on CpG dinucleotide in a nucleic acid comprising or consisting of the sequence of SEQ ID NO: 321, wherein methylation of cytidines or increased methylation of cytidines as compared to a non-cancer subject is indicative of lung cancer, cytidines being those defined in claim 3 and cytidine(s) in the sequence of SEQ ID NO: 321.

10. The method of claim 3, wherein said determining is effected in a sample taken from a subject, said sample preferably being a bodily fluid, more preferably plasma, serum or sputum; or a biopsy taken from said cancer; a biopsy taken from a metastasis; or a circulating tumor cell.

11. The method of claim 10, wherein prior to said determining,
(a) cell-free DNA comprised in said sample is enriched or isolated;
(b) cell-free DNA is subjected to digestion with one or more methylation-sensitive restriction enzymes; and/or
(c) cell-free DNA is pre-amplified.

12. The method of claim 3, wherein said determining is performed by one or more of the following procedures:
(a) a support-based methylation assay;
(b) amplification, preferably PCR, more preferably conventional or real-time PCR, of DNA digested in accordance with claim 8(b) and optionally pre-amplified in accordance with claim 8(c);
(c) a methylated CpG island recovery assay;
(d) a bisulfite assay or bisulfite sequencing;
(e) a proximity hybridization assay; and
(f) nanopore sequencing.

13. The method of claim 3, wherein
(a) cell-free DNA comprised in said sample is isolated;
(b) the isolated DNA obtained in (a) is subjected to digestion with one or more methylation-sensitive restriction enzymes;
(c) the digested DNA obtained in (b) is pre-amplified;
(d) the pre-amplified DNA obtained in (c) is amplified.

14. A kit comprising one or more of the following sets of primers:
(1) nucleic acids comprising or consisting of the sequences of SEQ ID NO: 229 and 230 or sequences having at least 80% sequence identity thereto;
(2) nucleic acids comprising or consisting of the sequences of SEQ ID NO: 231 and 232 or sequences having at least 80% sequence identity thereto;
(3) nucleic acids comprising or consisting of the sequences of SEQ ID NO: 233 and 234 or sequences having at least 80% sequence identity thereto;
(4) nucleic acids comprising or consisting of the sequences of SEQ ID NO: 235 and 236 or sequences having at least 80% sequence identity thereto;
(5) nucleic acids comprising or consisting of the sequences of SEQ ID NO: 237 and 238 or sequences having at least 80% sequence identity thereto;
(6) nucleic acids comprising or consisting of the sequences of SEQ ID NO: 239 and 240 or sequences having at least 80% sequence identity thereto;
(7) nucleic acids comprising or consisting of the sequences of SEQ ID NO: 241 and 242 or sequences having at least 80% sequence identity thereto;
(8) nucleic acids comprising or consisting of the sequences of SEQ ID NO: 243 and 244 or sequences having at least 80% sequence identity thereto;
(9) nucleic acids comprising or consisting of the sequences of SEQ ID NO: 245 and 246 or sequences having at least 80% sequence identity thereto;
(10) nucleic acids comprising or consisting of the sequences of SEQ ID NO: 247 and 248 or sequences having at least 80% sequence identity thereto;
(11) nucleic acids comprising or consisting of the sequences of SEQ ID NO: 249 and 250 or sequences having at least 80% sequence identity thereto;
(12) nucleic acids comprising or consisting of the sequences of SEQ ID NO: 251 and 252 or sequences having at least 80% sequence identity thereto;
(13) nucleic acids comprising or consisting of the sequences of SEQ ID NO: 253 and 254 or sequences having at least 80% sequence identity thereto;
(14) nucleic acids comprising or consisting of the sequences of SEQ ID NO: 255 and 256 or sequences having at least 80% sequence identity thereto;
(15) nucleic acids comprising or consisting of the sequences of SEQ ID NO: 257 and 258 or sequences having at least 80% sequence identity thereto;
(16) nucleic acids comprising or consisting of the sequences of SEQ ID NO: 259 and 260 or sequences having at least 80% sequence identity thereto;
(17) nucleic acids comprising or consisting of the sequences of SEQ ID NO: 261 and 262 or sequences having at least 80% sequence identity thereto;
(18) nucleic acids comprising or consisting of the sequences of SEQ ID NO: 263 and 264 or sequences having at least 80% sequence identity thereto;
(19) nucleic acids comprising or consisting of the sequences of SEQ ID NO: 265 and 266 or sequences having at least 80% sequence identity thereto;
(20) nucleic acids comprising or consisting of the sequences of SEQ ID NO: 267 and 268 or sequences having at least 80% sequence identity thereto;
(21) nucleic acids comprising or consisting of the sequences of SEQ ID NO: 269 and 270 or sequences having at least 80% sequence identity thereto;
(22) nucleic acids comprising or consisting of the sequences of SEQ ID NO: 271 and 272 or sequences having at least 80% sequence identity thereto;
(23) nucleic acids comprising or consisting of the sequences of SEQ ID NO: 273 and 274 or sequences having at least 80% sequence identity thereto;
(24) nucleic acids comprising or consisting of the sequences of SEQ ID NO: 275 and 276 or sequences having at least 80% sequence identity thereto;
(25) nucleic acids comprising or consisting of the sequences of SEQ ID NO: 277 and 278 or sequences having at least 80% sequence identity thereto;
(26) nucleic acids comprising or consisting of the sequences of SEQ ID NO: 279 and 280 or sequences having at least 80% sequence identity thereto;
(27) nucleic acids comprising or consisting of the sequences of SEQ ID NO: 281 and 282 or sequences having at least 80% sequence identity thereto;
(28) nucleic acids comprising or consisting of the sequences of SEQ ID NO: 283 and 284 or sequences having at least 80% sequence identity thereto;
(29) nucleic acids comprising or consisting of the sequences of SEQ ID NO: 285 and 286 or sequences having at least 80% sequence identity thereto;
(30) nucleic acids comprising or consisting of the sequences of SEQ ID NO: 287 and 288 or sequences having at least 80% sequence identity thereto;
(31) nucleic acids comprising or consisting of the sequences of SEQ ID NO: 289 and 290 or sequences having at least 80% sequence identity thereto;
(32) nucleic acids comprising or consisting of the sequences of SEQ ID NO: 291 and 292 or sequences having at least 80% sequence identity thereto;
(33) nucleic acids comprising or consisting of the sequences of SEQ ID NO: 293 and 294 or sequences having at least 80% sequence identity thereto;
(34) nucleic acids comprising or consisting of the sequences of SEQ ID NO: 295 and 296 or sequences having at least 80% sequence identity thereto;
(35) nucleic acids comprising or consisting of the sequences of SEQ ID NO: 297 and 298 or sequences having at least 80% sequence identity thereto;
(36) nucleic acids comprising or consisting of the sequences of SEQ ID NO: 299 and 300 or sequences having at least 80% sequence identity thereto;
(37) nucleic acids comprising or consisting of the sequences of SEQ ID NO: 301 and 302 or sequences having at least 80% sequence identity thereto;
(38) nucleic acids comprising or consisting of the sequences of SEQ ID NO: 303 and 304 or sequences having at least 80% sequence identity thereto;
(39) nucleic acids comprising or consisting of the sequences of SEQ ID NO: 305 and 306 or sequences having at least 80% sequence identity thereto;
(40) nucleic acids comprising or consisting of the sequences of SEQ ID NO: 307 and 308 or sequences having at least 80% sequence identity thereto;
(41) nucleic acids comprising or consisting of the sequences of SEQ ID NO: 309 and 310 or sequences having at least 80% sequence identity thereto;
(42) nucleic acids comprising or consisting of the sequences of SEQ ID NO: 311 and 312 or sequences having at least 80% sequence identity thereto;
(43) nucleic acids comprising or consisting of the sequences of SEQ ID NO: 313 and 314 or sequences having at least 80% sequence identity thereto;
(44) nucleic acids comprising or consisting of the sequences of SEQ ID NO: 315 and 316 or sequences having at least 80% sequence identity thereto;
(45) nucleic acids comprising or consisting of the sequences of SEQ ID NO: 317 and 318 or sequences having at least 80% sequence identity thereto;
(46) nucleic acids comprising or consisting of the sequences of SEQ ID NO: 319 and 320 or sequences having at least 80% sequence identity thereto.

15. The kit of claim 14, furthermore comprising one or more of the following:
(a) a manual with instructions for performing the method of claim 3;
(b) one or more buffered solutions or reagents for the preparation thereof;
(c) one or more reaction vessels.
